(19) United States
(12) Patent Application Publication
(10) Pub. No.: US 2024/0201203 A1
(43) Pub. Date: Jun. 20, 2024

(54) COMPOSITIONS AND METHODS FOR TREATING INDIVIDUALS WHO HAVE ONCOGENE-NEGATIVE CANCER

(71) Applicant: The Board of Trustees of the Leland Stanford Junior University, Stanford, CA (US)

(72) Inventors: Dmitri A. Petrov, San Francisco, CA (US); Monte M. Winslow, Stanford, CA (US); Gabor Boross, Redwood City, CA (US); Maryam Yousefi, Redwood City, CA (US)

(21) Appl. No.: 18/288,372

(22) PCT Filed: Apr. 29, 2022
(86) PCT No.: PCT/US2022/027087

§ 371 Date: Oct. 25, 2023

Related U.S. Application Data
(60) Provisional application No. 63/182,542, filed on Apr. 30, 2021.
(60) Provisional application No. 63/218,805, filed on Jul. 6, 2021.

(51) Int. Cl. G01N 33/68; A01K 67/0275; A61K 31/501; A61K 31/506; A61K 31/519; A61P 35/00; C12Q 1/6886; G01N 33/50

(52) U.S. Cl. CPC G01N 33/6893; A01K 67/0275; A61K 31/501; A61K 31/506; A61K 31/519; A61P 35/00; C12Q 1/6886; G01N 33/5011; G01N 33/5044; A01K 2227/105; A01K 2267/0331; G01N 2333/912; G01N 2800/52; G01N 2800/7057

(57) ABSTRACT

Methods and compositions are provided for treating individuals who have an oncogene-negative cancer. In some cases, such individuals have an oncogene-negative tumor(s). In some cases, they have an oncogene-negative lung cancer. In some cases, they have an oncogene-negative lung adenocarcinoma. In some cases, treatment includes administering an inhibitor of the Ras/MAPK pathway. In some cases, treatment includes administering an inhibitor of the Ras/MAPK pathway and an inhibitor of the PI3K-AKT pathway. Methods and compositions are also provided for testing candidate cancer therapeutics. In some cases, such methods include contacting an oncogene-negative cell, in culture or in vivo, with a candidate agent, where the cell has increased Ras/MAPK pathway activity and/or increased PI3K-AKT pathway activity.

### CROSS-REFERENCE

This application claims the benefit of U.S. Provisional Patent Application Nos. 63/182,542 filed Apr. 30, 2021, and 63/218,805 filed Jul. 6, 2021, each of which application is incorporated herein by reference in its entirety.

### STATEMENT REGARDING FEDERALLY SPONSORED RESEARCH

This invention was made with Government support under contracts CA230919, CA231253, and CA234349 awarded by the National Institutes of Health. The Government has certain rights in the invention.

### INTRODUCTION

Lung cancer is the leading cause of cancer death worldwide, with lung adenocarcinoma being the most common subtype. Genome sequencing has uncovered alterations in oncogenes and tumor suppressor genes in this cancer type. The discovery of oncogene alterations has led to the development of targeted therapies and better clinical outcomes. However, a significant fraction of lung adenocarcinomas lacks mutations in known oncogenes, and the genesis and treatment of these oncogene-negative tumors remain enigmatic.

### SUMMARY

Using a quantitative autochthonous mouse model system and performing iterative in vivo functional screens, the inventors uncovered genetic and biochemical changes that facilitate efficient tumor initiation (e.g., lung tumors) in the absence of oncogene alterations. Through the generation of hundreds of diverse combinatorial tumor suppressor alterations, the inventors demonstrated that activation of the RAS/MAPK and PI3K pathways (e.g., via inactivation of pathway suppressors) in combination allows for stepwise and efficient acquisition of growth advantage that can drive the development of cancer (e.g., a lung cancer such as lung adenocarcinoma) in the absence of an oncogenic mutation (i.e., oncogene-negative). By pathway-level human genomic data analysis and histology, the inventors identified Ras/MAPK and PI3K pathway activation as a common event in oncogene-negative cancer (e.g., human lung adenocarcinoma). Furthermore, the inventors demonstrate that oncogene-negative tumors are vulnerable to pharmacological inhibition of these signaling axes.

For example, in FIG. 1 of the working examples, it is shown that tumor suppressor inactivation can facilitate cancer development (e.g. lung tumor development) in the absence of oncogenes in vivo (i.e., in the absence of an oncogenic mutation in a proto-oncogene). In FIG. 2 of the working examples, it is shown that members of the Ras/MAPK pathway (such as Nf1 and Rasa1) and of the PI3K-AKT pathway (such as Pten) are potent and key drivers of oncogene-negative cancer (e.g., lung cancer such as lung adenocarcinoma). In FIG. 3 of the working examples, it is shown that activation of the Ras/MAPK pathway (e.g., via inactivation of Nf1 and/or Rasa1) and the PI3K-AKT pathway (e.g., via inactivation of Pten) allows a stepwise acquisition of growth advantage to facilitate cancer development (e.g., lung adenocarcinoma development). In FIG. 4 of the working examples, it is shown that oncogene-negative cancers (e.g., human lung adenocarcinomas) exhibit frequent activation of the Ras/MAPK and PI3K-AKT pathways. In FIG. 5 of the working examples, it is shown that inhibitors of Ras/MAPK pathway activity (e.g., an inhibitor of SHP2 such as RMC-4550) can reduce the growth of oncogene-negative cancer (e.g., lunge adenocarcinoma). Moreover, it is further shown that inhibitors of Ras/MAPK pathway activity (e.g., an inhibitor of SHP2 such as RMC-4550) can synergize with inhibitors of PI3K-AKT pathway activity (e.g., an inhibitor of AKT such as capivasertib) to reduce the growth of oncogene-negative cancer (e.g., lunge adenocarcinoma).

Provided are methods and compositions (e.g., kits) for treating individuals who have an oncogene-negative cancer. In some cases such individuals have an oncogene-negative tumor(s). In some cases, they have an oncogene-negative lung cancer. In some cases, they have an oncogene-negative lung adenocarcinoma.

In some cases, a subject composition includes an inhibitor of the Ras/MAPK pathway. In some cases a subject composition includes an inhibitor of the Ras/MAPK pathway and an inhibitor of the PI3K-AKT pathway, and is for use in the treatment of an oncogene-negative cancer (e.g., in the treatment of an individual who has an oncogene-negative lung adenocarcinoma). In some cases a subject composition includes an inhibitor of the Ras/MAPK pathway such as an inhibitor of SHP2, and an inhibitor of the PI3K-AKT pathway such as an inhibitor of AKT1/2, and is for use in the treatment of an oncogene-negative cancer (e.g., in the treatment of an individual who has an oncogene-negative lung adenocarcinoma). In some cases the inhibitor of AKT1/2 is capivasertib. In some cases the inhibitor of SHP2 is RMC-4550 or RMC-4630. For example, in some cases a subject composition includes an inhibitor of SHP2 such as RMC-4550 or RMC-4630 and is for use in the treatment of an oncogene-negative cancer (e.g., in the treatment of an individual who has an oncogene-negative lung adenocarcinoma). In some cases, a subject composition includes an inhibitor of SHP2 such as RMC-4550 or RMC-4630 and an inhibitor of AKT1/2 such as capivasertib, and is for use in the treatment of an oncogene-negative cancer (e.g., in the treatment of an individual who has an oncogene-negative lung adenocarcinoma).

In some cases, a subject method is a method of treatment of an individual who has previously been determined to have an oncogene-negative cancer (e.g., lung adenocarcinoma). In some such cases, the method includes administration to the individual of an inhibitor of the Ras/MAPK pathway (e.g., an inhibitor of SHP2 such as RMC-4550 or RMC-4630). In other such cases, the method includes administration to the individual of an inhibitor of the Ras/MAPK pathway (e.g., an inhibitor of SHP2 such as RMC-4550 or RMC-4630) and an inhibitor of the PI3K-AKT pathway (e.g., an inhibitor of AKT1/2 such as capivasertib). In some cases a subject method includes as step, prior to the administration, of determining that the individual has an oncogene-negative cancer (e.g., a lung cancer such as lung adenocarcinoma).

Also provided are methods (screening methods) and compositions (e.g., cells and/or non-human genetically modified mammal) for testing candidate therapies. For example, provided are methods in which a candidate agent is administered (e.g., systemically or locally, e.g., directly to a tumor) to a non-human genetically modified mammal that has an oncogene-negative genomic profile and comprises lung cells with one or more genomic alterations causing increased Ras/MAPK pathway activity and/or increased PI3K-AKT pathway activity. After the agent is administered, such methods can include a step of determining whether the candidate agent prevented or reduced lung cancer in the individual, e.g., relative to a control (such as a predetermined value; a different individual that is untreated or is treated with a control agent; or a control tumor of the same animal where the control tumor is one that is untreated or is treated with a control agent). The non-human genetically modified mammal in some cases is a rodent (e.g., mouse or rat) and in some cases is a non-human primate.

As another example, provided are methods (screening methods) in which a population of cells is contacted with a candidate agent, where the cells are mammalian cells that have an oncogene-negative genomic profile and comprise one or more genomic alterations causing increased Ras/MAPK pathway activity and/or increased PI3K-AKT pathway activity. After the cells are contacted, such methods can included a step of determining whether the candidate agent prevented or reduced proliferation of the cells relative to a control (e.g., such as a predetermined value; or a control population of cells that are untreated or treated with a control agent). In some cases the cells are lung cells. Examples of such cells include rodent cells (e.g., mouse or rat), non-human primate cells, and human cells.

For any of the above screening methods, in some cases the genomic alterations cause increased Ras/MAPK pathway activity and increased PI3K-AKT pathway activity. In some cases, increased Ras/MAPK pathway activity is caused by reduced expression of wild type Nf1 and/or wild type Rasa1. In some cases, increased PI3K-AKT pathway activity results from reduced expression of wild type Pten and/or a pathway-activating alteration of AKT (e.g., myristoylated AKT1).

Also provided are oncogene-negative non-human genetically modified mammals (e.g., mice, rats, non-human primates) as well as cells (e.g., lung cells, stem cells, ips cells, germ cells) isolated from such organisms. In some embodiments, the oncogene-negative non-human genetically modified mammals have an oncogene-negative genomic profile and genomic alterations causing increased Ras/MAPK pathway activity and increased PI3K-AKT pathway activity. In some cases, the increased Ras/MAPK pathway activity is caused by reduced expression of wild type Nf1 and/or wild type Rasa1. In some cases, the increased PI3K-AKT pathway activity is caused by reduced expression wild type Pten and/or by a pathway-activating alteration of AKT (e.g., myristoylated AKT1). In some cases Ras/MAPK pathway activity is increased due to genomic alterations that cause reduced expression of both wild type Nf1 and Rasa1, and increased PI3K-AKT pathway activity is increased due to a genomic alteration that causes reduced expression of Pten.

Reagents, compositions, and kits/systems that find use in practicing the subject methods are provided.

### BRIEF DESCRIPTION OF THE DRAWINGS

FIG. 1 (Panels a-f) Tumor suppressor inactivation enables lung tumor development in the absence of engineered oncogenes in vivo. a. Frequency of human lung adenocarcinomas with likely oncogenic alterations in proto-oncogenes (oncogene-positive), with alterations in known proto-oncogenes with unknown effects (oncogene-indeterminate), and without any alterations in proto-oncogenes (oncogene-negative). Data from TCGA. b. Schematic of combined Cre/lox and CRISPR/Cas9-mediated tumor suppressor gene inactivation to generate lung epithelial cells with diverse genotypes. R26LSL-Tom(T) mice lack Cas9 and control for normal epithelial expansion in the absence of engineered alterations. The number of mice in each group is indicated. c. Representative light and fluorescence (Tomato) images of lung lobes from the indicated genotypes of mice one year after transduction with the Lenti-sgTS102/Cre pool. Lung lobes are outlined with white dotted lines. Scale bar=4 mm d. The number of surface tumors (defined as Tomato-positive expansions greater than 0.5 mm in diameter) quantified by direct counting. Each dot represents a mouse, and the bar is the mean. e. Representative Hematoxylin and Eosin (H&E), TTF1, and TP63 images of lung tumors in the indicated genotypes of mice. Scale bar=100 um f. Heatmap showing two measures of tumor suppressor strengths in each genotype detected using Tuba-seq analysis on bulk lung lobes and dissected tumors from mice of the indicated genotype (see FIG. 9-11): (1) increase in sizes of clonal expansions in lung epithelial cells in the presence of indicated tumor suppressor alterations in rows labeled as “Lung”, and (2) occurrence of different tumor suppressor gene targeting vectors in tumors in rows labeled as “Tumors”. For lungs: all sgRNAs are significant (p<0.05) (red), 1 sgRNA is significant (pink), for tumors: p<0.001 (red), p<0.1 (pink). Gray boxes indicate redundant targeting of genes that are inactivated with floxed alleles in those models.

FIG. 2 (panels a-h) Nf1, Rasa1, and Pten emerge as potent key drivers of oncogene-negative lung adenocarcinoma. a. Schematic of tumor initiation with a pool of 14 barcoded Lenti-sgRNA/Cre vectors (Lenti-sgTS14/Cre) selected from the initial screens using Lenti-sgTS102/Cre. Each gene is targeted with a single sgRNA. Mouse genotype, mouse number, and titer of virus delivered to each mouse are indicated. Tuba-seq was performed on each tumor-bearing lung 4 months after tumor initiation. b. Representative light and fluorescence images of lung lobes from the indicated genotypes of mice. Lung lobes are outlined with white dotted lines. Scale bar=4 mm c. The number of tumors (defined as Tomato-positive cell masses larger than 0.5 mm in diameter) was quantified by direct counting under a fluorescent microscope. Each dot represents a mouse, and the bar is the mean. d,e. The number of tumors with a minimum size of 1000 neoplastic cells relative to the inert sgRNA is shown as a blue bar. 90th percentile of tumor sizes relative to the inert sgRNAs is shown as a red bar. sgRNAs resulting in significantly different tumor numbers or sizes than the inert sgRNAs (p<0.05) are shown in darker colors. Whiskers show 95% confidence intervals. Mouse genotypes are indicated on each plot. f,g. Barcodes with the highest cell counts in each mouse were investigated for coinfection with multiple Lenti-sgTS/Cre (i.e., tumors initiated from cells transduced with multiple viruses, which result in complex tumor suppressor mutant genotypes, see Methods). The top 15 pairs of tumor suppressors found co-mutated frequently in the largest tumors are shown. Combinations of sgRNAs that lead to the generation of Nf1, Rasa1, and Pten mutant cancer cells in a statistically significant manner are shown in a bold font. *p<0.05, **p<0.01, ***p<0.001 based on a permutation test. h. Total number of neoplastic cells in clonal cell expansions larger than 200 cells in Nf1f/f; TC, Ptenf/f; TC, Trp53f/f; TC, and TC mice 4 and 4.5 months after receiving Lenti-sgTS14/Cre and Lenti-sgTS11/Cre. The magnitude of neoplastic cell number reduction in each group in the absence of lentiviral vectors containing sgNf1, sgRasa1, and sgPten in Lenti-sgTS11/Cre pool is indicated on the graph.

FIG. 3 (panels a-l) Inactivation of Nf1, Rasa1, and Pten allows a stepwise acquisition of growth advantage to enable lung adenocarcinoma development. a. Schematic of 8 barcoded triple sgRNA vectors for CRISPR/Cas9-mediated inactivation of all combinations of Nf1, Rasa1, and Pten in TC and Trp53flox/flox; TC mice to assess genetic interactions between these tumor suppressors. sgNeo1 and sgNeo2 are active cutting, but inert sgRNAs that target NeoR in the R26LSL-tdTomato allele. sgNT is a non-targeting inert sgRNA. This vector design allows simultaneous inactivation of multiple tumor suppressors and quantification of the number of neoplastic cells by high-throughput sgID-BC sequencing. Mouse genotype, mouse number, and titer of virus delivered to each mouse are indicated. Tuba-seq was performed on tumor-bearing lungs 3 months after tumor initiation, followed by analysis to quantify the effect of tumor suppressor mutations and their interactions. ifu, infection unit b. Bright-field and fluorescence images of lungs from the indicated mouse genotypes. Lung lobes are outlined with a dashed white line. Scale bar=4 mm c. The number of surface tumors (defined as Tomato-positive expansions larger than 0.5 mm in diameter) quantified by direct counting. Each dot represents a mouse, and the bar is the mean. d. Numbers of tumors (with >1000 neoplastic cells) are shown relative to the Inert sgRNA. sgRNAs resulting in a significantly higher number of tumors than the inert vector (p<0.05) are shown in a darker color. Mean+/−95% confidence interval is shown. e. Adaptive landscape of Nf1, Rasa1, and Pten inactivation in TC mice is shown. Nodes represent genotypes shown as a string of +(wild-type) and −(inactivated) symbols representing Nf1, Rasa1, and Pten. Numbers in the nodes indicate fitness increase compared to wild-type. The relative probability of each beneficial mutation is shown as arrow widths (see Methods) f. Quantification of the ability of combined Nf1/Rasa1/Pten inactivation in TC mice and oncogenic KrasG12D in KT mice to initiate tumors. The number of tumors (with >1000 neoplastic cells) per infectious unit (ifu) is shown. The bar is the median, the box represents the interquartile range and the whiskers show minimum and maximum values. ns: non-significant g. Representative H&E and Tomato stained sections from TC and Tp53flox/flox; TC mice 3 months after transduction with Triple-Lenti-sgNf1-sgRasa1-sgPten/Cre. Scale bar=500 um h,i. RAS and PI3K-AKT pathway gene-set profiles estimated by single-sample Gene Set Enrichment Analysis (ssGSVA). Tumors from KrasG12D; TC (KTC+sgInert and KTC+sgPten: Kras and Kras/Pten) mice are compared with Nf1, Rasa1, and Pten mutant tumors(Nf1/Rasa1/Pten). The bar is the mean. ns: non-significant, *p<0.05 using Mann-Whitney U test. j-l. Representative immunohistochemistry for pERK and pAKT to determine activation of RAS and PI3K pathway in tumors with the indicated genotypes and quantification of these stainings. The bar is the mean. n.s: non-significant, ****<p<0.0001 using Mann-Whitney U test. Scale bar=40 um

FIG. 4 (panels a-e) Oncogene-negative human lung adenocarcinomas have frequent activation of RAS and PI3K pathways. a. Representative p-AKT and p-ERK-stained sections of oncogene-negative human tumors. H-scores for the whole section is indicated for each representative image. Scale bars=200 um, 40 um b. Quantification of pAKT and pERK staining on 35 oncogene-negative and 18 oncogene-positive human lung adenocarcinomas. Genotypes of oncogene-positive tumors with the lowest pERK and pAKT staining intensities are highlighted in red. Significance between groups was determined using Mann-Whitney U test, ns: non-significant, ****p<0.0001 c. pERK and pAKT H-scores for oncogene-negative human tumors are replotted from FIG. 4b. Red dotted lines: the thresholds for low versus medium pERK and pAKT stains based on the lowest pERK staining intensity of oncogene-positive lung adenocarcinomas and the lowest pAKT staining level of EGFR mutant lung adenocarcinomas. Black dotted lines: the thresholds for medium versus high pERK and pAKT staining based on the mean pERK and pAKT H-scores in oncogene-positive tumors. The number of tumors in each staining intensity group (low, medium, high) are indicated on each axis of the plot. d. Alteration frequency of well-established components of Ras and PI3K pathways (Table 2) that lead to the activation of these two pathways and assessment of their co-occurrences based on TCGA datasets, the p-value is calculated by two-sided Fisher's Exact Test. e. Cumulative distribution function (CDF) plot of the signature scores for human tumors stratified by genes upregulated in mouse oncogene-negative tumors generated by inactivation of Nf1, Rasa1, and Pten (FIG. 19f). The cohort size and the P-value calculated by Kolmogorov-Smirnov test are indicated on the plot.

FIG. 5 (panels a-o) SHP2 inhibitor synergizes with AKT inhibitor to reduce the growth of Nf1, Rasa1, and Pten mutant oncogene-negative lung tumors. a. Schematic of 6 barcoded triple sgRNA vectors for CRISPR/Cas9-mediated inactivation of combinations of Nf1, Rasa1, and Pten in TC mice to determine the response of oncogene-negative tumors to pharmacological inhibition of RAS and PI3K pathways. Indicated numbers of mice were treated with RMC-4550 (SHP2 inhibitor), capivasertib (AKT inhibitor), or combination of these two drugs for two weeks 3.5 months after tumor initiation. Tuba-seq and histological analysis were performed on tumor-bearing lungs followed by analysis of tumor response to therapies. ifu, infection unit b. Bright-field and fluorescence images of lungs from the indicated mice. Lung lobes are outlined with a dashed white line. Scale bar=4 mm c. Representative H&E and Tomato-stained sections of tumors from TC mice 3.5 months after transduction with Lenti-sgTSTripleTS6/Cre and two weeks after treatment with the indicated drugs. Scale bar=100 um d,e. Relative tumor burden in mice after treatment with capivasertib, RMC-4550, and combination of these two drugs compared with tumor burden in vehicle-treated mice. ns: non-significant, *p<0.05, ***p<0.001. Drug response is shown for all the tumors and tumors driven by inactivation of Nf1, Rasa1, and Pten. f,l,n. Representative dose-response matrix depicting growth inhibition of an oncogene-negative cell-line after treatment with different doses of capivasertib and RMC-4550 for four days. The average responses of three to four replicates are shown for each drug/drug combination. g,m,o. Loewe's synergy score calculated based on drug responses in FIG. 5f. Synergy scores indicate the percentage of response beyond expectation. h. Average Loewe's synergy score based on drug-dose response matrix of 3 independent oncogene-negative cell-lines after treatment with different doses of RMC-4550 and capivasertib +/− the 95% confidence intervals. i,j. Cell proliferation and apoptosis analysis using EdU incorporation and cleaved caspase 3 staining and flow-cytometry analysis. Three independent oncogene-negative cell-lines were treated with 10 uM of indicated drug/drugs for 2 days before the analysis. k. Model of biochemical progression and molecular drivers of oncogene-negative tumors.

FIG. 6 (panels a-e) Clinical and molecular features of oncogene-negative lung adenocarcinomas. a. Frequency of human oncogene-positive, oncogene-indeterminate, and oncogene-negative lung adenocarcinomas based on GENIE data sets. b. Overall survival and disease-related survival of oncogene-positive and oncogene-negative tumors based on the TCGA data. The numbers below the plots demonstrate the numbers of patients who are alive at each time point. c. Table shows clinical characteristics of oncogene-positive and oncogene-negative patients based on TCGA and GENIE data sets. SEM—standard error of the mean. N/A—information not furnished in this dataset. The p-value was calculated using Mann Whitney U test, *p<0.05. d,e. The number of total mutated genes (d, by point mutations (PMs) and indel) or total mutated tumor suppressor genes (e, by point mutation, indel, or deletion) in oncogene-positive and oncogene-negative tumors based on TCGA and GENIE data sets. The mean is represented by the dashed line, while the median by the straight line (*p<0.05 calculated using Mann Whitney U test).

FIG. 7 (panels a-d) Tumor suppressors targeting key signaling pathways are altered in oncogene-negative lung adenocarcinoma. a. Schematic of the pathways that are controlled by the five tumor suppressor genes inactivated using floxed alleles (“core” tumor suppressor genes) in this study. The tumor suppressors represent different key cancer pathways. b. Alteration frequency of “core” tumor suppressor genes (number of tumors with potentially inactivating missense or nonsense mutations or focal DNA copy number losses/total tumor number) in oncogene-negative lung adenocarcinomas based on GENIE and TCGA data sets. c,d. The ratio of the frequency of inactivating alterations of tumor suppressor genes (point mutations, indel, and copy number loss) of the genes in Lenti-sgTS102/Cre and Lenti-sgTS15/Cre in oncogene-negative versus oncogene-positive lung adenocarcinomas. Data from TCGA (c) and GENIE (d) data sets are shown. The dotted line represents equal frequency in oncogene-negative and oncogene-positive lung adenocarcinomas. The “Core” tumor suppressors are displayed with bold letters (*FDR<0.05 calculated using Fisher's exact test).

FIG. 8 (panels a-d) The majority of tumors in Nf1f/f; TC, Ptenf/f; TC, and Trp53f/f; TC mice arise the absence of mutations in the proto-oncogenes. a. Schematic of combined Cre/lox and CRISPR/Cas9-mediated tumor suppressor gene inactivation to generate lung epithelial cells with diverse genotypes. b. Representative light and fluorescence images of lung lobes from the indicated genotypes of mice. Lung lobes are outlined with white dotted lines. Scale bar=4 mm c. The number of tumors (defined as Tomato-positive expansions greater than 0.5 mm in diameter) was quantified by direct counting. Each dot represents a mouse, and the bar is the mean. d. Exons in known proto-oncogenes that were analyzed through targeted sequencing. Key codons are those that are often mutated in human cancer. Mutation frequency is the number of tumors with putative oncogenic mutations over the total number of samples analyzed. Putative oncogenic mutations identified are shown (see Methods).

FIG. 9 (panels a-f) Identification of tumor suppressor genes that constrain lung tumor formation in vivo. a-d. Relative frequency of sgRNAs targeting each tumor suppressor gene in tumors harvested from the indicated genotype of mice. Tumors were dissected under a fluorescent microscope based on their tdTomato fluorescent signal and were subjected to genomic DNA extraction. The sgID-BC region was PCR amplified and sequenced using Illumina high-throughput sequencing. The dotted lines represent relative frequency of sgIDs related to inert sgRNAs. Genes significantly overrepresented compared to inert sgRNAs are shown as: ***p<0.001, *p<0.05, ·p<0.1. Light gray bars indicate sgRNAs targeting the “core” tumor suppressor gene that is inactivated with floxed allele in each plot and the inert sgRNAs. e-f. Example plots indicating evidence of transduction with multiple barcoded lentiviral vectors. The 10 sgID-BCs with the highest read counts from two excised tumors are shown. Each dot represents a sgID-BC, the y-axis shows read count, and the sgID-BCs are sorted on the x-axis by decreasing read counts (the first 5 nucleotides of the random barcode are shown with the targeted gene symbol). The first two and three barcodes (sgID-BC) in subpanels e and f, respectively, that have very similar read counts likely represent a single clonal tumor initiated from a cell transduced with multiple barcoded Lenti-sgRNA/Cre vectors.

FIG. 10 (panels a-d) In vivo lung epithelial cell expansion is suppressed by diverse tumor suppressor genes. a-d. Median cell-expansion sizes (normalized to the sizes of inert sgRNA containing cell expansions) for each putative tumor suppressor targeting sgRNAs in one lung lobe harvested from the indicated mouse genotype. Dotted lines indicate the median value for inert sgRNAs. Cell expansions are defined as clonal expansions containing a minimum of 50 cells. Bootstrap 95% confidence intervals are shown as whiskers. sgRNAs with median sizes significantly (p<0.05) higher than the median effect for all sgRNAs are shown in red.

FIG. 11 (panels a-d) Inactivation of different tumor suppressor genes influences lung epithelial cell expansion based on mutational context. a-d. Expansion sizes at the indicated percentiles for the top 13 tumor suppressor genes (relative to the median value of sgInert-containing expansions). The dotted lines indicate the median values for inert sgRNAs. Cell expansions are defined as clonal expansions containing a minimum of 50 cells. Bootstrap 95% confidence intervals are shown as whiskers. sgRNAs with median sizes significantly (p<0.05) higher than the median effect for all sgRNAs are shown in red.

FIG. 12 (panels a-d) The largest oncogene-negative tumors are frequently generated through the inactivation of more than two tumor suppressor genes. a, b. The number of tumors with a minimum size of 1000 cells relative to the inert guide is shown as a blue bar. 90th percentile of tumor sizes relative to the inert sgRNA is shown as a red bar. sgRNAs resulting in a significantly higher number or larger tumors than the inert sgRNA (p<0.05) are shown in color. Whiskers show the 95% confidence intervals. Mouse genotypes are indicated on each plot. c,d. Depiction of the top 15 most frequently occurring triple tumor suppressor alterations in each indicated genotype. Barcodes with the highest cell count in each mouse were investigated for coinfection for multiple viruses (see Methods). The top 15 pairs of tumor suppressors found co-mutated the largest tumors are shown. *p<0.05, **p<0.01, ***p<0.001 based on a permutation test.

FIG. 13 (panels a-d) Nf1, Rasa1, and Pten are frequently mutated in the largest tumors of Nf1f/f; TC, Ptenf/f; TC, Trp53f/f; TC, and TC mice a-d. Depiction of the top 15 combinatorial alterations of three tumor suppressor genes in the largest tumors of indicated genotypes of mice (see Methods). *p<0.05, **p<0.01, ***p<0.001 based on a permutation test. Combinations of sgRNAs that lead to the generation of Nf1, Rasa1, and Pten mutant cancer cells in a statistically significant manner are shown in a bold font.

FIG. 14 (panels a-c) Very few tumors were developed in Nf1f/f; TC, Ptenf/f; TC, Trp53f/f; TC, and TC mice after receiving the Lenti-sgTS11/Cre pool. a. Schematic of tumor initiation with a pool of 11 barcoded Lenti-sgRNA/Cre vectors (Lenti-sgTS11/Cre) similar to Lenti-sgTS14/Cre but excluding sgNf1, sgRasa1 and sgPten. Each gene is targeted with a single sgRNA. Mouse genotype, mouse number, and titer of virus delivered to each mouse are indicated. Tuba-seq was performed on each tumor-bearing lung 4 months after tumor initiation. b. Representative light and fluorescence images of lung lobes from the indicated genotypes of mice. Lung lobes are outlined with white dotted lines. Scale bar=4 mm c. The number of surface tumors (defined as Tomato-positive cell masses larger than 0.5 mm in diameter) lungs of mice indicated above were quantified by direct counting. Each dot represents a mouse, and the bar is the mean.

FIG. 15 (panels a-e) Pairwise tumor suppressor gene inactivation is rarely sufficient for the efficient generation of lung tumors. a. Schematic of tumor initiation with lentiviral vectors (Lenti-sgTS/Cre) targeting a single tumor suppressor gene in Nf1f/f; TC and Ptenf/f; TC mice. Mice were analyzed histologically 10 months after tumor initiation. b,d. Representative light and fluorescence images of lung lobes from the indicated genotypes of mice. Lung lobes are outlined with white dotted lines. Scale bar=4 mm c,e. The number of tumors (defined as Tomato-positive cell masses larger than 0.5 mm in diameter) quantified by direct counting. 10 months after transduction with Lenti-sgRasa1/Cre, lung lobes of Nf1f/f; TC mice contained thousands of Tomato-positive hyperplasias, and tumor number was not quantifiable by direct counting. Each dot represents a mouse, and the bar is the mean.

FIG. 16 (panels a-c) Single tumor suppressor gene inactivation is rarely sufficient to generate lung tumors. a. Schematic of assessment of the potential of single tumor suppressor inactivation to generate lung tumors. Inactivation of a single tumor suppressor gene was done using introduction of lentiviral vectors containing sgRNAs targeting a single tumor suppressor gene into mice without any floxed tumor suppressor alleles or through Lenti-sgInert/Cre into mice carrying tumor suppressor floxed alleles. b. Representative light and fluorescence images of lung lobes from the indicated genotypes of mice. Lung lobes are outlined with white dotted lines. Scale bar=4 mm c. The number of tumors was quantified by direct counting. Each dot represents a mouse, and the bar is the mean. The genotype of the recipient mice and the gene targeted by sgRNA are indicated on the plot.

FIG. 17 (panels a-b) Pairwise inactivation of tumor suppressor genes rarely generates solid lung tumors. a, b. Representative H&E, Tomato, TTF1, TP63, and UCHL1-stained sections of tumors from Nf1f/f; TC and Ptenf/f; TC mice 10 months after transduction with Lenti-sgRasa1/Cre and Lenti-sgNf1/Cre. Scale bar=100 um

FIG. 18 (panels a-h) The relative contribution of Nf1, Rasa1, and Pten inactivation to oncogene-negative lung tumor development is not impacted by Trp53 inactivation. a. Tumor burden, represented by lung weight. Each dot represents a mouse and the bar is the mean. b. Quantification of tumor burden based on H&E images. Each dot represents one lung lobe from each mouse, and the bar is the mean. c. Schematic of the increase in the number of oncogene-negative lung tumors generated in mice by enriching sgRNAs targeting the most potent tumor suppressor genes in each round of functional genomic screening in vivo. The viral titer, number of months after tumor initiation, and average number of tumors are indicated. d. Representative H&E and Tomato stained sections of lungs from TC and Trp53flox/flox; TC mice 3 months after transduction with Lenti-sgTSTriple-pool/Cre. Scale bar=500 mm e. Representative H&E, Tomato, TTF1, TP63, and UCHL1-stained sections of tumors from TC and Trp53flox/flox; TC mice 3 months after transduction with Lenti-sgTSTriple-pool/Cre. Scale bar=100 um f. Numbers of tumors (with >1000 neoplastic cells) are shown relative to Inert sgRNA. sgRNAs resulting in a significantly higher number of tumors than sgInert (p<0.05) are shown in color. Mean+/−95% confidence interval is shown. g. Adaptive landscape of Nf1, Rasa1, and Pten inactivation in Trp53flox/flox; TC is shown. Nodes represent genotypes shown as a string of +(wild-type) and −(inactivated) symbols representing Nf1, Rasa1, and Pten. Numbers in the nodes indicate fitness increase compared to wild-type. The relative probability of each beneficial mutation is shown as arrow widths (see Methods). h. Quantification of the ability of combined Nf1/Rasa1/Pten inactivation in TC mice and oncogenic KrasG12D in KT mice to initiate tumors. Number of tumors (with >1000 neoplastic cells) per infectious unit (ifu) is shown. The bar is the median, the box represents the interquartile range, and the whiskers show minimum and maximum values. n.s: non-significant

FIG. 19 (panels a-g) Oncogene-negative lung tumors driven by inactivation of Nf1, Rasa1, and Pten are almost exclusively adenomas/adenocarcinoma. a. Schematic of inactivation of Nf1, Rasa1, and Pten in TC and Tp53flox/flox; TC mice utilizing triple guide vectors and CRISPR/Cas9-mediated gene-inactivation. Mouse genotype, mouse number, and titer of virus delivered to each mouse are indicated. ifu, infection unit b. Bright-field and fluorescence images of lungs from the indicated mice 3 months after tumor initiation with Lenti-sgNf1-sgRasa1-sgPten/Cre virus. Lung lobes are outlined with a dashed white line. Scale bar=4 mm c. Tumor burden, represented by lung weight. Each dot represents a mouse, and the bar is the mean. d. Quantification of tumor number based on H&E images of one lung lobe from each mouse. Each dot represents one lung lobe from each mouse and the bar is the mean. e. Representative H&E, Tomato, TTF1, TP63, and UCHL1-stained tumor sections from TC and Trp53flox/flox; TC mice 3 months after transduction with Lenti-sgNf1-sgRasa1-sgPten/Cre. Squamous cell lung cancer was only rarely observed in Trp53flox/flox; TC mice (3 out 264 tumors). Scale bar=100 um f. Summary of the mouse tumors sorted using FACS and analyzed by RNA-sequencing. g. Analysis of insertion and deletion in genomic DNA from FACS sorted tumors of 19 TC mice 4 months after transduction with 5×104 ifu of Lenti-sgNf1-sgRasa1-sgPten/Cre. sgRNA targeted regions were PCR amplified, and knockout scores, representing the proportion of cells that have either a frameshift-inducing indel or a large indel in a protein-coding region, were calculated using Synthego's ICE.

FIG. 20 (panels a-h) Oncogene-positive lung adenocarcinomas have higher levels of RAS activation than oncogene-negative tumors. a-e. Representative p-AKT and p-ERK-stained sections of tumors from human oncogene-negative and oncogene-positive tumors. H-score for the whole section is indicated on each representative image. Scale bars=200 μm, 40 μm f. Replotting of pAKT and pERK staining on 35 oncogene-negative and 18 oncogene-positive human lung adenocarcinomas (FIG. 4b, c). The tumors shown as IHC examples in FIG. 4a, and 20a-d are labeled on this plot. g, h. RAS and PI3K-AKT pathway gene-set profiles estimated by single-sample Gene Set Enrichment Analysis (ssGSVA). Tumors from KrasG12D; TC (KTC+sgInert and KTC+sgPten: Kras and Kras/Pten) mice are compared with Nf1, Rasa1, and Pten mutant tumors(Nf1/Rasa1/Pten). The bar is the mean. ns: non-significant, *p<0.05 using Mann-Whitney U test.

FIG. 21 (panels a-i) Alterations in RAS and PI3K pathways are enriched in oncogene-negative human lung adenocarcinomas. a. Frequency of alteration of well-established components of Ras and PI3K pathways (Table 2) queried in GENIE data set and assessment of their co-occurrences, the p-value is calculated by two-sided Fisher's Exact Test. b, c. Alteration frequencies of NF1, RASA1, and PTEN (point mutation, CNV, and indel) and assessment of their co-occurrences, the p-values were calculated by two-sided Fisher's Exact Test. 91 oncogene-negative tumors were from the TCGA datasets. 525, 995, and 525 tumors were analyzed for RASA1/PTEN, NF1/PTEN, and RASA1/NF1 alterations from the GENIE dataset. d-h. Representative PTEN-stained sections of oncogene-negative human tumors. H-score for the whole section is indicated for each representative image. Scale bar=200 μM (right), 50 μm (left) i. PTEN H-scores for oncogene-negative human lung adenocarcinoma tumors.

FIG. 22 (panels a-h) Nf1, Rasa1, and Pten mutant oncogene-negative lung tumors respond to inhibition of PI3K and RAS pathways. a. Schematic of RAS and PI3K pathways activated by alterations of Nf1, Rasa1, and Pten and targeted by SHP2 and AKT inhibitors. b. Drugs used to inhibit RAS and PI3K pathways in vivo and their dosages. c. Lung weight of mice described in FIG. 5a-b. d-f, h. Relative tumor burdens of mice after treatment with capivasertib, RMC-4550, and combination of these two drugs compared with tumor burden in vehicle-treated mice. ***p<0.001 Data are shown for pairwise inactivation of Nf1, Rasa1, and Pten. g. Representative pERK and pS6-stained sections of tumors from TC mice described in d after treatment with the indicated drugs. Scale bar=100 um

FIG. 23 (panels a-f) RMC-4550 and capivasertib treatment induce apoptosis gene signature and suppress G2/M gene signature in oncogene-negative tumors a. Schematic of generation of Nf1, Rasa1, and Pten mutant lung tumors in TC mice to determine their gene expression changes to pharmacological inhibition of RAS and PI3K pathways. Indicated number of mice were treated with vehicle or combination of RMC-4550 and capivasertib 4.5 months after tumor initiation for three days. RNA-sequencing was performed on libraries prepared from RNA extracted from sorted Tomato-positive and lineage-negative cells. b. Volcano plots depicting a global overview of differential gene expression in Nf1, Rasa1, Pten mutated tumors in the absence and presence of treatment with RMC-4550 and capivasertib for three days as described above. Significant differential expression is defined as an absolute log 2 (Fold Change) >1 and FDR<0.01. The numbers of significantly differentially expressed genes are indicated on the plot. c-f. Comparison of RAS, PI3K-AKT, apoptosis, and G2M gene-set profiles estimated by single-sample Gene Set Enrichment Analysis (ssGSVA) in mouse tumors from TC mice with inactivation of Nf1, Rasa1, and Pten after treatment with vehicle or RMC-4550 and capivasertib for three days. Each dot represents one tumor. ssGSVA data points shown for vehicle-treated tumors are the same as FIG. 3h-i as Nf1/Rasa1/Pten. The bar is mean. ns: non-significant, *p<0.05 using Mann-Whitney U test.

FIG. 24 (panels a-m) RMC-4550 synergizes with capivasertib to inhibit proliferation and induce cell-death in Nf1, Rasa1, and Pten-mutant lung adenocarcinoma cell-lines. a. Schematic of cell-line generation from oncogene-negative tumors generated in Trp53flox/flox; TC mice. b. Immunoblot of 3 distinct oncogene-negative cell-lines treated with 10 uM of indicated drugs for 24 hours. c,e, g. Drug dose-response matrix depicting % growth inhibition after treatment with various doses of RMC-4550 and capivasertib indicated on the plots. The cell-line used for the generation of each matrix is noted on top of each heatmap. d,f, h. Loewe's synergy score was calculated for each drug dose combination shown in c and e. Synergy score indicates the percentage of inhibition beyond what is expected if there is no interaction between the drugs. i. Immunoblot of 6 human oncogene-positive and 2 human oncogene-negative cell lines for markers of RAS and PI3K pathway activation. j. Drug dose-response matrix depicting % growth inhibition of H1623 human Onc-negative RAS/PI3K cell line. k. Loewe's synergy score calculated based on drug responses. 1. Indel analysis of 3 distinct mouse oncogene-negative RAS/PI3K cell lines described above. Regions targeted by sgNf1, sgRasa1, and sgPten were PCR amplified and analyzed using Synthego ICE after sanger sequencing. Knockout score represents indels causing frameshift mutations. m. Immunoblot of 2 murine oncogene-positive cell lines (MMW398T2 and HC494: KrasG12D and Trp53 mutant and Nf1, Rasa1, and Pten wild type) and 3 murine Onc-negative RAS/PI3K mouse cell lines (described above) to assess loss of RASA1 and PTEN in oncogene-negative cell lines.

FIG. 25 Example plots indicating strong evidence of infection with multiple lentiviral vectors in the largest tumors in each genotype. 30 sgID-BC with the highest read counts from representative mouse samples are shown. Indicated genotypes of mice were transduced with Lenti-sgTS14/Cre pool. Dots represent sgID-BCs, the y-axis shows read count, sgID-BCs are sorted on the x-axis by decreasing read count. Group of barcodes (sgID-BC) showing similar read counts can be a sign of multiple infections.

FIG. 26A-26C Table 4. Characteristics of lung adenocarcinoma patients with oncogene-negative tumors assessed for activation of RAS and PI3K pathways.

FIG. 27A-27B Table 4. Characteristics of lung adenocarcinoma patients with oncogene-positive tumors assessed for activation of RAS and PI3K pathways.

FIG. 28 Evaluation of oncogene-negative tumors. Sections were stained from 20 oncogene-negative human tumors that showed no genomic alterations for PTEN. As shown in this graph, despite the lack of genomic alterations for PTEN, the majority of the tumors exhibited low levels of PTEN protein. Of those, the vast majority (all but 3) exhibited medium to high levels of pAKT (a measure of PI3K-AKT pathway activity). In total, 50% of all tumors tested exhibited low levels of PTEN protein and medium to high levels of pAKT.

FIG. 29. Inactivation of Nf1, Rasa1, and Pten generates lung tumors with the ability to metastasize to other organs. a. Schematic of inactivation of Nf1, Rasa1, and Pten in Trp53flox/flox; TC mice using the Lenti-sgNf1-sgRasa1-sgPten/Cre vector. Mouse genotype, mouse number, and titer of virus delivered mice are indicated. ifu, infection unit. b. Bright-field and fluorescence images of lungs, diaphragm, and liver from the Trp53flox/flox; TC mice 12 months after tumor initiation with Lenti-sgNf1-sgRasa1-sgPten/Cre virus. Lung lobes are outlined with a dashed white line. Scale bars=5 and 0.5 mm (4 out of 32 mice had obvious metastasis). c. Representative H&E, Tomato, TTF1, HMGA2, TP63, UCHL1, SYNAPTOPHYSIN (SYP), and CGRP-stained tumor sections from Trp53flox/flox; TC mice 9-12 months after transduction with Lenti-sgNf1-sgRasa1-sgPten/Cre. Scale bars=100 μm. d. H&E and tdTomato staining of liver sections from one of the Trp53flox/flox; TC mice with metastasis. Scale bars=100 μm.

### DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It is noted that as used herein and in the appended claims, the singular forms “a,” “an,” and “the” include plural referents unless the context clearly dictates otherwise. Thus, for example, reference to “a cell” includes a plurality of such cells and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Compositions and Methods

As summarized above, methods and compositions (e.g., kits) for treating individuals who have an oncogene-negative cancer. In some embodiments, the subject methods and compositions are for treating an individual who has an oncogene-negative cancer, such as an oncogene-negative lung adenocarcinoma. Also provided are screening methods and compositions (e.g., cells and/or non-human genetically modified mammal) for testing candidate therapies, where the cells and non-human genetically modified mammals have an oncogene-negative genomic profile.

By the term “oncogene-positive” it is meant an individual who has a cancer (e.g., lung adenocarcinoma) with oncogene alterations in previously described proto-oncogenes, i.e., their cancer cells (e.g., assayed via biopsy, resection, and the like) harbor mutations in genes that have been shown to generate oncogenic activity. By the term “oncogene-negative” it is meant an individual who has a cancer (e.g., lung cancer such as lung adenocarcinoma) with no alterations in known proto-oncogenes, i.e., their cancer cells (e.g., assayed via biopsy, resection, and the like) do not harbor mutations in genes that have been shown to generate oncogenic activity. Table 1 provides a list of proto-oncogenes and known mutations that generate oncogenic activity. Thus, an oncogene-negative signature is one that is negative for the mutations listed in Table 1 (e.g., wild type for the listed genes).

In each of the pathways, specific mutations in the listed genes are known to be oncogenic, meaning the specific alteration alone is enough to transform normal cells into cancer cells. However, other mutations in that gene, or mutations genes that are known not to be oncogenes, alone are not sufficient to drive oncogenic transformation (they need ‘help’ from other mutations in other genes in order to transform a cell into a cancer cell). As an illustrative example, in the RAS pathway, KRAS(G12D) mutation alone can drive transformation and is an oncogene. However, NF1 mutation alone is not sufficient and it needs other genes to be mutated as well. In each of the protooncogenes, few alterations are known to be oncogenic mutations. For example, one may increase KRAS activity slightly by other mutations that are not oncogenic. However, that slight increase in activity is not sufficient to drive transformation and other alterations are needed to initiate oncogenic transformation.

In some cases a diagnostic kit such as GRAIL's diagnostic kit (Galleri) or similar technologies can be used to determine whether an individual's cancer is oncogene-negative. Any convenient method can be used to make such a determination. For example, next generation (high throughput) sequencing can be used.

TABLE 1
List of proto-oncogenes and known mutations that generate oncogenic activity

| Gene | Alteration(s) | Evidence |
|---|---|---|
| EGFR | Exon 18 G719C | Response to EGFR inhibition |
| | Exon 18 G719S | Response to EGFR inhibition |
| | Exon 18 G719A | Response to EGFR inhibition |
| | Exon 19 in-frame deletion | generation of lung |
| | around aa747 to aa750 | adenocarcinoma in mouse |
| | | models, response to EGFR |
| | | inhibition in patients and cell- |
| | | lines |
| | Exon 20 in-frame Insertions | Mostly resistant to TKI, |
| | and/or duplications of 3 to 21 | A763_Y764insFQEA respond to |
| | base pairs (bp)(between aa 762- | covalent quinazoline-based |
| | 774) | EGFR inhibitor dacomitinib |
| | Exon 21 L861Q | Response to gefitinib (EGFR |
| | | inhibition) |
| | Exon 21 L858R | Response to gefitinib and |
| | | erlotinib (EGFR inhibition), |
| | | generation of lung |
| | | adenocarcinoma in mouse |
| | | models, |
| | copy number alteration | Response to EGFR inhibition, |
| | | cell-line trasformation, |
| | | development of lung |
| | | adenocarcinoma in mouse |
| | | model |
| KRAS | Exon 2: G12X | transforming cells in culture, |
| | | Lung adenocarcinoma |
| | | generation in mouse models, |
| | Exon 2: G13X | transforming cells in culture, |
| | | Lung adenocarcinoma |
| | | generation in mouse models, |
| | Exon 3: Q61X | transforming cells in culture |
| HRAS | Exon 2: G12X | transforming cells in culture |
| | Exon 2: G13X | |
| | Exon 3: Q61X | transforming cells in culture |
| NRAS | Exon 2: G12X | |
| | Exon 2: G13X | |
| | Exon 3: Q61X | tumor generation in Monte's lab |
| | | (unpublished) |
| Braf | V600E | Generation of lung |
| | | adenocarcinoma in mouse |
| | | models, sensitivity to MEKi |
| MET | exon 14 skipping | Response to targeted therapy |
| | amplification | Response to targeted therapy |
| | KIF5B-MET fusion | response to targeted therapy |
| ALK | Fusion | transformation of mouse 3T3 |
| | | cells and tumor generation in |
| | | nude mice after transplantation, |
| | | response to ALK inhibitor |
| Ros1 | Fusion | Response to targeted therapy, |
| | | subcutaneous tumor formation |
| | | in nude mice, cell |
| | | transformation in culture |
| Ret | Fusion | generation of tumor in nude |
| | | mice (subcutaneous), cell |
| | | transformation in culture, |
| | | response to targeted therapy |
| Sos1 | N233Y | Cell transformation in vitro, |
| | | Tumor formation in mice |
| | | (allografts) |
| | D309Y | Cell transformation in vitro, |
| | | Tumor formation in mice |
| | | (allografts) |
| | P478L | Cell transformation in vitro, |
| | | Tumor formation in mice |
| | | (allografts) |
| | G604V | Cell transformation in vitro, |
| | | Tumor formation in mice |
| | | (allografts) |
| HER2/ERBB2 | Exon 20 in-frame insertion or | modest response to TKI, lung |
| | duplication (the most common, | adenocarcinoma tumor |
| | exon 20 makes amino acids 770- | development in mouse model, |
| | 831, A775_G776insYVMA is | cell transformation in vitro |
| | the most frequent exon 20 | |
| | insertion) | |
| | amplification/copy number | Response to TKI, lung |
| | alteration | adenocarcinoma generation in |
| | | mouse models |
| | Exon 8: S310F | Response to TKI, NIH 3T3 |
| | | colony transformation |
| | Exon 8: S310Y | cell transformation in vitro, |
| | G660D | Response to HER2 blockade in |
| | | cell culture, in vivo |
| | | (transplanted), patients |
| | Q709L | Response to HER2 blockade |
| | R678Q | Response to HER2 blockade |
| | V659E | Response to HER2 blockade, |
| | | germline expression of theHER2 |
| | | TMD V659E (neu) mutant |
| | | cDNA in the presence of endog- |
| | | enous mouse ERBB2 did not |
| | | affect the development of mice |
| | | (Andrechek et al., 2004) |
| AKT1 | E17K | transformation of lung epithelial |
| | | cell line in vitro and formation |
| | | of subcutanoeus tumors in vivo- |
| | | poorly differentiated |
| | | adenocarcinoma and squamous |
| | | cell carcinoma, patient response |
| | | to AKT inhibition |
| NTRK1 | Fusion | Response to targeted therapy, |
| | | NIH3T3 cells expressing CD74- |
| | | NTRK1 induces tumorigenesis |
| | | in nude mice |
| MAP2K1 (MEK1) | Exon 2: F53L | Cell transformation in vitro, |
| | | Response to MEKi |
| | exon 2: K57N | cell transformation in vitro, |
| | | formation of subcutaneous |
| | | tumors in mice, response to |
| | | MEKi |
| | exon 2: Q56P | Cell transformation in vitro, |
| | | Response to MEKi |
| | exon 2: D67N | Cell transformation in vitro, |
| | | Response to MEKi |
| | exon 3: C121S | cell transformation in vitro, |
| | | response to MEKi |
| | Exon 3: E102_I103del | cell transformation in vitro, |
| | | formation of subcutaneous |
| | | tumors in mice, response to |
| | | MEKi |
| | Exon 3: P105_A106del | cell transformation in vitro, |
| | | response to MEKi |
| | Exon 3: L98-I103del | cell transformation in vitro, |
| | | response to MEKi |
| | Exon 3: L99-K104del | cell transformation in vitro, |
| | | response to MEKi |
| | Exon 3: I103-K104del | cell transformation in vitro, |
| | | response to MEKi |
| | E203K | Response to MEKi |
| | L177M | Response to MEKi |
| | F53-Q58del | Response to MEKi |
| | K57E | Response to MEKi |
| | E51-Q58del | Response to MEKi |
| NRG1 | Fusion | response to TKI |
| RIT1 | Q79L | cell transformation, Tumor |
| | | growth of xenografts of NIH3T3 |
| | | cells |
| | T76_insTLDT | cell transformation |
| | A77P | cell transformation |
| | F82L | cell transformation |
| | M90I | cell transformation, Tumor |
| | | growth of xenografts of NIH3T3 |
| | | cells |
| | TA83del | cell transformation |
| | A77S | cell transformation, Tumor |
| | | growth of xenografts of NIH3T3 |
| | | cells |
| | Q40L | cell transformation, Tumor |
| | | growth of xenografts of NIH3T3 |
| | | cells |

In some cases, the method includes administration to the individual of an inhibitor of the Ras/MAPK pathway (e.g., an inhibitor of SHP2 such as RMC-4550). In some cases, the method includes administration to the individual of an inhibitor of the Ras/MAPK pathway (e.g., an inhibitor of SHP2 such as RMC-4550) and an inhibitor of the PI3K-AKT pathway (e.g., an inhibitor of AKT1/2 such as capivasertib). Table 2 below provides a list of genes from the Ras/MAPK and PI3K-AKT pathways that could be used to select targets for inhibition. Table 2 also includes examples of genetic alterations that lead to activation of these pathways. Table 3 below provides a list of examples of known agents that target members of these pathways. In some cases, an agent that includes an agent from Table 3 is administered to an individual. In some cases, an agent from Table 3 is administered.

As an illustrative example, examples of inhibitors of SHP2 include, but are not limited to: RMC-4550, RMC-4630, BBP-398, JAB-3068, RLY-1971, ERAS-601, and TNO155. In some cases, the inhibitor of the Ras/MAPK pathway that is administered is RMC-4550, RMC-4630, BBP-398, JAB-3068, RLY-1971, ERAS-601, or TNO155, or any combination thereof. In some cases, the inhibitor of the Ras/MAPK pathway that is administered is RMC-4550. Thus, in some cases, an inhibitor of SHP2 (e.g., RMC-4550, RMC-4630, BBP-398, JAB-3068, RLY-1971, ERAS-601, or TNO155, or any combination thereof) is administered. In some cases, RMC-4450 is administered.

As another illustrative example, examples of inhibitors of AKT include, but are not limited to: A-443654, AKT inhibitor VIII, AT13148, AT7867, Afuresertib, Capivasertib, GSK690693, Ipatasertib, MK-2206, and Uprosertib. In some cases, the inhibitor of the PI3K-AKT pathway that is administered is A-443654, AKT inhibitor VIII, AT13148, AT7867, Afuresertib, Capivasertib, GSK690693, Ipatasertib, MK-2206, or Uprosertibm, or any combination thereof. In some cases, the inhibitor of the PI3K-AKT pathway that is administered is capivasertib. Thus, in some cases, an inhibitor of AKT (e.g., A-443654, AKT inhibitor VIII, AT13148, AT7867, Afuresertib, Capivasertib, GSK690693, Ipatasertib, MK-2206, or Uprosertib, or any combination thereof) is administered. In some cases, Capivasertib is administered.

In some cases, an inhibitor of SHP2 (e.g., RMC-4550, RMC-4630, BBP-398, JAB-3068, RLY-1971, ERAS-601, or TNO155, or any combination thereof) is administered and an inhibitor of AKT (e.g., A-443654, AKT inhibitor VIII, AT13148, AT7867, Afuresertib, Capivasertib, GSK690693, Ipatasertib, MK-2206, or Uprosertib, or any combination thereof) is also administered. In some cases RMC-4550 and Capivasertib are administered.

TABLE 2
List of genes in Ras/MAPK and PI3K-AKT pathways. Listed
alterations lead to the activation of these pathways

| | | | |
|---|---|---|---|
| | Mutation | | |
| Gene | type | Alterations | Reference |

Ras/MAPK Pathway

| | | | |
|---|---|---|---|
| ABL1 | gain of | Y459H, A384V, I379V, V396M, D252N | Sanchez-Vega et al., Cell |
| | function | | 2018 |
| ARAF | gain of | Amplification, S214, P216, S214F, | Sanchez-Vega et al., Cell |
| | function | S214C, S214T, P216L | 2018 |
| ERBB3 | gain of | Amplification, V104, E928, D297, K329, | Sanchez-Vega et al., Cell |
| | function | A232, G284, S846, H228, E332, M91, | 2018 |
| | | T355, F219, M60, R103, R475, N126, | |
| | | A232V, E332K, E928G, G284R, R475W, | |
| | | F219V, V104M, N126K, R103H, K329E, | |
| | | V104L, K329T, M60R, F219L, D297N, | |
| | | D297V, D297Y, T355I, A232T, M91I, | |
| | | H228R, H228Q, E928K, E332Q, S846I, | |
| | | E928Q, P262H, T355P | |
| ERBB4 | gain of | Amplification, V104, E928, D297, K329, | Sanchez-Vega et al., Cell |
| | function | A232, G284, S846, H228, E332, M91, | 2018 |
| | | T355, F219, M60, R103, R475, N126 | |
| FGFR1 | gain of | Amplification, N577, K687, K687N, | Sanchez-Vega et al., Cell |
| | function | N577K | 2018 |
| FGFR2 | gain of | Amplification, S252, R203, F276, P253, | Sanchez-Vega et al., Cell |
| | function | S252W, P253R, T268dup, P253L, | 2018 |
| | | W290C, R203H, E537K, D101Y, R251Q, | |
| | | S267_T268insIS, E219G, I564M, F276C | |
| FGFR3 | gain of | Amplification,, S249, R248, P250, D280, | Sanchez-Vega et al., Cell |
| | function | R248C, Y375C, S249C, G382R, S373C, | 2018 |
| | | Y375H, M528T, G372C | |
| FGFR4 | gain of | Amplification, V550 | Sanchez-Vega et al., Cell |
| | function | | 2018 |
| FLT3 | gain of | S249, R248, P250, D280, R248C, Y375C, | Sanchez-Vega et al., Cell |
| | function | S249C, G382R, S373C, Y375H, M528T, | 2018 |
| | | G372C | |
| GRB2 | gain of | F95, G116, K117, S127, E130 | Sanchez-Vega et al., Cell |
| | function | | 2018 |
| IGF1R | gain of | Amplification | Sanchez-Vega et al., Cell |
| | function | | 2018 |
| JAK2 | gain of | Amplification, V617F, R683I, R683T | Sanchez-Vega et al., Cell |
| | function | | 2018 |
| KIT | gain of | Amplification, W557, V559, V560, L576, | Sanchez-Vega et al., Cell |
| | function | P573, N822, D816, K642, R888, D816V, | 2018 |
| | | D816Y, L637H, M638I, L793F, M552L, | |
| | | N822Y, A829P, D816H, N822K, W557C, | |
| | | W557G, H697D, T801A, E490D, K826N, | |
| | | V530I, P573L, K818R, A814S, R888Q, | |
| | | L706P, G498D, G487D, X788_splice, | |
| | | R686H, D52N, P627L, A659T, | |
| | | X627_splice, G664E, P551H, S821Y, | |
| | | N486I, L631F, G812V, L576P, S464L, | |
| | | K642N, L679F, K818N, P573Q, R804W, | |
| | | K642E, W582L, X514_splice, V559A, | |
| | | N680I, S590N, E562G, R804Q, K558E, | |
| | | F689C, P456Q, S451C, P467Q, W557R, | |
| | | N822I, L706F, S712F, G803C, D479H, | |
| | | K818T, T666I, Y503delinsFAH, V668A, | |
| | | S590R, R888L, A636V, D696Y, D458H, | |
| | | A482T, Y578C, V825D, V559G, | |
| | | G565_T574delinsA | |
| MAP2K2 | gain of | Amplification, V104, E928, D297, K329, | Sanchez-Vega et al., Cell |
| | function | A232, G284, S846, H228, E332, M91, | 2018 |
| | | T355, F219, M60, R103, R475, N126, | |
| | | A232V, E332K, E928G, G284R, R475W, | |
| | | F219V, V104M, N126K, R103H, K329E, | |
| | | V104L, K329T, M60R, F219L, D297N, | |
| | | D297V, D29 | |
| MAPK1 | gain of | Amplification,, E322K, E322, E81, R135, | Sanchez-Vega et al., Cell |
| | function | G136, D321, P319, Y316 | 2018 |
| NTRK2 | gain of | Fusion | Sanchez-Vega et al., Cell |
| | function | | 2018 |
| NTRK3 | gain of | Fusion, G623E | Sanchez-Vega et al., Cell |
| | function | | 2018 |
| PDGFRA | gain of | Amplification, D842, E229, N659, C235, | Sanchez-Vega et al., Cell |
| | function | E229K, C235R, N659K, D842Y, V536M, | 2018 |
| | | R748K, C235S, P577L, C235Y, E229V | |
| PTPN11 | gain of | A72, E76, Q510, E69, G503, N308, F285, | Sanchez-Vega et al., Cell |
| | function | T468, T507, D61, A461 | 2018 |
| RAC1 | gain of | Amplification, G15, C18, P29, A178, | Sanchez-Vega et al., Cell |
| | function | N111, Q61, P34, K135, P29S, K135N, | 2018 |
| | | A178V, C18F, P29T, P29L, Q61R, | |
| | | N111K, P34S, C18Y | |
| RAF1 | gain of | amplification,, S257, S259, S257W, | Sanchez-Vega et al., Cell |
| | function | S257L, S259F, P261R | 2018 |
| CBL | Loss of | Any loss of function mutation (point | Sanchez-Vega et al., Cell |
| | function | mutations or copy number loss) | 2018 |
| ERRFI1 | Loss of | Any loss of function mutation (point | Sanchez-Vega et al., Cell |
| | function | mutations or copy number loss) | 2018 |
| NF1 | Loss of | Any loss of function mutation (point | Sanchez-Vega et al., Cell |
| | function | mutations or copy number loss) | 2018 |
| RASA1 | Loss of | Any loss of function mutation (point | Sanchez-Vega et al., Cell |
| | function | mutations or copy number loss) | 2018 |
| EGFR | gain of | gain of function mutations except those | Sanchez-Vega et al., Cell |
| | function | known to be oncogenic | 2018 |
| ERBB2 | gain of | gain of function mutations except those | Sanchez-Vega et al., Cell |
| | function | known to be oncogenic | 2018 |
| MET | gain of | gain of function mutations except those | Sanchez-Vega et al., Cell |
| | function | known to be oncogenic | 2018 |
| FLT3 | gain of | gain of function mutations except those | Sanchez-Vega et al., Cell |
| | function | known to be oncogenic | 2018 |
| ALK | gain of | gain of function mutations except those | Sanchez-Vega et al., Cell |
| | function | known to be oncogenic | 2018 |
| RET | gain of | gain of function mutations except those | Sanchez-Vega et al., Cell |
| | function | known to be oncogenic | 2018 |
| ROS1 | gain of | gain of function mutations except those | Sanchez-Vega et al., Cell |
| | function | known to be oncogenic | 2018 |
| KIT | gain of | gain of function mutations except those | Sanchez-Vega et al., Cell |
| | function | known to be oncogenic | 2018 |
| NTRK1 | gain of | gain of function mutations except those | Sanchez-Vega et al., Cell |
| | function | known to be oncogenic | 2018 |
| SOS1 | gain of | gain of function mutations except those | Sanchez-Vega et al., Cell |
| | function | known to be oncogenic | 2018 |
| KRAS | gain of | gain of function mutations except those | Sanchez-Vega et al., Cell |
| | function | known to be oncogenic | 2018 |
| HRAS | gain of | gain of function mutations except those | Sanchez-Vega et al., Cell |
| | function | known to be oncogenic | 2018 |
| NRAS | gain of | gain of function mutations except those | Sanchez-Vega et al., Cell |
| | function | known to be oncogenic | 2018 |
| RIT1 | gain of | gain of function mutations except those | Sanchez-Vega et al., Cell |
| | function | known to be oncogenic | 2018 |
| BRAF | gain of | gain of function mutations except those | Sanchez-Vega et al., Cell |
| | function | known to be oncogenic | 2018 |
| MAP2K1 | gain of | gain of function mutations except those | Sanchez-Vega et al., Cell |
| | function | known to be oncogenic | 2018 |

PI3K-AKT pathway

| | | | |
|---|---|---|---|
| AKT2 | gain of | Amplification, point mutations: R170Q, | Yi et al., Oncotarget2016; |
| | function | I289M, R170W, R368H, H355Y, E17K | Dobashi et al., Cancer |
| | | | Science 2015; Arboleda et |
| | | | al., Cancer Research 2003 |
| AKT3 | gain of | E17K | Zhang et al., Cancer Cell |
| | function | | 2017 |
| PIK3CA | gain of | Amplification, point mutations: R38, E39, | Chaft et al., Molecular |
| | function | R88, C90, R93, E545, Q546, E547, | Cancer Therapeutics 2011; |
| | | H1047, E542, N345, M1043, C420, G118, | Sanchez-Vega et al., Cell |
| | | E726, K111, E453, V344, R108, G106, | 2018 |
| | | G1049, Y1021, N1044, E81, C901, | |
| | | G1007, T1025, C378, D350, P104, M1, | |
| | | E365, R115, N107, M1004, C604, P471, | |
| | | D939, T1052, H1065, G364, P539, E970, | |
| | | R357, E542K, N345K, E545K, | |
| | | L10_M16del, M1043V, R38H, | |
| | | E453_G460delinsDDF, E542V, E39K, | |
| | | E81K, R88Q, L455_G463del, R108H, | |
| | | E545A, G118D, C420R, E726K, V344M, | |
| | | K111R, H1047R, E545G, C90Y, R93W, | |
| | | Q546K, E1012Q, P539R, R93Q, P539S, | |
| | | R38L, E545Q, G106V, M1004I, C90G, | |
| | | D350N, M1043I, H1047L, K111N, | |
| | | D350G, R38C, D1045V, R108L, E453K, | |
| | | M1004V, E453Q, P104L, N107S, | |
| | | H1047Y, G1049R, H1065L, Q546R, | |
| | | R38S, Q546P, C378Y, W11_P18del, | |
| | | C604R, E453_L455del, T1025A, E365K, | |
| | | N345T, H450_P458del, P447_L455del, | |
| | | V344G, G106R, G364R, R357Q, E970K, | |
| | | V344A, Y1021C, T1052K, L989V, | |
| | | C378F, E542G, P471L, D1029H, | |
| | | D1045A, K111_L113del, K111E, N345I, | |
| | | P449S, R108C, L997I, T1025S, A1020T, | |
| | | N1044K, E545D, H1048R, P449L, | |
| | | C378R, G1007R, H1047Q, Y1021H, | |
| | | E109_I112delinsD, E39G, L113del, | |
| | | E542A, Q546E, G463_N465delinsD, | |
| | | G106_N107del, K111del, E726G, | |
| | | G106_R108del, E1037K, L452_G460del, | |
| | | E365V, E542Q, F1016C, D939G, | |
| | | E103_G106delinsD, M1040V, S1015Y, | |
| | | M1040I, I102del, C901F, V105del, | |
| | | G451_D454del, V105_R108del, G106D, | |
| | | P104T, R93P, A1035T, E453del, | |
| | | N1044Y, R992P, E103_P104del, L1006R, | |
| | | Q546H, M1004R, N1044I, F1002L, | |
| | | P471A, M1005V, L1026I, N345H, | |
| | | M1043T, D1017N, R979G, C90R, | |
| | | R992L, I1058M, P104R, G914R, M1055I, | |
| | | M1043L, N345Y, G106S, N1068Y | |
| PIK3CB | gain of | Amplification, point mutation: D1067, | Sanchez-Vega et al., Cell |
| | function | E1051, R321, D1067V, E1051K, | 2018; Dbouk et al., PLoS |
| | | | One 2013, Pazarentzos et al., |
| | | D1067A, R321Q, R48W, D1067Y, | Oncogene 2016; Whale et |
| | | E552K, R48L, N553S | al., Signal Transduction and |
| | | | Targeted Therapy 2017 |
| PIK3R2 | gain of | Amplification,, G373, D557, D557H, | Sanchez-Vega et al., Cell |
| | function | G373R, N561D, D557Y | 2018; Zhang et al., Cancer |
| | | | Cell 2017 |
| INPP4B | Loss of | Any loss of function mutation, copy | Zhang et al., Cancer Cell |
| | function | number loss, deletion | 2017 |
| NPRL2 | Loss of | Any loss of function mutation, copy | Zhang et al., Cancer Cell |
| | function | number loss, deletion | 2017 |
| NPRL3 | Loss of | Any loss of function mutation, copy | Zhang et al., Cancer Cell |
| | function | number loss, deletion | 2017 |
| PIK3R1 | Loss of | p85a-commonly mutated resulting in | Zhang et al., Cancer Cell |
| | function | reduced ability to inhibit PI3K p110a | 2017 |
| PIK3R3 | Loss of | Any loss of function mutation, copy | Zhang et al., Cancer Cell |
| | function | number loss, deletion | 2017 |
| PPP2R1A | Loss of | Any loss of function mutation, copy | Zhang et al., Cancer Cell |
| | function | number loss, deletion | 2017 |
| PTEN | Loss of | Any loss of function mutation, copy | Zhang et al., Cancer Cell |
| | function | number loss, deletion | 2017 |
| EIF4EBP1 | gain of | Gain of function mutations or | Sanchez-Vega et al., Cell |
| | function | upregulation | 2018 |
| AKT1 | gain of | gain of function mutations except those | Sanchez-Vega et al., Cell |
| | function | known to be oncogenic | 2018 |
| AKT1S1 | gain of | Gain of function mutations or | Sanchez-Vega et al., Cell |
| | function | upregulation | 2018 |
| DEPTOR | gain of | Gain of function mutations or | Sanchez-Vega et al., Cell |
| | function | upregulation | 2018 |
| INPP4B | Loss of | Any loss of function mutation, copy | Sanchez-Vega et al., Cell |
| | function | number loss, deletion | 2018 |
| MAPKAP1 | gain of | Gain of function mutations or | Sanchez-Vega et al., Cell |
| | function | upregulation | 2018 |
| MLST8 | gain of | Gain of function mutations or | Sanchez-Vega et al., Cell |
| | function | upregulation | 2018 |
| MTOR | gain of | p.L2427Q, p.C1483R, p.E1799K, | Sanchez-Vega et al., Cell |
| | function | p.A1971T, p.M2327I, p.S2215Y, | 2018 |
| | | p.I2500N, p.Q2223K, p.I2500F, | |
| | | p.T1977K, p.C1483Y, p.Y1463S, | |
| | | p.L1460P, p.T1977R, p.I2500M, | |
| | | p.V2006I, p.T1977I, p.L1433S, p.C1483F, | |
| | | p.A1459P, p.V2006L, p.A1519T, | |
| | | p.F1888L, p.K1452N, p.S2215F, | |
| | | p.E2419K, p.L2427R, p.L2230V, other | |
| | | Gain of function mutations or | |
| | | upregulation | |
| NPRL2 | Loss of | Any loss of function mutation, copy | Sanchez-Vega et al., Cell |
| | function | number loss, deletion | 2018 |
| NPRL3 | Loss of | Any loss of function mutation, copy | Sanchez-Vega et al., Cell |
| | function | number loss, deletion | 2018 |
| PDK1 | gain of | Gain of function mutations or | Sanchez-Vega et al., Cell |
| | function | upregulation | 2018 |
| RHEB | gain of | p.Y35C, p.Y35N, other Gain of function | Sanchez-Vega et al., Cell |
| | function | mutations or upregulation | 2018 |
| RICTOR | gain of | p.S1101L, other gain of function | Sanchez-Vega et al., Cell |
| | function | mutations or upregulation | 2018 |
| RPTOR | gain of | p.R139H, other gain of function mutations | Sanchez-Vega et al., Cell |
| | function | or upregulation | 2018 |
| RPS6 | gain of | Gain of function mutations or | Sanchez-Vega et al., Cell |
| | function | upregulation | 2018 |
| RPS6KB1 | gain of | Gain of function mutations or | Sanchez-Vega et al., Cell |
| | function | upregulation | 2018 |
| STK11 | Loss of | Any loss of function mutation, copy | Sanchez-Vega et al., Cell |
| | function | number loss, deletion | 2018 |
| TSC1 | Loss of | Any loss of function mutation, copy | Sanchez-Vega et al., Cell |
| | function | number loss, deletion | 2018 |
| TSC2 | Loss of | Any loss of function mutation, copy | Sanchez-Vega et al., Cell |
| | function | number loss, deletion | 2018 |

TABLE 3
List of Agents that target members of the Ras/MAPK and PI3K-AKT pathways

| Agent Name | Synonyms | Targets | Target pathway |
|---|---|---|---|
| A-443654 | KIN001-139 | AKT1, AKT2, AKT3 | PI3K/MTOR signaling |
| AKT inhibitor VIII | Akti-1/2, KIN001-102 | AKT1, AKT2, AKT3 | PI3K/MTOR signaling |
| AMG-319 | AMG319 | PI3K (beta sparing) | PI3K/MTOR signaling |
| AS605240 | KIN001-173, AS- | PI3Kgamma | PI3K/MTOR signaling |
| | 605240 | | |
| AST-1306 | | EGFR, ERBB4 | RTK signaling |
| AT13148 | | AKT1 | PI3K/MTOR signaling |
| AT7867 | | AKT | PI3K/MTOR signaling |
| AZ628 | AZ-628, AZ 628 | BRAF | ERK MAPK signaling |
| AZD1332 | SN1061387896 | NTRK1, NTRK2, | RTK signaling |
| | | NTRK3 | |
| AZD2014 | SN1103949359 | mTORC1, mTORC2 | PI3K/MTOR signaling |
| AZD3759 | | EGFR | EGFR signaling |
| AZD4547 | AZ6 | FGFR1, FGFR2, | RTK signaling |
| | | FGFR3 | |
| AZD4547 | 1035270-39-3 | FGFR1, FGFR2, | RTK signaling |
| | | FGFR3 | |
| AZD6094 | SN1042487586 | MET | RTK signaling |
| AZD6482 | AZD 6482, AZD-6482, | PI3Kbeta | PI3K/MTOR signaling |
| | AK-55409 | | |
| AZD8055 | AZD-8055 | MTORC1, MTORC2 | PI3K/MTOR signaling |
| AZD8186 | SN1028722848 | PI3Kbeta, PI3Kdelta | PI3K/MTOR signaling |
| AZD8186 | AZD 8186, AZD-8186 | PI3Kalpha, PI3Kbeta | PI3K/MTOR signaling |
| AZD8835 | SN1040214596 | PI3Kalpha, PI3Kdelta | PI3K/MTOR signaling |
| AZD8931 | SN0216062465 | EGFR, ERBB2, | RTK signaling |
| | | ERBB3 | |
| Afatinib | BIBW2992, Tovok, | ERBB2, EGFR | EGFR signaling |
| | Gilotrif | | |
| Afuresertib | GSK2110183, | AKT1, AKT2, AKT3 | PI3K/MTOR signaling |
| | GSK2110183C | | |
| Alectinib | CH5424802, CH | ALK | RTK signaling |
| | 542802, Alecensa | | |
| Alpelisib | BYL719, BYL-719, | PI3Kalpha | PI3K/MTOR signaling |
| | NVP-BYL719 | | |
| Amuvatinib | MP470, MP 470, MP- | KIT, PDGFRA, FLT3 | RTK signaling |
| | 470 | | |
| Apitolisib | GDC0980 | mTOR, PI3K | PI3K/MTOR signaling |
| Axitinib | AG-13736, Inlyta | PDGFR, KIT, VEGFR | RTK signaling |
| BMS-536924 | BMS 536924 | IGF1R, IR | IGF1R signaling |
| BMS-754807 | BMS754807, BMS | IGF1R, IR | RTK signaling |
| | 754807 | | |
| Brivanib, BMS-540215 | | VEGFR, PDGFR | RTK signaling |
| Buparlisib | BKM120, NVP- | PI3Kalpha, PI3Kdelta, | PI3K/MTOR signaling |
| | BKM120 | PI3Kbeta, PI3Kgamma | |
| CI-1033 | | EGFR, ERBB2, | RTK signaling |
| | | ERBB4 | |
| CI-1040 | CI 1040, PD-18435, | MEK1, MEK2 | ERK MAPK signaling |
| | PD-184352, 212631- | | |
| | 79-3 | | |
| CP724714 | CP-724714 | ERBB2 | RTK signaling |
| CZC24832 | GTPL6653 | PI3Kgamma | PI3K/MTOR signaling |
| Cabozantinib | BMS-907351, XL-184, | VEGFR, MET, RET, | RTK signaling |
| | Cometriq | KIT, FLT1, FLT3, | |
| | | FLT4, TIE2, AXL | |
| Capivasertib | AZ5363 | AKT | PI3K/MTOR signaling |
| Cediranib | AZD2171, AZD 2171, | VEGFR, FLT1, FLT2, | RTK signaling |
| | AZD-2171, Recentin | FLT3, FLT4, KIT, | |
| | | PDGFRB | |
| Cetuximab | Erbitux, IMC-C225, | EGFR | EGFR signaling |
| | C225, IMC-225, | | |
| | L01XC06 | | |
| Crizotinib | Xalkori, PF2341066, | MET, ALK, ROS1 | RTK signaling |
| | PF-2341066, PF | | |
| | 2341066 | | |
| Dabrafenib | GSK2118436, Tafinlar | BRAF | ERK MAPK signaling |
| Dactolisib | NVP-BEZ235, BEZ235 | PI3K (class 1), | PI3K/MTOR signaling |
| | | MTORC1, MTORC2 | |
| Dasatinib | BMS-354825-03, | ABL, SRC, Ephrins, | RTK signaling |
| | BMS-354825, Sprycel | PDGFR, KIT | |
| ERK_2440 | SN1051032892, | ERK1, ERK2 | ERK MAPK signaling |
| | ERK_2440 | | |
| ERK_6604 | SN1047587618, | ERK1, ERK2 | ERK MAPK signaling |
| | ERK_6604 | | |
| EphB4_9721 | SN1076287848 | EPHB4 | RTK signaling |
| Erlotinib | Tarceva, RG-1415, CP- | EGFR | EGFR signaling |
| | 358774, OSI-774, Ro- | | |
| | 508231, R-1415 | | |
| FGFR_0939 | SN1043317881 | FGFR4 | RTK signaling |
| FGFR_3831 | SN1037992934 | FGFR1, FGFR2, | RTK signaling |
| | | FGFR3, FGFR4 | |
| FR-180204 | FR 180204, FR180204, | ERK1, ERK2 | ERK MAPK signaling |
| | ERK Inhibitor II | | |
| Foretinib | GSK1363089, XL-880, | MET, KDR, TIE2, | RTK signaling |
| | EXEL-2880, GSK089 | VEGFR3/FLT4, RON, | |
| | | PDGFR, FGFR1, | |
| | | EGFR | |
| GNE-317 | GNE317, GNE 317 | PI3Kalpha | PI3K/MTOR signaling |
| GSK1059615 | | PI3K | PI3K/MTOR signaling |
| GSK1904529A | GSK-1904529A, GSK | IGF1R, IR | RTK signaling |
| | 1904529A | | |
| GSK1904529A | GSK-1904529A, GSK | IGF1R, IR | IGF1R signaling |
| | 1904529A | | |
| GSK690693 | GSK 690693, GSK- | AKT1, AKT2, AKT3 | PI3K/MTOR signaling |
| | 690693 | | |
| GW-2580 | GX2580, CFMS | CSF1R | RTK signaling |
| | receptor tyrosine kinase | | |
| | inhibitor | | |
| GW441756 | GW 441756 | NTRK1 | RTK signaling |
| Gefitinib | ZD-1839, Iressa | EGFR | EGFR signaling |
| HG6-64-1 | KIN001-206 | BRAF | ERK MAPK signaling |
| IC-87114 | | PI3Kdelta | PI3K/MTOR signaling |
| IGF1R_3801 | SN1051640224, | IGFR1 | IGF1R signaling |
| | IGF1R_3801 | | |
| IGFR_3801 | SN1056768819 | IGFR1 | IGF1R signaling |
| Idelalisib | CAL-101, Zydelig | PI3Kdelta | PI3K/MTOR signaling |
| Ipatasertib | GDC0068, GDC 0068, | AKT1, AKT, AKT3 | PI3K/MTOR signaling |
| | GDC-0068 | | |
| JNJ38877605 | | MET | RTK signaling |
| JW-7-52-1 | NA | MTOR | PI3K/MTOR signaling |
| KIN001-236 | | Angiopoietin-1 | RTK signaling |
| | | receptor | |
| KIN001-266 | | MAP3K8 | ERK MAPK signaling |
| KRAS (G12C) | GTPL8020 | KRAS (G12C) | ERK MAPK signaling |
| Inhibitor-12 | | | |
| Kobe2602 | | RAS effector | RTK signaling |
| LJI308 | | RSK2, RSK1, RSK3 | PI3K/MTOR signaling |
| Lapatinib | Tykerb, Tyverb | EGFR, ERBB2 | RTK signaling |
| Lapatinib | Tykerb, Tyverb | EGFR, ERBB2 | EGFR signaling |
| Linifanib | ABT-869, ABT 869 | VEGFR1, VEGFR2, | RTK signaling |
| | | VEGFR3, CSF1R, | |
| | | FLT3, KIT | |
| Linsitinib | OSI-906, ASP-7487 | IGF1R | IGF1R signaling |
| MK-2206 | MK 2206, MK2206 | AKT1, AKT2 | PI3K/MTOR signaling |
| Masitinib | AB1010, Masivet | KIT, PDGFRA, | RTK signaling |
| | | PDGFRB | |
| NVP-ADW742 | NVP ADW742, | IGF1R | IGF1R signaling |
| | NVPADW742 | | |
| NVP-BHG712 | BHG712 | EPHB4 | RTK signaling |
| NVP-TAE684 | NVP-TAE 684, | ALK | RTK signaling |
| | TAE684, TAE-684 | | |
| OSI-027 | A-1065-5 | MTORC1, MTORC2 | PI3K/MTOR signaling |
| OSI-930 | OSI 930 OSI930 | KIT | RTK signaling |
| Omipalisib | GSK2126458, GSK- | PI3K (class 1), | PI3K/MTOR signaling |
| | 2126458, EX-8678, | MTORC1, MTORC2 | |
| | GSK458 | | |
| Osimertinib | AZD9291, AZD 9291, | EGFR | EGFR signaling |
| | AZD-9291, Tagrisso, | | |
| | Mereletinib | | |
| PD0325901 | PD-0325901, PD | MEK1, MEK2 | ERK MAPK signaling |
| | 0325901 | | |
| PD173074 | PD-173074, PD | FGFR1, FGFR2, | RTK signaling |
| | 173074 | FGFR3 | |
| PF-00299804 | | EGFR, ERBB2, | RTK signaling |
| | | ERBB4 | |
| PF-4708671 | PF 4708671, | S6K1 | PI3K/MTOR signaling |
| | PF4708671 | | |
| PHA-665752 | PHA665752, PHA | MET | RTK signaling |
| | 665752 | | |
| PI3Ka_4409 | SN1037097676 | PI3Kalpha, PI3Kdelta | PI3K/MTOR signaling |
| PIK-93 | PIK 93, PIK93 | PI3Kgamma | PI3K/MTOR signaling |
| PLK_6522 | SN1077317349 | PLK1, PLK2, PLK3 | Cell cycle |
| PLX-4720 | PLX4720, PLX 4720 | BRAF | ERK MAPK signaling |
| Pazopanib | Votrient | CSF1R, KIT, | RTK signaling |
| | | PDGFRA, PDGFRB | |
| Pelitinib | EKB-569, EKB 569 | EGFR | EGFR signaling |
| Pictilisib | GDC-0941, GDC0941, | PI3K (class 1) | PI3K/MTOR signaling |
| | RG-7621 | | |
| Pilaralisib | XL-147 | PI3K | PI3K/MTOR signaling |
| Quizartinib | AC220, AC 220, AC- | FLT3 | RTK signaling |
| | 220, Asp-2689 | | |
| RAF_9304 | SN1034259943 | ARAF, BRAF, CRAF | ERK MAPK signaling |
| Rapamycin | AY-22989, Sirolimus, | MTORC1 | PI3K/MTOR signaling |
| | WY-090217, Torisel, | | |
| | Rapamune | | |
| Refametinib | RDEA119, BAY-86- | MEK1, MEK2 | ERK MAPK signaling |
| | 9766, BAY 869766 | | |
| SB505124 | SB 505124, SB505124 | TGFBR1, ACVR1B, | RTK signaling |
| | | ACVR1C | |
| SB590885 | SB-590885 | BRAF | ERK MAPK signaling |
| SCH772984 | CS-1421 | ERK1, ERK2 | ERK MAPK signaling |
| SU11274 | | MET | RTK signaling |
| Sapitinib | AZD8931 | EGFR, ERBB2, | EGFR signaling |
| | | ERBB3 | |
| Savolitinib | AZD6094, Volitinib, | MET | RTK signaling |
| | AZD-6094, AZD 6094 | | |
| Selumetinib | AZD6244, AZD-6244, | MEK1, MEK2 | ERK MAPK signaling |
| | ARRY-886 | | |
| Selumetinib | SN1103949345, | MEK1, MEK2 | ERK MAPK signaling |
| | AZD1480 | | |
| Sorafenib | Nexavar, 284461-73-0, | PDGFR, KIT, VEGFR, | RTK signaling |
| | BAY 43-9006 | RAF | |
| Staurosporine | | Broad spectrum kinase | RTK signaling |
| | | inhibitor | |
| Sunitinib | Sutent, Sunitinib | PDGFR, KIT, VEGFR, | RTK signaling |
| | Malate, SU-11248 | FLT3, RET, CSF1R | |
| TGX221 | TGX-221, Tgx 221 | PI3Kbeta | PI3K/MTOR signaling |
| Taselisib | GDC-0032, GDC0032, | PI3K (beta sparing) | PI3K/MTOR signaling |
| | RG7604 | | |
| Temsirolimus | CCI-779, Torisel | MTOR | PI3K/MTOR signaling |
| Tivozanib | AV-951, AV 951, | VEGFR1, VEGFR2, | RTK signaling |
| | KRN-951, KIL8951, | VEGFR3 | |
| | ASP-4130 | | |
| Torin 2 | | mTOR | PI3K/MTOR signaling |
| Trametinib | GSK1120212, Mekinist | MEK1, MEK2 | ERK MAPK signaling |
| Ulixertinib | BVD-523, VRT752271 | ERK1, ERK2 | ERK MAPK signaling |
| Uprosertib | GSK2141795, | AKT1, AKT2, AKT3 | PI3K/MTOR signaling |
| | GSK2141795C, GSK- | | |
| | 2141795 | | |
| VX-11e | VX11e, VX11e | ERK2 | ERK MAPK signaling |
| Voxtalisib | XL-765, SAR245409 | PI3K (class 1), | PI3K/MTOR signaling |
| | | DNAPK, MTOR | |
| WYE-125132 | | mTOR | PI3K/MTOR signaling |
| XMD8-92 | XMD 8-92 | MAPK7 | ERK MAPK signaling |
| YM201636 | YM-201636, YM | PIKFYVE | PI3K/MTOR signaling |
| | 201636 | | |
| ZSTK474 | KIN001-167, ZSTK- | PI3K (class 1) | PI3K/MTOR signaling |
| | 474, ZSTK 474 | | |
| RMC-4550 | | SHP2 inhibitor | |
| RMC-4630 | | SHP2 inhibitor | |
| BBP-398 | IACS-15509 | SHP2 inhibitor | ERK MAPK signaling |
| JAB-3068 | | SHP2 inhibitor | ERK MAPK signaling |
| RLY-1971 | | SHP2 inhibitor | ERK MAPK signaling |
| ERAS-601 | | SHP2 inhibitor | ERK MAPK signaling |
| TNO155 | | SHP2 inhibitor | ERK MAPK signaling |

In some cases, a subject method of treatment includes a step, prior to the step of administration, of determining that an individual's cancer (e.g., lung adenocarcinoma) is oncogene-negative. The biological sample used for such determining can be any convenient type of sample. In some cases, the sample will be a biopsy sample from the individual. In some cases, the sample will include all or a portion of a tissue resection (e.g., tumor resection).

An individual's cancer (e.g., lung adenocarcinoma) can be determined to be oncogene-negative using any convenient method (e.g., genome sequencing such as using next generation/high throughput sequencing). In some embodiments, a subject method includes making such a determination (e.g., via assaying a biological sample of the cancer such as a biopsy or resection). In some embodiment, a subject method does not include such as a step because the step was already performed prior to performing the subject method (e.g., administering).

In some cases, prior to the administering step, the individual's oncogene-negative cancer (e.g., lung adenocarcinoma) can be determined to exhibit increased Ras/MAPK pathway activity. In some cases, prior to the administering step, the individual's oncogene-negative cancer (e.g., lung adenocarcinoma) can be determined to exhibit increased Ras/MAPK pathway activity and PI3K-AKT pathway activity. Such a determination can be made using any convenient methodology.

For example, in some cases an assay such as sequencing (e.g., genome sequencing) is used to detect mutations in member(s) of the Ras/MAPK pathway—or member(s) of the Ras/MAPK and PI3K-AKT pathways (see Table 2). If mutations are detected that are known to lead to increased pathway activity, then such an assay can be said to have detected increased pathway activity. While Table 2 is a non-exhaustive list, this table provides examples of members of the Ras/MAPK and PI3K-AKT pathways that may be useful in such an assay.

In some cases, an individual to be treated exhibits one or more mutations (loss of function) in one or more negative regulators of the Ras/MAPK pathway. In some cases, an individual to be treated exhibits one or more mutations (loss of function) in Nf1. In some cases, an individual to be treated exhibits one or more mutations (loss of function) in Rasa1. In some cases, an individual to be treated exhibits one or more mutations (loss of function) in Nf1 and one or more mutations (loss of function) in Rasa1. In some cases, an individual to be treated exhibits one or more mutations (gain of function) in one or more positive regulators of the Ras/MAPK pathway.

In some cases, an individual to be treated exhibits one or more mutations (loss of function) in one or more negative regulators of the PI3K-AKT pathway. In some cases, an individual to be treated exhibits one or more mutations (loss of function) in Pten. In some cases, an individual to be treated exhibits one or more mutations (gain of function) in one or more positive regulators of the PI3K-AKT pathway. In some cases, an individual to be treated exhibits one or more mutations (gain of function) in AKT1. In some cases, an individual to be treated exhibits one or more mutations (gain of function) in AKT2. In some cases, an individual to be treated exhibits one or more mutations (gain of function) in AKT3.

In some cases, an individual to be treated exhibits one or more mutations in a regulator of the Ras/MAPK pathway (e.g., loss of function in one or more negative regulators and/or gain of function in one or more positive regulators) and one or more mutations in a regulator of the PI3K-AKT pathway (e.g., loss of function in one or more negative regulators and/or gain of function in one or more positive regulators). In some cases, an individual to be treated exhibits one or more mutations (loss of function) in Nf1 and one or more mutations (loss of function) in Pten. In some cases, an individual to be treated exhibits one or more mutations (loss of function) in Rasa1 and one or more mutations (loss of function) in Pten. In some cases, an individual to be treated exhibits one or more mutations (loss of function) in Nf1, one or more mutations (loss of function) in Rasa1, and one or more mutations (loss of function) in Pten.

As discussed above, increased pathway activity can be the direct consequence of genomic alterations (e.g., substitution, deletion, insertion mutations) in positive and/or negative regulators of these pathways (see Table 2). However, increased pathway activity can also be caused indirectly, e.g., by epigenetic modifications, such that no genomic alterations are present in positive and/or negative regulators of these pathways. As an illustrative example, the expression level (and therefore activity) of a negative regulator such as PTEN can be reduced in the absence of a mutation in the PTEN-encoding gene itself—and such a scenario can still lead to increased PI3K-AKT pathway activity. Thus, in some cases, Ras/MAPK pathway activity and/or PI3K-AKT pathway activity is increased. In some such cases, genomic alteration(s) in a member(s) of the pathway are detected. In other such cases, genomic alterations in a member(s) of the pathway are not detected. In some cases, expression of a positive or negative regulator of the pathway is altered (increased or decreased, respectively) in the absence of a genomic alteration in the sequence encoding that regulator.

As such, in some cases, pathway activity can be measured without necessarily having information related to genomic alterations of pathway members. As an example, pathway activity can be measured by assaying the level of a biomarker of pathway activation—which can be considered to be a more direct way to assay for pathway activity than detecting mutations in pathway members. For example, in some cases phosphorylated ERK (pERK) is used as a biomarker of Ras/MAPK pathway activity. In some cases, phosphorylated AKT (pAKT) is used as a biomarker of PI3K-AKT pathway activity. As such, the level of such a biomarker(s) can be compared to a control, and an increase in the level of biomarker (relative to the control) can be used to indicate an increase in pathway activity, in which case this can be referred to as a positive assessment of pathway activity. A level of a biomarker of pathway activation can be measured using any convenient methodology (e.g., immunohistochemistry, Western blot, ELISA, mass spectrometry, and the like). For example, in some cases immunohistochemistry is used to determine whether biomarker levels are increased relative to a control. In some cases, a value is assigned to a detected increase. In some cases, the assessment is qualitative—for example in some cases, it is clear from looking at the sample (e.g., immunohistochemistry, western blot, ELISA) that there is increased pathway activity relative to normal/control samples—and there is not necessarily a need to provide a particular value of increase.

In some cases, both approaches (e.g., sequencing and protein detection) are used to measure pathway activity. In some such cases, a positive assessment of one of the two approaches is enough to determine that the individual's cancer has pathway activity. In other such cases, a positive assessment of both approaches is used to determine that the individual's cancer has pathway activity. In some cases, one of the approaches (e.g., sequencing or protein detection), but not the other is used to measure pathway activity and a positive assessment using that one approach is enough to determine that the individual's cancer has pathway activity. A positive assessment means that an increase in activity is detected when compared to a control value (e.g., the activity measured in a non-cancerous control, which activity can be a predetermined value or can be measured from a control sample around the same time that the sample from the individual is assayed).

In some cases, a positive assessment means that the pathway activity from the biological sample from the individual is 1.2-fold or more (e.g., 1.5-fold or more, 2-fold or more, 3-fold or more, 5-fold or more, or 10-fold or more) compared to a control value (e.g., a value from a non-cancerous control sample). In some cases, a positive assessment means that the pathway activity from the biological sample from the individual is 1.5-fold or more (e.g., 2-fold or more, 3-fold or more, 5-fold or more, or 10-fold or more) compared to a control value (e.g., a value from a non-cancerous control sample). In some cases, a positive assessment means that the pathway activity from the biological sample from the individual is 2-fold or more (e.g., 3-fold or more, 5-fold or more, or 10-fold or more) compared to a control value (e.g., a value from a non-cancerous control sample). In some cases, a positive assessment means that the pathway activity from the biological sample from the individual is 5-fold or more (e.g., 10-fold or more) compared to a control value (e.g., a value from a non-cancerous control sample).

### Control

The terms “reference value” and “control value” or sometimes simply “reference” or “control” as used herein mean a standardized value (e.g., that represents a standardized level, e.g., of a particular level of pathway activity such as Ras/MAPK or PI3K-AKT pathway activity, of a particular level of protein such as phosphorylated ERK (pERK) or phosphorylated AKT (pAKT), which can act as a readout of pathway activity, and the like) to be used to interpret the measured level(s) from an individual (a test individual). The reference value or control value is typically a nucleic acid or protein level that is obtained from a biological sample (e.g., cell/tissue such as a cancer cell or tumor) from an individual or cell, or an average value from multiple individuals or cells, with a known phenotype, e.g., cancerous cell, lung cancer cell, tumor cell, cell with increased pathway activity, cell with decreased pathway activity, cell with normal pathway activity, and the like.

For example, a level of pathway activity (e.g., Ras/MAPK pathway activity, PI3K-AKT pathway activity) of a test individual or cell can be compared with a reference value. In some cases the level of pathway activity can be determined by measuring the level of a particular readout protein (e.g., pERK for the Ras/MAPK pathway and/or pAKT for the PI3K-AKT pathway). In some cases the reference value is a predetermined threshold value (e.g., based on previous characterization of individuals/cells with normal, decreased, and/or increased pathway activity). In some cases the reference value is a value that is measured (e.g., a level of pathway activity) from an individual/cell with a known phenotype. In some cases the reference value is a value that is measured (e.g., a level of pathway activity) from an individual/cell with a known cancer phenotype (has a known cancer of interest such as a lung cancer and/or known to have increased pathway activity). In some cases the reference value is a value that is measured (e.g., a level of pathway activity) from an individual/cell known not to have a cancer phenotype (known not to have a cancer of interest such as a lung cancer and/or known to not exhibit increased pathway activity).

In some cases, e.g., if the reference is a measurement from an individual/cell without a cancer phenotype (and/or without increased pathway activity), then the test individual can be predicted to be a responder to treatment with a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) if the measured level(s) of the test individual is greater than the reference; and the test individual can be predicted not to be a responder to treatment with a subject agent if the measured level(s) of the test individual is less than or equal to the reference value.

In some cases, e.g., if the reference is a measurement from an individual/cell with a cancer phenotype (and/or with increased pathway activity), then the test individual can be predicted to be a responder to treatment with a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) if the measured level(s) of the test individual is greater than or equal to the reference (or roughly equal to, e.g., within 20%, 15%, 10%, or 5% of the reference); and the test individual can be predicted not to be a responder to treatment with a subject agent if the measured level(s) of the test individual is less than the reference value (e.g., less than and not within a 20% difference).

In some cases, a prognosis can be made by comparing a measured level of the individual with a reference value that is a known threshold (predetermined threshold value). For example, a measured level of an individual can be compared to reference values that are threshold values, where a score above (or in some cases equal to) the threshold is associated with a particular outcome (e.g., responder) and/or a score below (or in some cases equal to) the threshold is associated with a particular outcome (e.g., non-responder). In some cases, a measured level may be compared to two different reference values (e.g., one known to be associated with responders and one known to be associated with non-responders) to obtain confirmed information regarding whether the individual is a responder or a non-responder.

In some cases a control value is measured from different cells in the same individual. For example, in some cases (such as when performing screening methods as discussed elsewhere herein) a control value is measured from a tumor that is untreated or treated with a known placebo/vehicle/control agent, while the test value is measured from a different tumor in the same individual (e.g., a tumor into which a test/candidate agent was injected).

In some cases a control value is measured from a different individual. For example, in some cases (such as when performing screening methods as discussed elsewhere herein) a control value is measured from a control subject that is untreated or treated with a known placebo/vehicle/control agent, while the test value is measured from a different individual (an individual to whom a test/candidate agent was administered).

In some cases, a prognosis is a statistical likelihood of predicted responsiveness to treatment with a subject agent. Such statistical likelihoods can be obtained by comparing a measured level from an individual to reference values from a set of individuals with varying levels of responsiveness to treatment with a subject agent. Such comparisons can be used to correlate a range of measured levels to a range of responsiveness likelihoods. Thus, measured levels from an individual can be used to determine a statistical likelihood of responsiveness to a subject agent for the individual.

As another example, measured level may be employed to monitor treatment with a subject agent. By “monitor treatment” with a subject agent, it is generally meant monitoring a subject's condition, e.g. to provide information as to the effect or efficacy of a treatment.

### Delivery/Administration

A subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) can be administered by any suitable means (e.g., systemic or local), including topical, oral, parenteral, intravenous, intracranial, intratumoral, intrapulmonary, and intranasal. Parenteral infusions include intramuscular, intravenous (bollus or slow drip), intraarterial, intraperitoneal, intrathecal or subcutaneous administration. A subject agent can be administered in any manner which is medically acceptable. This may include by injection (e.g., by parenteral routes such as intravenous, intravascular, intraarterial, subcutaneous, intramuscular, intratumoral, intraperitoneal, intraventricular, intracranial, or intraepidermal), or others as well as oral, nasal, ophthalmic, rectal, or topical. Sustained release administration is also specifically included in the disclosure, by such means as depot injections or erodible implants. Some agents can also applied directly to the area after a tumor is resected, e.g., by local injection, or by placing drug infused patties. In some cases a subject agent will be delivered systemically. In some cases a subject agent will be delivered locally (e.g., direct injection such as into a tumor, i.e., intratumoral injection).

As noted above, a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) can be formulated with a pharmaceutically acceptable carrier (one or more organic or inorganic ingredients, natural or synthetic, with which a subject agent is combined to facilitate its application). A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. In some cases, an effective amount is an amount that reduces tumor size (e.g., lung tumor size) in the individual. An effective amount can be determined on an individual basis and can be based, in part, on consideration of the symptoms to be treated and results sought. An effective amount can be determined by one of ordinary skill in the art employing such factors and using no more than routine experimentation.

A subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) can be administered as a pharmaceutical composition comprising an active therapeutic agent(s) and another pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In some embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. A carrier may also bear a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) by non-covalent associations, such as non-covalent bonding or by encapsulation. The nature of the carrier can be either soluble or insoluble for purposes of the disclosure. Those skilled in the art will know of other suitable carriers for binding a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib), or will be able to ascertain such, using routine experimentation.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) can be determined by standard pharmaceutical procedures in cell cultures and/or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

### Formulations

A subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) can be prepared as a dosage unit, with a pharmaceutically acceptable excipient, with pharmaceutically acceptable salts and esters, etc. Compositions can be provided as pharmaceutical compositions.

Pharmaceutical Compositions. A suitable subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) can be provided in pharmaceutical compositions suitable for therapeutic use, e.g. for human treatment. In some embodiments, pharmaceutical compositions of the present disclosure include one or more therapeutic entities of the present disclosure (e.g., one or subject agents) and can include a pharmaceutically acceptable carrier, a pharmaceutically acceptable salt, a pharmaceutically acceptable excipient, and/or esters or solvates thereof. In some embodiments, the use of a subject agent includes use in combination with (co-administration with) another therapeutic agent (e.g., another agent for preventing or treating cancer such as lung cancer, e.g., lung adenocarcinoma). Therapeutic formulations comprising a subject agent can be prepared by mixing the agent(s) having the desired degree of purity with a physiologically acceptable carrier, a pharmaceutically acceptable salt, an excipient, and/or a stabilizer (Remington's Pharmaceutical Sciences 16th edition, Osol, A. Ed. (1980)) (e.g., in the form of lyophilized formulations or aqueous solutions). A composition having a subject agent can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this disclosure can be present in unsalified or unesterified form, or in salified and/or esterified form, and the naming of such compounds is intended to include both the original (unsalified and unesterified) compound and its pharmaceutically acceptable salts and esters.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

A “therapeutically effective dose” or “therapeutically effective amount” or “therapeutic dose” is an amount sufficient to effect desired clinical results (i.e., achieve therapeutic efficacy), e.g., reduced tumor size, stabilization of tumor size (e.g., prevention of increased tumor size), reduction or stabilization in the number of cancer cells present in the individual, prevention of metastasis, and the like. A therapeutically effective dose can be administered in one or more administrations. For purposes of this disclosure, a therapeutically effective dose of a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) is an amount that is sufficient to palliate, ameliorate, stabilize, reverse, prevent, slow or delay the progression of the disease state (e.g., lung cancer). Thus, in some cases, a therapeutically effective dose of a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) reduces the size of a tumor (e.g., lung tumor such as a lung adenocarcinoma tumor). In some cases, a therapeutically effective dose of a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) stabilizes the size of a tumor (e.g., lung tumor such as a lung adenocarcinoma tumor). In some cases, a therapeutically effective dose of a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) reduces or stabilized the growth rate of a tumor (e.g., lung tumor such as a lung adenocarcinoma tumor). In some cases, a therapeutically effective dose of a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) increases the life span of the individual being treated. In some cases, a therapeutically effective dose of a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) improves the quality of life for the individual being treated. In some case, treatment using a subject method results in long term regression of the cancer such as lung cancer (e.g., increases the chance of survival of the individual being treated).

A single therapeutically effective dose or a series of therapeutically effective doses would be able to achieve a desired result in an individual (e.g., reducing or stabilizing lung tumor size). A therapeutically effective dose of a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) can depend on the specific agent used, and in some cases can be 0.5 mg/kg body weight or more (e.g., 1 mg/kg or more, 2 mg/kg or more, 3 mg/kg or more, 4 mg/kg or more, 5 mg/kg or more, 6 mg/kg or more, 7 mg/kg or more, 8 mg/kg or more, 9 mg/kg or more, 10 mg/kg or more, 15 mg/kg or more, 20 mg/kg or more, 25 mg/kg or more, 30 mg/kg or more, 35 mg/kg or more, 40 mg/kg or more, 45 mg/kg or more, 50 mg/kg or more, 55 mg/kg or more, 60 mg/kg or more, 65 mg/kg or more, or 70 mg/kg or more) independently for each agent.

In some cases, a therapeutically effective dose of a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) can be in a range of from 0.5 mg/kg to 200 mg/kg (e.g., from 1 to 150 mg/kg, from 1 to 100 mg/kg, from 1 to 90 mg/kg, from 1 to 90 mg/kg, from 1 to 85 mg/kg, from 1 to 80 mg/kg, from 1 to 70 mg/kg, from 1 to 60 mg/kg, from 1 to 50 mg/kg, from 1 to 40 mg/kg, from 1 to 30 mg/kg, from 1 to 20 mg/kg, from 1 to 10 mg/kg, from 5 to 200 mg/kg, from 5 to 150 mg/kg, from 5 to 100 mg/kg, from 5 to 90 mg/kg, from 5 to 90 mg/kg, from 5 to 85 mg/kg, from 5 to 80 mg/kg, from 5 to 70 mg/kg, from 5 to 60 mg/kg, from 5 to 50 mg/kg, from 5 to 40 mg/kg, from 5 to 30 mg/kg, from 5 to 20 mg/kg, from 5 to 10 mg/kg, from 10 to 200 mg/kg, from 10 to 150 mg/kg, from 10 to 100 mg/kg, from 10 to 90 mg/kg, from 10 to 85 mg/kg, from 10 to 80 mg/kg, from 10 to 70 mg/kg, from 10 to 60 mg/kg, from 10 to 50 mg/kg, from 10 to 40 mg/kg, from 10 to 30 mg/kg, from 10 to 20 mg/kg, from 20 to 200 mg/kg, from 20 to 150 mg/kg, from 20 to 100 mg/kg, from 20 to 90 mg/kg, from 20 to 85 mg/kg, from 20 to 80 mg/kg, from 20 to 70 mg/kg, from 20 to 60 mg/kg, from 20 to 50 mg/kg, from 20 to 40 mg/kg, from 20 to 30 mg/kg, from 40 to 200 mg/kg, from 40 to 150 mg/kg, from 40 to 100 mg/kg, from 40 to 90 mg/kg, from 40 to 85 mg/kg, from 40 to 80 mg/kg, from 40 to 70 mg/kg, from 55 to 200 mg/kg, from 55 to 150 mg/kg, from 55 to 100 mg/kg, from 55 to 90 mg/kg, from 55 to 85 mg/kg, from 55 to 80 mg/kg, or from 55 to 70 mg/kg) independently for each agent.

In some cases, a therapeutically effective dose of a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) can be in a range of from 1 mg/kg to 50 mg/kg (e.g., from 1 to 40 mg/kg, from 1 to 30 mg/kg, from 1 to 20 mg/kg, from 5 to 50 mg/kg, from 5 to 40 mg/kg, from 5 to 30 mg/kg, from 5 to 20 mg/kg, from 10 to 50 mg/kg, from 10 to 40 mg/kg, from 10 to 30 mg/kg, from 10 to 20 mg/kg, or from 20 mg/kg to 40 mg/kg) independently for each agent. In some cases, a therapeutically effective dose of a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) can be in a range of from 10 mg/kg to 40 mg/kg (e.g., from 10 to 35 mg/kg, or from 10 to 30 mg/kg, or from 20 mg/kg to 40 mg/kg) independently for each agent.

In some cases, a therapeutically effective dose of a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) can be in a range of from 25 mg/kg to 100 mg/kg (e.g., from 25 to 100 mg/kg, from 40 to 100 mg/kg, from 50 to 100 mg/kg, from 60 to 100 mg/kg, from 25 to 90 mg/kg, from 40 to 90 mg/kg, from 50 to 90 mg/kg, or from 60 to 90 mg/kg) independently for each agent. In some cases, a therapeutically effective dose of a subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) can be in a range of from 60 mg/kg to 90 mg/kg (e.g., from 65 to 85 mg/kg, or from 70 to 80 mg/kg) independently for each agent.

A subject agent (e.g., an inhibitor of Ras/MAPK pathway activity, an inhibitor of PI3K-AKT pathway activity, or both, e.g., a SHP2 inhibitor such as RMC-4550 or RMC-4630 and/or an AKT inhibitor such as capivasertib) can be administered in any convenient amount using any convenient dosing regimen. The following are additional illustrative examples.

In some embodiments, an inhibitor of PI3K-AKT pathway activity (e.g., an inhibitor of AKT such as A-443654, AKT inhibitor VIII, AT13148, AT7867, Afuresertib, Capivasertib, GSK690693, Ipatasertib, MK-2206, or Uprosertib—e.g., capivasertib) is administered orally, twice daily, e.g., in some cases for 4 days on followed by 3 days off. In some cases the subject is dosed on Days 2 to 5 of Weeks 1, 2, and 3 followed by 1 week off-treatment within each 28-day treatment cycle.

In some cases (e.g., for either of the dosing regimens described above), 100-600 mg are administered per dose (e.g., 100-550, 100-500, 100-450, 100-400, 150-600, 150-550, 150-500, 150-450, 200-600, 200-550, 200-500, 200-450, 350-600, 350-550, 350-500, 350-450, 400-600, 400-550, 400-500, 400-450, 450-600, 450-550, 450-500, about 200 mg, about 320 mg, about 400 mg, or about 480 mg). In some such cases, capivasertib is administered. In some cases, the dose is about 200 mg. In some cases, the dose is about 300 mg. In some cases, the dose is about 420 mg. In some cases, the dose is about 480 mg. In some cases, the dose is 50-200 mg/kg (e.g., 50-150, 50-120, 75-200, 75-150, 75-120, or about 100 mg/kg).

In some embodiments, an inhibitor of Ras/MAPK pathway activity (e.g., a SHP2 inhibitor such as RMC-4550, RMC-4630, BBP-398, JAB-3068, RLY-1971, ERAS-601, or TNO155—e.g., RMC-4550 or RMC-4630) is administered on a Day 1/Day 2 (D1D2) weekly schedule. In some cases 100-600 mg are administered per dose (e.g., 100-550, 100-500, 100-450, 100-400, 150-600, 150-550, 150-500, 150-450, 200-600, 200-550, 200-500, 200-450, 350-600, 350-550, 350-500, 350-450, 400-600, 400-550, 400-500, 400-450, 450-600, 450-550, 450-500, about 200 mg, about 320 mg, about 400 mg, or about 480 mg). In some cases, RMC-4550 is administered. In some cases, RMC-4630 is administered. In some cases, the dose is 5-50 mg/kg (e.g., 5-40, 10-50, 10-40, 20-50, 20-40, or about 30 mg/kg).

The dose required to achieve a desired result can be proportional to the amount of time between doses and inversely proportional to the number of doses administered. Thus, as the frequency of dosing increases, the required dose decreases. The optimization of dosing strategies will be readily understood and practiced by one of ordinary skill in the art.

Dosage and frequency may vary depending on the half-life of the agent in the patient. It will be understood by one of skill in the art that such guidelines will be adjusted for the molecular weight of the active agent. The dosage may also be varied for localized administration, e.g. intracranial, or for systemic administration, e.g. i.m., i.p., i.v., and the like.

### Co-Administration

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

Treatment with subject agent(s) (e.g., an inhibitor of Ras/MAPK pathway activity, an inhibitor of PI3K-AKT pathway activity) can be combined with another therapy such as chemotherapy, radiotherapy, and/or immunotherapies to enhance effect. For example, for any of the below scenarios, agents (e.g., ‘agent 1’ and/or ‘agent 2’) can be co-administered with another agent such as a cancer therapeutic drug (e.g., a tumor-directed antibody).

In some cases, a first agent 1 (agent 1) (e.g., formulated as a pharmaceutical composition) is co-administered with another agent (agent 2). In some cases agent 1 is an inhibitor of Ras/MAPK pathway activity (e.g., a SHP2 inhibitor such as RMC-4550) and agent 2 is another (different) inhibitor of Ras/MAPK pathway activity. In some cases agent 1 is an inhibitor of Ras/MAPK pathway activity (a SHP2 inhibitor such as RMC-4550) and agent 2 is an inhibitor of PI3K-AKT pathway activity (e.g., an AKT inhibitor such as capivasertib). In some cases agent 1 is an inhibitor of PI3K-AKT pathway activity (e.g., an AKT inhibitor such as capivasertib) and agent 2 is another (different) inhibitor of PI3K-AKT pathway activity.

Co-administration may involve concurrent (i.e. at the same time), prior, or subsequent administration of the drug/antibody with respect to the administration of an agent or agents of this disclosure. In some cases, a subject agent (e.g., an inhibitor of Ras/MAPK pathway activity, an inhibitor of PI3K-AKT pathway activity, or both) is formulated with one or more agents that potentiate activity, or that otherwise increase the therapeutic effect (such as an immunomodulatory agent, a tumor-directed antibody, and the like).

In some cases, a subject agent (e.g., an inhibitor of Ras/MAPK pathway activity, an inhibitor of PI3K-AKT pathway activity, or both) is used in a combination therapy (is co-administered) with a cancer targeting agent (e.g., an agent that specifically binds a cancer antigen, e.g., a cell-specific antibody selective for a tumor cell marker). Any convenient cancer cell targeting agent can be used. In some cases, the cancer cell targeting agent is a specific binding agent (e.g., a polypeptide such as an antibody that includes an antigen binding region specific for a cancer antigen) that specifically binds a cancer antigen of cancer cells (e.g., CD19, CD20, CD22, CD24, CD25, CD30, CD33, CD38, CD44, CD47, CD52, CD56, CD70, CD96, CD97, CD99, CD123, CD279 (PD-1), CD274 (PD-L1), EpCam, EGFR, 17-1A, HER2, CD117, C-Met, PTHR2, HAVCR2 (TIM3), and SIRPA). As such, in some cases, a subject method includes co-administering a subject agent (e.g., an inhibitor of Ras/MAPK pathway activity, an inhibitor of PI3K-AKT pathway activity, or both) and a cancer cell targeting agent that is a specific binding agent (e.g., a polypeptide such as an antibody that includes an antigen binding region specific for a cancer antigen) that specifically binds an antigen (e.g., a cancer antigen) selected from: CD19, CD20, CD22, CD24, CD25, CD30, CD33, CD38, CD44, CD47, CD52, CD56, CD70, CD96, CD97, CD99, CD123, CD279 (PD-1), CD274 (PD-L1), EpCam, EGFR, 17-1A, HER2, CD117, C-Met, PTHR2, HAVCR2 (TIM3), and SIRPA.

In some cases, a subject agent (e.g., an inhibitor of Ras/MAPK pathway activity, an inhibitor of PI3K-AKT pathway activity, or both) is used in a combination therapy (is co-administered) with: cetuximab (binds EGFR), panitumumab (binds EGFR), rituximab (binds CD20), trastuzumab (binds HER2), pertuzumab (binds HER2), alemtuzumab (binds CD52), brentuximab (binds CD30), tositumomab, ibritumomab, gemtuzumab, ibritumomab, or edrecolomab (binds 17-1A), or any combination thereof.

In some cases, a subject agent (e.g., an inhibitor of Ras/MAPK pathway activity, an inhibitor of PI3K-AKT pathway activity, or both) is used in a combination therapy (is co-administered) with a lung cancer drug—for example in some cases with: carboplatin, cisplatin, docetaxel (taxotere), gemcitabine (gemzar), nab-paclitaxel (abraxane), paclitaxel (taxol), pemetrexed (alimta), or vinorelbine (navelbine), or any combination thereof.

In some cases, a subject agent (e.g., an inhibitor of Ras/MAPK pathway activity, an inhibitor of PI3K-AKT pathway activity, or both) is used in a combination therapy (is co-administered) with a lung cancer drug—for example in some cases with: Abraxane (Paclitaxel Albumin-stabilized Nanoparticle Formulation), Afatinib Dimaleate, Afinitor (Everolimus), Afinitor Disperz (Everolimus), Alecensa (Alectinib), Alectinib, Alimta (Pemetrexed Disodium), Alunbrig (Brigatinib), Atezolizumab, Avastin (Bevacizumab), Bevacizumab, Brigatinib, Capmatinib Hydrochloride, Carboplatin, Cemiplimab-rwlc, Ceritinib, Crizotinib, Cyramza (Ramucirumab), Dabrafenib Mesylate, Dacomitinib, Docetaxel, Doxorubicin Hydrochloride, Durvalumab, Entrectinib, Erlotinib Hydrochloride, Everolimus, Gavreto (Pralsetinib), Gefitinib, Gilotrif (Afatinib Dimaleate), Gemcitabine Hydrochloride, Gemzar (Gemcitabine Hydrochloride), Imfinzi (Durvalumab), Infugem (Gemcitabine Hydrochloride), Ipilimumab, Iressa (Gefitinib), Keytruda (Pembrolizumab), Libtayo (Cemiplimab-rwlc), Lorbrena (Lorlatinib), Lorlatinib, Mekinist (Trametinib Dimethyl Sulfoxide), Methotrexate Sodium, Mvasi (Bevacizumab), Necitumumab, Nivolumab, Opdivo (Nivolumab), Osimertinib Mesylate, Paclitaxel, Paclitaxel Albumin-stabilized Nanoparticle Formulation, Paraplat (Carboplatin), Paraplatin (Carboplatin), Pembrolizumab, Pemetrexed Disodium, Portrazza (Necitumumab), Pralsetinib, Ramucirumab, Retevmo (Selpercatinib), Rozlytrek (Entrectinib), Selpercatinib, Tabrecta (Capmatinib Hydrochloride), Tafinlar (Dabrafenib Mesylate), Tagrisso (Osimertinib Mesylate), Tarceva (Erlotinib Hydrochloride), Taxotere (Docetaxel), Tecentriq (Atezolizumab), Tepmetko (Tepotinib Hydrochloride), Tepotinib Hydrochloride, Trametinib Dimethyl Sulfoxide, Trexall (Methotrexate Sodium), Vizimpro (Dacomitinib), Vinorelbine Tartrate, Xalkori (Crizotinib), Yervoy (Ipilimumab), Zirabev (Bevacizumab), Zykadia (Ceritinib), Etopophos (Etoposide Phosphate), Etoposide, Hycamtin (Topotecan Hydrochloride), or Zepzelca (Lurbinectedin), or any combination thereof.

In some cases, a subject agent (e.g., an inhibitor of Ras/MAPK pathway activity, an inhibitor of PI3K-AKT pathway activity, or both), is used in a combination therapy (is co-administered) with an immunomodulatory agent. Any convenient immunomodulatory agent can be used. In some cases, the immunomodulatory agent is selected from: an anti-CTLA4 antibody; an anti-PD-1/PD-L1 agent (e.g., an anti-PD-1 antibody, a PD-1-binding reagent such as a PD-L1 or PD-L2 ectodomain, an anti-PD-L1 antibody, a PD-L1-binding reagent such as a PD-1 ectodomain, and the like); a CD40 agonist (e.g., CD40L); a 4-1BB modulator (e.g., a 4-1BB-agonist); an anti-CD47/SIRPA agent (e.g., an anti-CD47 antibody, a CD47-binding reagent such as a SIRPA ectodomain, an anti-SIRPA antibody, a SIRPA-binding reagent such as a CD47 ectodomain, and the like); an inhibitor of TIM3 and/or CEACAM1; an inhibitor of TIM3 and/or CEACAM1; an inhibitor of BTLA and/or CD160; and the like.

### Treatment

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it (prophylactic); (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment can include those already inflicted (e.g., those with cancer, e.g. those having tumors) as well as those in which prevention is desired (e.g., those with increased susceptibility to cancer; those with pre-cancerous tumors, lesions; those suspected of having cancer; etc.).

The terms “recipient”, “individual”, “subject”, “host”, and “patient”, are used interchangeably herein and refer to any mammalian subject for whom diagnosis, treatment, or therapy is desired (e.g., humans). “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human. In some embodiments, the mammal is a rodent (e.g., rat, mouse). In some embodiments, the mammal is a non-human primate.

A therapeutic treatment is one in which the subject is inflicted prior to administration and a prophylactic treatment is one in which the subject is not inflicted prior to administration. In some embodiments, the subject has an increased likelihood of becoming inflicted or is suspected of having an increased likelihood of becoming inflicted (e.g., relative to a standard, e.g., relative to the average individual, e.g., a subject may have a genetic predisposition to cancer and/or a family history indicating increased risk of cancer), in which case the treatment can be a prophylactic treatment.

### Individuals to be Treated and ‘Cancer Cells’

In some embodiments, the individual to be treated is an individual with cancer. As used herein “cancer” includes any form of cancer (e.g., leukemia; acute myeloid leukemia (AML); acute lymphoblastic leukemia (ALL); lymphomas; mesothelioma (MSTO); minimal residual disease; solid tumor cancers, e.g., lung, prostate, breast, bladder, colon, ovarian, pancreas, kidney, glioblastoma, medulloblastoma, leiomyosarcoma, and head & neck squamous cell carcinomas, melanomas; etc.), including both primary and metastatic tumors; and the like. In some cases, the individual has recently undergone treatment for cancer (e.g., radiation therapy, chemotherapy, surgical resection, etc.) and are therefore at risk for recurrence. Any and all cancers are suitable cancers to be treated by the subject methods, compositions, and kits. In some cases, the individual to be treated has lung cancer (e.g., lung adenocarcinoma).

For example, when taking the above into account, in some cases the individual to be treated has an oncogene-negative cancer. In some such cases, the individual to be treated has an oncogene-negative lung cancer (e.g., lung adenocarcinoma). In some cases, the individual to be treated has an oncogene-negative lung adenocarcinoma.

In some cases the individual to be treated is susceptible to, or is suspected of having an increased risk of acquiring (suspected of being susceptible to) cancer. In some such cases, the individual to be treated is susceptible to, or is suspected of having an increased risk of acquiring (suspected of being susceptible to) lung cancer (e.g., lung adenocarcinoma). In some cases, the individual to be treated is susceptible to, or is suspected of having an increased risk of acquiring (suspected of being susceptible to) lung adenocarcinoma.

The terms “cancer,” “neoplasm,” and “tumor” are used herein to refer to cells which exhibit autonomous, unregulated growth, such that they exhibit an aberrant growth phenotype characterized by a significant loss of control over cell proliferation. Cells of interest for detection, analysis, and/or treatment in the present disclosure include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell (e.g., from any of the cancers for which an individual can be treated, e.g., isolated from an individual having cancer) or is derived from a cancer cell e.g. clone of a cancer cell. For example, a cancer cell can be from an established cancer cell line, can be a primary cell isolated from an individual with cancer, can be a progeny cell from a primary cell isolated from an individual with cancer, and the like. In some cases, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, myelomas, etc., and circulating cancers such as leukemias.

In some cases a subject cancer cell is a lung cell. In some cases a subject cancer cell is a cell of (or from) a lung adenocarcinoma. In some cases a subject cancer cell is a cell of (or from) a lung tumor.

As used herein “cancer” includes any form of cancer, including but not limited to solid tumor cancers (e.g., lung, prostate, breast, bladder, colon, ovarian, pancreas, kidney, liver, glioblastoma, medulloblastoma, leiomyosarcoma, head & neck squamous cell carcinomas, melanomas, neuroendocrine; etc.) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors. Any cancer is a suitable cancer to be treated by the subject methods and compositions.

Carcinomas are malignancies that originate in the epithelial tissues. Epithelial cells cover the external surface of the body, line the internal cavities, and form the lining of glandular tissues. Examples of carcinomas include, but are not limited to: adenocarcinoma (cancer that begins in glandular (secretory) cells), e.g., cancers of the breast, pancreas, lung, prostate, and colon can be adenocarcinomas; adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, skin, etc.

Soft tissue tumors are a highly diverse group of rare tumors that are derived from connective tissue. Examples of soft tissue tumors include, but are not limited to: alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; and pleomorphic adenoma of soft tissue; and neoplasias derived from fibroblasts, myofibroblasts, histiocytes, vascular cells/endothelial cells and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to: askin's tumor; sarcoma botryoides; chondrosarcoma; ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi's sarcoma; leiomyosarcoma; liposarcoma; lymphangiosarcoma; malignant peripheral nerve sheath tumor (MPNST); neurofibrosarcoma; synovial sarcoma; undifferentiated pleomorphic sarcoma, and the like).

A teratoma is a type of germ cell tumor that may contain several different types of tissue (e.g., can include tissues derived from any and/or all of the three germ layers: endoderm, mesoderm, and ectoderm), including for example, hair, muscle, and bone. Teratomas occur most often in the ovaries in women, the testicles in men, and the tailbone in children.

Melanoma is a form of cancer that begins in melanocytes (cells that make the pigment melanin). It may begin in a mole (skin melanoma), but can also begin in other pigmented tissues, such as in the eye or in the intestines.

Leukemias are cancers that start in blood-forming tissue, such as the bone marrow, and causes large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to: Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One kind is Hodgkin lymphoma (HL), which is marked by the presence of a type of cell called the Reed-Sternberg cell. There are currently 6 recognized types of HL. Examples of Hodgkin lymphomas include: nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to: AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to: gliomas (e.g., glioblastomas, astrocytomas, oligodendrogliomas, ependymomas, and the like), meningiomas, pituitary adenomas, vestibular schwannomas, primitive neuroectodermal tumors (medulloblastomas), etc.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body.

### Genetically Modified Cells and Non-Human Genetically Modified Organisms

The present disclosure provides genetically modified cells and non-human genetically modified organisms (e.g., mammal, rodent, mouse, rat, pig, horse, sheep, cow, ungulate, non-human primate) that have an oncogene-negative profile and one or more genomic alterations causing increased Ras/MAPK pathway activity and/or increased PI3K-AKT pathway activity.

Examples of specific genes referred to herein include Nf1, Rasa1, Pten, and AKT (AKT1, AKT2, AKT3). As would be known to one of ordinary skill in the art, the human orthologs are as follows:

### Ras/MAPK Pathway

Neurofibromin (NF1) (UniProtKB P21359)
Ras GTPase-activating protein 1 (RASA1) (UniProtKB P20936)
   PI3K-AKT pathway
PTEN (phosphatidylinositol 3,4,5-trisphosphate 3-phosphatase and dual-specificity protein phosphatase PTEN) (phosphatase and tensin homolog) (also referred to as PTEN1) (UniProtKB P60484)
AKT1 (RAC-alpha serine/threonine-protein kinase) (UniProtKB P31749)
AKT2 (RAC-beta serine/threonine-protein kinase) (UniProtKB P31751)
AKT3 (RAC-gamma serine/threonine-protein kinase) (UniProtKB Q9Y243)

Table 2 above provides additional genes/proteins from these pathways.

In some cases a subject genetically modified cell or organism (e.g., rodent such as a mouse) is modified such that it has one or more genomic alterations (e.g., via mutation such as substitution, deletion, insertion) causing increased Ras/MAPK pathway activity. In some cases a subject cell or organism (e.g., rodent such as a mouse) is modified such that it has one or more genomic alterations (e.g., via mutation such as substitution, deletion, insertion) causing increased Ras/MAPK pathway activity and increased PI3K-AKT pathway activity.

In some cases, the one or more genomic alterations is present throughout the organism's body (e.g., was present in the germline of one or both parents such that the alteration is distributed throughout the entire body). In some cases, a subject genetically modified organism is a chimeric animal in which some cells harbor the genomic alteration(s) and some cells do not. Chimeric animals can be generated in a number of different ways. For example, a chimeric animal can be generated at the embryo stage by injecting a stem cell with the genomic alteration(s) into an embryo that does not include the genomic alteration(s) (e.g., injection of an embryonic stem cell (ESC) into the cavity (blastocoel) of a blastocyst). Another common method for generating a chimeric animal at the embryo stage includes well sandwich aggregation between zona pellucida (ZP) removed (denuded) post-coitum embryos and ESC clumps. Chimeric animals can also be generated post-development, e.g., in juveniles and adults by generating the genomic alteration(s) in some cells but not others. For example, a desired combination of tools such as cell specific delivery (e.g., aerosol delivery to the lungs, local injection, ligand target delivery vehicles, and the like), Cre/Lox and/or Flp/FRT recombination, site-specific genome targeting effectors (e.g., CRISPR/Cas effectors such as type II effectors (e.g., Cas9), type V effectors (e.g., Cas12), Zinc finger nucleases, TALENs, and the like), and the like, can be deployed to introduce genomic alterations to targeted cells such as lung cells. As another non-limiting example, haematopoietic cells (HSCs) such as HSCs that harbor the one or more genomic alterations can be introduced into the blood (e.g., via HSC transplant into a host such as an irradiated host).

As would be understood by one of ordinary skill in the art, increased Ras/MAPK pathway activity can be caused by reducing activity and/or expression (‘loss of function mutation’) of a negative regulator of the Ras/MAPK pathway or by increasing activity/expression (‘gain of function mutation’) of a positive regulator of the Ras/MAPK pathway. Likewise, increased PI3K-AKT pathway activity can be caused by reducing activity and/or expression (‘loss of function mutation’) of a negative regulator of the PI3K-AKT pathway or by increasing activity/expression (‘gain of function mutation’) of a positive regulator of the PI3K-AKT pathway. Table 2 includes a non-exhaustive list of examples of genetic alterations that lead to activation of these pathways.

In some cases, the increased Ras/MAPK pathway activity is caused by a genomic alteration that causes reduced expression and/or activity of wild type Nf1 or Rasa1 or any combination of negative regulators of the Ras/MAPK pathway (see, e.g., Table 2). In some cases, the increased Ras/MAPK pathway activity is caused by a genomic alteration that causes increased expression and/or activity of any combination of positive regulator(s) of the Ras/MAPK pathway (see, e.g., Table 2). In some cases, the increased Ras/MAPK pathway activity is caused by a genomic alteration that causes reduced expression and/or activity of wild type Nf1. In some cases, the increased Ras/MAPK pathway activity is caused by a genomic alteration that causes reduced expression and/or activity of wild type Rasa1. In some cases, the increased Ras/MAPK pathway activity is caused by a genomic alteration that causes reduced expression and/or activity of wild type Nf1 and a genomic alteration that causes reduced expression and/or activity of wild type wild type Rasa1. In some cases, the increased Ras/MAPK pathway activity is caused by a combination of genomic alterations that cause reduced expression and/or activity of a negative regulator (e.g., wild type Nf1 and/or Rasa1) and that cause increased expression and/or activity of a positive regulator of the Ras/MAPK pathway (see, e.g., Table 2).

In some cases, increased PI3K-AKT pathway activity is caused by a genomic alteration that causes reduced expression and/or activity of wild type Pten (also referred to as Pten1) or any combination of negative regulators of the PI3K-AKT pathway (see, e.g., Table 2). In some cases, increased PI3K-AKT pathway activity is caused by a genomic alteration that causes reduced expression and/or activity of wild type Pten. In some cases, the increased PI3K-AKT pathway activity is caused by a genomic alteration that causes increased expression and/or activity of any combination of positive regulator(s) of the PI3K-AKT pathway (see, e.g., Table 2). In some cases, the increased PI3K-AKT pathway activity is caused by a pathway-activating alteration of a positive pathway regulator such as AKT (e.g., myristoylated AKT1). In some cases, the increased PI3K-AKT pathway activity is caused by a combination of genomic alterations that cause reduced expression and/or activity of a negative regulator (e.g., wild type Pten) and that cause increased expression and/or activity of a positive regulator (e.g., AKT) of the PI3K-AKT pathway (see, e.g., Table 2).

In cases where a subject genetically modified cell or organism (e.g., rodent such as a mouse) is modified such that it has genomic alterations (e.g., via mutation such as substitution, deletion, insertion) causing increased Ras/MAPK pathway activity and increased PI3K-AKT pathway activity, any convenient combination of alterations is envisioned. For example, in some cases such cells and/or organisms include genomic alterations that cause reduced expression and/or activity of a negative regulator of Ras/MAPK pathway activity (e.g., wild type Nf1 and/or Rasa1) and also include genomic alterations that cause reduced expression and/or activity of a negative regulator of PI3K-AKT pathway activity (e.g., wild type Pten). As an example, in some cases, a subject genetically modified cell or organism includes a genomic alteration that causes reduced expression and/or activity of Nf1, a genomic alteration that causes reduced expression and/or activity of Rasa1, and a genomic alteration that causes reduced expression and/or activity of Pten. As another example, in some cases, a subject genetically modified cell or organism includes a genomic alteration that causes reduced expression and/or activity of Nf1, a genomic alteration that causes reduced expression and/or activity of Rasa1, and a pathway-activating alteration of AKT (e.g., myristoylated AKT1).

In some cases a cell or organism includes an agent that targets expression of a gene/protein encoded by a locus of interest (e.g., Nf1, Rasa1, Pten). For example in some cases a cell or organism includes a nucleic acid (such as a DNA that encodes an RNAi agent) that targets expression from the locus of interest (e.g., thereby affecting protein levels such as Nf1, Rasa1, Pten, and the like).

Reducing (inhibiting) expression and/or function of a gene herein refers to reducing protein production (the gene's expression) from the endogenous locus and/or inhibiting the function of the protein that is produced from the endogenous locus (e.g., via genetic mutation resulting in partial or total loss of function allele(s), via small molecule drug, antibody, and the like). Reducing function of an endogenous gene can be considered to encompass inhibiting/reducing expression of the gene (e.g., by reducing the total amount of protein produced) as well inhibiting/reducing function of a gene product (e.g., protein) encoded/produced by the endogenous gene (e.g., using a small molecule drug, antibody, etc.)—either way, the overall level of function provided by the endogenous locus is reduced/inhibited/blocked.

As would be readily understood by one of ordinary skill in the art, one can reduce expression (protein production) of an endogenous gene at the DNA, RNA, or protein level. For example, expression can be reduced by reducing the total amount of wild type protein made by the endogenous locus, and this can be accomplished either by changing the nature of the protein produced (e.g., via gene mutation to generate a loss of function allele such as a null allele or an allele that encodes a protein reduced function) or by reducing the overall levels of protein produced without changing the nature of the protein itself.

Reducing (inhibiting) expression and/or function of an endogenous gene can be accomplished using any convenient method and one of ordinary skill in the art will be aware of multiple suitable methods. For example, in order to reduce/inhibit expression, one can reduce protein levels post-translationally; one can block production of protein by blocking/reducing translation of mRNA (e.g., using an RNAi agent such as an shRNA or siRNA that targets the mRNA of an endogenous gene); one can reduce mRNA levels post-transcriptionally (e.g., using an RNAi agent such as an shRNA or siRNA that targets the mRNA of an endogenous gene); one can reduce mRNA levels by blocking transcription (e.g., using gene editing tools to either alter a promoter and/or enhancer sequence or to modulate transcription, or by using modified gene editing tools, e.g., CRISPRi, that can modify transcription without cutting the target DNA). Additionally, one can alter the nature of the protein made from an endogenous locus by inducing (e.g., using gene editing technology) a loss of function mutation, which can range from an allele with reduced wild type activity to a dead protein or no protein (e.g., catalytically inactive mutant, a frameshift allele, a gene knockout, etc). Moreover, one can reduce mRNA levels via gene editing methods that result in low net transcript levels (e.g., frameshift mutations can trigger nonsense mediated mRNA decay).

Examples of agents that inhibit expression and/or function of an endogenous gene (see above) include but are not limited to: (a) an RNAi agent such as an shRNA or siRNA that specifically targets mRNA encoded by the endogenous gene; (b) a genome editing agent (e.g., a Zinc finger nuclease, a TALEN, a CRISPR/Cas genome editing agent such as Cas9, Cpf1, CasX, CasY, and the like) that cleaves the target cell's genomic DNA at a locus encoding the endogenous gene—thus inducing a genome editing event (e.g., null allele, partial loss of function allele) at the locus of the endogenous gene; (c) a modified genome editing agent such as a nuclease dead zinc finger, TALE, or CRISPR/Cas nuclease fused to a transcriptional repressor protein that modulates (e.g. reduces) transcription at the locus encoding the endogenous gene (e.g., Nf1, Rasa1, Pten) (see, e.g., Qi et al., Cell. 2013 Feb. 28; 152(5):1173-83′; Gilbert et al, Cell. 2014 Oct. 23; 159(3):647-61; Larson et al., Nat Protoc. 2013 November; 8(11):2180-96); and (d) a small molecule/drug that directly blocks/reduces/inhibits the function of the protein produced by the endogenous locus.

When the agent is a CRISPR/Cas editing agent, the agent can include both the protein and guide RNA component. The guide nucleic acid (e.g., guide RNA) can be introduced into the cell as an RNA or as a DNA encoding the RNA (e.g., encoded by a DNA vector—on a plasmid, virus, and the like). The CRISPR/Cas protein can be introduced into the cell as a protein or as a nucleic acid (mRNA or DNA) encoding the protein. For additional information related to programmable gene editing agents and their guide nucleic acids (e.g., CRISPR/Cas RNa-guided proteins such as Cas9, CasX, CasY, and Cpf1, Zinc finger proteins such as Zinc finger nucleases, TALE proteins such as TALENs, CRISPR/Cas guide RNAs, and the like) refer to, for example, Dreier, et al., (2001) J Biol Chem 276:29466-78; Dreier, et al., (2000) J Mol Biol 303:489-502; Liu, et al., (2002) J Biol Chem 277:3850-6); Dreier, et al., (2005) J Biol Chem 280:35588-97; Jamieson, et al., (2003) Nature Rev Drug Discov 2:361-8; Durai, et al., (2005) Nucleic Acids Res 33:5978-90; Segal, (2002) Methods 26:76-83; Porteus and Carroll, (2005) Nat Biotechnol 23:967-73; Pabo, et al., (2001) Ann Rev Biochem 70:313-40; Wolfe, et al., (2000) Ann Rev Biophys Biomol Struct 29:183-212; Segal and Barbas, (2001) Curr Opin Biotechnol 12:632-7; Segal, et al., (2003) Biochemistry 42:2137-48; Beerli and Barbas, (2002) Nat Biotechnol 20:135-41; Carroll, et al., (2006) Nature Protocols 1:1329; Ordiz, et al., (2002) Proc Natl Acad Sci USA 99:13290-5; Guan, et al., (2002) Proc Natl Acad Sci USA 99:13296-301; Sanjana et al., Nature Protocols, 7:171-192 (2012); Zetsche et al, Cell. 2015 Oct. 22; 163(3):759-71; Makarova et al, Nat Rev Microbiol. 2015 November; 13(11):722-36; Shmakov et al., Mol Cell. 2015 Nov. 5; 60(3):385-97; Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al, Cell. 2013 Feb. 28; 152(5):1173-83; Wang et al., Cell. 2013 May 9; 153(4):910-8; Auer et. al., Genome Res. 2013 Oct. 31; Chen et. al., Nucleic Acids Res. 2013 Nov. 1; 41(20):e19; Cheng et. al., Cell Res. 2013 October; 23(10):1163-71; Cho et. al., Genetics. 2013 November; 195(3):1177-80; DiCarlo et al., Nucleic Acids Res. 2013 April; 41(7):4336-43; Dickinson et. al., Nat Methods. 2013 October; 10(10):1028-34; Ebina et. al., Sci Rep. 2013; 3:2510; Fujii et. al, Nucleic Acids Res. 2013 Nov. 1; 41(20):e187; Hu et. al., Cell Res. 2013 November; 23(11):1322-5; Jiang et. al., Nucleic Acids Res. 2013 Nov. 1; 41(20):e188; Larson et. al., Nat Protoc. 2013 November; 8(11):2180-96; Mali et. at., Nat Methods. 2013 October; 10(10):957-63; Nakayama et. al., Genesis. 2013 December; 51(12):835-43; Ran et. al., Nat Protoc. 2013 November; 8(11):2281-308; Ran et. al., Cell. 2013 Sep. 12; 154(6):1380-9; Upadhyay et. al., G3 (Bethesda). 2013 Dec. 9; 3(12):2233-8; Walsh et. al., Proc Natl Acad Sci USA. 2013 Sep. 24; 110(39):15514-5; Xie et. al., Mol Plant. 2013 Oct. 9; Yang et. al., Cell. 2013 Sep. 12; 154(6):1370-9; Briner et al., Mol Cell. 2014 Oct. 23; 56(2):333-9; Burstein et al., Nature. 2016 Dec. 22—Epub ahead of print; Gao et al., Nat Biotechnol. 2016 July 34(7):768-73; as well as international patent application publication Nos. WO2002099084; WO00/42219; WO02/42459; WO2003062455; WO03/080809; WO05/014791; WO05/084190; WO08/021207; WO09/042186; WO09/054985; and WO10/065123; U.S. patent application publication Nos. 20030059767, 20030108880, 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; 20140377868; 20150166983; and 20160208243; and U.S. Pat. Nos. 6,140,466; 6,511,808; 6,453,242 8,685,737; 8,906,616; 8,895,308; 8,889,418; 8,889,356; 8,871,445; 8,865,406; 8,795,965; 8,771,945; and 8,697,359; all of which are hereby incorporated by reference in their entirety.

As noted, the present disclosure provides oncogene-negative genetically modified cells with increased Ras/MAPK pathway activity, increased PI3K-AKT pathway activity, or both. Such cells can have a reduced wild type protein level from the endogenous locus (e.g., due to an altered nucleotide sequence at an endogenous genomic locus, due to an RNAi agent that specifically targets expression of an HR gene, etc.) of any desired combination of the negative regulator genes selected from Table 2 (i.e., those that state ‘loss of function’, which indicates that a loss of function mutation in that gene leads to increased pathway activity). In some cases, a genetically modified cell with increased Ras/MAPK pathway activity has a reduced wild type protein level from the endogenous locus of any of the genes selected from: CBL, ERRFI1, NF1, RASA1, or any combination thereof. In some cases, a genetically modified cell with increased PI3K-AKT pathway activity has a reduced wild type protein level from the endogenous locus of any of the genes selected from: INPP4B, NPRL2, NPRL3, PIK3R1, PIK3R3, PPP2R1A, PTEN, INPP4B, NPRL2, NPRL3, STK11, TSC1, TSC2, or any combination thereof. In some cases, a genetically modified cell with increased Ras/MAPK pathway activity and increased PI3K-AKT pathway activity has a reduced wild type protein level from the endogenous locus of any of the genes selected from: CBL, ERRFI1, NF1, RASA1, or any combination thereof, and has a reduced wild type protein level from the endogenous locus of any of the genes selected from: INPP4B, NPRL2, NPRL3, PIK3R1, PIK3R3, PPP2R1A, PTEN, INPP4B, NPRL2, NPRL3, STK11, TSC1, TSC2, or any combination thereof.

Genetically modified cells (including isolated genetically modified cells) can a include foreign nucleic acid such as a foreign DNA that includes a nucleotide sequence encoding an RNAi agent and/or can have an altered sequence in the genome (e.g., a loss of function allele, a knock-out/null allele, etc.) at one or more endogenous loci.

In some embodiments, a subject genetically cell includes a foreign nucleic acid such as an RNAi agent (e.g., shRNA, siRNA, microRNA) or a DNA encoding an RNAi agent (e.g., episomally, integrated into the genome) where the RNAi agent specifically targets one or more of the cell's endogenous gene/proteins—in some cases selected from: CBL, ERRFI1, NF1, RASA1, or any combination thereof; in some cases selected from: INPP4B, NPRL2, NPRL3, PIK3R1, PIK3R3, PPP2R1A, PTEN, INPP4B, NPRL2, NPRL3, STK11, TSC1, TSC2, or any combination thereof. In some cases, a subject genetically cell includes an RNAi agent (e.g., shRNA, siRNA, microRNA) or a nucleic acid encoding an RNAi agent (e.g., episomally, integrated into the genome). In some cases the foreign nucleic acid (e.g., DNA encoding an RNAi agent) is incorporated into the cell's genome. In some cases, the foreign nucleic acid (e.g., DNA encoding an RNAi agent) is maintained episomally. In some cases, the foreign nucleic acid (e.g., RNAi agent) is transiently present in the cell.

### Cells of Interest

Any cell type can be a genetically modified cell. Cells of interest are typically vertebrate cells (e.g., mammalian cells). Mammalian cells refers to cells of any animal classified as a mammal, including humans, domestic and farm animals, and zoo, laboratory, sports, or pet animals, such as dogs, horses, cats, cows, rodents (e.g., mice, rats), rabbits, primates, non-human primates etc. In some embodiments, a subject cell is a human cell. In some cases a subject cell is in vivo. In some cases, a subject cell is removed from an individual (e.g., a “primary” cell) (e.g., a cell ex vivo). In some cases, a subject cell is a cell in culture (e.g., from an established cell line) (e.g., a cell in vitro).

Exemplary cells include, but are not limited to, liver cells, pancreatic cells (e.g., islet cells: alpha cells, beta cells, delta cells, gamma cells, and/or epsilon cells), skeletal muscle cells, heart muscle cells, kidney cells, fibroblasts, retinal cells, synovial joint cells, lung cells, T cells, neurons, glial cells, stem cells, blood cells, leukocytes, hematopoietic stem cells, hematopoietic progenitor cells, myeloid cells, immune cells, neural progenitor cells, endothelial cells, and cancer cells. Exemplary stem cell target cells include, but are not limited to, hematopoietic stem cells, neural stem cells, neural crest stem cells, embryonic stem cells, induced pluripotent stem cells (iPS cells), mesenchymal stem cells, mesodermal stem cells, liver stem cells, pancreatic stem cells, muscle stem cells, and retinal stem cells

In some embodiments, a subject genetically modified cell is a vertebrate cell or is derived from a vertebrate cell. In some embodiments, a subject genetically modified cell is a mammalian cell or is derived from a mammalian cell. In some embodiments, a subject genetically modified cell is a rodent cell (e.g., a mouse cell, a rat cell, and the like) or is derived from a rodent cell. In some embodiments, a subject genetically modified cell is a human cell or is derived from a human cell. In some embodiments, a subject genetically modified cell is a genetically modified stem cell or progenitor cell. Suitable cells include, e.g., stem cells (adult stem cells, embryonic stem cells, iPS cells, etc.) and progenitor cells (e.g., cardiac progenitor cells, neural progenitor cells, etc.). Suitable cells include mammalian stem cells and progenitor cells, including, e.g., rodent stem cells, rodent progenitor cells, human stem cells, human progenitor cells, etc. Suitable cells include in vitro cells, e.g., isolated cells.

The present disclosure further provides progeny of a subject genetically modified cell, where the progeny can comprise the same exogenous nucleic acid and/or genomic alteration as the subject genetically modified cell from which it was derived. The present disclosure further provides a composition comprising a subject genetically modified cell.

In some cases a subject genetically modified cell is a cell (e.g., a liver cell, stem cell, germ cell, etc.) isolated from a subject genetically modified non-human organism.

### Genetically Modified Non-Human Mammals

Provided are non-human genetically modified organisms (e.g., mammal, rodent, mouse, rat, pig, horse, sheep, cow, ungulate, non-human primate) that (1) has an oncogene-negative profile, and (2) as described above, has one or more genomic alterations causing increased Ras/MAPK pathway activity and/or increased PI3K-AKT pathway activity. For example, in some cases, a subject non-human genetically modified organism has a mutation at the endogenous locus encoding an endogenous gene for a negative regulator (loss of function mutation) and/or at the endogenous locus encoding an endogenous gene for a positive regulator (gain of function mutation) of Ras/MAPK pathway activity. Likewise, in some cases, a subject non-human genetically modified organism has a mutation at the endogenous locus encoding an endogenous gene for a negative regulator (loss of function mutation) and/or at the endogenous locus encoding an endogenous gene for a positive regulator (gain of function mutation) of PI3K-AKT pathway activity. And in some cases, a subject non-human genetically modified organism has: (i) a mutation at the endogenous locus encoding an endogenous gene for a negative regulator (loss of function mutation) and/or at the endogenous locus encoding an endogenous gene for a positive regulator (gain of function mutation) of Ras/MAPK pathway activity; and (ii) a mutation at the endogenous locus encoding an endogenous gene for a negative regulator (loss of function mutation) and/or at the endogenous locus encoding an endogenous gene for a positive regulator (gain of function mutation) of PI3K-AKT pathway activity.

Examples of suitable non-human genetically modified organisms include but are not limited to: mammals, rodents (e.g., mice, rats), pigs, horses, sheep, cows, ungulates, and non-human primates. In some cases, a subject non-human genetically modified organism is an oncogene-negative mouse (i.e., the mouse has an oncogene-negative genomic profile) for use as a lung cancer model, where the mouse has genomic alterations that cause increased Ras/MAPK pathway activity and increased PI3K-AKT pathway activity. In some cases the increased Ras/MAPK pathway activity is caused by reduced expression wild type Nf1 and/or wild type Rasa1. In some cases the increased PI3K-AKT pathway activity is caused by reduced expression wild type Pten. In some cases the increased PI3K-AKT pathway activity is caused by a pathway-activating alteration of AKT. In some cases, a subject non-human genetically modified organism (e.g., a mouse) has an oncogene-negative genomic profile and has genomic alterations in Nf1 and Rasa1 that cause increased Ras/MAPK pathway activity and a genomic alteration in Pten that causes increased PI3K-AKT pathway activity

In some cases a non-human genetically modified organism includes an exogenous nucleic acid comprising a nucleotide sequence encoding an agent (e.g., RNAi agent) that inhibits expression and/or function of one or more of the endogenous genes listed in Table 2. In such cases, the exogenous nucleic acid can be extrachromosomal (e.g., episomal) or can be integrated into the genome. In some cases the exogenous nucleic acid is operably linked to a functioning promoter.

In some embodiments, a cell that has an altered genomic sequence can be used to generate a subject genetically modified non-human organism (e.g., a rodent, a rat, a mouse, a non-human primate, a mammal, etc.). For example, if the genetically modified cell is a pluripotent stem cell (i.e., PSC) or a germ cell (e.g., a spermatogonium, a sperm, an oogonium, an oocyte, etc.), an entire genetically modified organism can be derived from the genetically modified cell. In some embodiments, the genetically modified cell is a pluripotent stem cell (e.g., ESC, iPSC, pluripotent plant stem cell, etc.) or a germ cell (e.g., a spermatogonium, a sperm, an oogonium, an oocyte, etc.) either in vivo or in vitro that can give rise to a genetically modified organism. In some embodiments the genetically modified cell is a vertebrate pluripotent stem cell (PSC) (e.g., ESC, iPSC, etc.) and is used to generate a genetically modified organism (e.g. by injecting a PSC into a blastocyst to produce a chimeric/mosaic animal, which could then be mated to generate non-chimeric/non-mosaic genetically modified organisms; grafting in the case of plants; etc.).

In some cases a subject genetically modified cell (e.g., a germ cell, a stem cell, a cancer cell, a lung cell) can be isolated from a subject non-human genetically modified organism.

It is also to be understood that in some cases a subject genetically modified non-human organism is not necessarily genetically altered in all cells of its body, but in some cases can be chimeric. For example, in some cases, a subject genetically modified non-human organism will include the genomic alterations in particular cells (e.g., lung cells) but not other cells of the body. So, for example, in some cases a subject genetically modified non-human organism can have lung cells that are genetically modified, and may therefore have one or more lung tumors that include genetically modified cells—but other cells of the animal's body may be genetically unaltered. The generation of such animals can be performed using any of a number of methods known to one of ordinary skill in the art, e.g., using tissue specific expression via promoters that drive CRE (Cre/Lox system) and/or that drive expression of gene-editing tools, using local administration such as using inhaled viruses to target the lungs, local injections, etc.

### Screening Methods

In some cases, a subject method is a method of identifying an agent (e.g., a therapeutic agent for treating a cancer such as a lung cancer, e.g., lung adenocarcinoma). When performing a screening method, the genetically modified cells and organisms described above can be used. For example, a population of oncogene-negative genetically modified cells with increased Ras/MAPK pathway activity and/or increased PI3K-AKT pathway activity can be contacted with a candidate agent. Likewise, a candidate agent can be administered to a subject oncogene-negative non-human genetically modified organism (e.g. a mouse or rat or non-human primate). Any convenient combination of the genomic alterations discussed above for achieving increased Ras/MAPK pathway activity and/or increased PI3K-AKT pathway activity can be suitable for use in a screening method.

A candidate agent (e.g., any convenient type of agent, e.g., a protein, a small peptide, a small molecule, a nucleic acid agent, etc.) can be administered in any of the ways discussed above for delivery of agents (e.g., local, systemic, oral, intravenous, local injection, and the like).

In general, a population of genetically modified cells is contacted with a candidate agent (or a candidate agent is administered to a non-human genetically modified organism) and the efficacy of the agent is then determined.

When contacting a population of cells, it can be determined whether the candidate therapeutic agent prevented or reduced proliferation of the cells relative to a control—and if so, then the candidate agent can said to have been determined to be a therapeutic agent. A discussion of controls can be found elsewhere herein. In short, in some cases the control is a predetermined threshold value, and in some cases the control is a control population of cells that are untreated or treated with a control agent (an agent known to be inert or an agent with a known level of activity).

Any level of reduction can be considered a success (i.e., can render the candidate agent a therapeutic agent). In some cases, an agent is determined to be a success (a therapeutic agent) if it prevented or reduced proliferation of the cells relative to a control by 5% or more (e.g., 10% or more, 15% or more, 20% or more, 25% or more, 30% or more, 40% or more, or 50% or more). In some cases, an agent is determined to be a success (a therapeutic agent) if it prevented or reduced proliferation of the cells relative to a control by 20% or more (e.g., 25% or more, 30% or more, 40% or more, or 50% or more).

In some cases, an agent is determined to be a success (a therapeutic agent) if it prevented or reduced proliferation of the cells relative to a control such that after the method, the test population has 95% or less (e.g., 90% or less, 85% or less, 80% or less, 75% or less, 70% or less, 60% or less, 50% or less, or 40% or less) cells compared to the cells present in the control population. In some cases, an agent is determined to be a success (a therapeutic agent) if it prevented or reduced proliferation of the cells relative to a control such that after the method, the test population has 80% or less (e.g., 75% or less, 70% or less, 60% or less, 50% or less, or 40% or less) cells compared to the cells present in the control population.

In some cases, an agent is determined to be a success (a therapeutic agent) if it prevented or reduced proliferation of the cells relative to a control such that control population has 1.1 fold or more (e.g., 1.1 fold or more, 1.2 fold or more, 1.5 fold or more, 2 fold or more, 3 fold or more, 5 fold or more, or 10 fold or more) the number of cells. In some cases, an agent is determined to be a success (a therapeutic agent) if it prevented or reduced proliferation of the cells relative to a control such that control population has 1.5 fold or more (e.g., 2 fold or more, 3 fold or more, 5 fold or more, or 10 fold or more) the number of cells.

Thus any of the above screening methods can include a step of measuring cell proliferation, counting the number of cells in a given population, monitoring cell death, and the like, in order to determine whether the candidate agent was a success (i.e., can be deemed to be a therapeutic agent).

When administering to an organism, it can be determined whether the candidate therapeutic agent prevented or reduced lung cancer in the individual relative to a control—and if so, then the candidate agent can said to have been determined to be a therapeutic agent. A discussion of controls can be found elsewhere herein. In short, in some cases the control is a predetermined threshold value; in some cases the control is a control tumor in the same individual, wherein the control tumor is an untreated tumor or a tumor treated with a control agent (an agent known to be inert or an agent with a known level of activity); and in some cases the control is a cancer in a different individual, wherein said different individual is an untreated control animal or a control animal treated with a control agent (an agent known to be inert).

Any level of reduction can be considered a success (i.e., can render the candidate agent a therapeutic agent). In some cases, an agent is determined to be a success (a therapeutic agent) if it prevented or reduced lung cancer in the individual relative to a control by 5% or more (e.g., 10% or more, 15% or more, 20% or more, 25% or more, 30% or more, 40% or more, or 50% or more). In some cases, an agent is determined to be a success (a therapeutic agent) if it prevented or reduced lung cancer in the individual relative to a control by 20% or more (e.g., 25% or more, 30% or more, 40% or more, or 50% or more).

In some cases, an agent is determined to be a success (a therapeutic agent) if it prevented or reduced lung cancer in the individual relative to a control such that after the method, the test tumor(s) or individual has 95% or less (e.g., 90% or less, 85% or less, 80% or less, 75% or less, 70% or less, 60% or less, 50% or less, or 40% or less) of the cancer cells (or tumors) present in the control (e.g., control tumor or control individual). In some cases, an agent is determined to be a success (a therapeutic agent) if it prevented or reduced lung cancer in the individual relative to a control such that after the method, the test tumor(s) or individual has 80% or less (e.g., 75% or less, 70% or less, 60% or less, 50% or less, or 40% or less) of the cancer cells (or tumors) present in the control (e.g., control tumor or control individual).

In some cases, an agent is determined to be a success (a therapeutic agent) if it prevented or reduced lung cancer in the individual relative to a control such that control has 1.1 fold or more (e.g., 1.1 fold or more, 1.2 fold or more, 1.5 fold or more, 2 fold or more, 3 fold or more, 5 fold or more, or 10 fold or more) the number of cancer cells (or tumors). In some cases, an agent is determined to be a success (a therapeutic agent) if it prevented or reduced lung cancer in the individual relative to a control such that control has 1.5 fold or more (e.g., 12 fold or more, 3 fold or more, 5 fold or more, or 10 fold or more) the number of cancer cells (or tumors).

In some cases, an agent is determined to be a success (a therapeutic agent) if it extended the average lifespan of treated individuals relative to controls.

Any of the above screening methods can include a step of measuring cancer cells, measuring tumor size, counting tumor number, measuring life span, measuring cell proliferation, and the like, in order to determine whether the candidate agent was a success (i.e., can be deemed to be a therapeutic agent).

### Kits/Systems

The present disclosure provides kits/systems for carrying out a subject method. In some embodiments a subject kit includes an inhibitor of the Ras/MAPK pathway (e.g., an inhibitor of SHP2 such as RMC-4550) and an inhibitor of the PI3K-AKT pathway (e.g., an inhibitor of AKT1/2 such as capivasertib).

In some embodiments a subject kit includes genetically modified cells amendable for screening methods as discussed above. Such kits and also include a control agent(s) for comparison (e.g., a positive and/or negative control agent for which it's effect on cell proliferation and/or cancer/tumor formation is known).

A kit can further include one or more additional reagents, where such additional reagents can be any convenient reagent. Components of a subject kit can be in separate containers; or can be combined in a single container. In some cases one or more of a kit's components are pharmaceutically formulated for administration to a human.

In addition to above-mentioned components, a subject kit can further include instructions for using the components of the kit to practice the subject methods (e.g., dosing instructions, instructions to administer the component(s) to an individual with an oncogene-negative cancer such as a lung cancer (e.g., lung adenocarcinoma). The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In some embodiments, the instructions are present as an electronic storage data file present on a suitable computer readable storage medium, e.g. CD-ROM, diskette, flash drive, etc. In some embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Computer-Implemented Methods, Systems and Devices

The methods of the present disclosure can be computer-implemented, such that method steps (e.g., assaying (e.g., measuring), calculating, comparing, predicting, reporting, and the like) can be automated in whole or in part. Accordingly, the present disclosure provides methods, computer systems, devices and the like in connection with computer-implemented methods of determining whether an individual will respond to treatment with a subject agent (e.g., whether they have increased Ras/MAPK pathway activity and/or increased PI3K-AKT pathway activity.

For example, method steps such as determining whether a candidate therapeutic agent prevented or reduced lung cancer in the individual relative to a control, determining whether a candidate therapeutic agent prevented or reduced proliferation of said population of cells relative to a control, determining whether an individual will be responsive to treatment with a subject agent (e.g. after measuring a level of pathway activity such as measuring pERK and/or pAKT), and the like, can be completely or partially performed by a computer program product. Values obtained can be stored electronically, e.g., in a database, and can be subjected to an algorithm executed by a programmed computer.

For example, the methods of the present disclosure can involve inputting the measured levels (e.g. raw values, normalized values, weighted values, and/or normalized and weighted values) of pERK and/or pAKT, of cell proliferation, of cell counts, and the like, and generate a report as described herein, e.g., by displaying or printing a report to an output device at a location local or remote to the computer.

The present invention thus provides a computer program product including a computer readable storage medium (e.g., a nontransitory computer-readable storage medium) having a computer program stored on it. The program can, when read by a computer, execute relevant calculations based on values obtained from analysis of one or more biological samples from an individual. The computer program product has stored therein a computer program for performing the calculation(s).

### Systems

The present disclosure provides systems for executing the program described above, which system generally includes: (i) a central computing environment; (ii) an input device, operatively connected to the computing environment, to receive data (e.g., expression level data, clinical data from the patient/individual, etc. as described above); (iii) an output device, connected to the computing environment, to provide information to a user (e.g., medical personnel, clinician, and the like); and (iv) an algorithm executed by the central computing environment (e.g., a processor), where the algorithm is executed based on the data received by the input device, and where the algorithm can in some cases calculate a value and/or category, which value and/or category is indicative of (can be used to predict) whether an individual will be responsive to a subject agent.

In some cases, a subject system includes (I) a first system (e.g., a biomolecule analyzing system) that performs a measuring/detection step to generate a value which represents a measured level of a subject expression product (e.g., pERK and/or pAKT), of cell proliferation, of cell death, of cell count, and the like, and (II) a second system that is a computer system. The first and second systems are integrated into a system by virtue of the first system passing the measured expression level data to the second system for analysis. Any convenient measuring/detection system can be used and many suitable systems will be known to one of ordinary skill in the art. While some biomolecule analyzing systems can be considered to be a nucleic acid analyzing system (e.g., a thermocyler, a nucleic acid sequencing machine, and the like), and other biomolecule analyzing systems can be considered to be a protein analyzing system (e.g., an automated ELISA analyzer such as a plate reader, a mass spectrometer, and the like), yet other biomolecule analyzing systems can be used as both a nucleic acid and protein analyzing system (e.g., a flow cytometer). Thus, the term “biomolecule analyzing system” encompasses systems that analyze nucleic acids (e.g., measure levels of nucleic acids in a sample) and systems that analyze proteins (e.g., measure levels of proteins in a sample), as well as systems that analyze both nucleic acids and proteins (e.g., measure levels of nucleic acids and/or proteins in a sample).

A biomolecule analyzing system (e.g., a nucleic acid analyzing system, a protein analyzing system) includes (a) a detector for measuring/detecting a target biomolecule (e.g., an RNA, a protein)(e.g., for measuring an expression level of an RGS1 expression product and/or an expression level of an IL11 expression product), where the detector is coupled to a computer system (e.g., a computer system that can process the data measured by the detector). Thus, the biomolecule analyzing system can measure a level of pERK and/or a level of pAKT, a level of cell proliferation, a level of cell death, a cell count, and the like, and can then send the measured levels to the computer system (the second system).

A biomolecule analyzing system can included a wide variety of different detectors, depending on the labels and assays. Examples of useful detectors include but are not limited to: a microscope(s) (e.g., with multiple channels of fluorescence); a plate reader (e.g., to provide fluorescent, ultraviolet, and/or visible spectrophotometric detection); a CCD camera that can capture data images and transform them into quantifiable formats; etc.

A biomolecule analyzing system can further include liquid handling components (e.g., a robotic system that includes any number of components). Liquid handling components can be partially or fully automated. A wide variety of components which can be used, including, but not limited to, one or more robotic arms; plate handlers for the positioning of microplates; automated lid or cap handlers to remove and replace lids for wells; tip assemblies for sample distribution with disposable tips; washable tip assemblies for sample distribution; 96 well loading blocks; cooled reagent racks; microtiter plate pipette positions (optionally cooled); stacking towers for plates and tips; etc. Fully robotic or microfluidic systems can include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration.

Examples of biomolecule analyzing systems include but are not limited to: a flow cytometer (which can function as a nucleic acid analyzing system and/or a protein analyzing system), a thermocycler (e.g., a nucleic acid analyzing system for assays such as qRT-PCR), a mass spectrophotometer (a protein analyzing system), and a Next Generation high-throughput sequencer (a nucleic acid analyzing system).

The present disclosure provides computer program products that, when executed on a programmable computer such as that described above, can carry out the methods of the present disclosure. As discussed above, the subject matter described herein may be embodied in systems, apparatus, methods, and/or articles depending on the desired configuration. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device (e.g. video camera, microphone, joystick, keyboard, and/or mouse), and at least one output device (e.g. display monitor, printer, etc.).

Computer programs (also known as programs, software, software applications, applications, components, or code) include instructions for a programmable processor, and may be implemented in a high-level procedural and/or object-oriented programming language, and/or in assembly/machine language. As used herein, the term “machine-readable medium” refers to any nontransitory computer program product, apparatus and/or device (e.g., magnetic discs, optical disks, memory, etc.) used to provide machine instructions and/or data to a programmable processor, including a machine-readable medium that receives machine instructions as a machine-readable signal.

It will be apparent from this description that aspects of the present invention may be embodied, at least in part, in software, hardware, firmware, or any combination thereof. Thus, the techniques described herein are not limited to any specific combination of hardware circuitry and/or software, or to any particular source for the instructions executed by a computer or other data processing system. Rather, these techniques may be carried out in a computer system or other data processing system in response to one or more processors, such as a microprocessor, executing sequences of instructions stored in memory or other computer-readable medium including any type of ROM, RAM, cache memory, network memory, floppy disks, hard drive disk (HDD), solid-state devices (SSD), optical disk, CD-ROM, and magnetic-optical disk, EPROMs, EEPROMs, flash memory, or any other type of media suitable for storing instructions in electronic format.

In addition, the processor(s) may be, or may include, one or more programmable general-purpose or special-purpose microprocessors, digital signal processors (DSPs), programmable controllers, application specific integrated circuits (ASICs), programmable logic devices (PLDs), trusted platform modules (TPMs), or the like, or a combination of such devices. In alternative embodiments, special-purpose hardware such as logic circuits or other hardwired circuitry may be used in combination with software instructions to implement the techniques described herein

### Additional Definitions

Unless defined otherwise, all technical and scientific terms used herein have the same meaning as commonly understood by one of ordinary skill in the art to which the invention pertains. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass non-limiting variations of ±40% or ±20% or ±10%, ±5%, ±1%, or ±0.1% from the specified value, as such variations are appropriate.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “biological sample” encompasses a variety of sample types obtained from an organism and can be used in a diagnostic, prognostic, or monitoring assay. The term encompasses blood and other liquid samples of biological origin or cells derived therefrom and the progeny thereof. The term encompasses samples that have been manipulated in any way after their procurement, such as by treatment with reagents, solubilization, or enrichment for certain components. The term encompasses a clinical sample, and also includes cell supernatants, cell lysates, serum, plasma, biological fluids, and tissue samples (e.g., tissue taken from a site of inflammation, a biopsy, and the like). Clinical samples for use in the methods of the invention may be obtained from a variety of sources including, but not limited to tissue from a site of inflammation, a biopsy sample, a thoracentesis sample, a fine needle aspirate, and the like. Exemplary biological samples include, but are not limited to: a suspension of cells (e.g., from a peripheral blood sample, an aspirate, a cell suspension from tissue isolated from a site of inflammation, a cell suspension from a biopsy sample, etc.), a biopsy, an aspirate (e.g., a fine needle aspirate, a thoracentesis sample, etc.), a fixed tissue sample (e.g., a formalin-fixed paraffin embedded (FFPE) tissue sample, an FFPE biopsy sample, etc.), and a homogenized tissue (e.g., a homogenized tissue sample where the tissue is from a site of inflammation, a homogenized biopsy sample, a homogenized paraffin- or OCT-embedded sample, etc.).

Once a sample is isolated (i.e., collected), it can be used directly, frozen, or maintained in appropriate culture medium for a period of time (e.g., in some cases, an extended period of time). Typically the samples will be from human patients, although animal models may find use, e.g. equine, bovine, porcine, canine, feline, rodent, e.g. mice, rats, hamster, non-human primate, etc.

The subject sample can be treated in a variety of ways so as to enhance detection of the expression products. For example, where the sample is taken from a site of inflammation, non-immune cells (or particular types of immune cells) may be removed from the sample (e.g., by differential centrifugation, by differential binding and/or labeling, e.g., FACs sorting and/or magnetic separation techniques) prior to assaying. For example, where the sample is a tumor sample (e.g., a biopsy), non-tumor cells may be removed from the sample (e.g., by differential centrifugation, by differential binding and/or labeling, e.g., FACs sorting and/or magnetic separation techniques) prior to assaying. Where the sample is blood, the red blood cells may be removed from the sample (e.g., by centrifugation) prior to assaying. Such a treatment may serve to reduce the non-specific background levels of detecting an expression level of an expression product. Measurement of an expression level may also be enhanced by concentrating the sample using procedures well known in the art (e.g. acid precipitation, alcohol precipitation, salt precipitation, hydrophobic precipitation, filtration (using a filter which is capable of retaining molecules greater than 30 kD, e.g. Centrim 30™), affinity purification, etc.). In some embodiments, the pH of the test and control samples can be adjusted to, and maintained at, a pH which approximates neutrality (i.e. pH 6.5-8.0). Such a pH adjustment can prevent complex formation, thereby providing a more accurate quantitation of the level of expression product in the sample. In embodiments where the sample is urine, the pH of the sample can be adjusted and the sample can be concentrated in order to enhance the detection.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, synthetic antibodies, chimeric antibodies, and a humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, an “immunoassay” refers to any binding assay that uses an antibody capable of binding specifically to a target molecule to detect and quantify the target molecule.

The term “coding sequence,” as used herein, means a sequence of a nucleic acid or its complement, or a part thereof, that can be transcribed and/or translated to produce the mRNA and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the coding sequence can be deduced therefrom. In contrast, the term “non-coding sequence,” as used herein, means a sequence of a nucleic acid or its complement, or a part thereof, that is not translated into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, and the like.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic, prophylactic, or other desired benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., guide RNA, rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides; at least about 1000 nucleotides to about 1500 nucleotides; about 1500 nucleotides to about 2500 nucleotides; or about 2500 nucleotides (and any integer value in between). As used herein, the term “fragment,” as applied to a protein, polypeptide or peptide, refers to a subsequence of a larger protein, polypeptide or peptide. A “fragment” of a protein, polypeptide, or peptide can be at least about 5 amino acids in length; for example, at least about 10 amino acids in length; at least about 20 amino acids in length; at least about 50 amino acids in length; at least about 100 amino acids in length; at least about 200 amino acids in length; or at least about 300 amino acids in length (and any integer value in between).

The term “gene” refers to a nucleic acid (e.g., DNA) sequence that includes coding sequences necessary for the production of a polypeptide, precursor, or RNA (e.g., mRNA). The polypeptide may be encoded by a full-length coding sequence or by any portion of the coding sequence so long as the desired activity or functional property (e.g., enzymatic activity, receptor binding, signal transduction, immunogenicity, etc.) of the full-length or fragment is retained. The term also encompasses the coding region of a structural gene and the sequences located adjacent to the coding region on both the 5′ and 3′ ends for a distance of about 2 kb or more on either end such that the gene corresponds to the length of the full-length mRNA and 5′ regulatory sequences which influence the transcriptional properties of the gene. Sequences located 5′ of the coding region and present on the mRNA are referred to as 5′-untranslated sequences. The 5′-untranslated sequences usually contain the regulatory sequences. Sequences located 3′ or downstream of the coding region and present on the mRNA are referred to as 3′-untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

“Homologous”, “identical,” or “identity” as used herein in the context of two or more nucleic acids or polypeptide sequences means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Instructional material,” as that term is used herein, includes a publication, a recording, a diagram, or any other medium of expression which can be used to communicate the usefulness of the nucleic acid, peptide, polypeptide, and/or compound of the invention in the kit for identifying or alleviating or treating the various diseases or disorders recited herein. Optionally, or alternately, the instructional material may describe one or more methods of identifying or alleviating the diseases or disorders in a cell or a tissue of a subject. The instructional material of the kit may, for example, be affixed to a container that contains the nucleic acid, polypeptide, and/or compound of the invention or be shipped together with a container that contains the nucleic acid, polypeptide, and/or compound. Alternatively, the instructional material may be shipped separately from the container with the intention that the recipient uses the instructional material and the compound cooperatively.

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

As used herein, the terms “purify” and “purified” in the context of a protein refers to level of purity that allows for the effective use of the protein, e.g., in vitro, ex vivo, or in vivo. For a protein to be useful for a given application, it should be substantially free of contaminants, other proteins, and/or chemicals that could interfere with the use of that protein in such application, or that at least would be undesirable for inclusion with the protein of interest. Such applications include that preparation of therapeutic compositions, the administration of the protein in a therapeutic composition, and other methods disclosed herein. Preferably, a “purified” protein, as referenced herein, is a protein that can be produced by any method (i.e., by direct purification from a natural source, recombinantly, or synthetically), and that has been purified from other protein components such that the protein comprises at least about 75% weight/weight of the total protein in a given composition, 80% weight/weight of the total protein in a given composition, and more preferably, at least about 85%, and more preferably at least about 90%, and more preferably at least about 91%, and more preferably at least about 92%, and more preferably at least about 93%, and more preferably at least about 94%, and more preferably at least about 95%, and more preferably at least about 96%, and more preferably at least about 97%, and more preferably at least about 98%, and more preferably at least about 99% weight/weight of the total protein in a given composition. As an example, a purified polypeptide is a polypeptide which has been separated from other components with which it might normally be associated in its naturally occurring state (e.g., if the protein is a naturally existing protein) and from components with which it may be associated while inside of a cell or in extracellular milieu. For example, in some cases a protein can be purified from a cellular lysate (e.g., from a lysate of bacterial cells in which the protein was exogenously expressed). As another example a protein can be purified from an extracellular medium, e.g., from culture medium into which cells (e.g., yeast cells) have secreted the protein.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

The term “label” when used herein refers to a detectable compound or composition that is conjugated directly or indirectly to a probe to generate a “labeled” probe. The label may be detectable by itself (e.g., radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

By the term “modulating,” as used herein, is meant mediating a detectable increase or decrease in the activity and/or level of a mRNA, polypeptide, or a response in a subject compared with the activity and/or level of the mRNA, polypeptide or response in the subject in the absence of a treatment or compound, and/or compared with the activity and/or level of the mRNA, polypeptide, or response in an otherwise identical but untreated subject. The term encompasses activating, inhibiting and/or otherwise affecting a native signal or response thereby mediating a beneficial therapeutic, prophylactic, or other desired response in a subject, for example, a human.

A “mutation,” “mutant,” or “variant,” as used herein, refers to a change in nucleic acid or amino acid sequence relative to a reference sequence (which may be a naturally-occurring normal/“wild-type” sequence), and includes translocations, deletions, insertions, and substitutions/point mutations. A “mutant” or “variant” as used herein, refers to either a nucleic acid or protein comprising a mutation.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, mutant polypeptides, variant polypeptides, or a combination thereof.

As used herein, the term “wild-type” refers to a gene or gene product having a naturally occurring sequence. In contrast, the term “modified,” “variant,” or “mutant” refers to a gene or gene product that possesses modifications in sequence and/or functional properties (i.e., altered characteristics) when compared to the wild-type gene or gene product.

Ranges: throughout this disclosure, various aspects can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 2, 1 to 3, 1 to 4, 1 to 5, 2 to 3, 2 to 4, 2 to 5, 2 to 6, etc., as well as individual numbers within that range, for example, 1, 2, 2.7. 3, 4, 5, 5.3, and 6. This applies regardless of the breadth of the range.

### Example Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below as Set A and Set B. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.

### Set A

1. A method of treatment, comprising:
   administering a composition comprising an inhibitor of Ras/MAPK pathway activity to an individual who has a cancer previously determined to be oncogene-negative.
2. The method of 1, wherein the inhibitor of Ras/MAPK pathway activity comprises an inhibitor of SHP2.
3. The method of 2, wherein the inhibitor of SHP2 is RMC-4550 or RMC-4630.
4. The method of any one of 1-3, further comprising administering an inhibitor of PI3K-AKT pathway activity to the individual.
5. The method of 4, wherein the inhibitor of PI3K-AKT pathway activity comprises an inhibitor of AKT1/2.
6. The method of 5, wherein the inhibitor of AKT1/2 is capivasertib.
7. The method of any one of 1-6, wherein the oncogene-negative cancer is a lung cancer.
8. The method of 7, wherein the lung cancer is lung adenocarcinoma.
9. The method of any one of 1-8, wherein said cancer was previously determined to be oncogene-negative by a method comprising genome sequencing.
10. The method of any one of 1-8, comprising a step of providing said previous oncogene-negative determination by assaying a biological sample of said cancer.
11. The method of 10, wherein said assaying comprises genome sequencing.
12. The method of any one of 1-11, wherein, prior to said administering, said cancer of the individual was previously determined to exhibit increased: (i) Ras/MAPK pathway activity or (ii) Ras/MAPK pathway activity and PI3K-AKT pathway activity, compared to a control value.
13. The method of 12, wherein said method comprises a step of providing said previous determination of increased pathway activity by assaying a biological sample of said cancer.
14. The method of 13, wherein said assaying comprises genome sequencing and/or biomarker analysis.
15. The method of 14, wherein said biomarker analysis comprises measuring levels of: (i) phosphorylated ERK (pERK) or (ii) pERK and phosphorylated AKT (pAKT).
16. The method of 15, wherein said measuring comprises immunohistochemistry.
17. A composition formulated for use in a method of treating an individual who has an oncogene-negative cancer, wherein the composition comprises an inhibitor of Ras/MAPK pathway activity.
18. The composition of 17, wherein the inhibitor of Ras/MAPK pathway activity comprises an inhibitor of SHP2.
19. The composition of 18, wherein the inhibitor of SHP2 is RMC-4550 or RMC-4630.
20. The composition of any one of 17-19, further comprising an inhibitor of PI3K-AKT pathway activity.
21. The composition of 20, wherein the inhibitor of PI3K-AKT pathway activity comprises an inhibitor of AKT1/2.
22. The composition of 21, wherein the inhibitor of AKT1/2 is capivasertib.
23. The composition of any one of 17-22, wherein the oncogene-negative cancer is a lung cancer.
24. The composition of 23, wherein the lung cancer is lung adenocarcinoma.
25. The composition of any one of 17-24, wherein the individual exhibits increased: (i) Ras/MAPK pathway activity or (ii) Ras/MAPK pathway activity and PI3K-AKT pathway activity, compared to a control value.
26. A kit for use in a method of treating an individual who has an oncogene-negative cancer, the kit comprising: (a) an inhibitor of Ras/MAPK pathway activity; and (b) an inhibitor of PI3K-AKT pathway activity, wherein the components of the kit are separated from one another.
27. The kit of 26, wherein the inhibitor of Ras/MAPK pathway activity comprises an inhibitor of SHP2.
28. The kit of 27, wherein the inhibitor of SHP2 is RMC-4550 or RMC-4630.
29. The kit of any one of 26-28, wherein the inhibitor of PI3K-AKT pathway activity comprises an inhibitor of AKT1/2.
30. The kit of 29, wherein the inhibitor of AKT1/2 is capivasertib.
31. The kit of any one of 26-30, wherein the oncogene-negative cancer is a lung cancer.
32. The kit of 31, wherein the lung cancer is lung adenocarcinoma.
33. The kit of any one of 26-32, wherein the individual exhibits increased: (i) Ras/MAPK pathway activity or (ii) Ras/MAPK pathway activity and PI3K-AKT pathway activity, compared to a control value.
34. An oncogene-negative mouse for use as a cancer model, comprising an oncogene-negative genomic profile and reduced expression of wild type Nf1, Rasa1, and Pten.
35. The oncogene-negative mouse of 34, wherein the reduced expression of wild type Nf1, Rasa1, and Pten is caused by a genetic loss-of-function mutation in each of the Nf1, Rasa1, and Pten loci.

### Set B

1. A method of treatment, comprising:
   administering a composition comprising an inhibitor of Ras/MAPK pathway activity to an individual who has a cancer previously determined to be oncogene-negative.
2. The method of 1, wherein the inhibitor of Ras/MAPK pathway activity comprises an inhibitor of SHP2.
3. The method of 2, wherein the inhibitor of SHP2 is RMC-4550 or RMC-4630.
4. The method of any one of 1-3, further comprising administering an inhibitor of PI3K-AKT pathway activity to the individual.
5. The method of 4, wherein said administering of the inhibitor of PI3K-AKT pathway activity comprises local administration to a tumor of the individual.
6. The method of 4 or 5, wherein the inhibitor of PI3K-AKT pathway activity comprises an inhibitor of AKT1/2.
7. The method of 6, wherein the inhibitor of AKT1/2 is capivasertib.
8. The method of any one of 1-7, wherein the oncogene-negative cancer is a lung cancer.
9. The method of 8, wherein the lung cancer is lung adenocarcinoma.
10. The method of any one of 1-9, wherein said cancer was previously determined to be oncogene-negative by a method comprising genome sequencing.
11. The method of any one of 1-9, comprising a step of providing said previous oncogene-negative determination by assaying a biological sample of said cancer.
12. The method of 11, wherein said assaying comprises genome sequencing.
13. The method of any one of 1-12, wherein, prior to said administering, said cancer of the individual was previously determined to exhibit increased: (i) Ras/MAPK pathway activity or (ii) Ras/MAPK pathway activity and PI3K-AKT pathway activity, compared to a control value.
14. The method of 13, wherein said method comprises a step of providing said previous determination of increased pathway activity by assaying a biological sample of said cancer.
15. The method of 14, wherein said assaying comprises genome sequencing and/or biomarker analysis.
16. The method of 15, wherein said biomarker analysis comprises measuring levels of: (i) phosphorylated ERK (pERK) or (ii) pERK and phosphorylated AKT (pAKT).
17. The method of 16, wherein said measuring comprises immunohistochemistry.
18. A composition formulated for use in a method of treating an individual who has an oncogene-negative cancer, wherein the composition comprises an inhibitor of Ras/MAPK pathway activity.
19. The composition of 18, wherein the inhibitor of Ras/MAPK pathway activity comprises an inhibitor of SHP2.
20. The composition of 19, wherein the inhibitor of SHP2 is RMC-4550 or RMC-4630.
21. The composition of any one of 18-20, further comprising an inhibitor of PI3K-AKT pathway activity.
22. The composition of 21, wherein the inhibitor of PI3K-AKT pathway activity comprises an inhibitor of AKT1/2.
23. The composition of 22, wherein the inhibitor of AKT1/2 is capivasertib.
24. The composition of any one of 18-23, wherein the oncogene-negative cancer is a lung cancer.
25. The composition of 24, wherein the lung cancer is lung adenocarcinoma.
26. The composition of any one of 18-25, wherein the individual exhibits increased: (i) Ras/MAPK pathway activity or (ii) Ras/MAPK pathway activity and PI3K-AKT pathway activity, compared to a control value.
27. A kit for use in a method of treating an individual who has an oncogene-negative cancer, the kit comprising: (a) an inhibitor of Ras/MAPK pathway activity; and (b) an inhibitor of PI3K-AKT pathway activity, wherein the components of the kit are separated from one another.
28. The kit of 27, wherein the inhibitor of Ras/MAPK pathway activity comprises an inhibitor of SHP2.
29. The kit of 28, wherein the inhibitor of SHP2 is RMC-4550 or RMC-4630.
30. The kit of any one of 27-29, wherein the inhibitor of PI3K-AKT pathway activity comprises an inhibitor of AKT1/2.
31. The kit of 30, wherein the inhibitor of AKT1/2 is capivasertib.
32. The kit of any one of 27-31, wherein the oncogene-negative cancer is a lung cancer.
33. The kit of 32, wherein the lung cancer is lung adenocarcinoma.
34. The kit of any one of 17-33, wherein the individual exhibits increased: (i) Ras/MAPK pathway activity or (ii) Ras/MAPK pathway activity and PI3K-AKT pathway activity, compared to a control value.
35. A method for testing candidate therapies, the method comprising:
   (a) administering a candidate therapeutic agent to a non-human genetically modified mammal that has an oncogene-negative genomic profile and comprises lung cells with one or more genomic alterations causing increased Ras/MAPK pathway activity and/or increased PI3K-AKT pathway activity; and
   (b) determining whether the candidate therapeutic agent prevented or reduced lung cancer in the individual relative to a control.
36. The method of 35, wherein said one or more genomic alterations cause increased Ras/MAPK pathway activity and increased PI3K-AKT pathway activity.
37. The method of 35 or 36, wherein the increased Ras/MAPK pathway activity results from reduced expression of wild type Nf1 and/or wild type Rasa1.
38. The method of any one of 35-37, wherein the increased PI3K-AKT pathway activity results from reduced expression of wild type Pten.
39. The method of any one of 35-37, wherein the increased PI3K-AKT pathway activity results from a pathway-activating alteration of AKT.
40. The method of any one of 35-39, wherein the candidate therapeutic agent is administered locally to a tumor.
41. The method of any one of 35-39, wherein the candidate therapeutic agent is administered systemically to the individual.
42. The method of any one of 35-41, wherein said control is a predetermined threshold value.
43. The method of any one of 35-41, wherein said control is a control tumor in the same individual, wherein the control tumor is an untreated tumor or a tumor treated with a control agent.
44. The method of any one of 35-41, wherein said control is a cancer in a different individual, wherein said different individual is an untreated control animal or a control animal treated with a control agent.
45. The method of any one of 35-44, wherein the non-human genetically modified mammal is a rodent.
46. The method of any one of 35-44, wherein the non-human genetically modified mammal is a non-human primate.
47. A method for testing candidate therapies, the method comprising:
   (a) contacting a population of cells with a candidate therapeutic agent, wherein said cells are mammalian cells that have an oncogene-negative genomic profile and comprise one or more genomic alterations causing increased Ras/MAPK pathway activity and/or increased PI3K-AKT pathway activity; and
   (b) determining whether the candidate therapeutic agent prevented or reduced proliferation of said population of cells relative to a control.
48. The method of 47, wherein said cells are from a non-human genetically modified mammal or are progeny of such cells, wherein said non-human genetically modified mammal has an oncogene-negative genomic profile and comprises one or more genomic alterations causing increased Ras/MAPK pathway activity and/or increased PI3K-AKT pathway activity.
49. The method of 47 or 48, wherein said cells are rodent cells.
50. The method of 47 or 48, wherein said cells are non-human primate cells.
51. The method of 47, wherein said cells are human cells.
52. The method of any one of 47-51, wherein said cells are lung cells.
53. The method of any one of 47-52, wherein said one or more genomic alterations cause increased Ras/MAPK pathway activity and increased PI3K-AKT pathway activity.
54. The method of any one of 47-53, wherein the increased Ras/MAPK pathway activity results from reduced expression of wild type Nf1 and/or wild type Rasa1.
55. The method of any one of 47-54, wherein the increased PI3K-AKT pathway activity results from reduced expression of wild type Pten and/or from a pathway-activating alteration of AKT.
56. The method of any one of 47-55, wherein said control is a predetermined threshold value.
57. The method of any one of 47-55, wherein said control is a control population of cells that are untreated or treated with a control agent.
58. An oncogene-negative mouse for use as a lung cancer model, comprising:
   an oncogene-negative genomic profile and genomic alterations causing increased Ras/MAPK pathway activity and increased PI3K-AKT pathway activity.
59. The oncogene-negative mouse of 58, wherein the increased Ras/MAPK pathway activity is caused by reduced expression wild type Nf1 and/or wild type Rasa1.
60. The oncogene-negative mouse of 58 or 59, wherein the increased PI3K-AKT pathway activity is caused by reduced expression wild type Pten.
61. The oncogene-negative mouse of 58 or 59, wherein the increased PI3K-AKT pathway activity is caused by a pathway-activating alteration of AKT.
62. The oncogene-negative mouse of any one of 58-61, wherein the increased Ras/MAPK pathway activity is caused by reduced expression wild type Nf1 and wild type Rasa1, and the increased PI3K-AKT pathway activity is caused by reduced expression wild type Pten.
63. The oncogene-negative mouse of 62, wherein the reduced expression of wild type Nf1, Rasa1, and Pten is caused by a genetic loss-of-function mutations in each of the Nf1, Rasa1, and Pten loci.
64. A cell, or progeny thereof, isolated from the oncogene-negative mouse of 58.
65. The cell, or progeny thereof, of 64, wherein the cell is a germ cell.
66. The cell, or progeny thereof, of 64, wherein the cell is a stem cell.
67. The cell, or progeny thereof, of 64, wherein the cell is a lung cell.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1—Combinatorial Tumor Suppressor Inactivation Efficiently Initiates Lung Adenocarcinoma with Therapeutic Vulnerabilities

### Introduction

The identification of driver oncogene alterations has fundamentally advanced our understanding of cancer initiation, progression, and maintenance. The classification of cancer types based upon their driver oncogenes has enabled an ongoing shift from generally toxic chemotherapies to more effective therapies that most often target the protein products of oncogenes[1]. Tumors typically contain multiple driver gene alterations in both proto-oncogenes and tumor suppressor genes [2]. While oncogenes have clear roles in tumor initiation and maintenance in many cancer types, a significant fraction of tumors lacks identifiable alterations in proto-oncogenes [3, 4]. Consequently, developing targeted therapies for these tumors remains a major clinical challenge.

Lung cancer is the leading cause of cancer death worldwide [5]. Genomic analyses of lung adenocarcinoma, the most frequent subtype of lung cancer, have detected alterations in receptor tyrosine kinase and RAS/RAF pathway oncogenes in ˜55-70% of tumors, including frequent mutations in EGFR and KRAS (“oncogene-positive” tumors) [6]. A significant fraction (˜30-45%) of lung adenocarcinomas is thought to lack a driving oncogene, which presents a major unmet challenge for precision thoracic oncology [4, 7, 8]. Oncogene mutations in these tumors might have been missed due to technical reasons, including insufficient sequencing depth and low cancer cell purity [4, 9]. Additionally, as of yet unidentified oncogenes not included in sequencing panels could drive the development of lung adenocarcinomas independent from established oncogene alterations. However, each newly discovered oncogene such as ALK, ROS1, RET, NTRK1, and NRG1 rearrangements sequentially explain increasingly smaller subsets of cases [10-13]. Therefore, it is unlikely that undiscovered driver oncogenes will explain the large and clinically significant population of patients with oncogene-negative lung adenocarcinoma [4]. Thus, despite the diagnosis of more than 150,000 patients per year with oncogene-negative lung adenocarcinomas worldwide and exhaustive efforts to discover new oncogene alterations in this prevalent subtype of lung adenocarcinoma, the events that drive the initiation and growth of these tumors remain unclear.

Combinatorial alterations in tumor suppressor genes could co-operate to drive these likely oncogene-negative lung tumors. Genomic analyses of human lung adenocarcinoma have identified complex patterns of mutations in diverse putative tumor suppressor genes [7]. However, the ability to predict which combinations of genetic alterations drive cancer in the absence of oncogene activation based on human genomic data alone remains a challenging task. While human genomic data can predict combinations of genomic mutations as likely cancer drivers when the mutations co-occur at very high frequencies (such as in the inactivation of TP53 and RB1 in small cell lung cancer [14-17]), identifying lower frequency alterations in genes across multiple pathways poses a much harder challenge. Finally, non-genomic mechanisms that inactivate tumor suppressor genes [18-20] and the large numbers of mutations generated by mutagens such as smoking further reduce the ability of human cancer genomic studies to identify combinatorial genetic alterations that drive lung cancer [21].

Oncogenes and tumor suppressor genes are parts of broader signaling networks that generate and sustain the biochemical changes that drive tumor initiation and growth [2, 22-24]. Determining the specific pathways involved in tumor initiation can be aided by functional analysis in experimental cancer models [25].

In the experiments described below, a quantitative mouse model system is leveraged to assess the ability of hundreds of combinatorial alterations of tumor suppressor genes to generate oncogene-negative lung tumors in vivo. Genetic drivers are uncovered and pathway-level changes that drive lung cancer in the absence of oncogene mutations are shown and discussed. These findings are leveraged for therapeutic purposes.

### Results

### Clinical Features of Oncogene-Negative and Oncogene-Positive Human Lung Adenocarcinomas are Broadly Similar

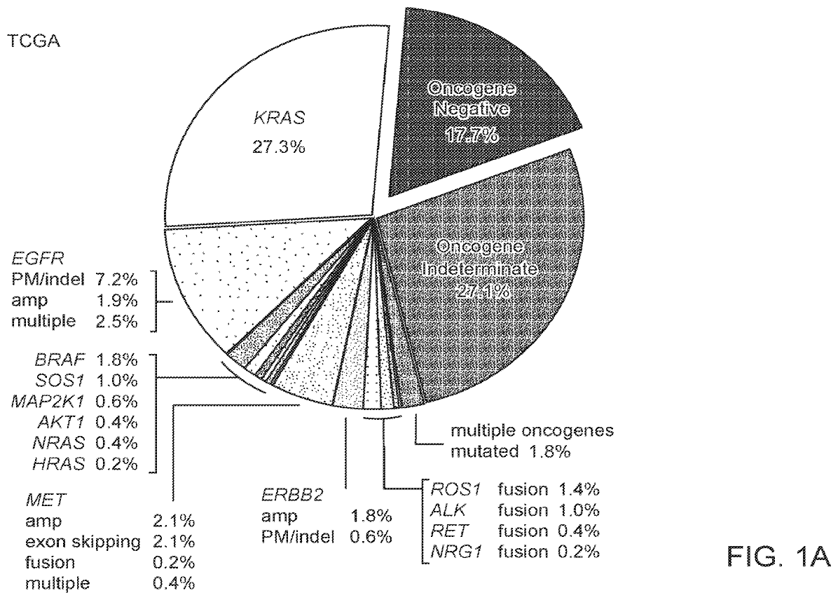

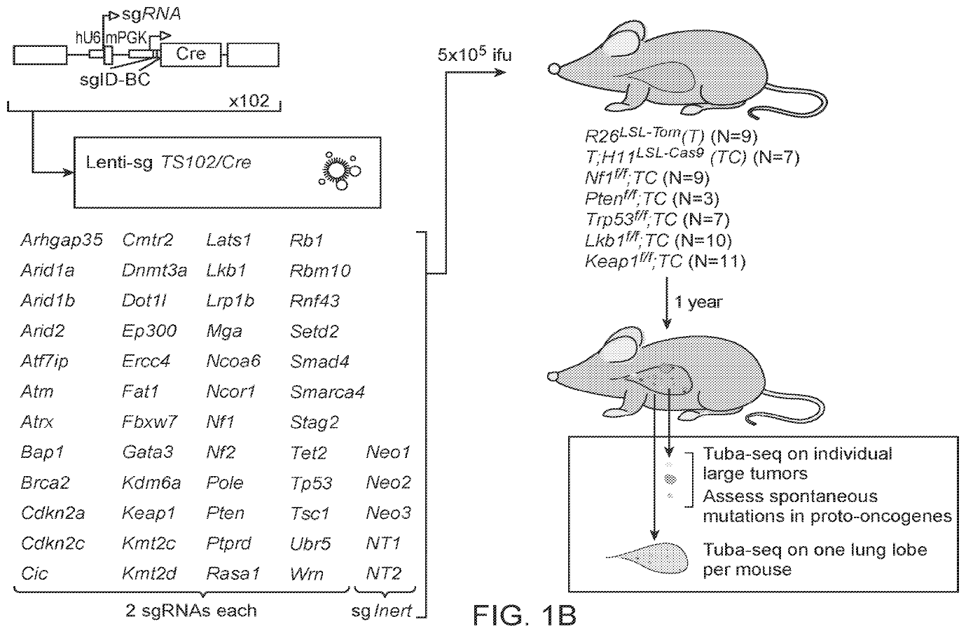

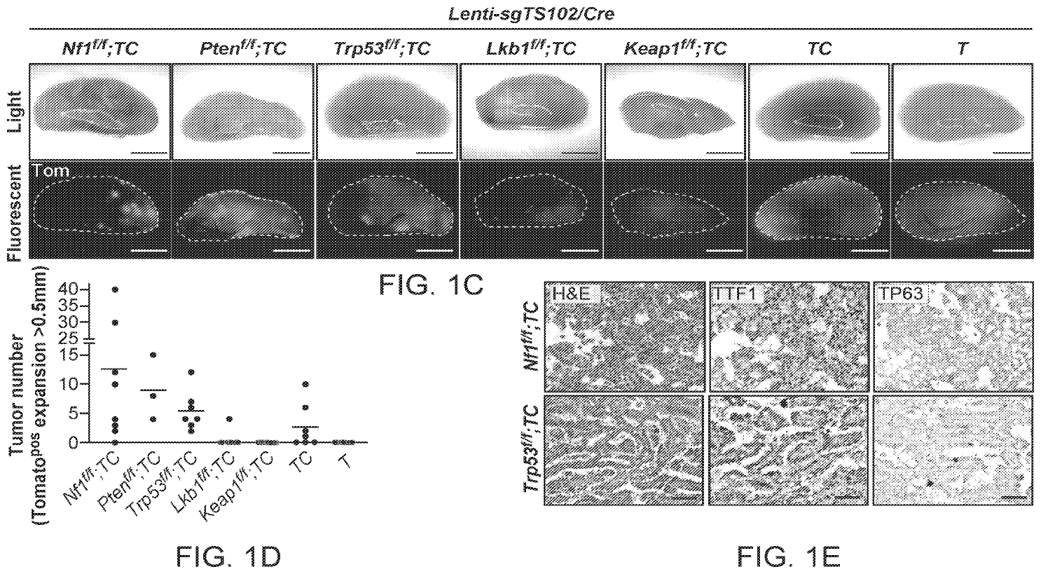

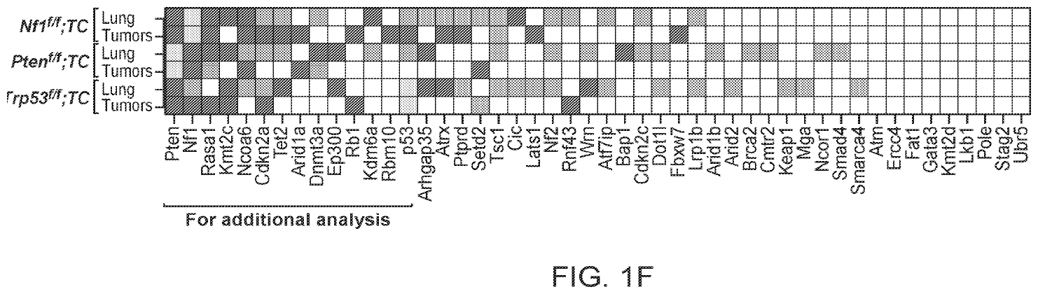

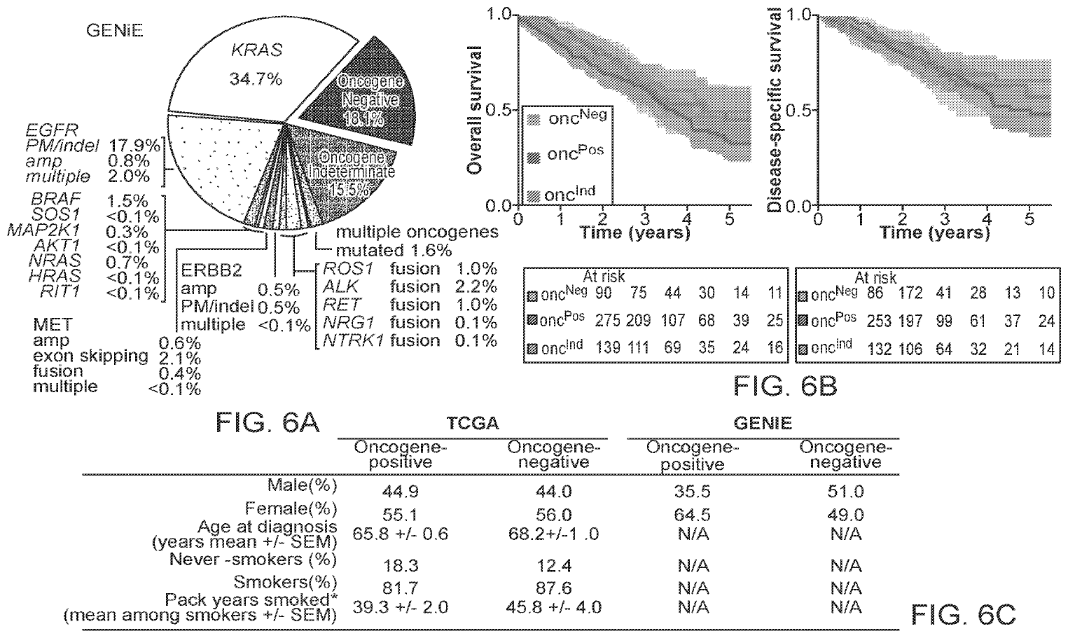

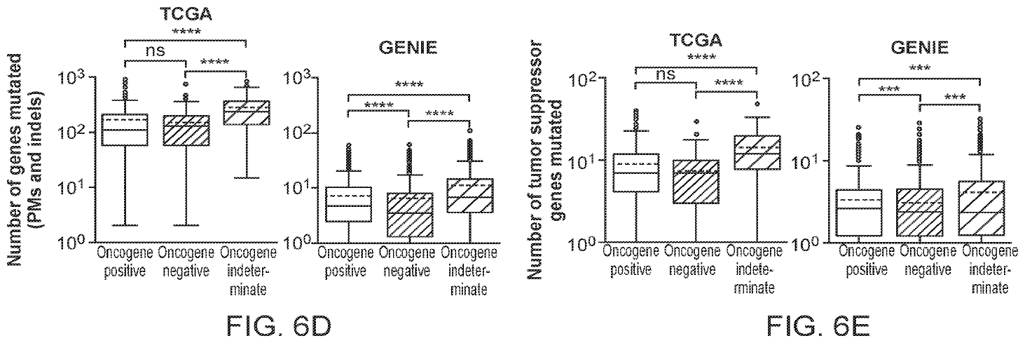

To better understand the genomics of lung adenocarcinomas that lack oncogene mutations, we analyzed data from The Cancer Genome Atlas (TCGA) and AACR Genomics Evidence Neoplasia Information Exchange (GENIE) [26, 27]. We stratified tumors as oncogene-positive if they had high-confidence oncogene alterations in previously described proto-oncogenes, oncogene-indeterminate if they had alterations in known proto-oncogenes that have not been established to generate oncogenic activity in this context, and oncogene-negative if they had no alterations in known proto-oncogenes (Methods). We found that a significant fraction (17-18%) of lung adenocarcinomas had no activating alterations in known proto-oncogenes (FIG. 1a, 6a) [28-30]. Additionally, 15-27% of lung adenocarcinomas were oncogene-indeterminate. These two groups of lung adenocarcinomas without known oncogene mutations accounted for 32-45% of lung adenocarcinoma cases. We further determined that patients with oncogene-negative and oncogene-positive lung adenocarcinomas have a broadly similar mutational burden and clinical characteristics (FIG. 6b-e).

### Combinatorial Tumor Suppressor Gene Inactivation Enables Lung Tumor Development in the Absence of Engineered Oncogenes

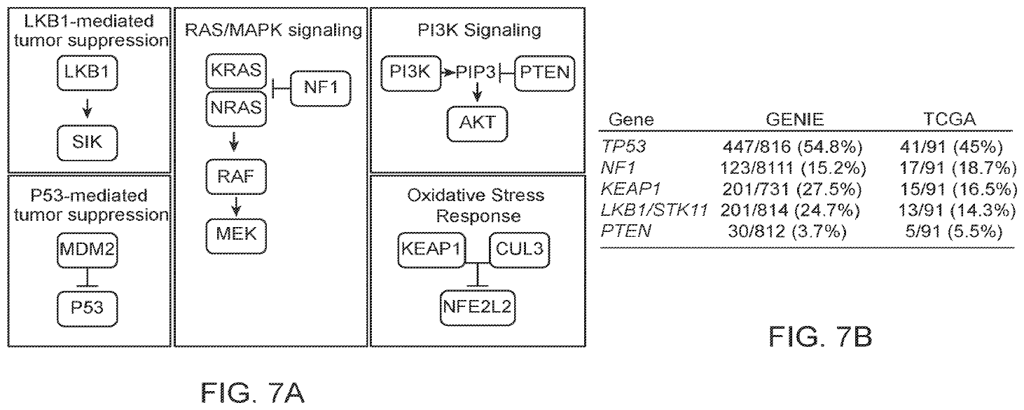

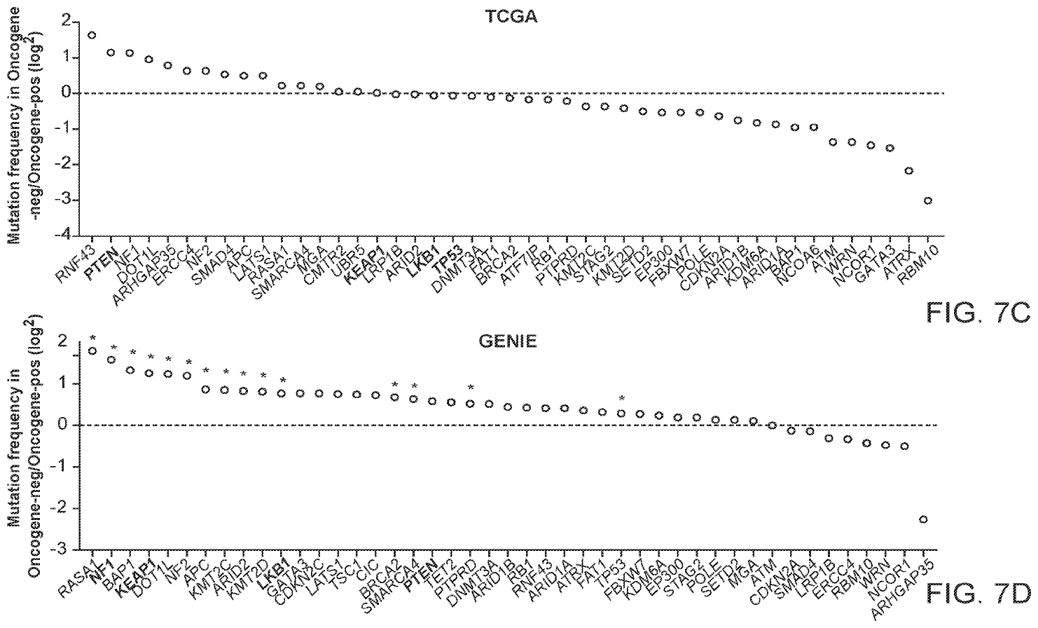

Given the presence of widespread genetic and epigenetic alterations in human tumors [2], genomic analyses have limited ability to delineate causal relationships between genomic alterations and tumor development. To determine whether combinatorial tumor suppressor gene inactivation alone can drive lung tumor initiation in the absence of oncogene activation, we coupled Cre/loxP-based genetically engineered mouse models and somatic CRISPR/Cas9-based genome editing with tumor barcoding and high-throughput barcode sequencing (Tuba-seq) [31-35]. We used Cre/loxP to inactivate each of five “core” tumor suppressor genes (Trp53, Lkb1/Stk11, Keap1, Nf1, and Pten). These genes operate within diverse pathways and are frequently inactivated in human lung cancers, including oncogene-negative lung adenocarcinomas (FIG. 7a, b) [35-38]. We further used CRISPR/Cas9 to coincidentally inactivate panels of additional tumor suppressor genes in lung epithelial cells in mice with floxed alleles (f/f) of each of the “core” tumor suppressors, a Cre-reporter allele (R26^{LSL-Tom} (T) [36]), and a Cre-regulated Cas9 allele (H11^{LSL-Cas9} (C) [37]).

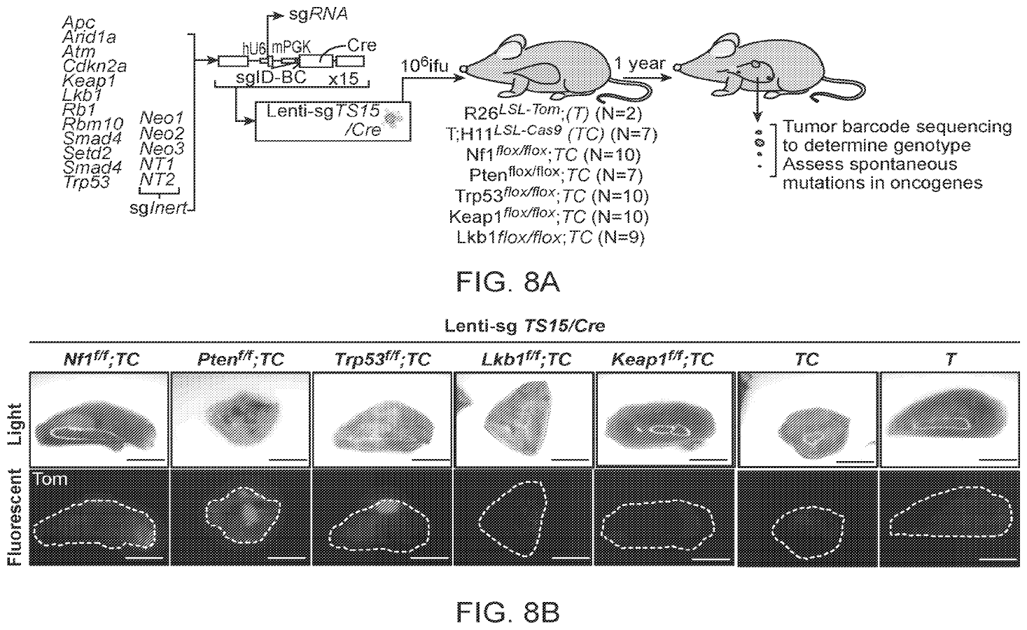

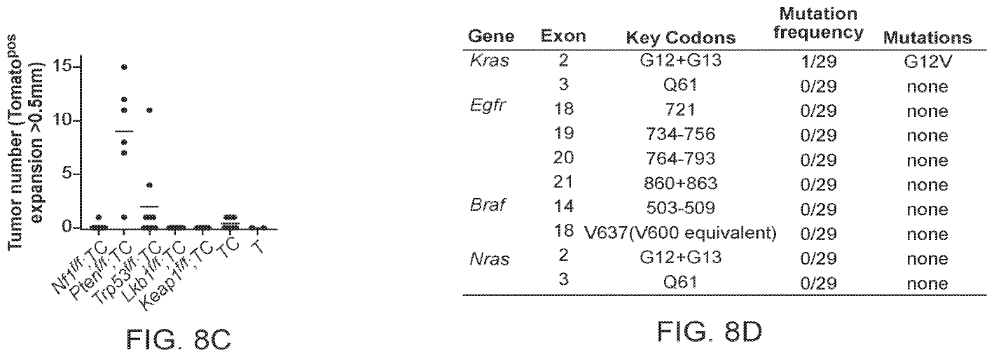

Specifically, we transduced Nf1^{f/f}; TC, Pten1^{f/f}; TC, Trp53^{f/f}; TC, Lkb1^{f/f}; TC, Keap1^{f/f}; TC, TC, and T mice with high titers of two pools of barcoded Lenti-sgRNA/Cre vectors targeting 49 different tumor suppressor genes that have been previously studied in the context of Kras^{G12D}-driven tumors [31, 32, 35]. One pool contained vectors targeting 11 tumor suppressors with one sgRNA per gene in addition to four inert sgRNAs (Lenti-sgTS15/Cre) (FIG. 8a) [31, 32]. The other pool included vectors targeting 48 tumor suppressors, including the “core” tumor suppressors and most of the tumor suppressors targeted in Lenti-sgTS15/Cre at two or three sgRNAs per gene in addition to five inert sgRNAs (102 sgRNA in total, Lenti-sgTS102/Cre) (FIG. 1c) [35]. Thus, the combination of Cre/LoxP and CRISPR/Cas9-based genome editing should generate more than 200 combinations of two or more tumor suppressor alterations in lung epithelial cells. We previously found that a small percent of lung tumors initiated with Lenti-sgRNA/Cre vectors in other lung cancer models contained multiple sgRNAs, consistent with the transduction of the initial cell with multiple Lenti-sgRNA/Cre vectors [31, 32]. Thus, using a high titer of Lenti-sgRNA/Cre pools in this study increases the potential of finding higher-order interactions, in addition to pairwise interactions, that increase the growth advantage of the transduced cells.

We determined the alteration frequency of hundreds of tumor suppressor genes [22, 38], including those targeted using our pre-existing Lenti-sgTS15/Cre and Lenti-sgTS102/Cre pools, in oncogene-positive and oncogene-negative tumors from TCGA and GENIE. Mutations in tumor suppressor genes occur in both oncogene-positive and oncogene-negative tumors, with only 17 tumor suppressor alterations being significantly enriched in oncogene-negative tumors in GENIE. 12 out of these 17 tumor suppressor genes were targeted using Lenti-sgTS15/Cre and Lenti-sgTS102/Cre pools (FIG. 7c, d).

One year after transduction with the Lenti-sgTS15/Cre or Lenti-sgTS102/Cre pools, Nf1^{f/f}; TC, Pten1^{f/f}; TC, and Trp53^{f/f}; TC mice developed a modest number of tumors (defined as Tomato^{positive} expansion >0.5 mm in diameter), while Lkb1^{f/f}; TC and Keap1^{f/f}; TC mice rarely developed any tumors (FIG. 1c-d, 8b-c). Interestingly, Nf1^{f/f}; TC, Pten^{f/f}; TC, and Trp53^{f/f}; TC mice transduced with the larger Lenti-sgTS102/Cre pool developed more or larger tumors than the mice transduced with the Lenti-sgTS15/Cre pool. This observation suggests the generation of more potent combinatorial tumor suppressor alterations capable of driving tumor development using Lenti-sgTS102/Cre pool. Tumors in Nf1^{f/f}; TC, Pten^{f/f}; TC, and Trp53^{f/f}; TC mice transduced with the Lenti-sgTS102/Cre pool were positive for TTF1, a marker for lung adenocarcinoma, and were negative for the squamous cell and small cell lung cancer markers P63 and UCHL1, respectively (FIG. 1e). These results are consistent with these tumors being lung adenomas and adenocarcinomas.

The low efficiency of tumor generation upon inactivation of tumor suppressor genes could suggest that some of these tumors contained spontaneous mutations in known proto-oncogenes and therefore were not oncogene-negative. To investigate this possibility, we PCR-amplified and sequenced 10 genomic regions that include the hotspot oncogene mutation sites in Kras, Braf, Nras, and Egfr (FIG. 8d and Methods) [33, 39-47]. Across 29 samples containing at least one tumor, we detected only one oncogene mutation (a Kras^{G12V} mutation in a tumor from a Pten1^{f/f}; TC mouse. Thus, the majority of these tumors arise in the absence of the hotspot mutations in the aforementioned proto-oncogenes. This observation is consistent with the low spontaneous mutation rate in mouse models of lung cancer [48] and suggests that the inactivation of combinations of specific tumor suppressor genes in Nf1^{f/f}; TC, Pten^{f/f}; TC, and Trp53^{f/f}; TC mice could be sufficient to drive the development of lung cancer in vivo. However, the overall low number of tumors indicates that inactivation of the “core” tumor suppressor genes alone, and most combinations of tumor suppressors tested, are insufficient to generate lung tumors.

### Identification of Top Candidate Tumor Suppressor Genes Involved in Oncogene-Negative Lung Tumor Formation

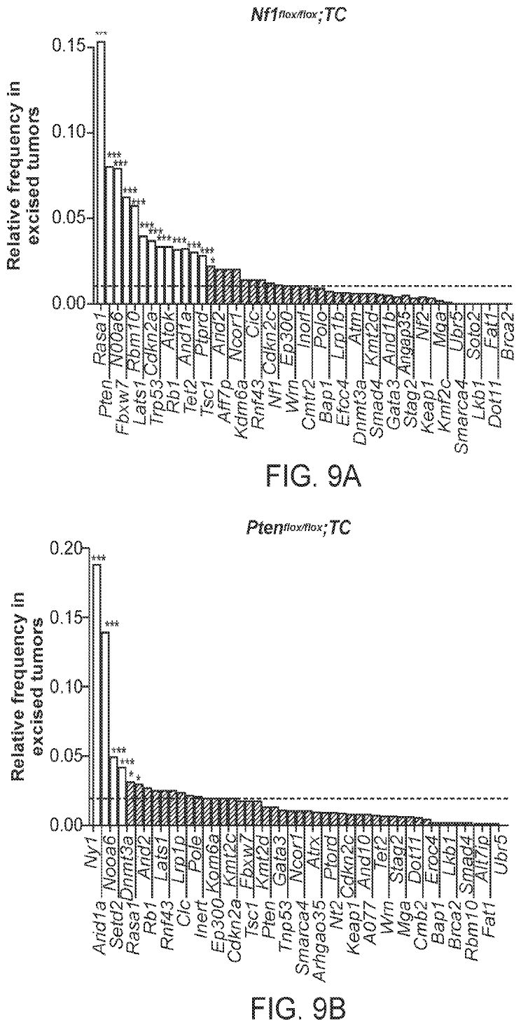

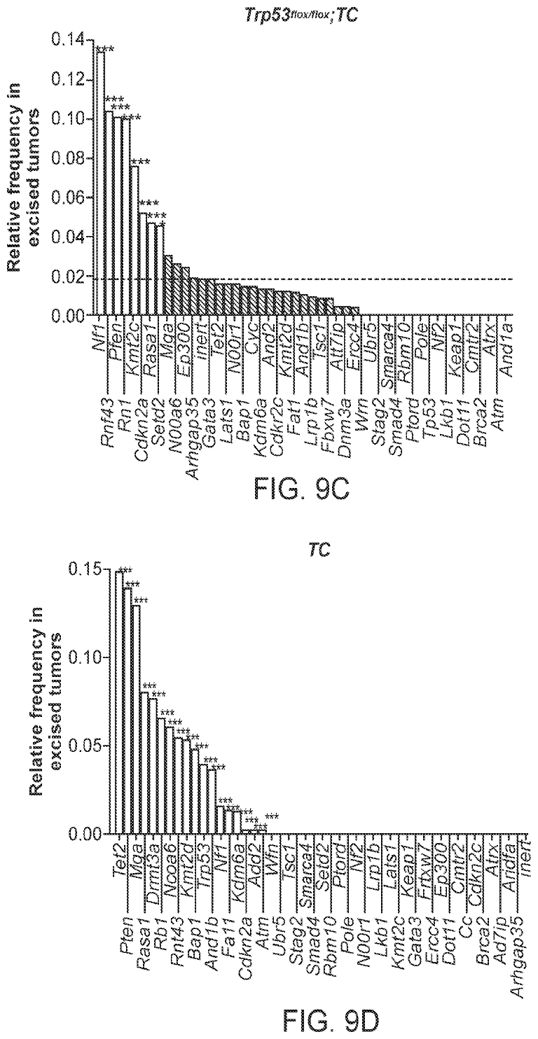

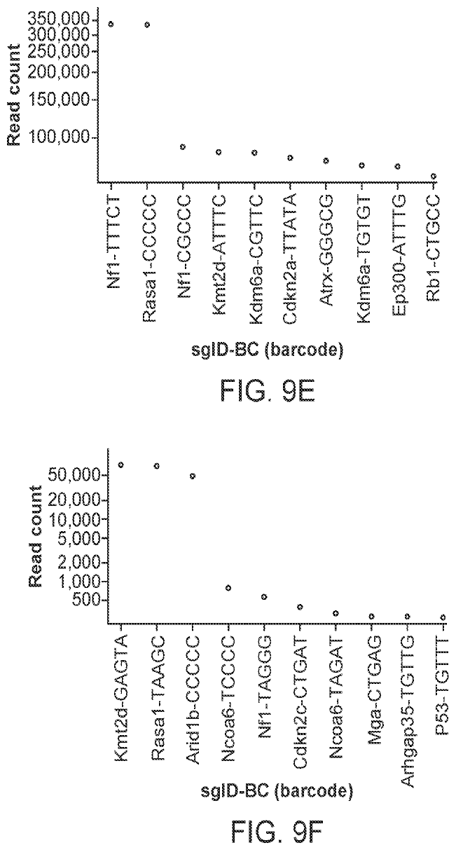

To determine which sgRNAs were present in the largest tumors in mice transduced with Lenti-sgTS102/Cre pool, we PCR-amplified the barcode region of lentiviral backbones from genomic DNA of dissected tumors and performed high-throughput DNA sequencing. These barcodes contain two components: a DNA sequence that identifies the sgRNA targeting a tumor suppressor (sgID) as well as a random barcode region that uniquely tags each clonal tumor (BC) and can be used to quantify the number of cells in each tumor (FIG. 1a). Our sequencing results demonstrated that most large tumors contained multiple Lenti-sgRNA/Cre vectors targeting different tumor suppressors. Therefore, we ranked sgRNAs in each mouse genotypic background based on their relative representation in the dissected tumors (FIG. 1f and 9, see Methods).

To further quantify the impact of inactivating each tumor suppressor gene on increasing growth advantage in vivo, we performed tumor barcode sequencing (Tuba-seq) on bulk DNA from one lung lobe of each Nf1^{f/f}; TC, Pten1^{f/f}; TC, Trp53^{f/f}; TC, and TC mouse with Lenti-sgTS102/Cre-initiated tumors (FIG. 1c). Analysis of the number of cells in clonal expansions of various genotypes further nominated tumor suppressor genes that potentially contribute to tumor initiation and growth (FIG. 1f, 10, and 11). Based on these two analyses, we identified 13 tumor suppressor genes that most likely contribute to the initiation and growth of oncogene-negative tumors (FIG. 1f). The potential importance of these top tumor suppressor genes was often supported by both sgRNAs targeting each gene, suggesting on-target effects. Finally, the presence of sgRNAs targeting our “core” tumor suppressors allowed us to cross-validate our screen. For example, Lenti-sgPten/Cre was enriched in tumors in Nf1^{f/f}; TC mice, and Lenti-sgNf1/Cre was enriched in tumors in Pten1^{f/f}; TC mice (FIG. 1f and 9-6).

### Inactivation of Top Candidate Tumor Suppressors Efficiently Generates Lung Tumors

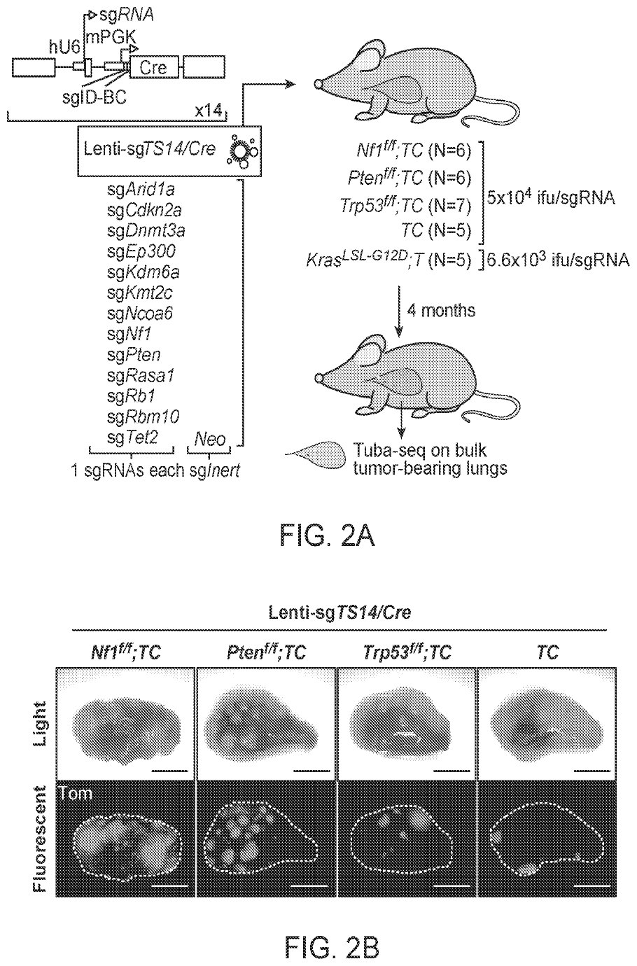

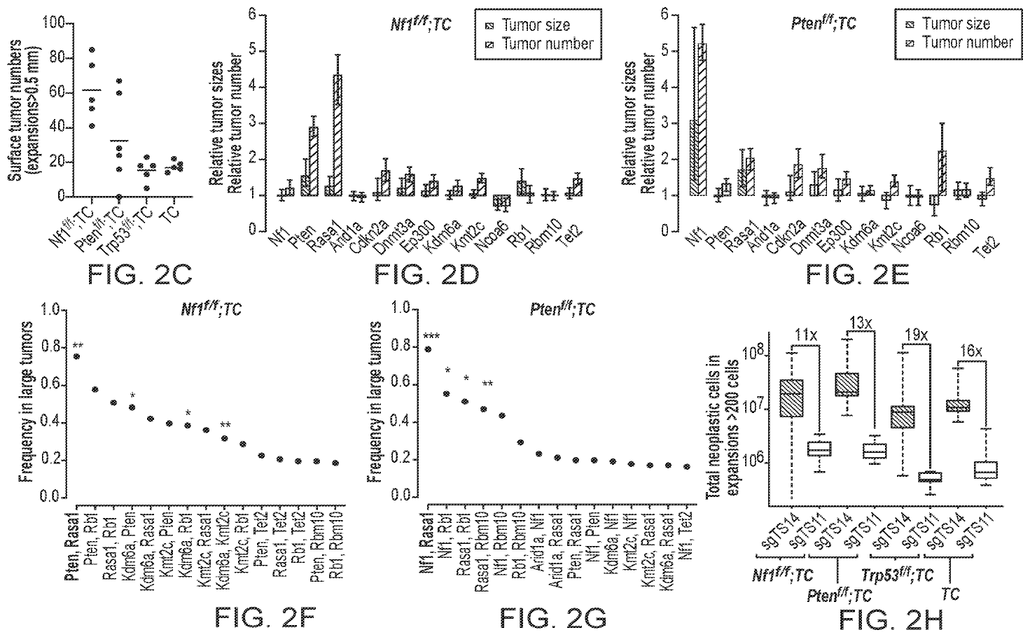

To determine the potential of the 13 top tumor suppressor genes to initiate oncogene-negative tumors, we generated a pool of Lenti-sgRNA/Cre vectors targeting each of these 13 suppressor genes and one vector with an inert sgRNA (Lenti-sgTS14/Cre pool; FIG. 2a). To increase our statistical power to detect tumor suppressor interactions, we targeted each gene with the sgRNA that produced the most significant effect on tumor growth and used five times more of each lentiviral vector per mouse than what was used in Lenti-sgTS102/Cre pool. We initiated tumors with Lenti-sgTS14/Cre in Nf1^{f/f}; TC, Pten^{f/f}; TC, Trp53^{f/f}; TC, TC, and KT mice. Less than four months after tumor initiation, several Nf1^{f/f}; TC and Pten^{f/f}; TC mice showed signs of extensive tumor burden. These mice developed on average four times more tumors than mice of the identical genotypes one year after transduction with Lenti-sgTS102/Cre targeting 48 tumor suppressors (compare FIG. 2b-c with FIGS. 1c-d and 18c). This result confirmed that the tumor suppressor genes identified by enrichment in tumors initiated by the original Lenti-sgTS102/Cre pool were capable of efficiently generating oncogene-negative lung tumors in a shorter time.

We performed Tuba-seq on DNA from bulk tumor-bearing lungs from all mice to determine the number and size of each tumor with each barcoded Lenti-sgRNA/Cre vector. To quantify the exact representation of each vector in the Lenti-sgTS14/Cre pool, which enables a more precise calculation of tumor suppression effects using Tuba-seq, we initiated tumors in Kras^{LSL-G12D}; T (KT) mice with this pool. In the absence of Cas9 in these mice, all sgRNAs act as inert sgRNAs. Therefore, the relative abundance of tumors with each Lenti-sgTS/Cre vector equals the relative frequency of each virus within the pool (see Methods). Inactivation of Nf1, Pten, and Rasa1 using sgRNAs increased tumor size and/or tumor number across all mouse genotypes (FIG. 2d-e, 12a-b, and Methods). Inactivation of each of the other ten tumor suppressor genes had less dramatic and more genotype-specific effects, suggesting that additional molecular pathways altered by these remaining tumor suppressor genes can also likely lead to early epithelial expansions.

Analysis of the largest tumors in Nf1^{f/f}; TC, Pten^{f/f}; TC, Trp53^{f/f}; TC, and TC mice with Lenti-sgTS14/Cre initiated tumors suggested that these tumors were frequently generated through inactivation of more than two tumor suppressor genes. Vectors targeting Nf1, Rasa1, and/or Pten were often present in the largest tumors, and the co-incident alteration of Nf1, Rasa1, and Pten was the most frequent combinatorial alterations observed in the largest tumors of Nf1^{f/f}; TC, Pten^{f/f}; TC, and TC mice (FIGS. 2f-g, 12c-d, and 13).

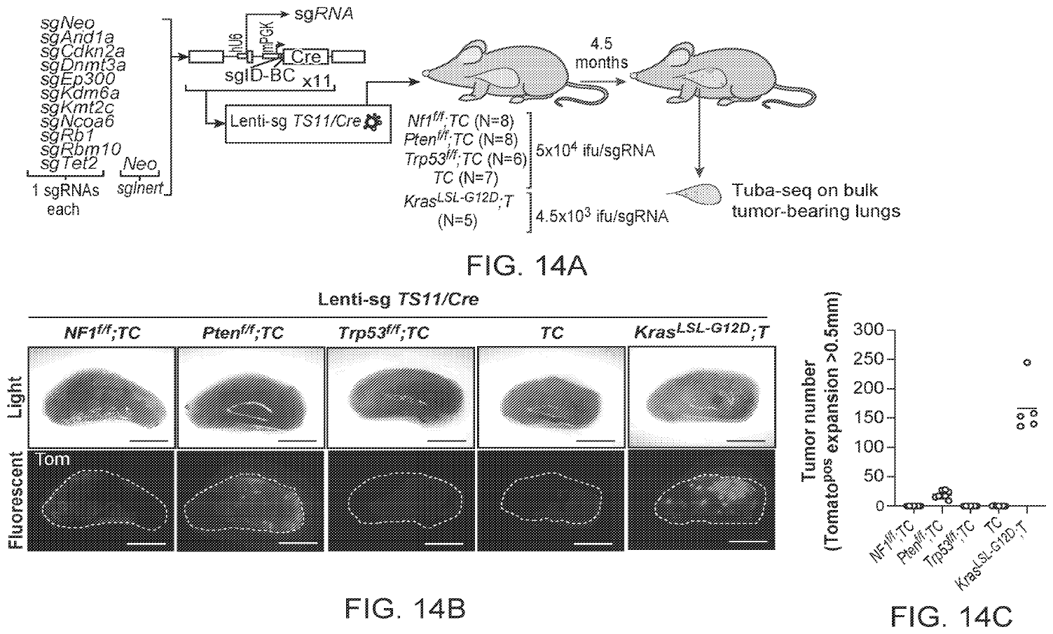

To gain greater insight into the contribution of Nf1, Rasa1, and Pten inactivation to the generation of oncogene-negative tumors, we transduced Nf1^{f/f}; TC, Pten^{f/f}; TC, Trp53^{f/f}; TC, TC, and KT mice with a pool of Lenti-sgRNA/Cre vectors that excluded the vectors targeting Nf1, Rasa1, and Pten (Lenti-sgTS11/Cre) (FIG. 14a). Approximately 4 months after transduction, these mice had very few tumors (FIG. 14b-c). Tuba-seq on DNA from bulk tumor-bearing lungs from these mice uncovered a drastic decrease in tumor burden across all genotypes of mice that received the Lenti-sgTS11/Cre pool relative to Lenti-sgTS14/Cre pool (FIG. 2h). Thus, the inactivation of Nf1, Rasa1, and Pten emerged as the most potent contributors to the generation of oncogene-negative lung tumors.

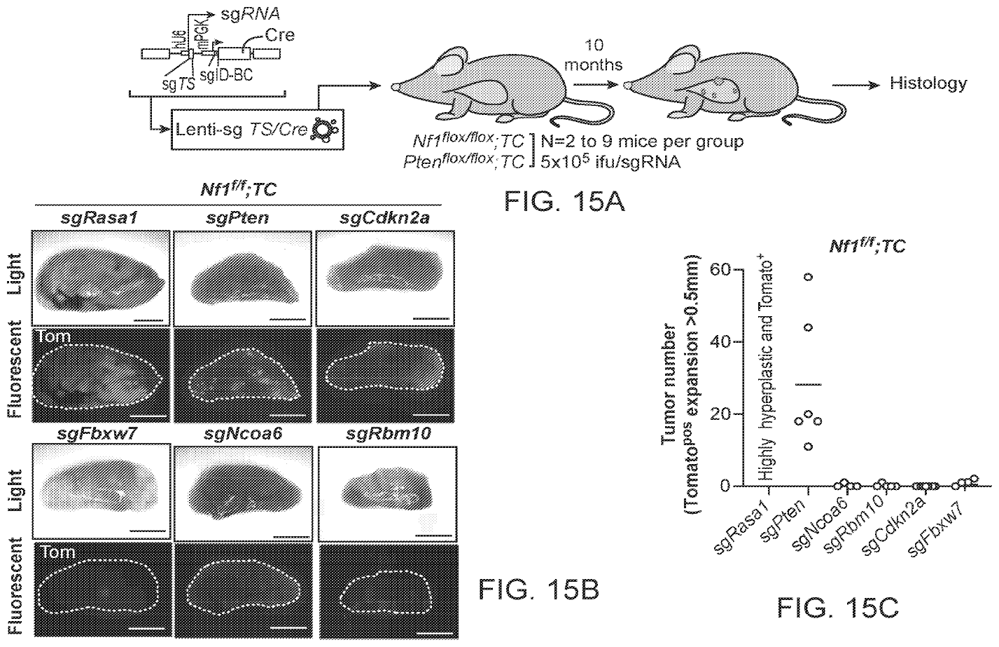

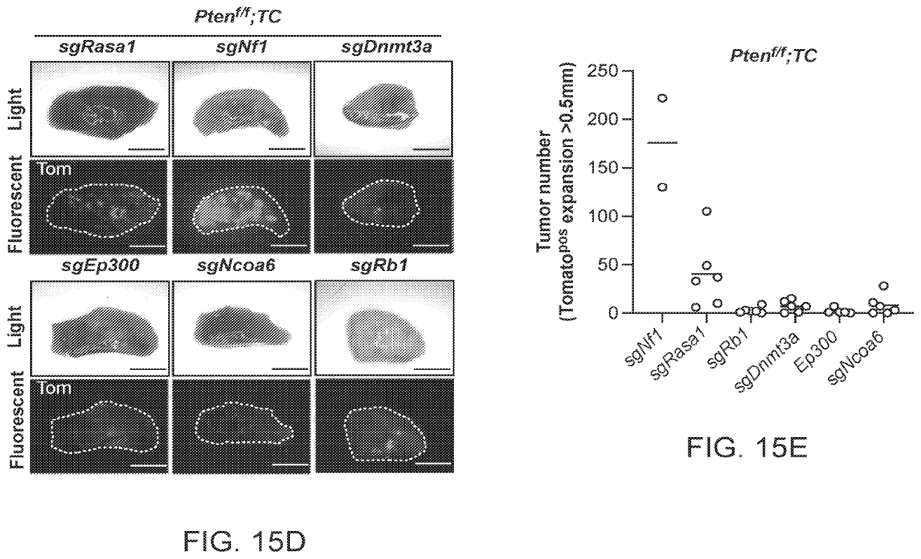

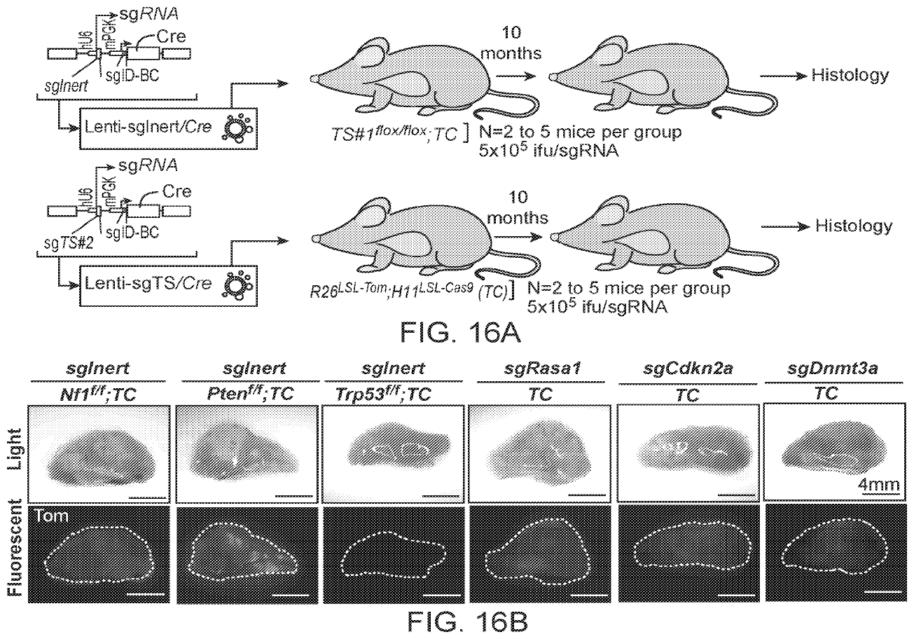

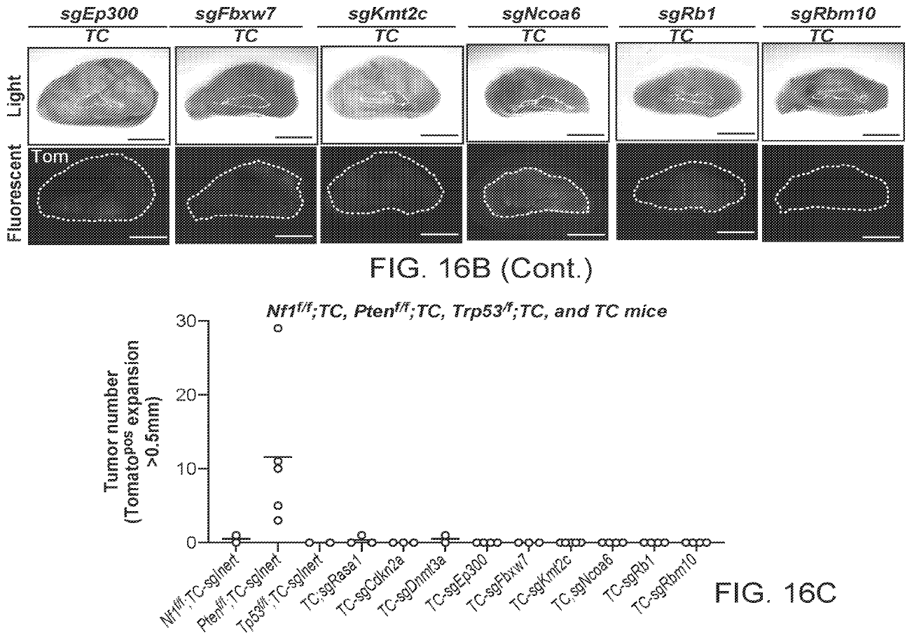

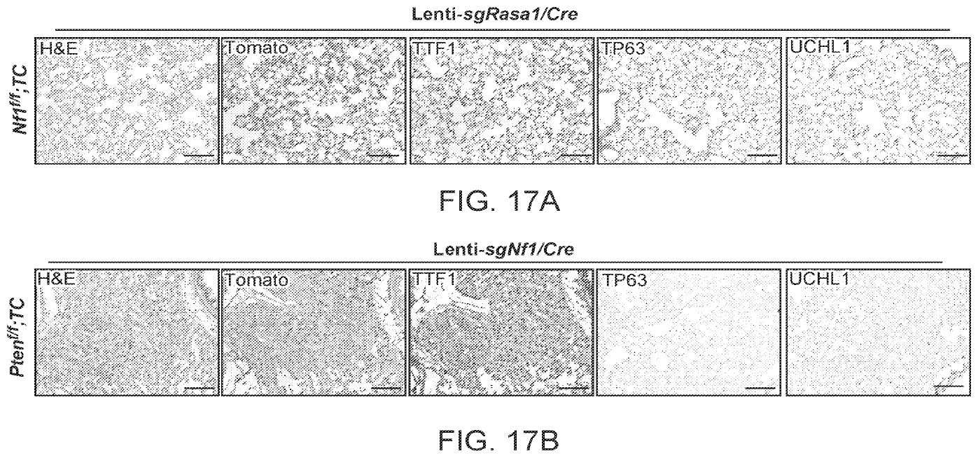

To further assess the ability of single and pairwise tumor suppressor inactivation after a longer time, we transduced Nf1; TC, Pten^{f/f}; TC, and TC mice with higher titers of Lenti-sgRNA/Cre vectors targeting single tumor suppressor genes (10 and 50 times higher titer than in the experiments with Lenti-sgTS14/Cre or Lenti-sgTS102/Cre, respectively). Analysis of these mice ten months after lentiviral transduction (versus four months after transduction in mice transduced with Lenti-sgTS14/Cre or Lenti-sgTS11/Cre, FIG. 15a) suggested relatively modest ability of pairwise inactivation of tumor suppressor genes to drive overt tumor development. Inactivation of Rasa1 in Nf1^{f/f}; TC mice led to extensive epithelial hyperplasia but not to the growth of solid tumors, while single alteration of Nf1 or Rasa1 alone led to small expansions (FIG. 15-17), demonstrating that NF1 and RASA1 are not functionally redundant despite both encoding RAS GTPase activation proteins (GAP). Combined inactivation of Nf1 and Pten or Rasa1 and Pten generated very few solid tumors (FIGS. 15b-e and 17a-b). Also, inactivation of other tumor suppressor genes in Nf1^{f/f}; TC, Pten^{f/f}; TC, and TC mice rarely generated lung tumors (FIGS. 15 and 16). Collectively, these observations suggest that single and pairwise tumor suppressor gene inactivation is rarely sufficient to generate large lung tumors and that combinatorial inactivation of three or more tumor suppressor genes increases the efficiency of tumor development and/or accelerates the growth of oncogene-negative lung tumors.

### Combinatorial Inactivation of Nf1, Rasa1, and Pten Promotes Lung Adenocarcinoma Development Comparably to Oncogenic Kras Mutation

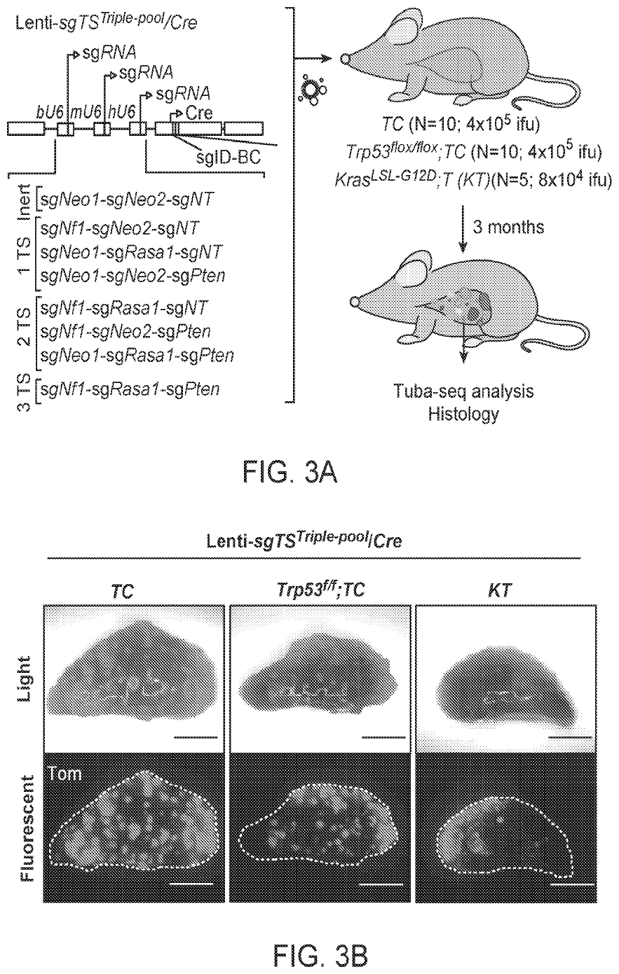

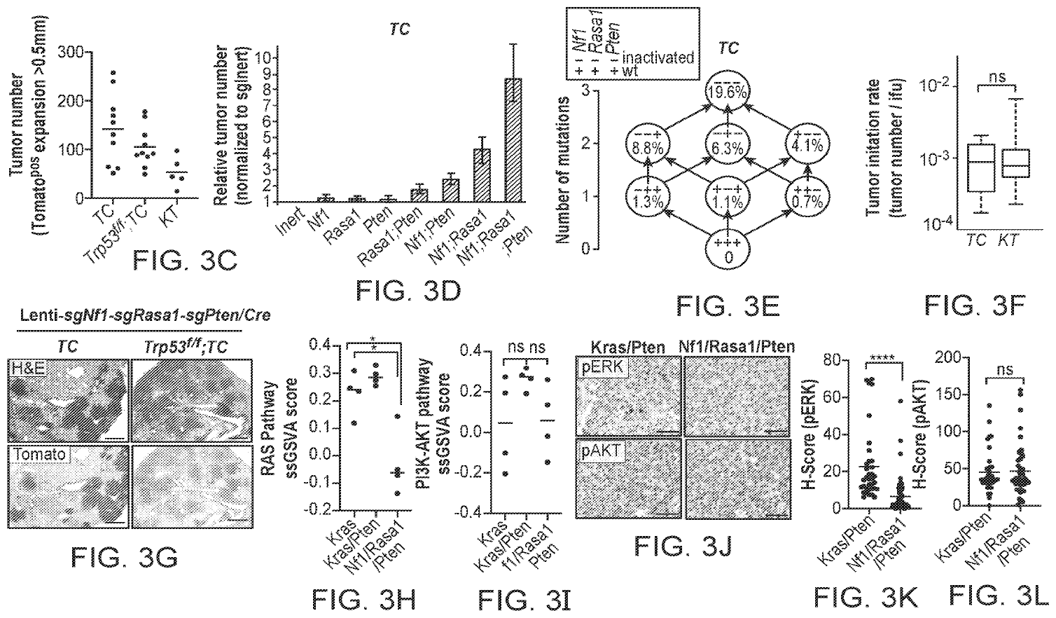

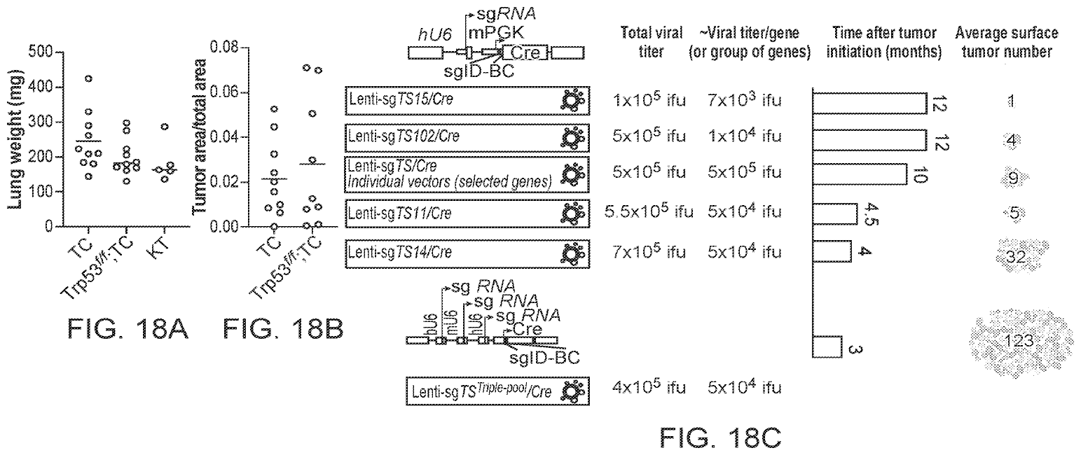

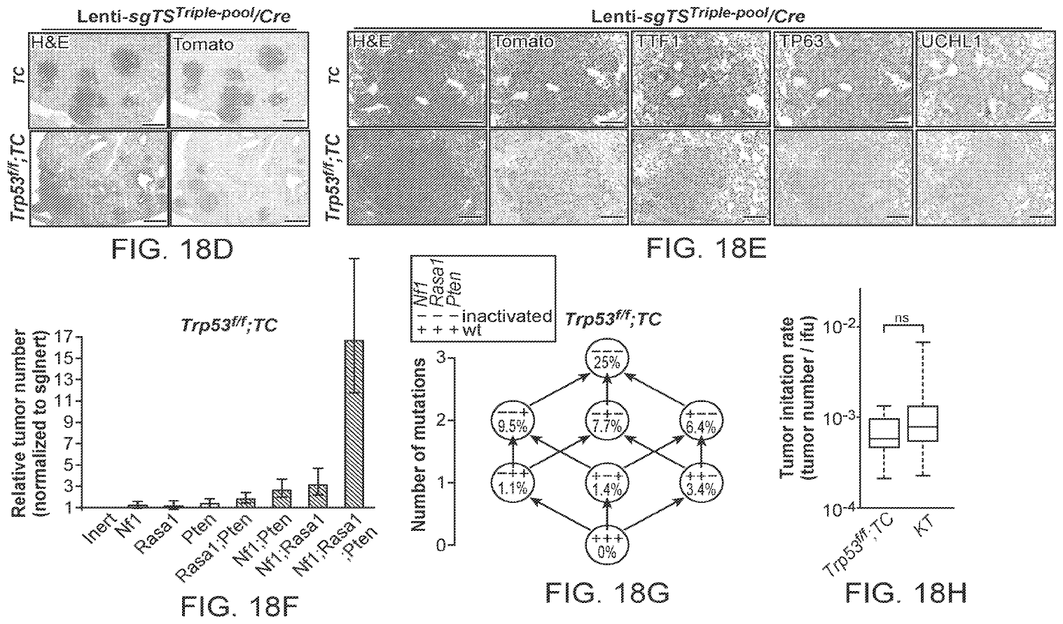

Next, we dissected the higher-order genetic interactions between Nf1, Rasa1, and Pten. We transduced TC mice with a pool of eight lentiviral vectors, each carrying three sgRNA cassettes to inactivate all single, double and triple combinations of Nf1, Rasa1, and Pten individually, in pairwise combinations, and all three simultaneously (Lenti-sgTS^{Triple-pool}/Cre, FIG. 3a). Three months after tumor initiation, TC mice had hundreds of large tumors (FIG. 3b-c and FIG. 18a-d). These tumors were adenomas and adenocarcinoma that were positive for TTF1 and negative for markers of squamous and small cell carcinoma, TP63 and UCHL1 (FIG. 18e). Tuba-seq analysis on these lungs showed that the majority of tumor burden in TC mice arose as a consequence of concomitant inactivation of all three tumor suppressors (FIG. 3d).

In molecular evolution studies, generating combinatorial genomic alterations and measuring each genotype's fitness (growth rate) is often used to infer the possible and the most probable paths from a wild-type state to a complex genotype [49]. Through the generation of all the possible combinatorial alterations of Nf1, Rasa1, and Pten, we quantitatively determined the fitness conferred by each mutation and the relative probabilities of different adaptive paths leading from a wild-type state to the triple mutant genotype (see Methods). Our data suggest that inactivation of these three genes can occur in any order, with each additional alteration further increasing the fitness (FIG. 3e). The Nf1→Rasa1→Pten mutation sequence is the most probable path of all six possible paths.

Next, we investigated the potency of simultaneous inactivation of Nf1, Rasa1, and Pten on lung tumor initiation. To compare the tumor initiation potential of combinatorial Nf1, Rasa1, and Pten mutations with that of a known oncogene, we transduced Kras^{LSL-G12D}; T (KT) mice along with TC mice with Lenti-sgTS^{Triple-pool}/Cre (FIG. 3a). Strikingly, co-incident inactivation of Nf1, Rasa1, and Pten in TC and Trp53^{f/f}; TC mice was nearly as potent as oncogenic KRAS^{G12D} in driving lung tumor initiation in vivo (FIG. 3f and Methods).

Additionally, we assessed the effect of Trp53 deficiency, which is the most frequently altered tumor suppressor gene in oncogene-negative human lung adenocarcinomas (FIG. 7b), on the ability of Nf1, Rasa1, and Pten inactivation to generate lung tumors utilizing Lenti-sgTS^{Triple-pool}/Cre (FIGS. 3a-c and 18a-g). Interestingly, additional inactivation of Trp53 did not increase the tumor initiation potential of combinatorial alterations of Nf1, Rasa1, and Pten (compare FIG. 3f and FIG. 18h). This observation suggests that Trp53 is not a major suppressor of oncogene-negative tumor development, at least at these early stages of malignant transformation.

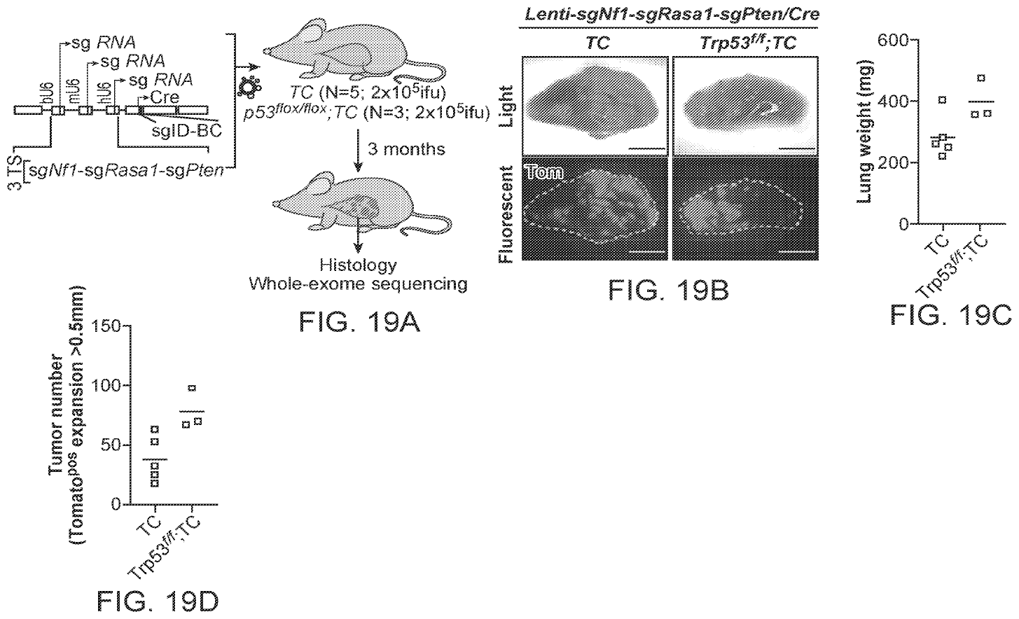

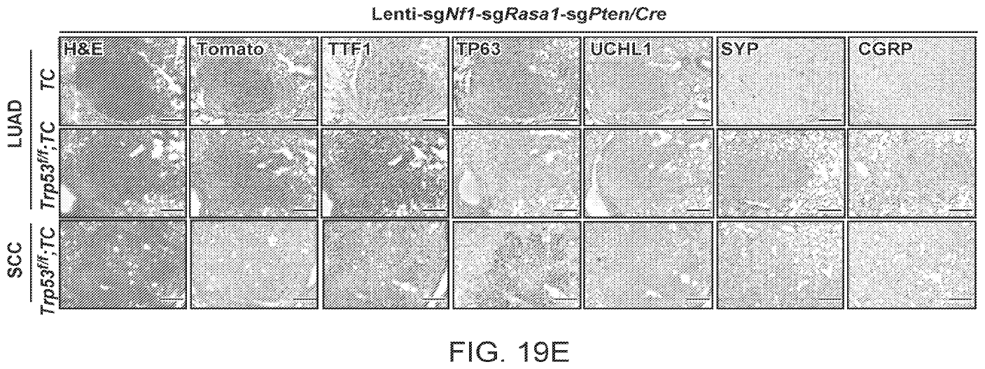

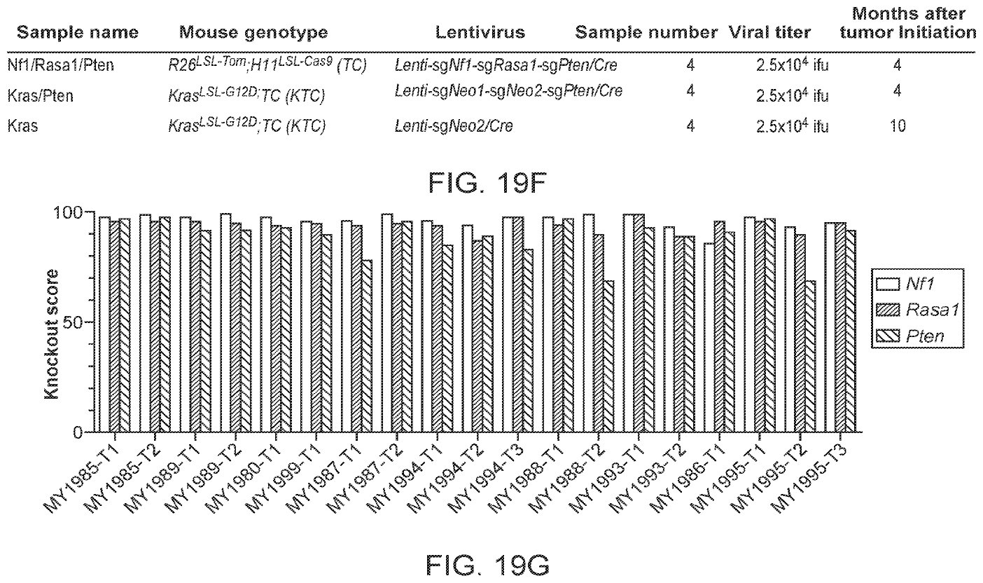

To further analyze tumors driven by inactivation of Nf1, Rasa1, and Pten, we initiated tumors in TC and Trp53^{f/f}; TC mice using only the lentiviral vector that targets all three tumor suppressor genes (Lenti-sgNf1-sgRasa1-sgPten/Cre) (FIG. 3g, FIG. 19a-d). This generated very large numbers of lung tumor in only three months. Histological examination and immunohistochemical staining for TTF1, TP63, and UCHL1 indicated that TC and Trp53^{f/f}; TC mice developed almost exclusively lung adenomas/adenocarcinomas (FIG. 19e).

Finally, to comprehensively investigate the extent to which Nf1, Rasa1, and Pten mutant tumors develop spontaneous mutations, we performed whole-exome sequencing. This analysis uncovered no putative oncogene mutations and only a few putative tumor suppressor mutations, none of which occurred in more than one tumor.

### Oncogene-Negative Lung Adenocarcinomas Activate RAS and PI3K Pathways in Mouse Models

NF1 and RASA1 are best characterized as negative regulators of RAS, while PTEN is a negative regulator of the PI3K-AKT pathway. Therefore, we investigated the impact of inactivating these three tumor suppressor genes on RAS and PI3K pathway activation and compared the level of activation to that elicited by oncogenic KRAS^{G12D} using gene expression profiling. We isolated cancer cells from autochthonous tumors in which Nf1, Rasa1, and Pten were simultaneously inactivated (TC mice with Lenti-sgNf1-sgRasa1-sgPten/Cre; Nf1/Rasa1/Pten tumors), KRAS^{G12D} was expressed (KT; H11^{LSL-Cas9} mice with Lenti-sgInert/Cre; Kras tumors), and KRAS^{G12D} was expressed in the context of Pten inactivation (KT; H11^{LSL-Cas9} mice with Lenti-sgPten/Cre; Kras/Pten tumors) and performed RNA sequencing (RNA-seq)-based gene expression profiling (FIG. 19f, and Methods).

To compare the expression of genes downstream of the RAS and PI3K-AKT pathway activation in Nf1/Rasa1/Pten tumors to tumors driven by activation of KRAS^{G12D}, we performed single-sample gene set variation analysis (ssGSVA) to derive enrichment scores for individual tumors based on previously reported gene sets representing RAS and PI3K-AKT regulated genes [50, 51]. Interestingly, Nf1/Rasa1/Pten tumors exhibited a lower RAS pathway gene signature than KRAS^{G12D}-driven tumors (FIG. 3h). PI3K-AKT pathway gene signature was almost equally expressed in Nf1/Rasa1/Pten, Kras, Kras, and Kras/Pten tumors (FIG. 3i). We also examined PI3K and RAS pathway activation by performing immunohistochemistry on Nf1/Rasa1/Pten and Kras/Pten tumors. Consistent with gene expression analysis, Nf1/Rasa1/Pten tumors had positive staining for pERK (indicative of RAS pathway activation), however, the level of pERK staining was less intense than KRAS^{G12D}-driven tumors (FIG. 3j-k). pAKT staining intensity in Nf1/Rasa1/Pten tumors was similar to Kras/Pten tumors (FIG. 3j,l). Thus, we uncovered pathway-level changes that drive lung cancer in the absence of oncogene mutations in vivo.

### Oncogene-Negative Human Lung Adenocarcinomas Frequently Exhibit Activation of RAS and PI3K Pathways

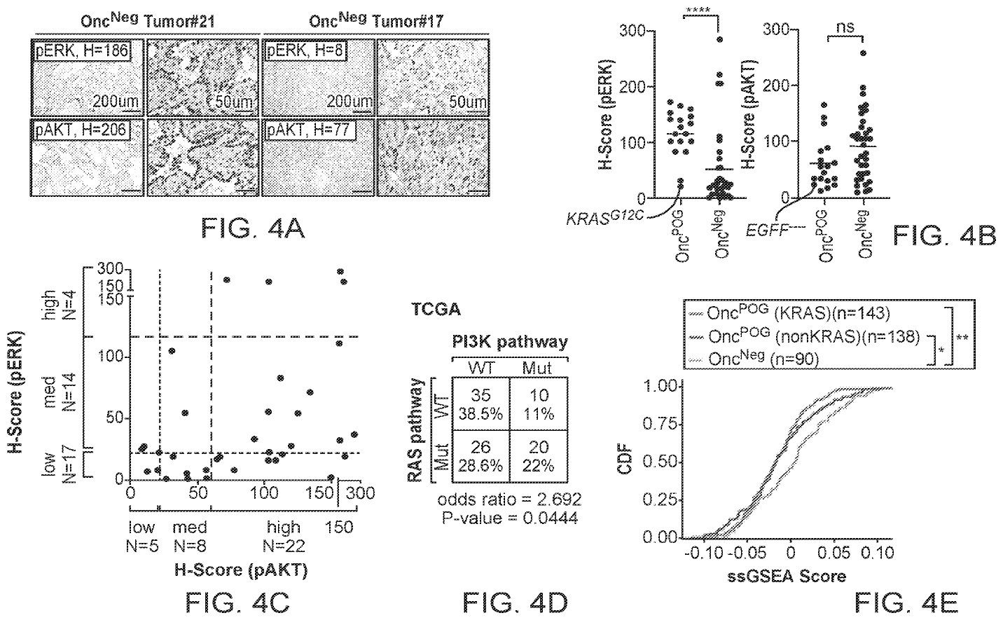

To investigate the activation of RAS and PI3K pathways in human oncogene-negative lung adenocarcinomas, we assessed RAS and PI3K activation in histological specimens from oncogene-negative (N=35) and oncogene-positive (N=18) lung adenocarcinomas. These tumors were genomically characterized by Stanford's Solid Tumor Actionable Mutation Panel (STAMP), which screens 130 genes, either in part or fully [52]. We stained sections from each tumor for pERK and pAKT as markers of RAS and PI3K pathway activation. We observed that both pathways are broadly activated in most oncogene-negative lung adenocarcinomas (FIG. 4a,b, and 20). In general, the average RAS pathway activation was lower, and PI3K pathway activation was similar in oncogene-negative tumors compared with oncogene-positive tumors, which is consistent with our observations in our mouse models (FIG. 3h-i). Over 90% of oncogene-negative human tumors had moderate to strong activation of RAS and/or PI3K pathways, and ˜45% of these patients had moderate to strong activation of both RAS and PI3K pathways (FIG. 4c). Interestingly, RAS and PI3K pathway activation was only explained by mutations in NF1, PTEN, and a few other genes profiled by STAMP for a small fraction of cases (Table 4). This observation could be due to the noncomprehensive gene panel characterized in these tumors (Table 4) or the presence of additional unknown genetic and/or epigenetic mechanisms of RAS and PI3K pathway activation.

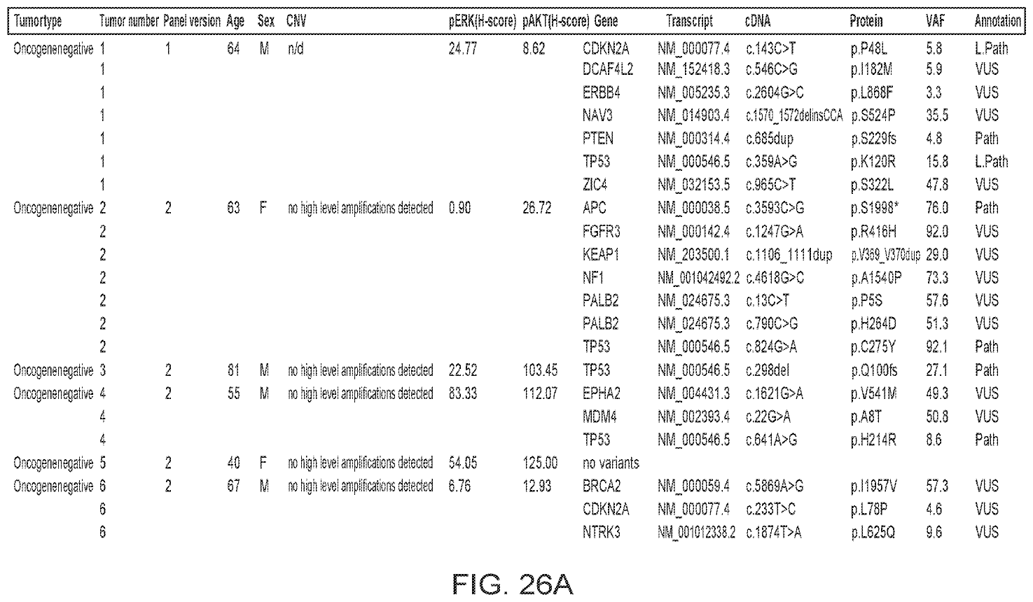

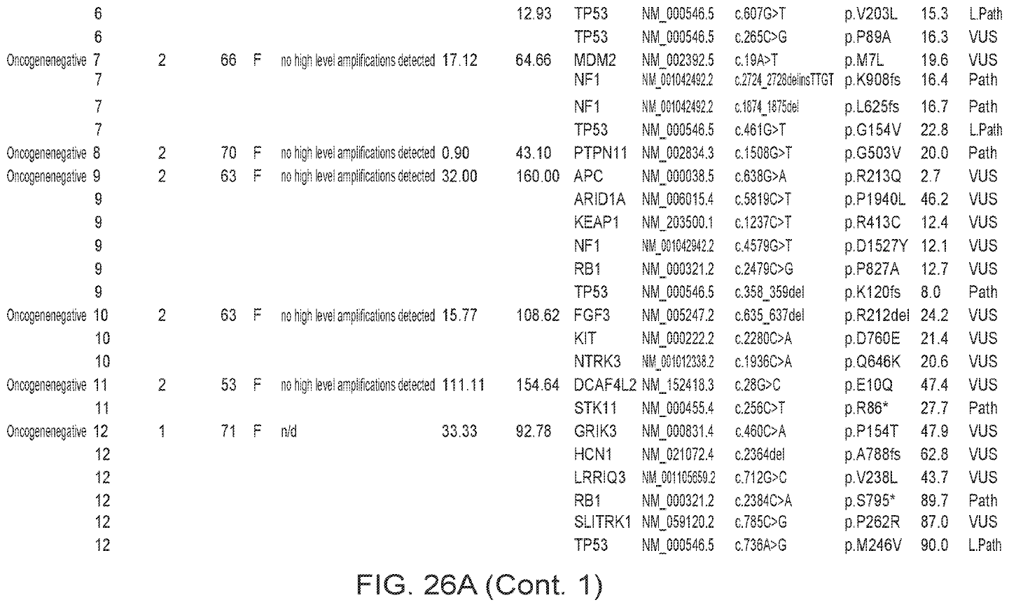

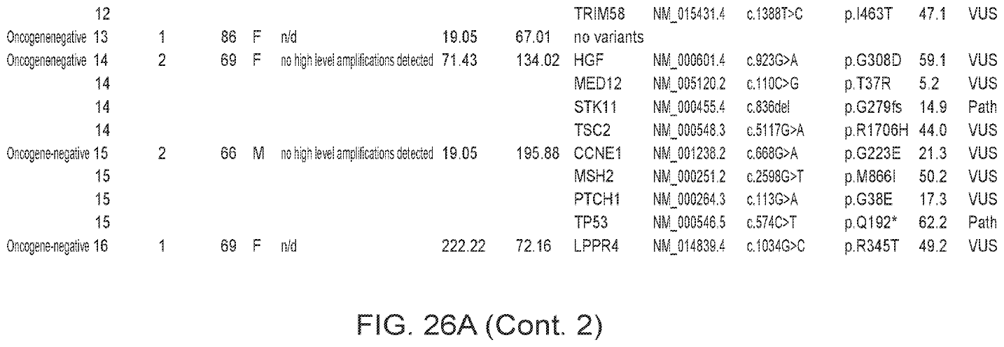

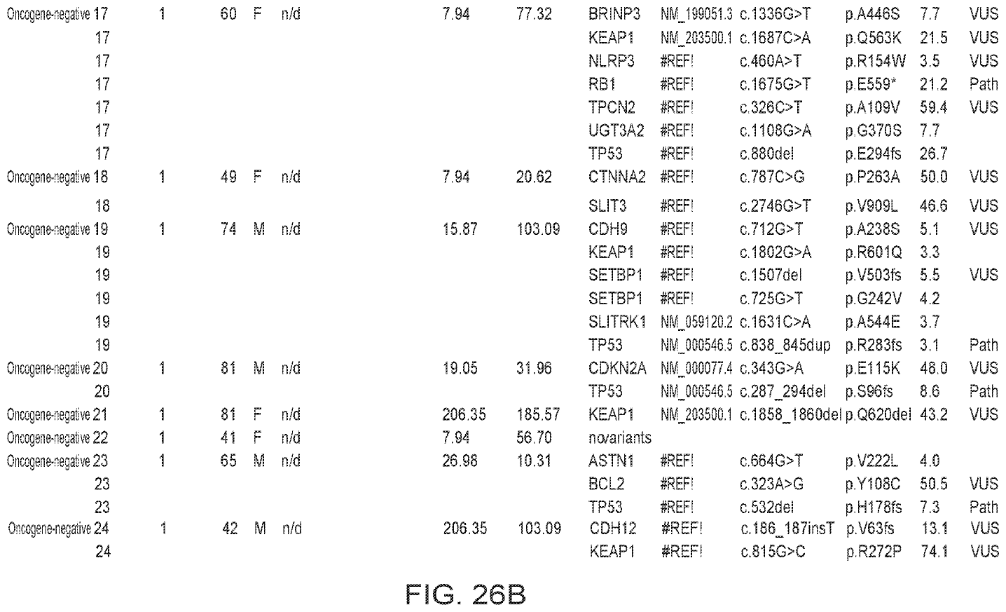

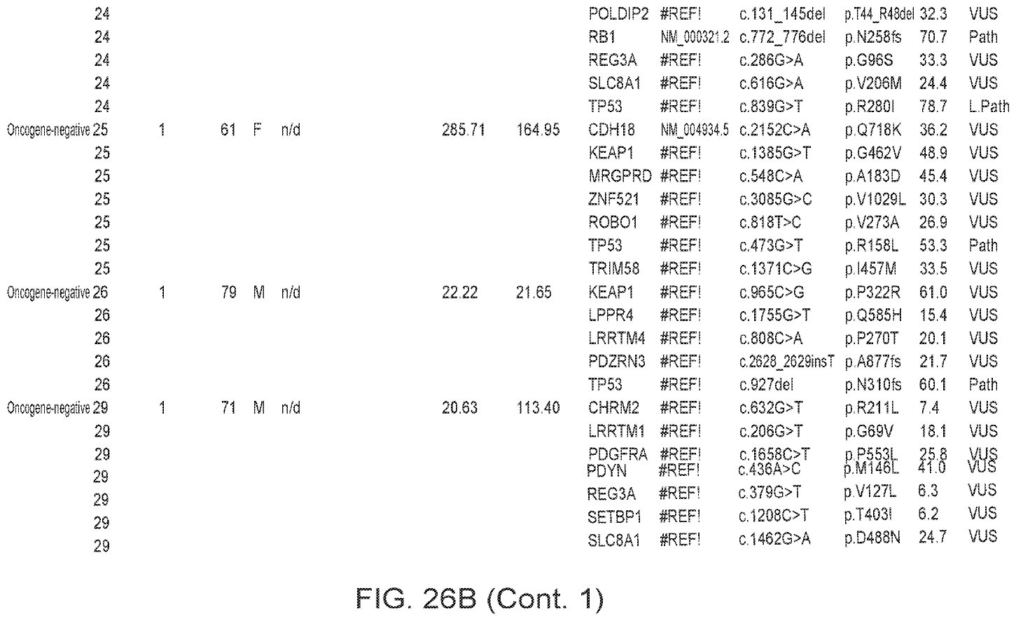

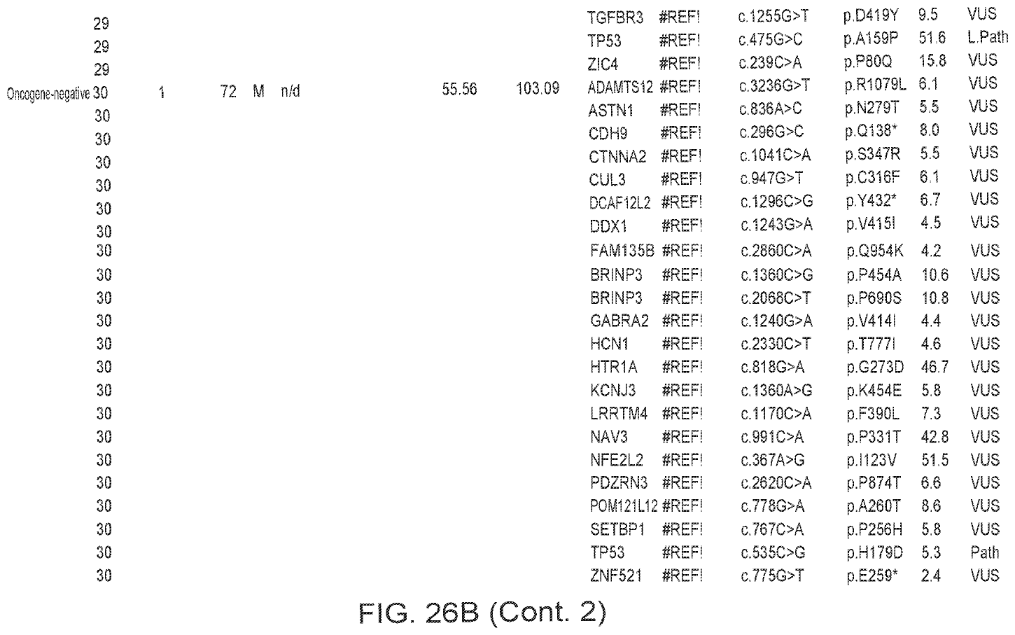

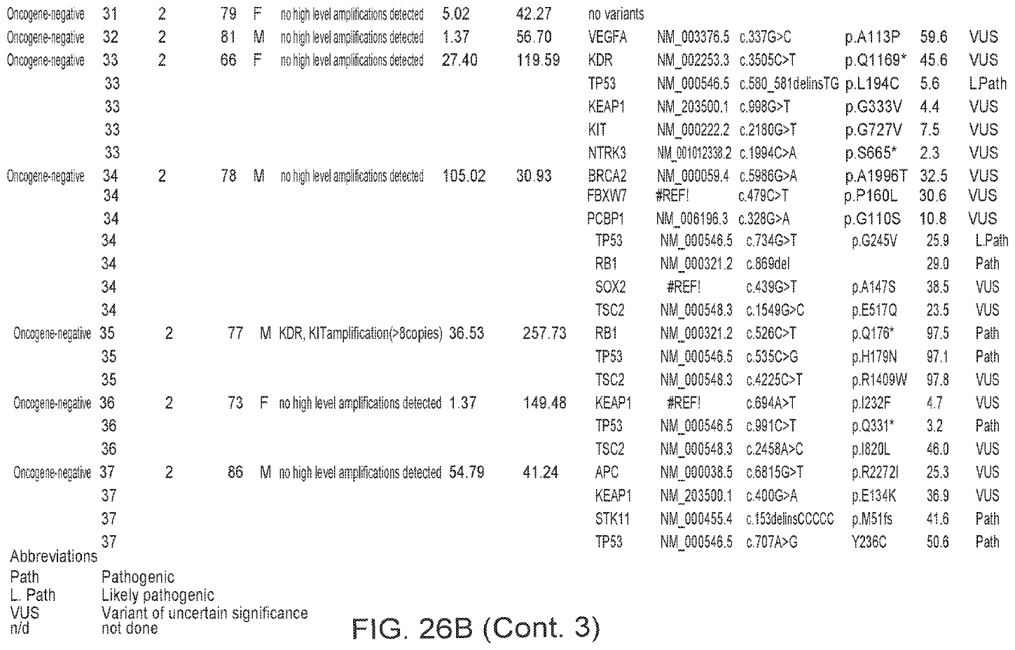

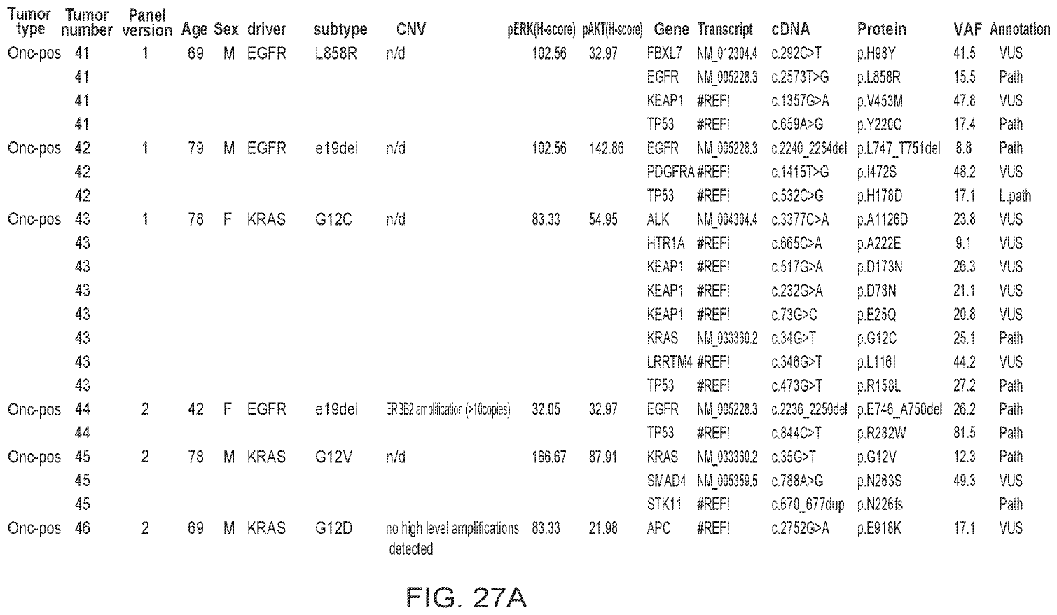

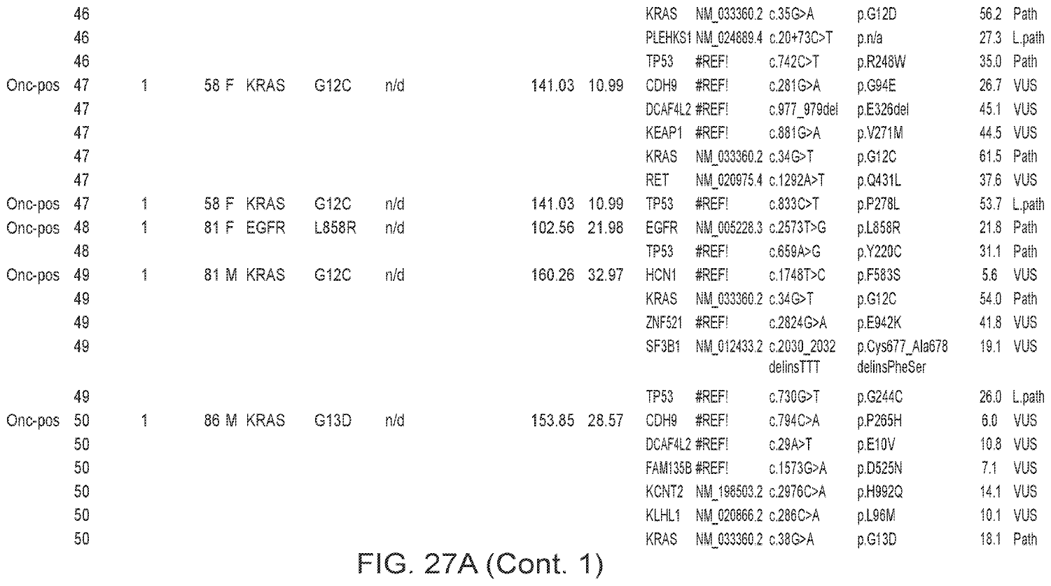

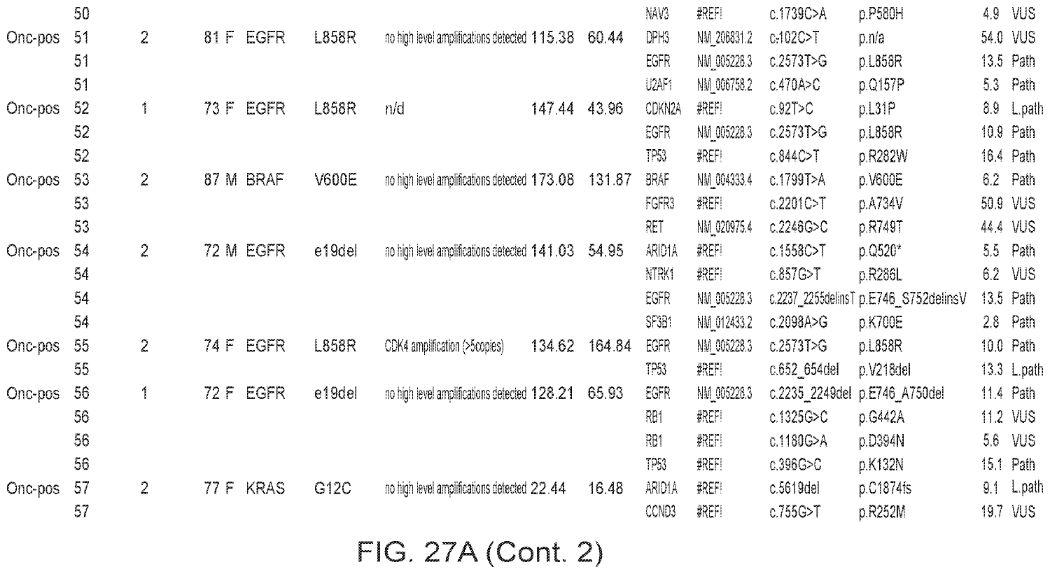

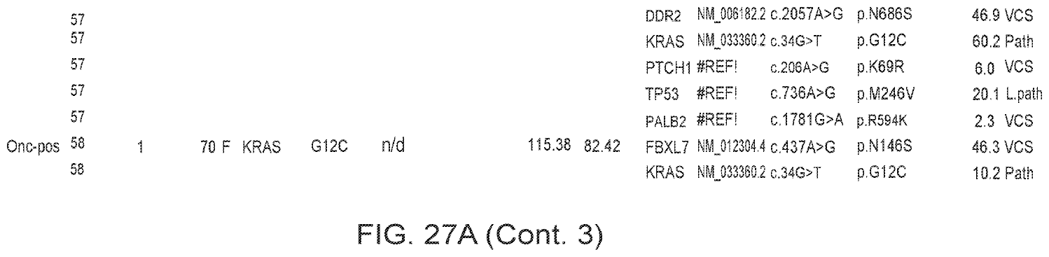

TABLE 4
Characteristics of lung adenocarcinoma patients with oncogene-negative and
oncogene-positive tumors assessed for activation of RAS and PI3K pathways
(See also - FIG. 26 and FIG. 27). These tumors are genomically characterized
by Stanford's Solid Tumor Actionable Mutation Panel (STAMP). Genes in each
version of STAMP are listed in “STAMP v1 gene list” and “STAMP v2 gene list”
STAMP Version 1 (198 genes)

| | | | | | |
|---|---|---|---|---|---|
| ABCC9 | CTSS | HRAS | MDM2 | PIK3CA | SMAD4 |
| ABL1 | CUL3 | HTR1A | MECOM | PKLR | SOX2 |
| ADAMTS12 | DCAF12L1 | HTR3E | MET | POLDIP2 | SPTA1 |
| AKT1 | DCAF12L2 | IFLTD1 | MKRN3 | POM121L12 | ST6GAL2 |
| ALK | DCAF4L2 | IGFL3 | MPHOSPH8 | PSPC1 | STK11 |
| AMOT | DCC | IL6R | MRGPRD | PTEN | SYT4 |
| ASB18 | DDR2 | IQCJ | MS4A3 | PTPRD | TARS2 |
| ASTN1 | DDX1 | KCND2 | MYC | RAF1 | TERT |
| ASTN2 | DENND4B | KCNJ3 | MYCL | RB1 | TG |
| ATP11B | DMD | KCNT2 | MYCN | REG3A | TGFBR3 |
| BCL2 | EGFLAM | KDR | MYD88 | RET | TNN |
| BRAF | EGFR | KEAP1 | MYEOV | RFX5 | TNR |
| BRINP3 | ERBB2 | KIT | MYNN | RIT2 | TP53 |
| C14orf177 | ERBB4 | KLHL1 | NAV3 | RLF | TP63 |
| C6orf118 | FAM135B | KLHL6 | NCAM1 | ROBO1 | TPCN2 |
| C9 | FBN2 | KRAS | NETO1 | ROS1 | TPTE2 |
| CA10 | FBXL7 | LANCL2 | NFE2L2 | RPS4Y1 | TRIM58 |
| CCND1 | FBXW7 | LELP1 | NKD2 | S100A7 | TRIP13 |
| CD226 | FGD1 | LPAR6 | NLRP3 | SAMD7 | TRPC5 |
| CDH12 | FGF3 | LPL | NOTCH1 | SEMA6C | TUBA3C |
| CDH18 | FGFR1 | LPPR4 | NRAS | SERPINB3 | U2AF1 |
| CDH7 | FGFR2 | LRFN5 | NT5C1A | SETBP1 | UGT3A2 |
| CDH9 | FOXP1 | LRP1B | NTM | SF3B1 | VSTM2A |
| CDKN2A | FRYL | LRRIQ3 | NUP155 | SGCZ | WDR7 |
| CHRM2 | GABRA2 | LRRTM1 | PABPC4 | SKP2 | WHSC1L1 |
| CLDN11 | GABRA6 | LRRTM4 | PACRG | SLC14A2 | ZAN |
| CNTNAP2 | GRID1 | MACF1 | PAK7 | SLC1A3 | ZFY |
| CNTNAP5 | GRIK3 | MAP2K1 | PARK2 | SLC2A2 | ZIC1 |
| COL22A1 | GRM8 | MAP2K2 | PDGFRA | SLC45A2 | ZIC4 |
| CSMD1 | HAPLN1 | MARCH1 | PDGFRB | SLC6A19 | ZMYM5 |
| CSMD3 | HAX1 | MCCC1 | PDYN | SLC8A1 | ZNF236 |
| CTNNA2 | HCN1 | MCF2L2 | PDZRN3 | SLIT3 | ZNF521 |
| CTNNB1 | HDAC9 | MCL1 | PHC3 | SLITRK1 | ZNF713 |

STAMP Version 2 (130 genes)

| | | | |
|---|---|---|---|
| ABL1 | EGFR | MAP2K1 | PPP2R1A |
| AKT1 | EP300 | MAP2K2 | PTCH1 |
| ALK | EPHA2 | MDM2 | PTEN |
| APC | EPHA3 | MDM4 | PTPN11 |
| AR | ERBB2 | MED12 | RAC1 |
| ARAF | ERBB3 | MET | RAF1 |
| ARID1A | ERBB4 | MLH1 | RB1 |
| AURKA | ESR1 | MPL | RET |
| BAP1 | EZH2 | MSH2 | RHEB |
| BCL2 | FBXW7 | MTOR | RHOA |
| BCR | FGF3 | MYC | RIT1 |
| BRAF | FGF4 | MYCL | ROS1 |
| BRCA1 | FGFR1 | MYCN | SDHD- |
| BRCA2 | FGFR2 | MYD88 | promoter |
| CASP8 | FGFR3 | NF1 | SETBP1 |
| CCND1 | FLT3 | NF2 | SETD2 |
| CCND2 | FOXO1 | NFE2L2 | SF3B1 |
| CCND3 | GATA3 | NKX2-1 | SMAD4 |
| CCNE1 | GNA11 | NOTCH1 | SMO |
| CDH1 | GNAQ | NRAS | SOX2 |
| CDK12 | GNAS | NTRK1 | SPOP |
| CDK4 | HGF | NTRK2 | SRC |
| CDK6 | HNF1A | NTRK3 | SRSF2 |
| CDKN1B | HRAS | PALB2 | STK11 |
| CDKN2A | IDH1 | PCBP1 | TERT- |
| CDKN2B | IDH2 | PDGFRA | promoter |
| CHEK2 | IGF1R | PDGFRB | TP53 |
| CREBBP | JAK2 | PIK3CA | TP63 |
| CTNNB1 | JAK3 | PIK3R1 | TSC2 |
| CUL3 | KDR | PLEKHS1- | VEGFA |
| DDR2 | KEAP1 | promoter | VHL |
| DNMT3A | KIT | POLD1 | YAP1 |
| DPH3- | KRAS | POLE | |
| promoter | | | |

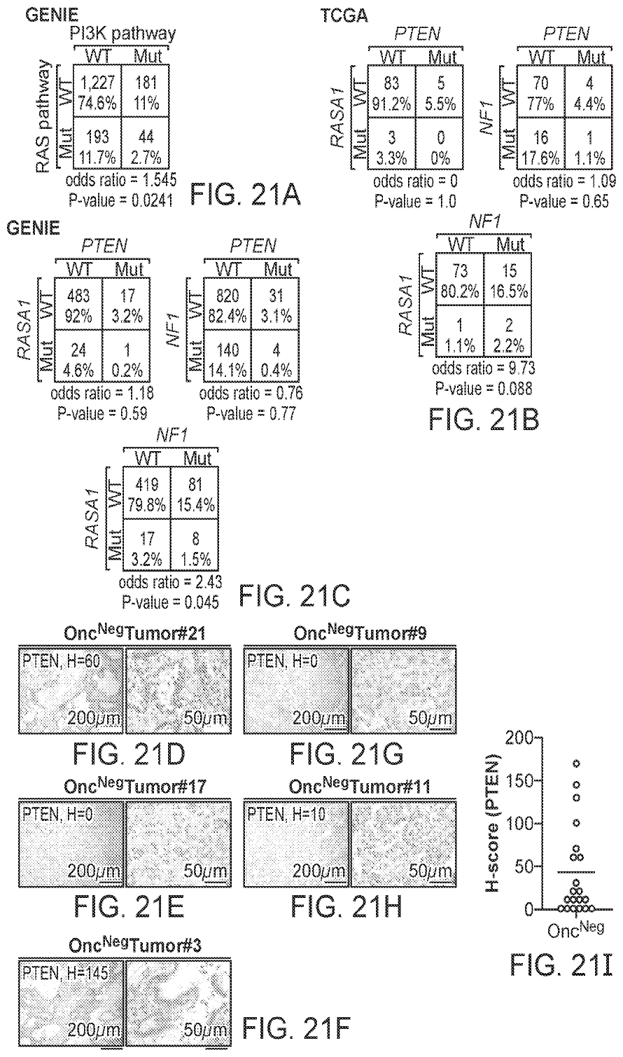

We next assessed oncogene-negative lung adenocarcinomas in TCGA and GENIE for genomic alterations that could lead to the activation of RAS and PI3K. We generated a list of well-established components of that RAS and PI3K pathways that are not known to be oncogenes in lung adenocarcinoma (see Methods). We queried these genes for alterations in oncogene-negative tumors in TCGA and GENIE datasets. Consistent with activation of RAS and/or PI3K pathways in a large fraction of oncogene-negative lung adenocarcinomas, over 40% of oncogene-negative lung adenocarcinomas in TCGA had alterations in RAS and/or PI3K pathways, and 12% of these patients exhibited enrichment of alterations in both pathways (FIG. 4d). These frequencies were lower when we queried genetic alterations in GENIE since only a fraction of the known genes in these two pathways has been analyzed in these patients (FIG. 21a). Consistent with previous reports, NF1 and RASA1 alterations were enriched in oncogene-negative tumors; however, coincident alterations in NF1, RASA1, and PTEN were rare, supporting the importance of pathway-level analysis of human genomic data (FIG. 21b, c) [53, 54]. Our observations are consistent with a model in which the biochemical changes in RAS and PI3K pathways can be generated by diverse genomic and epigenomic alterations.

Finally, we assessed whether oncogene-negative tumors in our mouse model exhibit transcriptional features that overlap with those of oncogene-negative human lung adenocarcinoma. We generated a gene expression signature of oncogene-negative tumors comprised of genes that are higher in Nf1/Rasa1/Pten tumors relative to KRAS^{G12D} tumors in mice. We then assigned gene signature activity scores to TCGA lung adenocarcinoma patients on the basis of our mouse-derived oncogene-negative gene expression signature using single-sample GSEA. Interestingly, upon stratification of TCGA patients on the basis of whether they harbor oncogene mutations, we find that our oncogene-negative signature exhibited the highest activity among oncogene-negative human lung adenocarcinomas relative to lung adenocarcinomas driven by oncogenic KRAS or other known oncogene alterations (FIG. 4e). In conclusion, the molecular and biochemical profiles of NF1/RASA1/PTEN mouse tumors agreed with those of oncogene-negative human lung adenocarcinomas. These observations underline the importance of in vivo functional genomics in identifying the biochemical changes that drive tumor development.

### Oncogene-Negative Lung Tumors are Vulnerable to Inhibition of RAS and PI3K-AKT Pathways

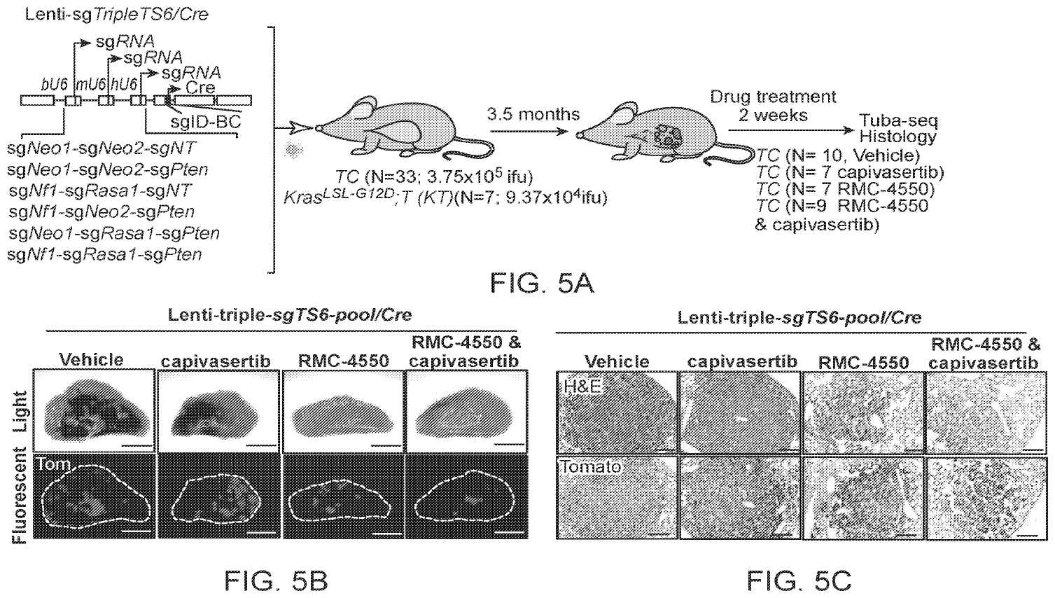

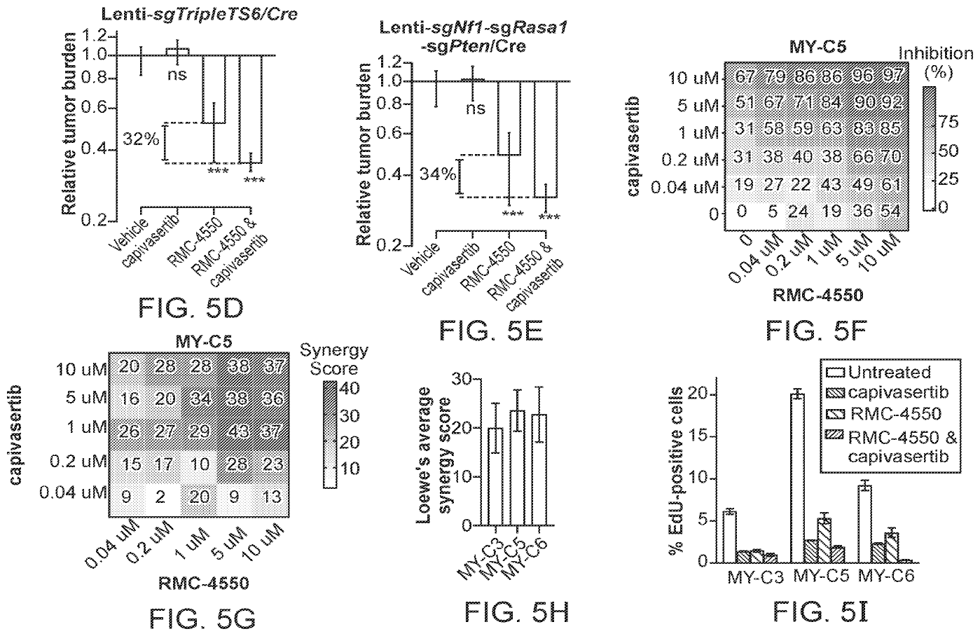

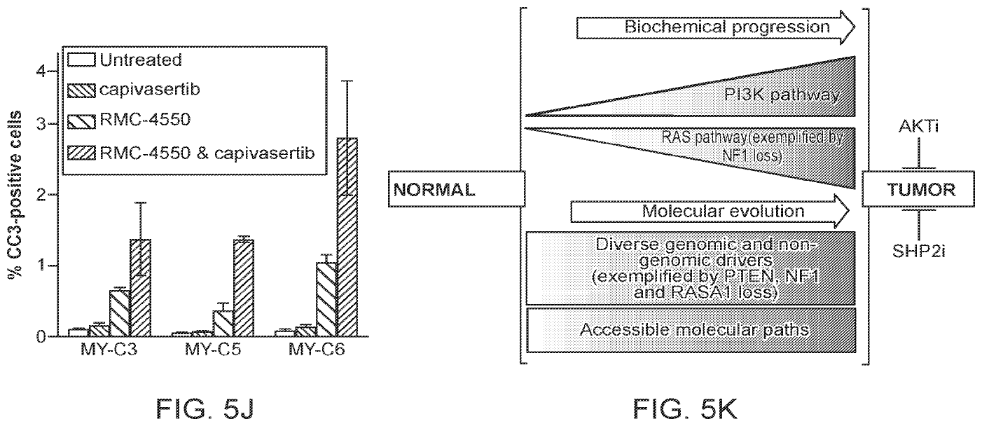

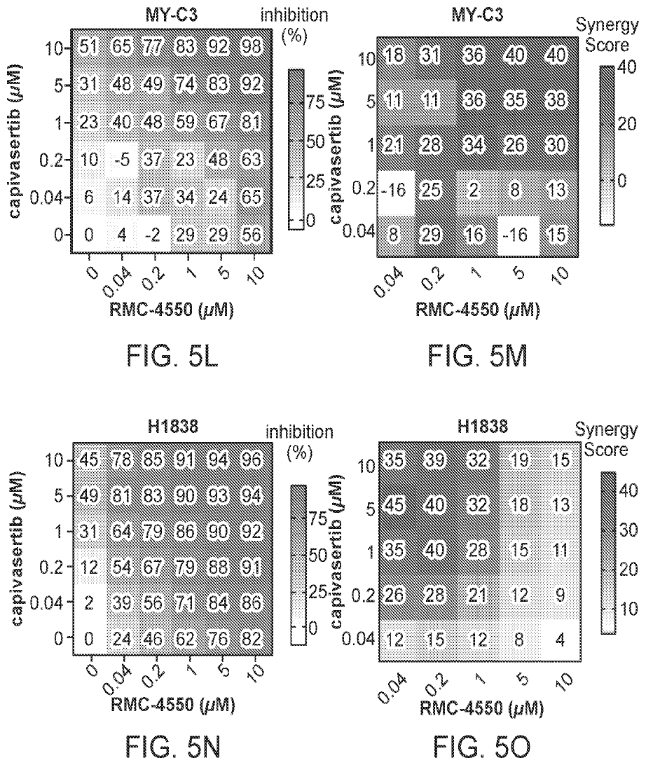

Understanding the biochemical changes that drive tumor development can nominate potential therapeutic strategies [39]. To investigate the therapeutic benefit of targeting key nodes in oncogene-negative lung cancer, we initiated tumors with a smaller pool of single, double, and triple sgRNA viral vectors targeting Nf1, Rasa1, and Pten in TC mice to generate oncogene-negative tumors with activated RAS and/or PI3K pathways. We treated these mice with the AKT1/2 inhibitor capivasertib [55, 56], SHP2 inhibitor RMC-4550 [57], or a combination of RMC-4550 and capivasertib (FIG. 5a, 22a,b). These drugs were chosen based on their ongoing clinical development and ability to counteract activation RAS and/or PI3K pathways [56, 57].

Direct fluorescence imaging and histology indicated that SHP2 inhibition and combined SHP2 and AKT1/2 inhibition greatly impacted tumor burden (FIGS. 5b-c, and 22c). Tuba-seq analysis allowed us to gain greater insight into the overall and genotype-specific responses of tumors to the therapeutic interventions. Capivasertib monotherapy was ineffective while RMC-4550, and the combination of RMC-4550 and capivasertib reduced the total tumor burden significantly, with the combination therapy trending towards being the most efficient therapeutic approach (˜24-34% reduction in tumor burden in combination therapy compared with RMC-4550 alone), consistent with our imaging observations (FIG. 5d, e, and 22d-f).

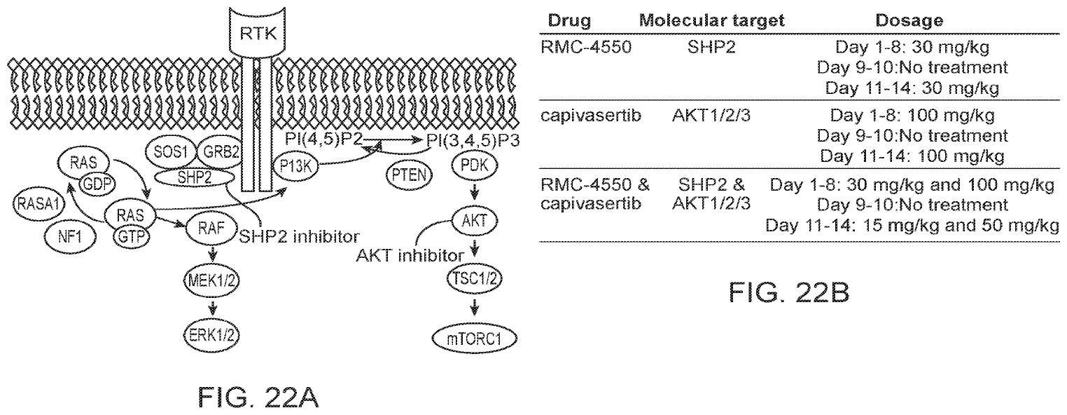

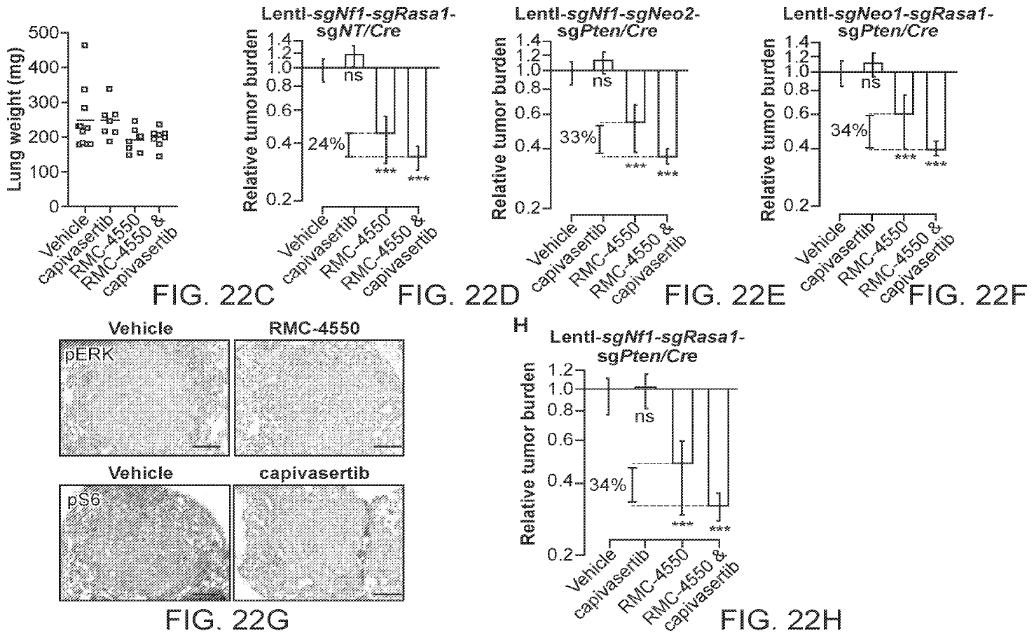

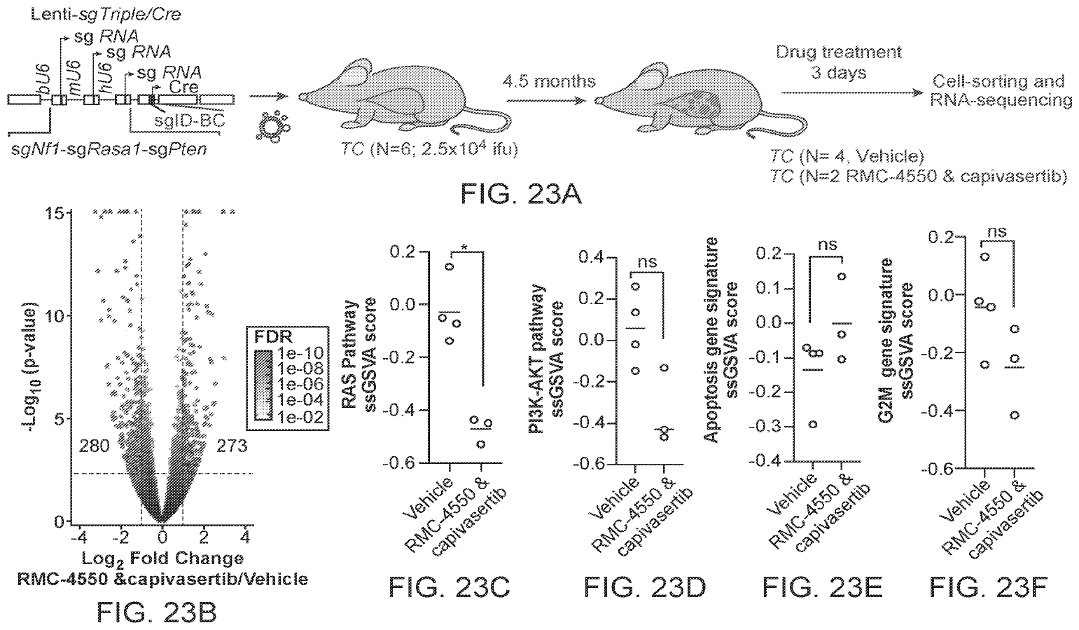

We confirmed the inhibition of RAS and PI3K pathways in lung tumors of mice treated with RMC-4550 and capivasertib by immunohistochemistry (FIG. 22g). Furthermore, global gene expression analysis confirmed the downregulation of RAS and PI3K-AKT gene expression signatures after coincident SHP2 and AKT1/2 inhibition (FIG. 23a-d). Treated tumors exhibited higher expression of an apoptosis gene expression signature and lower expression of a G2/M gene expression signature, suggesting that this combination treatment induces broad cellular changes in oncogene-negative tumors (FIG. 23e, f).

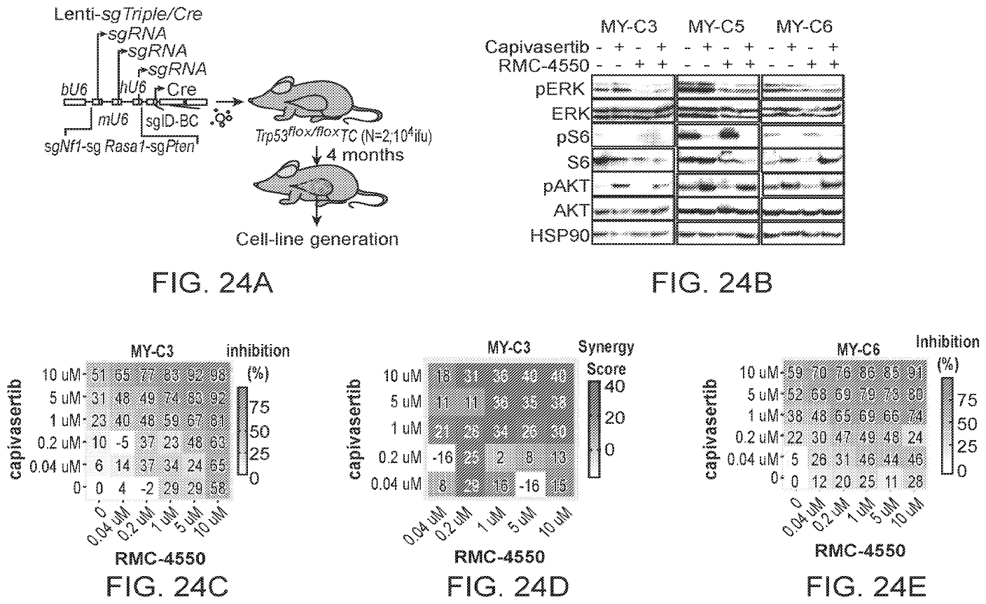

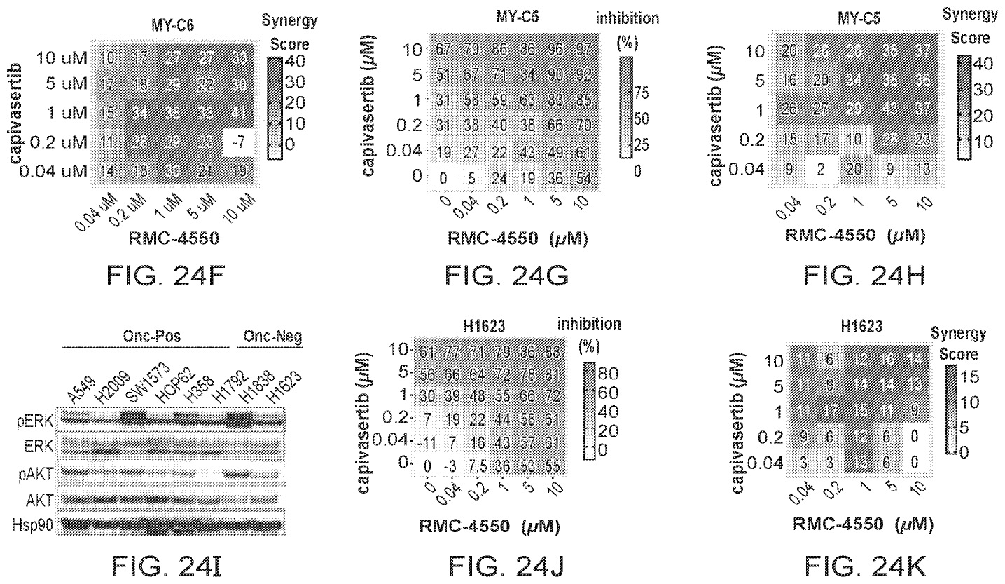

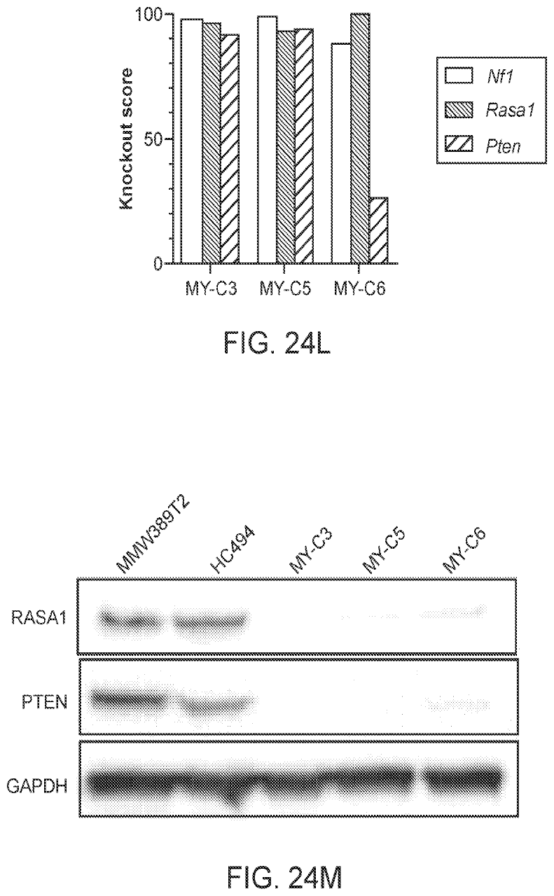

To more extensively characterize the responses of the cancer cells to these treatments, we generated cancer cell lines from tumors initiated with Lenti-sgNf1-sgRasa1-sgPten/Cre in Trp53^{flox-flox}; TC mice (FIG. 24a). As anticipated, RAS and PI3K signaling was reduced in response to treatment with RMC-4550 and capivasertib, respectively (FIG. 24b). Both RMC-4550 and capivasertib decreased overall growth of three oncogene-negative cell lines in a dose-dependent manner (FIGS. 5f and 24c, e). Consistent with our in vivo observations, RMC-4550 and capivasertib synergized to inhibit the growth of oncogene-negative lung adenocarcinoma cell lines (FIGS. 5g, h, and 24d, f). RAS and PI3K signaling promote cell growth and survival [58, 59], and treatment of oncogene-negative cell lines with RMC-4550 and capivasertib inhibited proliferation and induced apoptosis to a greater extent than either RMC-4550 or capivasertib alone (FIG. 5i, j). Collectively, these in vivo and cell culture analyses indicate that oncogene-negative tumors with activated RAS and PI3K pathways are vulnerable to therapeutic inhibition of these pathways.

### DISCUSSION

Lung adenocarcinomas that lack defined oncogene alterations afflict as many patients as those driven by either oncogenic KRAS or EGFR. To identify whether combinatorial loss of multiple tumor suppressor genes can drive the initiation and growth of lung adenocarcinoma in the absence of oncogene activation, we performed a series of multiplexed in vivo functional genomic screens to search through a large set of combinatorial tumor suppressor gene alterations. We discovered that inactivation of single tumor suppressor genes, as well as pairwise alteration of the majority of tumor suppressors that we assessed, are insufficient to generate lung tumors. Importantly, we uncovered higher-order interactions between tumor suppressor genes as key drivers of oncogene-negative lung adenocarcinomas, with combinatorial inactivation of Nf1, Rasa1, and Pten being as potent as oncogenic Kras^{G12D} in initiating lung tumors in vivo.

While NF1 inactivation is sometimes suggested to be an “oncogenic driver” in lung adenocarcinoma [7, 29, 60], Nf1 inactivation alone is insufficient to initiate lung tumors (FIG. 16). Coincident mutations in NF1 and RASA1 are mutually exclusive with other oncogene alterations [53, 54]. However, pairwise alterations of Nf1 and Rasa1 and all other tumor suppressor genes that we tested exhibited weak propensities to initiate tumors. The potent generation of lung adenocarcinomas after combinatorial inactivation of Nf1, Rasa1, and Pten suggests that alterations in multiple genes within and across pathways may be required to surpass thresholds necessary for tumor initiation and growth. We speculate that these thresholds may also be influenced by tumor suppressor genes independent from RAS and PI3K pathways, as well as by environmental factors.

Selection for molecular alterations during cancer evolution is a non-random and tightly constrained process that is strongly influenced by genetic and molecular network interactions [62]. The mouse models that we employed are uniquely able to quantify the likely trajectory of selection for tumor suppressor alterations and their possible evolutionary paths. Our data suggest that inactivation of Nf1, Rasa1, or Pten each increases cellular fitness, thus making each next step more likely (FIGS. 3e and 18g). Interestingly, within other evolutionary systems, this is not always the case, and generation of complex genotypes can be constrained.

In this study, we assessed the ability of hundreds of complex tumor suppressor genotypes to generate lung tumors. Knowledge of the genes underlying human cancers is a pillar of cancer diagnostics, personalized medicine, and the selection of rational combination therapies. Our data demonstrate that biochemical assessment of oncogenic pathways in tumors is a strong foundation for rational selection of therapies and clinical trial designs for cancer patients.

### Methods

### Analysis of Human Lung Adenocarcinoma Datasets

Somatic mutation data (SNPs and indels, including silent mutations) for 513 TCGA lung adenocarcinoma (LUAD) tumors were downloaded from the UCSC Xena Browser (http: followed by //xena followed by .ucsc followed by edu/), specifically at Link 1. TCGA-LUAD clinical and exposure data were downloaded from the GDC Data Portal (http followed s://porta followed by l.gdc.cancer. followed by gov/projects/TCGA followed by—LUAD) and the UCSC Xena Browser (Link 2). Gistic2 thresholded copy number variation (CNV) data were downloaded from the UCSC Xena Browser (Link 3). Amplifications were defined as “2” and deletions as “−2”. Genes with conflicting CNV values within a single tumor were ignored. Fusion data were obtained from [1]. Fusion and CNV data were filtered to include only data from the 513 samples within the somatic mutation set. Duplicate fusions were collapsed into single fusions. Samples with MET-exon skipping were taken from [2]. Curated survival data from [3] were downloaded from the UCSC Xena Browser (Link 4).

### Links:

htt followed by ps:/followed by/tcga.xenahubs followed by.net/download/mc3/LUAD followed by mc3.txt.gz
htt followed by ps://tcga.xenahubs.net/dow followed by nload/TCGA.LUAD.sample followed by Map/LUAD_clinical followed by Matrix
htt followed by ps://tcga.xena followed by hubs.net/download/TCGA.LUAD.sampleMap/Gistic2_CopyNum followed by ber_Gistic2_all_thresh followed by olded.by_genes.gz
ht followed by tps://tcga.xen followed by ahubs.net/download/sur followed by vival/LUAD_sur followed by vival.txt followed by .gz

Data from AACR Project GENIE (hereinafter referred to as GENIE) v8 were downloaded from ht followed by tps://ww followed by w.synapse.org/#!Sy followed by napse:syn222 followed by 28642 [3], specifically: somatic mutations, copy number alteration (CNA) data, fusion data, panel information (genomic_information.txt), and clinical data (both sample- and patient-level). All data were filtered to only include LUAD tumors. A single tumor was kept for patients with multiple different tumor samples, with priority on younger (earlier sequenced) patients and primary (non-metastasized) tumors. If tumor samples appeared identical within the clinical meta-data, the related patient data were excluded.

### Determination of Oncogenes

To have a conservative estimate of the fraction of lung adenocarcinomas without known oncogenic drivers (oncogene-negative tumors), we included any gene that meets at least one of these criteria as an oncogene: 1) Genes that have hotspot mutations or specific alterations where cancers or cancer cells with that mutation respond to therapies targeted to the protein product of that mutant gene in patients, 2) The particular alteration in that gene can generate lung adenocarcinoma in genetically-engineered mouse models, 3) The altered gene can generate tumors in other tissues in genetically-engineered mouse models, and 4) Alteration of the indicated gene can lead to the transformation of cells in vitro or can predict response to targeted therapies. Additionally, we excluded genes if their oncogenic alterations co-occur with alterations in other proto-oncogenes (listed below) in more than 50% of cases.

| | | | | | Cellular | |
|---|---|---|---|---|---|---|
| | | Patients | Sufficient | Oncogene | transformation | Co-occurring |
| | | respond to | to generate | in GEMMs | and/or drug | with other |
| | | oncogene | lung tumors | of other | response in | protooncogenes |
| Gene | Oncogenic alterations | inhibition | in GEMMs | tumor types | cultured cells | listed here |
| KRAS | Mutations at codons | [4, 5] | [6-8] | [7, 9, 10] | | 4% |
| | 12, 13, and 61 | | | | | |
| EGFR | Diverse mutations and | [11, 12] | [13-15] | [16] | | 9% |
| | deletions | | | | | |
| BRAF | Diverse mutations and | [17] | [18, 19] | [20, 21] | [22] | 18% |
| | fusions | | | | | |
| HRAS | Mutations at codons | | | [23] | [24, 25] | 13% |
| | 12, 13, and 61 | | | | | |
| NRAS | Mutations at codons | | | [26, 27] | | 14% |
| | 12, 13, and 61 | | | | | |
| MET | Exon skipping | [28, 29] | [2] | | [2, 30, 31] | 47% |
| MEK1 | Diverse mutations | | | | [32] | 24% |
| SOS1 | Diverse mutations | | | | [33] | 17% |
| ALK | Translocations/fusions | [34] | [35, 36] | [37] | [38] | 2% |
| RET | Translocations/fusions | [39] | [40, 41] | | | 10% |
| ROS1 | Translocations/fusions | [42] | | [43, 44] | | 4% |
| NTRK1 | Translocations/fusions | [45] | | | [46] | 29% |
| NRG1 | Translocations/fusions | [47] | | | | 0% |
| AKT1 | Point mutation | [48] | | | [49] | 50% |
| | (E17K) | | | | | |
| RIT1 | Diverse mutations | | | | [50] | 33% |
| ERBB2 | Amplification, point | [51] | [52, 53] | [54, 55] | [56] | 19% |
| | mutation, deletion | | | | | |
| PIK3CA | Diverse mutations | | Opposing | | | 68% |
| | | | evidence | | | |
| | | | [57, 58] | | | |

### Classification of Mutations and Tumors

Mutations (somatic mutations, fusions, CNVs, and MET exon skipping [TCGA only]) were classified as within proto-oncogenes (described above) or not. Mutations within these proto-oncogenes were classified as “accepted oncogenic” mutations if those alterations can meet at least one of the criteria described above. Any tumor with one accepted oncogenic alteration was classified as “oncogene positive”. Tumors with accepted oncogenic mutations in more than one gene were classified as “multiple oncogenes mutated”. Any tumor with alterations in a proto-oncogene that was not considered an accepted oncogenic alteration based on the four criteria above was classified as “oncogene indeterminate”. The remaining tumors, without any mutations in any proto-oncogene, were classified as “oncogene-negative”.

### Tumor Type Counts Per Database:

| | TCGA | GENIE |
|---|---|---|
| Total | 513 | 9,099 |
| Oncogene-negative | 91 | 1,645 |
| Oncogene-positive | 283 | 6,041 |
| Oncogene-indeterminate | 139 | 1,413 |

Oncogene positive tumors were further classified by the type of accepted oncogenic mutation they had (FIG. 1a, 6a).

### Clinical Characteristics

Tumors were divided into males or females based on the sex reported by either TCGA or GENIE, if provided. For TCGA, the arithmetic mean for age at diagnosis was computed and reported with a standard error of the mean (SEM). Non-smokers were defined as having tobacco smoking history values of 1 (see public ID 2181650 at https://cdebrowser.nci.nih.gov), while smokers were defined as anything >1 (current or reformed smokers). The arithmetic mean pack-years smoked for smokers, if reported, was reported, along with SEM.

### Pan-Cancer Tumor Suppressor Genes

We generated a list of tumor suppressor genes based on two previously published reports to compare the number of altered tumor suppressor genes in oncogene-negative tumors with oncogene-positive tumors [59, 60]. We manually removed genes with conflicting evidence as a tumor suppressor gene in LUAD.

### Calculation of Mutation Frequencies and Absolute Number of Genes Mutated

In general, mutation frequencies for a given gene were calculated as the number of tumors with that gene mutated, divided by the number of tumors screened for mutations in that gene (for TCGA: all tumors were screened for all genes, for GENIE: the panel sequencing information was obtained from genomic_information.txt to determine which tumors were screened for which genes). Mutation frequencies were calculated for point mutations (PM), insertion/deletions (indels), and deletions separately. Additionally, the frequency for a combination of PMs, indels, and deletions was also calculated. The screened set of tumors in GENIE for the latter included only those tumors which were screened for both PMs/indels as well as CNVs for each gene. Reported in FIG. 7b are oncogene-negative tumors with either point mutations, indels, or deletions in the indicated gene. In FIG. 7c/d, for each gene, a ratio of enrichment of mutations in oncogene-negative over oncogene positive tumors was calculated as:

$$\frac{mutation {frequency}_{oncogene - negative}}{mutation {frequency}_{oncogene positive}}$$

A P-value for this enrichment was calculated using the two-sided Fisher's Exact test as implemented by SciPy. For a given set of genes with at least a single tumor screened, the false discovery rate (FDR) was calculated using the Benjamini-Hochberg method on the Fisher's Exact P-values.

To measure the total number of genes mutated (FIG. 6d), a gene was considered mutated if it had at least one point mutation or indel. All these mutations in a tumor were collated, and the number of the unique set of genes was counted as the total number of genes mutated. For counting the number of unique tumor suppressors mutated (FIG. 6e), deletions were also included, and the list of pan-cancer tumor suppressors as defined above was used. The Mann-Whitney U test was conducted on the number of respective genes mutated in either oncogene-negative or oncogene-positive tumors.

### Survival Analysis

Survival data from [3] were obtained as described above. Kaplan-Meier analysis was performed to estimate probability curves for overall survival (OS) and disease-specific survival (DSS), and the logrank test was used to compare oncogene-negative and oncogene-positive tumors. A P-value of less than 0.05 was considered significant.

### Gene and Pathway Alteration Co-Occurrences

For analysis of simultaneous pairwise alterations of NF1, RASA1, or PTEN within oncogene-negative tumors, we determined the number of tumors with no mutation in NF1, RASA1, or PTEN, mutation(s) in one gene, or mutations in two genes simultaneously. Point mutations, indels, and deletions in each gene were included. A tumor needed one or more mutations in that gene to be considered mutated. For GENIE, only those tumors screened for both genes for point mutations and indels (according to the panel information file) were investigated. For TCGA, all oncogene-negative tumors were considered. A one-sided Fisher's exact test was conducted to determine if there were more than the expected number of tumors with both genes mutated.

Gene lists and their acceptable alterations were generated as being in RAS or PI3K pathways [59, 61-68] and not known to be an oncogene alteration (Table 2). We determined the list of all tumors screened for each gene on each list for each respective type of mutation (point mutation/indel, amplification, deletion, or fusion). For each alteration within each pathway, we determined whether it could activate the corresponding pathway or not according to the above list. A gene was considered mutated if it had at least one accepted mutation within it. A tumor was considered mutated in a given pathway if it had at least one gene mutated in that pathway.

### Animal Studies

Kras^{LSL-G12D}/+(Jax #008179 (K)), R26^{LSL-tdTomato}(ai9) (Jax #007909 (T)), and H11^{LSL-Cas9} (Jax #026816 (C)), Keap1^{flox}, Pten^{flox} (Jax #006440), Lkb^{flox} (Jax #014143), Nf1^{flox} (017640), and Trp53^{flox} (Jax #008462) mice have been previously described [6, 69-75]. All mice were on a C57BL/6:129 mixed background except the mice used for derivation of oncogene-negative Nf1, Rasa1, and Pten mutant cell-lines.

For drug efficacy studies, TC mice (8-12 weeks old) were divided into 4 groups randomly. They received the vehicle, capivasertib (100 mg/kg, Med Chem Express), RMC-4550 (30 mg/kg, Med Chem Express), or a combination of both dissolved in 10% DMSO, 40% PEG, 5% Tween 80, and 45% PBS through a gavage needle. Mice were treated daily with drugs for eight days, and the treatment was stopped for two days for recovery, and it continued for two more days before the tissue harvest. The last two doses of combination therapy were half of the initial doses as signs of weakness were observed in some mice.

Cell lines were generated from tumors initiated in Trp53^{flox/flox}; TC BL6 mice four months after transduction with Lenti-sgNf1-sgRasa1-sgPten/Cre. After dissociation of tumors (described below), cells were cultured in the regular media.

### Lentiviral Generation, Barcoding, and Packaging

The guide RNA sequences, cloning, and barcoding of Lenti-sgRNA/Cre and Lenti-TriplesgRNA/Cre vectors have been previously described [76-78]. The sgRNA sequences used in each experiment are summarized below:

| Pool | Pool composition |
|---|---|
| Lenti-sgTS15/Cre | The exact pool used in [77, 79] |
| Lenti-sgTS102/Cre | The exact pool used in [76] |
| Lenti-sgTS14/Cre | Version 1 of sgEP300, sgKmt2c, sgNcoa6, |
| | sgRbm10, sgNeo, sgNf1, and sgPten, and |
| | version 2 of sgArid1a, sgCdkn2a, sgDnmt3a, |
| | sgKdm6a, sgRb1, sgTet2, sgRasa1 from [76] |
| Lenti-sgTS11/Cre | Lenti-sgTS14/Cre pool excluding sgNf1, sgRasa1, |
| | and sgPten |
| Lenti-sgTripleTS8/Cre | Version 1 of sgNf1, sgNeo, and sgNT, and |
| | version 2 of sgRasa1, sgPten, and sgNeo from |
| | [76] |

To generate lentivirus, Lenti-sgRNA/Cre vectors were individually co-transfected into 293T cells with pCMV-VSV-G (Addgene #8454) envelope plasmid and pCMV-dR8.2 dvpr (Addgene #8455) packaging plasmid using polyethylenimine. Supernatants were collected 36 and 48 hours after transfection, passed through a 0.45 μm syringe filter (Millipore SLHP033RB) to remove cells and cell debris, concentrated by ultracentrifugation (25,000 g for 1.5 hours at 4° C.) and resuspended in PBS overnight. Each virus was titered against a standard of known titer using LSL-YFP Mouse Embryonic Fibroblasts (MEFs) (a gift from Dr. Alejandro Sweet-Cordero/UCSF). All lentiviral vector aliquots were stored at −80° C. and were thawed and pooled at equal ratios immediately prior to delivery to mice.

### Tumor Initiation

Tumors were initiated by intratracheal delivery of pooled or individual Lenti-sgRNA/Cre vectors. Barcoded Lenti-sgRNA/Cre vectors within each viral pool are indicated in each figure. Tumors were initiated with the indicated titer and allowed to develop between 3 and 12 months after viral delivery, as indicated in each figure.

### Tumor Barcode Sequencing and Analysis

For DNA extraction from single dissected tumors to generate libraries for Tuba-seq, targeted sequencing of selected oncogenes, and whole-exome sequencing, we used Qiagen AllPrep DNA/RNA Micro kit. For Tuba-seq on bulk lungs, genomic DNA was isolated from bulk tumor-bearing lung tissue from each mouse as previously described [77]. Briefly, benchmark control cell lines were generated from LSL-YFP MEFs transduced by a barcoded Lenti-sgNT3/Cre vector (NT3: an inert sgRNA with a unique sgRNA identifying barcode (sgID) and a random barcode (BC)) and purified by sorting YFP⁺ cells using BD FACS Aria™ II Cell Sorter. Three cell lines (100,000 to 500,000 cells each) were added to each mouse lung sample before lysis to enable the calculation of the absolute number of neoplastic cells in each tumor from the number of sgID-BC reads. Following homogenization and overnight protease K digestion, genomic DNA was extracted from the lung lysates using standard phenol-chloroform and ethanol precipitation methods. Subsequently, Q5 High-Fidelity 2× Master Mix (New England Biolabs, M0494X) was used to amplify the sgID-BC region from 50 ng of DNA from dissected tumors or 32 μg of bulk lung genomic DNA. The unique dual-indexed primers used were Forward: AATGATACGGCGACCACCGAGATCTACAC-8 nucleotides for i5 index-ACACTCTTTCCCTACACGACGCTCTTCCGATCT-6 to 9 random nucleotides for increasing the diversity-GCGCACGTCTGCCGCGCTG and Reverse: CAAGCAGAAGACGGCATACGAGAT-6 nucleotides for i7 index-GTGACTGGAGTTCAGACGTGTGCTCTTCCGATCT-9 to 6 random nucleotides for increasing the diversity-CAGGTTCTTGCGAACCTCAT. The PCR products were purified with Agencourt AMPure XP beads (Beckman Coulter, A63881) using a double size selection protocol. The concentration and quality of the purified libraries were determined using the Agilent High Sensitivity DNA kit (Agilent Technologies, 5067-4626) on the Agilent 2100 Bioanalyzer (Agilent Technologies, G2939BA). The libraries were pooled based on lung weights to ensure even reading depth, cleaned up again using AMPure XP beads, and sequenced (read length 2×150 bp) on the Illumina HiSeq 2500 or NextSeq 500 platform (Admera Health Biopharma Services).

### Tuba-Seq Analysis of Tumor Barcode Reads

The FASTQ files were parsed to identify the sgID and barcode (BC) for each read. Each read is expected to contain an 8-nucleotide sgID region followed by a 30-nucleotide barcode (BC) region (GCNNNNNTANNNNNGCNNNNNTANNNNNGC), and each of the 20 Ns represents random nucleotides. The sgID region identifies the putative tumor suppressor gene being targeted, for which we require a perfect match between the sequence in the forward read and one of the forward sgIDs with known sequences. Note that all sgID sequences differ from each other by at least three nucleotides. Therefore, the incorrect assignment of sgID due to PCR or sequencing error is extremely unlikely. All cells generated from the clonal expansion of an original cell transduced with a lentiviral vector carry the same BC sequence. To minimize the effects of sequencing errors on calling the BC, we require the forward and reverse reads to agree completely within the 30-nucleotide sequence to be further processed. In our pipeline, any tumor that is within a Hamming distance of two from a larger tumor is assigned as a “spurious tumor”, which likely results from sequencing or PCR errors and the tumor is removed from subsequent analysis. Reads with the same sgID and barcode are assigned to be the same tumor. The tumor size (number of neoplastic cells) is calculated by normalizing the number of reads to the three benchmarks “spike-in” cell lines added to each sample prior to lysis of the lung and DNA extraction step. The median sequencing depth was ˜1 reads per 4.8 cells, and the minimum sequencing depth is ˜1 reads per 16.5 cells. We have high statistical power in identifying tumors with more than 200 cells, which was used as the minimum cell number cutoff for calling tumors. A minimum cell number of 50 was used for calling expansions in FIGS. S5 and 11). Minimizing the influence of GC amplification bias on tumor-size calling was done as previously described [77].

### Measures of Tumor Size and Growth

Three summary statistics were used in this study to characterize the growth of a given genotype. Different percentiles of tumor sizes were calculated by sorting tumor sizes above 200 cells for each genotype and calculating the percentiles of the tumor size distribution. Tumor burden was calculated as the sum of neoplastic cells per mouse averaged over all mice. Tumor numbers above a given size threshold (e.g., 1000 cells) were determined by calculating the number of tumors above the threshold per mouse and averaging over all mice. Tumor burden and tumor number are affected linearly by lentiviral titer. When applicable we used data on the number of tumors from KT mice lacking Cas9 to approximate the representation of each Lenti-sgRNA/Cre vectors in the lentiviral pool. Therefore, when calculating tumor burden and tumor number metrics, we normalized the metric to the effective titer based on KT mice to account for the viral titer differences among different Lenti-sgRNA/Cre vectors. All three of these measures were later normalized to the values of the same metric for tumors with inert sgRNAs.

Confidence intervals and p values were calculated by a nested bootstrap resampling approach to account for variation in sizes of tumors of a given genotype both across and within mice. First, tumors of each mouse were grouped, and these groups (mice) were resampled. Second, all tumors of a given mouse resampling were bootstrapped on an individual basis (10,000 repetitions).

To assess tumor suppressor strength in dissected tumors (FIGS. 1f and 9), the relative frequency of each sgRNA was calculated in each sample (one sample can contain multiple sgRNAs due to multiple transduction or multiple tumors being present in the sample) and averaged for each sgRNA over all samples for a given mouse genotype. Inert sgRNAs have no tumor suppressor effect and serve as a baseline. Tumors were bootstrap resampled 10,000 times, and the distribution of inert sgRNA frequencies was used to calculate p-values for enrichment of all other sgRNAs.

### Multiple Transduction

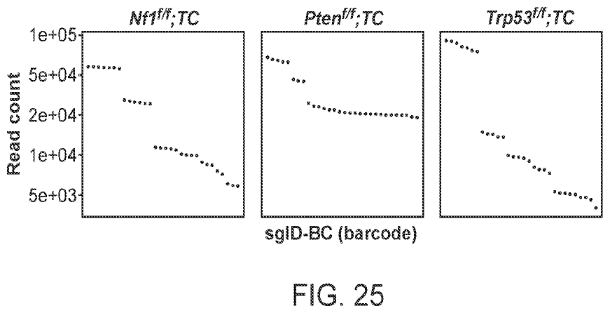

A fraction of lung tumors initiated with Lenti-sgRNA/Cre vectors contained multiple barcoded Lenti-sgRNA/Cre vectors. If multiple barcodes (sgID-BCs) have unexpectedly similar read counts (as shown in the example plots below), we suspect transduction of the initial cell with multiple Lenti-sgRNA/Cre vectors. See FIG. 25.

To capitalize on multiple transductions as a way to find higher-order interactions between tumor suppressor genes, we developed a method to identify the combinations of sgRNA that appear to cooperate as potent drivers of tumor growth. Accurate identification of coinfected tumors and grouping of barcodes without over grouping was not a trivial task. We developed methods to identify tumors with likely multiple transductions (i.e., those tumors with complex genotypes with multiple tumor suppressor genes inactivated). For each sgID-BC, we listed all other sgID-BCs from the same mouse with read counts in the 10% vicinity (90%-111.11%) as possible multiple infecting events. A tumor with multiple transductions can be most easily identified among the largest tumors in each mouse as smaller tumors of similar sizes are in too high an abundance. Multiple transductions that lead to synergistic combinatorial tumor suppressor alterations would confer a growth advantage to the cancer cells. Thus, synergistic combinatorial alterations of tumor suppressor genes would be expected to be overrepresented among the largest tumors.

To have a dataset with a higher signal-to-noise ratio, we analyzed the largest tumors that were co-infected with up to 6 Lenti-sgRNA/Cre (out of 14 different sgIDs used in the experiment). With this method, for each tumor, we assembled a list of genes that were possibly co-mutated. We then ranked all possible combinations of genes by their frequency in the largest tumors (FIG. 2f/g and 13).

An inherent problem with this analysis is that the genotypes that result in high tumor growth will be overrepresented amongst the largest tumors even without multiple transductions and specific synergistic interactions. To account for the different number of tumors with different sgIDs, we performed a permutation test, where we control for the number of tumors of each genotype but randomize the sizes of tumors by randomly matching the genotypes with tumor sizes (10,000 repetitions). Synergistic tumor suppressor combinations will occur at significantly higher than expected frequencies based on this permutation test (FIG. 2f/g and 13). Reassuringly, while our analysis resulted in significant enrichment of complex genotypes based on the permutation test, a control analysis performed on smaller tumors within the same mice with high noise to signal ratio resulted in a loss of statistical significance, showing that the permutation test controls for the bias of different frequency of sgIDs among the tumors.

### Fitness Landscape and Adaptive Paths

To investigate the possible adaptive steps that can lead to the complex genotype of coincident inactivation of Nf1, Rasa1, and Pten, we first measured the fitness of all possible combinations of Nf1, Rasa1, and Pten mutations. Relative (Malthusian) fitness was calculated based on the number of individuals (cells) at the end (N₁) and the beginning of (N₀) of a time period [80]. For each genotype, the overall sum of neoplastic cells at the end of the experiment (N₁) was calculated as the sum of cells from all tumors in each mouse. As we use KT mice (which lack Cas9 and all sgRNAs have no effect) to approximate the effective titer of our virus pool (see section Measures of tumor size and growth), the initial number of cells transduced (No) was calculated from the number of tumors generated in control KT mice. Next, the relative fitness for genotype A compared to wild type (wt) was calculated as:

$$\frac{log 2 \frac{{N}_{1 , A}}{{N}_{0 , A}}}{log 2 \frac{{N}_{1 , wt}}{{N}_{0 , wt}}}$$

Fitness values relative to wild type are displayed as nodes on the adaptive landscape (FIGS. 3e. 18g), where genotypes one mutation away from each other are connected by arrows that represent mutations. In the case of the Nf1; Rasa1; Pten triple mutant state, six adaptive paths can lead from wild type to that triple mutant genotype (FIG. 3e, 18g). Arrows are shown if the mutation increases the fitness. In FIGS. 3e and 18g all arrows are shown since all mutations increase fitness.

Next, we set out to approximate the relative probabilities of different adaptive paths leading from wild type to the triple mutant genotype with a simple population genetic model. In the model, cell populations start from the wild-type genotype, and they can acquire any of the three mutations present in the triple genotype. In the population of cells a mutation can arise and then change in frequency until one of two outcomes happens: (i) the frequency of the mutation drops to zero, and the mutation is lost from the population or (ii) the frequency of the mutation reaches 1, when it is present in all the cells and hence is fixed in the population. When a mutation fixes in a population, we consider the genotype of the population to change and that constitutes a “step” on the fitness landscape. We assume a “strong selection weak mutation” regime, where there is no more than one mutation simultaneously present with a frequency less than 1. We also assume that mutations appear randomly and with equal probabilities. Mutations can appear and get lost multiple times in a population, and as long as populations have at least one mutation that increases fitness, one of those mutations will fix in the population eventually.

With the model we are estimating the probability of each adaptive step given that the population starts from the wild-type state. Therefore, the probability of each adaptive step will be influenced by the probabilities of previous step(s) and the sum of probabilities of adaptive steps originating from a given genotype must equal the sum of probabilities of all adaptive steps terminating in the given genotype. If there are multiple adaptive steps originating from the same genotype, they will have probabilities proportional to the fixation probabilities of their respective mutations. The fixation probability of a mutation is proportional to its selective advantage

$$( \frac{fitness after mutation}{fitness before mutation} - 1 ) . \qquad [ 81 ]$$

As an example, if there are two adaptive steps originating from a genotype with fitness 1.00, one terminating in a genotype with fitness 1.1, the other in a genotype with fitness 1.2, then they have 10% and 20% selective advantage, respectively. Therefore, one adaptive step will happen half as likely as the other, as the selective advantages and therefore the relative fixation probabilities are in a ratio of 1:2.

### Targeted Sequencing of Oncogenic Loci for Potential Spontaneous Oncogenic Mutation

To determine whether the tumors that develop contained spontaneous oncogenic mutations, we performed Sanger Sequencing and Illumina sequencing (HiSeq 2500 platform; read length 2×150 bp, Admera Health Biopharma Services) on select regions of Kras, Egfr, Braf, and Nras (the 4 frequently mutated oncogenes in lung adenocarcinoma).

PCR products were obtained through amplification with primers listed below on DNA extracted from dissected tumors (described above) and cleaned up using ExoSAP (ThermoFisher Scientific, Cat #78-201) treatment before Sanger.

| Oncogene and associated codon | Primer |
|---|---|
| Kras Codon 12 + 13 Forward | ACACTCTTTCCCTACACGACGCTCTTCCGATCTNN |
| | NNNNTTATTTTTATTGTAAGGCCTGCT |
| Kras Codon 12 + 13 Reverse | GTGACTGGAGTTCAGACGTGTGCTCTTCCGATCTN |
| | NNNNNNTTACAAGCGCACGCAGA |
| Kras Codon 61 Forward | ACACTCTTTCCCTACACGACGCTCTTCCGATCTNN |
| | NNNNNNCCTGTCTCTTGGATATTCTCGAC |
| Kras Codon 61 Reverse | GTGACTGGAGTTCAGACGTGTGCTCTTCCGATCTN |
| | NNNNNNNNCAGTTCTCATGTACTGGTCCCT |
| Egfr Codon 721 Forward | ACACTCTTTCCCTACACGACGCTCTTCCGATCTNN |
| | NNNNNNCCAGCGGAGAAGCTCCAAAC |
| Egfr Codon 721 Reverse | GTGACTGGAGTTCAGACGTGTGCTCTTCCGATCTN |
| | NNNNNNNNATACACTGTGCCAAATGCTCCC |
| Egfr Codon 734-756 Forward | ACACTCTTTCCCTACACGACGCTCTTCCGATCTNN |
| | NNNNTCTTCTTAATCTCAGGGTCTCTGG |
| Egfr Codon 734-756 Reverse | GTGACTGGAGTTCAGACGTGTGCTCTTCCGATCTN |
| | NNNNNNCACGTCAAGGATTTCTTTGTTGGC |
| Egfr Codon 764-793 Forward | ACACTCTTTCCCTACACGACGCTCTTCCGATCTNN |
| | NNNNNNTTACCCAGAAAGGGATATGCGTG |
| Egfr Codon 764-793 Reverse | GTGACTGGAGTTCAGACGTGTGCTCTTCCGATCTN |
| | NNNNNNNNGGCAACCGTAGGGCATGAG |
| Egfr Codon 860 + 863 Forward | ACACTCTTTCCCTACACGACGCTCTTCCGATCTNN |
| | NNNNGTGAAGACACCACAGCATGTCAAG |
| Egfr Codon 860 + 863 Reverse | GTGACTGGAGTTCAGACGTGTGCTCTTCCGATCTN |
| | NNNNNNGCTTCCTGATCTACTCCCAGGAC |
| Braf Codon 503-509 Forward | ACACTCTTTCCCTACACGACGCTCTTCCGATCTNN |
| | NNNNNNGACTGGGAGATTCCTGATGGAC |
| Braf Codon 503-509 Reverse | GTGACTGGAGTTCAGACGTGTGCTCTTCCGATCTN |
| | NNNNNNNNcgtgttatacataccatgtcccac |
| Braf Codon 637 Forward | ACACTCTTTCCCTACACGACGCTCTTCCGATCTNN |
| | NNNNGACCTCACGGTAAAAATAGGTGAC |
| Braf Codon 637 Reverse | GTGACTGGAGTTCAGACGTGTGCTCTTCCGATCTN |
| | NNNNNNAACTGTTCAAACTGATGGGACC |
| Nras Codon 12 + 13 Forward | ACACTCTTTCCCTACACGACGCTCTTCCGATCTNN |
| | NNNNNNTTCTACAGGTTTTTGCTGGTGTG |
| Nras Codon 12 + 13 Reverse | GTGACTGGAGTTCAGACGTGTGCTCTTCCGATCTN |
| | NNNNNNNNGATTAGCTGGATCGTCAAGGC |
| Nras Codon 61 Forward | ACACTCTTTCCCTACACGACGCTCTTCCGATCTNN |
| | NNNNCGAAAGCAAGTGGTGATTGATGG |
| Nras Codon 61 Reverse | GTGACTGGAGTTCAGACGTGTGCTCTTCCGATCTN |
| | NNNNNNAAATACACAGAGGAACCCTTCG |

### N: Random Nucleotides Added to Increase the Diversity of PCR Products for Illumina Sequencing.

Illumina sequencing was performed on pools of amplicons. The libraries were pooled based on band intensity to ensure even read depth and cleaned up using Sera-Mag Select beads (Thermo Fisher Scientific, Cat #09-928-107) before undergoing a second round of PCR to attach the sequencing adaptors needed for the HiSeq platform. Second round PCR products were then purified with Sera-Mag Select beads before sequencing.

| | |
|---|---|
| P5 adapter with i5 Index | AATGATACGGCGACCACCGAGATCTACACNNNNNNNNa |
| | cactctttccctacacgac |
| P7 adapter with i7 Index | CAAGCAGAAGACGGCATACGAGATNNNNNNgtgactggagttc |
| | agacgtg |

N's represent i5 and i7 indices.

### Analysis of Targeted DNA-Sequencing of Kras, Egfr, Braf, and Nras Oncogenic Loci

Sequenced reads were analyzed using Genome Analysis Toolkit (GATK, Broad Institute [82]). “Somatic short variant discovery” best practices pipeline for tumor samples similarly as for whole exome sequencing (see below). However, for targeted sequencing, identification of duplicate reads (Picard MarkDuplicates algorithm) was omitted as that would result in the loss of reads with matching start and end position, which is normal in targeted sequencing and is not a sign of duplicate artifacts. A mean coverage of 6665-7584 reads was achieved for all samples with 90% of regions having a coverage over 275 reads in all samples. Variant calls made and filtered by GATK Mutect2 function were annotated with Ensembl Variant Effect Predictor [83]. Pick-allele-gene option was used to filter results on the most relevant transcript for each variation. We filtered the results for the known oncogenic codons listed above and variants with a minimum of 5% allele frequency.

### Whole Exome Sequencing

DNA was extracted from 4 individual tumors dissected from TC mice, transduced with Lenti-sgNf1-sgRasa1-sgPten, three months after tumor initiation using Qiagen AllPrep DNA/RNA Micro kit. Whole-exome sequencing library preparation was done by Admera Health using SureSelect XT Mouse All Exon Kit (Agilent).

DNA was extracted from bulk tumors dissected from TC mice, transduced with Lenti-sgNf1-sgRasa1-sgPten 3 months after tumor initiation using Qiagen AllPrep DNA/RNA Micro kit. Whole-exome sequencing library preparation was done by Admera Health using SureSelect XT Mouse All Exon Kit (Agilent).

Sequenced reads on autosomes were analyzed using Genome Analysis Toolkit (GATK, Broad Institute [82]) “Somatic short variant discovery” best practices pipeline for tumor samples. Mean coverage of 50-72 reads was achieved for all samples, with 90% of regions having coverage over 20 reads in all samples. Variant calls made and filtered by GATK Mutect2 function and were annotated with Ensembl Variant Effect Predictor (VEP [83]). The pick-allele-gene option was used to filter results on the most relevant transcript for each variant. The same exact variants appearing in multiple tumor samples were flagged as germline variant and were removed. We filtered the results for protein-coding variation, variants with a minimum of 5% allele frequency, and removed variations in the olfactory OLFR gene family that are likely germline variations.

### Histology and Immunohistochemistry

Lung lobes were inflated with 4% paraformaldehyde and fixed for 24 hours, stored in 70% ethanol, paraffin-embedded, and sectioned. 4 μm thick sections were used for Hematoxylin and Eosin (H&E) staining and immunohistochemistry.

Primary antibodies used for IHC were anti-RFP (Rockland, 600-401-379), anti-TTF1 (Abcam, ab76013), anti-UCHL1 (Sigma, HPA005993), anti-TP63 (Cell Signaling Technology, 13109), anti-phospho-S6 (Cell Signaling Technology, 4858), anti-phospho-ERK (Cell Signaling Technology, 4370), and anti-phospho-AKT (Thermo Fisher Scientific, 44-621G). IHC was performed using Avidin/Biotin Blocking Kit (Vector Laboratories, SP-2001), Avidin-Biotin Complex kit (Vector Laboratories, PK-4001), and DAB Peroxidase Substrate Kit (Vector Laboratories, SK-4100) following standard protocols.

Images of the H&E-stained slides were obtained using a dissecting scope and analyzed with ImageJ. Tumor areas were converted from pixels to mm² via a ruler. To quantify the positivity of phospho-ERK and phospho-AKT stained slides, H-scores were calculated using Qupath. The H-score is determined by adding the results of multiplication of the percentage of cells with staining intensity ordinal value (scored from 0 for “no signal” to 3 for “strong signal”) with 300 possible values [84]. To normalize potential variations between different rounds of immunohistochemistry, one patient sample was included and stained for both pERK and pAKT in all rounds of staining as a control.

### Immunoblotting

For apoptosis and proliferation assays, 3×10⁵ cells were seeded into 6-well plates and allowed to adhere overnight in regular growth media and cultured in the presence or absence 10 uM of Capivasertib, RMC-4550, or a combination of both drugs. After 24 hours, the protein was extracted using RIPA lysis buffer (Thermo Fisher Scientific, 89900) and proteinase/phosphatase inhibitor cocktail (Thermo Fisher Scientific, 78442). Protein concentration was measured using BCA protein assay kit (Thermo Fisher Scientific, 23250). Proteins (30 ug from each sample) were separated by SDS-PAGE and immunoblotted and transferred to polyvinyl difluoride (PVDF) membranes (BioRad, 162-0177) according to standard protocols. Membranes were immunoblotted with antibodies against phosphor-ERK (Cell Signaling Technology, 4370), ERK (Cell Signaling Technology, 9102), phosphor-AKT (Thermo Fisher Scientific, 44-621G), AKT (Cell Signaling Technology, 4691), phospho-S6 (Cell Signaling Technology, 4858), S6 (Cell Signaling Technology, 2217), and HSP90 (BD Bioscience, 610418). Immunoblots were developed using Supersignal® West Dura Extended Duration Chemiluminescent Substrate (Thermo Fisher Scientific, 37071). Initially, the membranes were immunoblotted against non-phosphorylated targets, and after stripping these antibodies using Western Blot Stripping Buffer (Thermo Fisher Scientific, 46430), they were immunoblotted against phosphorylated antibodies. Developing the signal was done using Dura Extended Duration Chemiluminescent Substrate (Thermo Fisher Scientific, 37071). All immunoblots were performed at least three times independently.

### Clonogenic, Apoptosis, and Proliferation Assays

For clonogenic assays, cells were seeded in triplicate into 24-well plates (4000 cells per well) and allowed to adhere overnight in regular growth media. Cells were then cultured in the absence or presence of the drug as indicated on each figure panel in complete media for 4 days. Growth media with or without drugs was replaced every 2 days. The remaining cells were stained with 0.5% crystal violet in 20% methanol and photographed using a digital scanner. Relative growth was quantified by densitometry after extracting crystal violet from the stained cells using 100% methanol[85].

Drug synergism was analyzed using SynergyFinder (https://synergyfinder.fimm.fi) web application[86]. The degree of combination synergy, or antagonism, is quantified by comparing the observed drug combination response against the expected response, calculated using Loewe's model that assumes no interaction between drugs [87].

For apoptosis and proliferation assays, 3×10⁵ cells were seeded into 6-well plates, and allowed to adhere overnight in regular growth media, and cultured in the presence or absence of 10 uM of Capivasertib, RMC-4550, or a combination of both drugs. After 24 hours, apoptosis and cell proliferation were determined through staining with Fixable Viability Dye eFluor™ 450 (Thermo Fisher Scientific, 65-0863-14), cleaved caspase 3 Antibody (Cell Signaling Technology, 9669), and Click-iT™ EdU Alexa Fluor™ 647 Flow Cytometry Assay Kit (Thermo Fisher Scientific, C-10424) according to the manufacturer's instructions. Data were acquired using a BD LSR II Flow Cytometer. All experiments were performed independently two times on 3 different cell lines.

### Tumor Dissociation, Cell Sorting, and RNA-Sequencing

Primary tumors were dissociated using collagenase IV, dispase, and trypsin at 37° C. for 30 min. After dissociation, the samples remained continually on ice, were in contact with ice-cold solutions, and were in the presence of 2 mM EDTA and 1 U/ml DNase to prevent aggregation. Cells were stained with antibodies to CD45 (BioLegend, 103112), CD31 (BioLegend, 303116), F4/80 (BioLegend, 123116), and Ter119 (BioLegend, 116212) to exclude hematopoietic and endothelial cells (lineage-positive (Lin⁺) cells). DAPI was used to exclude dead cells. FACSAria sorters (BD Biosciences) were used for cell sorting.

RNA was purified using RNA/DNA All Prep kit (Qiagen, 80284). RNA quality of each tumor sample was assessed using the RNA6000 PicoAssay for the Agilent 2100 Bioanalyzer as per the manufacturer's recommendation. 4.4 ng total RNA per sample was used for cDNA synthesis and library preparation using Trio RNA-Seq, Mouse rRNA kit (Tecan, 0507-32), according to the manufacturer's instructions. The purified cDNA library products were evaluated using the Agilent bioanalyzer and sequenced on NextSeq High Output 1×75 (Admera Health Biopharma Services).

### Analysis of Mouse Model-Derived RNA-Seq Datasets

Paired-end RNA-seq reads were aligned to the mm10 mouse genome using STAR (v2.6.1d) 2-pass mapping and estimates of transcript abundance were obtained using RSEM (v1.2.30) [88, 89]. The differentially expressed genes between different tumor genotypes and treatment groups were called by DESeq2 using transcript abundance estimates via tximport [90, 91]. The DESeq2-calculated fold changes were used to generate ranked gene lists for input into GSEA [92].

The upregulated genes with absolute log 2 fold change greater than 1 and a false discovery rate less than 0.05 in the comparison of Nf1, Rasa1, and Pten mutant oncogene-negative tumors with Kras^{G12D}-driven tumors (KTC+sgInert and KTC+sgPten)

were compiled into a signature reflecting the oncogene-negative adenocarcinoma state. This gene signature was utilized in the analysis of human oncogene-positive and oncogene-negative tumors. Scaled estimates of transcript abundance for TCGA LUAD samples were obtained from the GDC data portal (gdc-portal.nci.nih.gov). Each expression profile was then scored on the basis of the mouse-derived gene signature using single-sample GSEA within the Gene Set Variation Analysis (GSVA) package[93].

### REFERENCES

1. Gao, Q., et al., Driver Fusions and Their Implications in the Development and Treatment of Human Cancers. Cell Rep, 2018. 23(1): p. 227-238 e3.
2. Lu, X., et al., MET Exon 14 Mutation Encodes an Actionable Therapeutic Target in Lung Adenocarcinoma. Cancer Res, 2017. 77(16): p. 4498-4505.
3. Liu, J., et al., An Integrated TCGA Pan-Cancer Clinical Data Resource to Drive High—Quality Survival Outcome Analytics. Cell, 2018. 173(2): p. 400-416 e11.
4. Hallin, J., et al., The KRAS(G12C) Inhibitor MRTX849 Provides Insight toward Therapeutic Susceptibility of KRAS-Mutant Cancers in Mouse Models and Patients. Cancer Discov, 2020. 10(1): p. 54-71.
5. Canon, J., et al., The clinical KRAS(G12C) inhibitor AMG 510 drives anti-tumour immunity. Nature, 2019. 575(7781): p. 217-223.
6. Jackson, E. L., et al., Analysis of lung tumor initiation and progression using conditional expression of oncogenic K-ras. Genes Dev, 2001. 15(24): p. 3243-8.
7. Winters, I. P., et al., Multiplexed in vivo homology-directed repair and tumor barcoding enables parallel quantification of Kras variant oncogenicity. Nat Commun, 2017. 8(1): p. 2053.
8. Guerra, C., et al., Tumor induction by an endogenous K-ras oncogene is highly dependent on cellular context. Cancer Cell, 2003. 4(2): p. 111-20.
9. Hingorani, S. R., et al., Preinvasive and invasive ductal pancreatic cancer and its early detection in the mouse. Cancer Cell, 2003. 4(6): p. 437-50.
10. Zafra, M. P., et al., An In Vivo Kras Allelic Series Reveals Distinct Phenotypes of Common Oncogenic Variants. Cancer Discov, 2020. 10(11): p. 1654-1671.
11. Paez, J. G., et al., EGFR mutations in lung cancer: correlation with clinical response to gefitinib therapy. Science, 2004. 304(5676): p. 1497-500.
12. Lynch, T. J., et al., Activating mutations in the epidermal growth factor receptor underlying responsiveness of non-small-cell lung cancer to gefitinib. N Engl J Med, 2004. 350(21): p. 2129-39.
13. Politi, K., et al., Lung adenocarcinomas induced in mice by mutant EGF receptors found in human lung cancers respond to a tyrosine kinase inhibitor or to down-regulation of the receptors. Genes Dev, 2006. 20(11): p. 1496-510.
14. Li, D., et al., Bronchial and peripheral murine lung carcinomas induced by T790M-L858R mutant EGFR respond to HKI-272 and rapamycin combination therapy. Cancer Cell, 2007. 12(1): p. 81-93.
15. Ji, H., et al., The impact of human EGFR kinase domain mutations on lung tumorigenesis and in vivo sensitivity to EGFR-targeted therapies. Cancer Cell, 2006. 9(6): p. 485-95.
16. Zhu, H., et al., Oncogenic EGFR signaling cooperates with loss of tumor suppressor gene functions in gliomagenesis. Proc Natl Acad Sci USA, 2009. 106(8): p. 2712-6.
17. Lin, Q., et al., The association between BRAF mutation class and clinical features in BRAF-mutant Chinese non-small cell lung cancer patients. J Transl Med, 2019. 17(1): p. 298.
18. van Veen, J. E., et al., Mutationally-activated PI3-kinase-alpha promotes de-differentiation of lung tumors initiated by the BRAF(V600E) oncoprotein kinase. Elife, 2019. 8.
19. Dankort, D., et al., A new mouse model to explore the initiation, progression, and therapy of BRAFV600E-induced lung tumors. Genes Dev, 2007. 21(4): p. 379-84.
20. Dankort, D., et al., Braf(V600E) cooperates with Pten loss to induce metastatic melanoma. Nat Genet, 2009. 41(5): p. 544-52.
21. Charles, R. P., et al., Mutationally activated BRAF(V600E) elicits papillary thyroid cancer in the adult mouse. Cancer Res, 2011. 71(11): p. 3863-71.
22. Davies, H., et al., Mutations of the BRAF gene in human cancer. Nature, 2002. 417(6892): p. 949-54.
23. Chin, L., et al., Essential role for oncogenic Ras in tumour maintenance. Nature, 1999. 400(6743): p. 468-72.
24. Seeburg, P. H., et al., Biological properties of human c-Ha-ras1 genes mutated at codon 12. Nature, 1984. 312(5989): p. 71-5.
25. Der, C. J., T. Finkel, and G. M. Cooper, Biological and biochemical properties of human rasH genes mutated at codon 61. Cell, 1986. 44(1): p. 167-76.
26. Kwong, L. N., et al., Oncogenic NRAS signaling differentially regulates survival and proliferation in melanoma. Nat Med, 2012. 18(10): p. 1503-10.
27. Ackermann, J., et al., Metastasizing melanoma formation caused by expression of activated N-RasQ61K on an INK4a-deficient background. Cancer Res, 2005. 65(10): p. 4005-11.
28. Paik, P. K., et al., Response to MET inhibitors in patients with stage IV lung adenocarcinomas harboring MET mutations causing exon 14 skipping. Cancer Discov, 2015. 5(8): p. 842-9.
29. Drilon, A., et al., Antitumor activity of crizotinib in lung cancers harboring a MET exon 14 alteration. Nat Med, 2020. 26(1): p. 47-51.
30. Kim, M., et al., Patient-derived lung cancer organoids as in vitro cancer models for therapeutic screening. Nat Commun, 2019. 10(1): p. 3991.
31. Gow, C. H., et al., Oncogenic Function of a KIF5B-MET Fusion Variant in Non-Small Cell Lung Cancer. Neoplasia, 2018. 20(8): p. 838-847.
32. Gao, Y., et al., Allele-Specific Mechanisms of Activation of MEK1 Mutants Determine Their Properties. Cancer Discov, 2018. 8(5): p. 648-661.
33. Cai, D., et al., Identification and Characterization of Oncogenic SOS1 Mutations in Lung Adenocarcinoma. Mol Cancer Res, 2019. 17(4): p. 1002-1012.
34. Shaw, A. T., et al., Crizotinib versus chemotherapy in advanced ALK-positive lung cancer. N Engl J Med, 2013. 368(25): p. 2385-94.
35. Maddalo, D., et al., In vivo engineering of oncogenic chromosomal rearrangements with the CRISPR/Cas9 system. Nature, 2014. 516(7531): p. 423-7.
36. Pyo, K. H., et al., Establishment of a Conditional Transgenic Mouse Model Recapitulating EML4-ALK-Positive Human Non-Small Cell Lung Cancer. J Thorac Oncol, 2017. 12(3): p. 491-500.
37. Chiarle, R., et al., NPM-ALK transgenic mice spontaneously develop T-cell lymphomas and plasma cell tumors. Blood, 2003. 101(5): p. 1919-27.
38. Soda, M., et al., Identification of the transforming EML4-ALK fusion gene in non-small—cell lung cancer. Nature, 2007. 448(7153): p. 561-6.
39. Subbiah, V., et al., Precision Targeted Therapy with BLU-667 for RET-Driven Cancers. Cancer Discov, 2018. 8(7): p. 836-849.
40. Huang, Q., et al., Preclinical Modeling of KIF5B-RET Fusion Lung Adenocarcinoma. Mol Cancer Ther, 2016. 15(10): p. 2521-2529.
41. Saito, M., et al., A mouse model of KIF5B-RET fusion-dependent lung tumorigenesis. Carcinogenesis, 2014. 35(11): p. 2452-6.
42. Shaw, A. T., et al., Crizotinib in ROS1-rearranged non-small-cell lung cancer. N Engl J Med, 2014. 371(21): p. 1963-71.
43. Arai, Y., et al., Mouse model for ROS1-rearranged lung cancer. PLoS One, 2013. 8(2): p. e56010.
44. Inoue, M., et al., Mouse models for ROS1-fusion-positive lung cancers and their application to the analysis of multikinase inhibitor efficiency. Carcinogenesis, 2016. 37(5): p. 452-60.
45. Farago, A. F., et al., Durable Clinical Response to Entrectinib in NTRK1-Rearranged Non—Small Cell Lung Cancer. J Thorac Oncol, 2015. 10(12): p. 1670-4.
46. Vaishnavi, A., et al., Oncogenic and drug-sensitive NTRK1 rearrangements in lung cancer. Nat Med, 2013. 19(11): p. 1469-1472.
47. Drilon, A., et al., Response to ERBB3-Directed Targeted Therapy in NRG1-Rearranged Cancers. Cancer Discov, 2018. 8(6): p. 686-695.
48. Hyman, D. M., et al., AKT Inhibition in Solid Tumors With AKT1 Mutations. J Clin Oncol, 2017. 35(20): p. 2251-2259.
49. De Marco, C., et al., Mutant AKT1-E17K is oncogenic in lung epithelial cells. Oncotarget, 2015. 6(37): p. 39634-50.
50. Berger, A. H., et al., Oncogenic RIT1 mutations in lung adenocarcinoma. Oncogene, 2014. 33(35): p. 4418-23.
51. Pillai, R. N., et al., HER2 mutations in lung adenocarcinomas: A report from the Lung Cancer Mutation Consortium. Cancer, 2017. 123(21): p. 4099-4105.
52. Perera, S. A., et al., HER2YVMA drives rapid development of adenosquamous lung tumors in mice that are sensitive to BIBW2992 and rapamycin combination therapy. Proc Natl Acad Sci USA, 2009. 106(2): p. 474-9.
53. Liu, S., et al., Targeting HER2 Aberrations in Non-Small Cell Lung Cancer with Osimertinib. Clin Cancer Res, 2018. 24(11): p. 2594-2604.
54. Weinstein, E. J., D. I. Kitsberg, and P. Leder, A mouse model for breast cancer induced by amplification and overexpression of the neu promoter and transgene. Mol Med, 2000. 6(1): p. 4-16.
55. Guy, C. T., et al., Expression of the neu protooncogene in the mammary epithelium of transgenic mice induces metastatic disease. Proc Natl Acad Sci USA, 1992. 89(22): p. 10578-82.
56. Greulich, H., et al., Functional analysis of receptor tyrosine kinase mutations in lung cancer identifies oncogenic extracellular domain mutations of ERBB2. Proc Natl Acad Sci USA, 2012. 109(36): p. 14476-81.
57. Engelman, J. A., et al., Effective use of PI3K and MEK inhibitors to treat mutant Kras G12D and PIK3CA H1047R murine lung cancers. Nat Med, 2008. 14(12): p. 1351-6.
58. Trejo, C. L., et al., Mutationally activated PIK3CA(H1047R) cooperates with BRAF(V600E) to promote lung cancer progression. Cancer Res, 2013. 73(21): p. 6448-61.
59. Sanchez-Vega, F., et al., Oncogenic Signaling Pathways in The Cancer Genome Atlas. Cell, 2018. 173(2): p. 321-337 e10.
60. Bailey, M. H., et al., Comprehensive Characterization of Cancer Driver Genes and Mutations. Cell, 2018. 173(2): p. 371-385 e18.
61. Yi, K. H. and J. Lauring, Recurrent AKT mutations in human cancers: functional consequences and effects on drug sensitivity. Oncotarget, 2016. 7(4): p. 4241-51.
62. Dobashi, Y., et al., Diverse involvement of isoforms and gene aberrations of Akt in human lung carcinomas. Cancer Sci, 2015. 106(6): p. 772-81.
63. Arboleda, M. J., et al., Overexpression of AKT2/protein kinase Bbeta leads to up-regulation of beta1 integrins, increased invasion, and metastasis of human breast and ovarian cancer cells. Cancer Res, 2003. 63(1): p. 196-206.
64. Zhang, Y., et al., A Pan-Cancer Proteogenomic Atlas of PI3K/AKT/mTOR Pathway Alterations. Cancer Cell, 2017. 31(6): p. 820-832 e3.
65. Chaft, J. E., et al., Coexistence of PIK3CA and other oncogene mutations in lung adenocarcinoma-rationale for comprehensive mutation profiling. Mol Cancer Ther, 2012. 11(2): p. 485-91.
66. Dbouk, H. A., et al., Characterization of a tumor-associated activating mutation of the p110beta PI3-kinase. PLoS One, 2013. 8(5): p. e63833.
67. Pazarentzos, E., et al., Oncogenic activation of the PI3-kinase p110beta isoform via the tumor-derived PIK3Cbeta(D1067V) kinase domain mutation. Oncogene, 2016. 35(9): p. 1198-205.
68. Whale, A. D., et al., Functional characterization of a novel somatic oncogenic mutation of PIK3CB. Signal Transduct Target Ther, 2017. 2: p. 17063.
69. Liu, C., et al., Mosaic analysis with double markers reveals tumor cell of origin in glioma. Cell, 2011. 146(2): p. 209-21.
70. Zhu, Y., et al., Ablation of NF1 function in neurons induces abnormal development of cerebral cortex and reactive gliosis in the brain. Genes Dev, 2001. 15(7): p. 859-76.
71. Madisen, L., et al., A robust and high-throughput Cre reporting and characterization system for the whole mouse brain. Nat Neurosci, 2010. 13(1): p. 133-40.
72. Chiou, S. H., et al., Pancreatic cancer modeling using retrograde viral vector delivery and in vivo CRISPR/Cas9-mediated somatic genome editing. Genes Dev, 2015. 29(14): p. 1576-85.
73. Okawa, H., et al., Hepatocyte-specific deletion of the keap1 gene activates Nrf2 and confers potent resistance against acute drug toxicity. Biochem Biophys Res Commun, 2006. 339(1): p. 79-88.
74. Bardeesy, N., et al., Loss of the Lkb1 tumour suppressor provokes intestinal polyposis but resistance to transformation. Nature, 2002. 419(6903): p. 162-7.
75. Jonkers, J., et al., Synergistic tumor suppressor activity of BRCA2 and p53 in a conditional mouse model for breast cancer. Nat Genet, 2001. 29(4): p. 418-25.
76. Cai, H., et al., A functional taxonomy of tumor suppression in oncogenic KRAS-driven lung cancer. Under consideration, 2021.
77. Rogers, Z. N., et al., A quantitative and multiplexed approach to uncover the fitness landscape of tumor suppression in vivo. Nat Methods, 2017. 14(7): p. 737-742.
78. Murray, C. W., et al., An LKB1-SIK Axis Suppresses Lung Tumor Growth and Controls Differentiation. Cancer Discov, 2019. 9(11): p. 1590-1605.
79. Rogers, Z. N., et al., Mapping the in vivo fitness landscape of lung adenocarcinoma tumor suppression in mice. Nat Genet, 2018. 50(4): p. 483-486.
80. Orr, H. A., Fitness and its role in evolutionary genetics. Nat Rev Genet, 2009. 10(8): p. 531-9.
81. J. B. S., H., The mathematical theory of natural and artificial selection. Mathematical Proceedings of the Cambridge Philosophical Society, 1927. 23(5): p. 607-615.
82. Geraldine A. Van der Auwera, B. D. O. C., Genomics in the Cloud. 2020.
83. McLaren, W., et al., The Ensembl Variant Effect Predictor. Genome Biol, 2016. 17(1): p. 122.
84. Fedchenko, N. and J. Reifenrath, Different approaches for interpretation and reporting of immunohistochemistry analysis results in the bone tissue—a review. Diagn Pathol, 2014. 9: p. 221.
85. Feoktistova, M., P. Geserick, and M. Leverkus, Crystal Violet Assay for Determining Viability of Cultured Cells. Cold Spring Harb Protoc, 2016. 2016(4): p. pdb prot087379.
86. Ianevski, A., A. K. Giri, and T. Aittokallio, SynergyFinder 2.0: visual analytics of multi—drug combination synergies. Nucleic Acids Res, 2020. 48(W1): p. W488-W493.
87. Loewe, S., The problem of synergism and antagonism of combined drugs. Arzneimittelforschung, 1953. 3(6): p. 285-90.
88. Dobin, A., et al., STAR: ultrafast universal RNA-seq aligner. Bioinformatics, 2013. 29(1): p. 15-21.
89. Li, B. and C. N. Dewey, RSEM: accurate transcript quantification from RNA-Seq data with or without a reference genome. BMC Bioinformatics, 2011. 12: p. 323.
90. Soneson, C., M. I. Love, and M. D. Robinson, Differential analyses for RNA-seq: transcript-level estimates improve gene-level inferences. F1000Res, 2015. 4: p. 1521.
91. Love, M. I., W. Huber, and S. Anders, Moderated estimation of fold change and dispersion for RNA-seq data with DESeq2. Genome Biol, 2014. 15(12): p. 550.
92. Subramanian, A., et al., Gene set enrichment analysis: a knowledge-based approach for interpreting genome-wide expression profiles. Proc Natl Acad Sci USA, 2005. 102(43): p. 15545-50.
93. Hanzelmann, S., R. Castelo, and J. Guinney, GSVA: gene set variation analysis for microarray and RNA-seq data. BMC Bioinformatics, 2013. 14: p. 7.

### Example 2—Evaluation of Oncogene-Negative Tumors

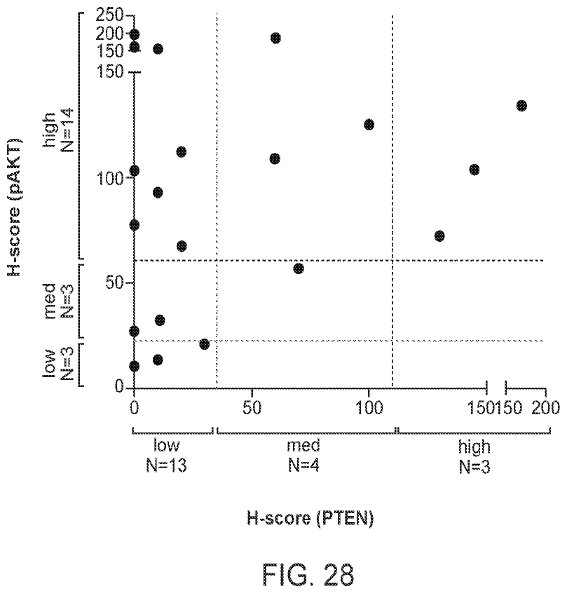

FIG. 28 Sections were stained from 20 oncogene-negative human tumors that showed no genomic alterations for PTEN. As shown in this graph, despite the lack of genomic alterations for PTEN, the majority of the tumors exhibited low levels of PTEN protein. Of those, the vast majority (all but 3) exhibited medium to high levels of pAKT (a measure of PI3K-AKT pathway activity). In total, 50% of all tumors tested exhibited low levels of PTEN protein and medium to high levels of pAKT. These data provide evidence that the pathways discussed herein (Ras/MAPK pathway and PI3K-AKT pathway) can be upregulated (can exhibit increased activity) in non-genomic ways (e.g., via epigenomic effects). For example, expression and/or activity of the positive and negative regulators of these pathways can modulated without direct genomic modification (e.g., substitution, deletion, insertion mutations).

### Example 3—Updated Version of Example 1

### Introduction

Lung cancer is the leading cause of cancer death1. Lung adenocarcinoma, the most prevalent subtype of lung cancer, has frequent alterations in receptor tyrosine kinase and RAS/RAF pathway oncogenes, including mutations in EGFR and KRAS2. The identification of driver oncogenes has enabled a shift from toxic chemotherapies to less toxic and more effective therapies that often target the oncogenes3. However, approximately 30 percent of lung adenocarcinomas are thought to lack a driving oncogene4-6. Consequently, developing targeted therapies for these tumors remains a major unmet challenge for precision thoracic oncology.

Extensive genomic and transcriptomic studies suggest that neither technical reasons nor the presence of novel oncogenes likely explain this large and clinically significant population of lung cancer patients1, 2, 4-12. Thus, despite the diagnosis of more than 150,000 patients per year with oncogene-negative lung adenocarcinomas worldwide, the genetic events and biochemical pathway changes that drive the initiation and growth of these tumors remain almost entirely unknown.

Oncogenes and tumor suppressor genes are parts of signaling networks that generate and sustain the biochemical changes that drive tumor initiation and growth13-16. Combinatorial alterations in tumor suppressor genes could co-operate to activate pathways driving oncogene71 negative lung tumors. Human lung adenocarcinoma have complex patterns of mutations across many putative tumor suppressor genes4. However, the ability to predict which combinations of genomic alterations drive cancer in the absence of oncogene activation based on human genomic data alone remains challenging. While human genomic data can predict combinations of genomic mutations as likely cancer drivers when the mutations co-occur at very high frequencies 17-20, identifying pathogenic combinations of less frequently mutated genes poses a nearly insurmountable statistical challenge. Furthermore, the large numbers of mutations in lung cancers, non-genomic mechanism that often inactivate tumor suppressor genes, and generation of similar biochemical effects through inactivation of different genes further reduce the ability of human cancer genomic studies to identify combinatorial alterations that activate driver pathways in lung cancer 21-24.

Functional genomic studies within autochthonous cancer models can help identify the pathways involved in tumorigenesis in vivo 25. Here, we leveraged quantitative mouse model systems to assess the ability of hundreds of combinatorial alterations of tumor suppressor genes, acting across many different signaling pathways, to generate oncogene-negative lung adenocarcinomas in vivo. We uncover pathway-level changes that drive lung cancer in the absence of oncogene mutations, translate these findings to human oncogene-negative lung adenocarcinoma, and leveraged these results to identify therapeutic vulnerabilities.

### Results

### A Large Fraction of Human Lung Adenocarcinomas Lack Oncogene Mutations

To better understand the genomics of lung adenocarcinomas that lack oncogene mutations, we analyzed data from The Cancer Genome Atlas (TCGA) and AACR Genomics Evidence Neoplasia Information Exchange (GENIE) 26, 27. We classified tumors as oncogene positive if they had high-confidence oncogenic alterations in previously described proto oncogenes, oncogene-indeterminate if they had alterations of unknown significance in known proto-oncogenes, and oncogene-negative if they had no alterations in known proto-oncogenes (Methods). Consistent with previous publications, we found that 17-18% of lung adenocarcinomas were oncogene-negative (FIGS. 1 and 6) 28-30. Additionally, 15-27% of lung adenocarcinomas were oncogene-indeterminate and thus 32-45% of lung adenocarcinomas lack known oncogene mutations. Patients with oncogene-negative, oncogene-indeterminate, and oncogene-positive lung adenocarcinomas have broadly similar mutational burden and clinical characteristics (FIG. 6).

### Combinatorial Tumor Suppressor Gene Inactivation Enables Lung Tumor Development

To determine whether combinatorial tumor suppressor gene inactivation can drive lung tumor initiation in the absence of oncogene activation, we coupled Cre/loxP-based genetically engineered mouse models and somatic CRISPR/Cas9-based genome editing with tumor barcoding and high-throughput barcode sequencing (Tuba-seq) 31-35. We used Cre/loxP to inactivate each of five “core” tumor suppressor genes (Trp53, Lkb1/Stk11, Keap1, Nf1, and Pten). These genes are within diverse pathways and are frequently inactivated in human lung cancers, including oncogene-negative lung adenocarcinomas (FIG. 7) [35-38]. We used CRISPR/Cas9 to coincidentally inactivate panels of additional tumor suppressor genes in lung epithelial cells in mice with floxed alleles of each of the “core” tumor suppressors, a Cre-reporter allele (R26LSL-Tom (T) 36), and a Cre-regulated Cas9 allele (H11LSL-Cas9 (C) 37).

We transduced Nf1f/f; TC, Ptenf/f; TC, Trp53f/f; TC, Lkb1f/f; TC, Keap1f/f; TC, TC, and T mice with two pools of barcoded Lenti-sgRNA/Cre vectors that target ˜50 putative tumor suppressor genes that we previously investigated in KRASG12D-driven lung tumors (Lenti-sgTS15/Cre and Lenti-sgTS102/Cre) (FIG. 1, 7, 8) 31, 32, 35. The mutation frequency of these genes varied, and mutations in some were enriched in oncogene-negative human lung adenocarcinomas (FIG. 7). The combination of Cre/LoxP and CRISPR/Cas9-based genome editing should generate hundreds of combinations of genomic alterations in lung epithelial cells. We previously found that a small percent of lung tumors initiated with Lenti sgRNA/Cre vectors in other lung cancer models contained multiple sgRNAs, consistent with the transduction of the initial cell with multiple Lenti-sgRNA/Cre vectors 31, 32. Thus, we used a high titer of the Lenti-sgRNA/Cre pools in these experiments to increases the likelihood of finding higher-order genetic interactions that drive tumorigenesis.

One year after transduction with the Lenti-sgRNA/128 Cre pools, Nf1f/f; TC, Ptenf/f; TC, and Trp53f/f; TC mice developed a modest number of tumors (defined as Tomatopositive expansion >0.5 mm in diameter), while Lkb1f/f; TC and Keap1f/f; TC mice rarely developed any tumors (FIGS. 1,8). Interestingly, Nf1f/f; TC, Ptenf/f; TC, and Trp53f/f; TC, and TC mice transduced with the larger Lenti-sgTS102/Cre pool developed many more tumors than those transduced with the Lenti-sgTS15/Cre pool. These tumors were positive for TTF1/NKX2-1, a marker for lung adenocarcinoma, and negative for P63 and UCHL1, markers for squamous cell and small cell lung cancer, respectively (FIG. 1).

To determine whether these tumors contained spontaneous oncogene mutations, we PCR amplified and sequenced 10 genomic regions in Kras, Braf, Nras, and Egfr (FIG. 8, and Methods) 33, 38-46. Across 29 samples, we detected only one oncogene mutation (a KrasG12V mutation in a tumor from a Ptenf/f; TC mouse). Thus, the majority of these tumors arose in the absence of hotspot mutations in these proto-oncogenes. This is consistent with the low mutation rate in mouse models of lung cancer 47 and suggests that the inactivation of combinations of specific tumor suppressor genes in Nf1f/f; TC, Ptenf/f; TC, and Trp53f/f; TC mice drives the development of lung cancer in vivo. Notably, the overall low number of tumors indicates that inactivation of the “core” tumor suppressor genes alone, and most combinations of tumor suppressor genes tested, are insufficient to generate lung tumors.

### Identification of Top Candidate Tumor Suppressor Genes Involved in Oncogene-Negative Lung Tumor Formation

The Lenti-sgRNA/Cre vectors contain two-component barcodes in which an sgID identifies the sgRNA and a random barcode (BC) uniquely tags each clonal tumor. Thus, high throughput sequencing of the sgID-BC region can identify the sgRNA(s) present in each tumor and quantify the number of cancer cells in each tumor (FIG. 1). To determine which sgRNAs were present in the largest tumors, we PCR-amplified the sgID-BC region from genomic DNA from dissected tumors and performed high-throughput sgID-BC sequencing. Most large tumors contained multiple Lenti-sgRNA/Cre vectors therefore, we calculated the statistical enrichment of each sgRNA based on their relative representation in the dissected tumors (FIGS. 1,9, see Methods).

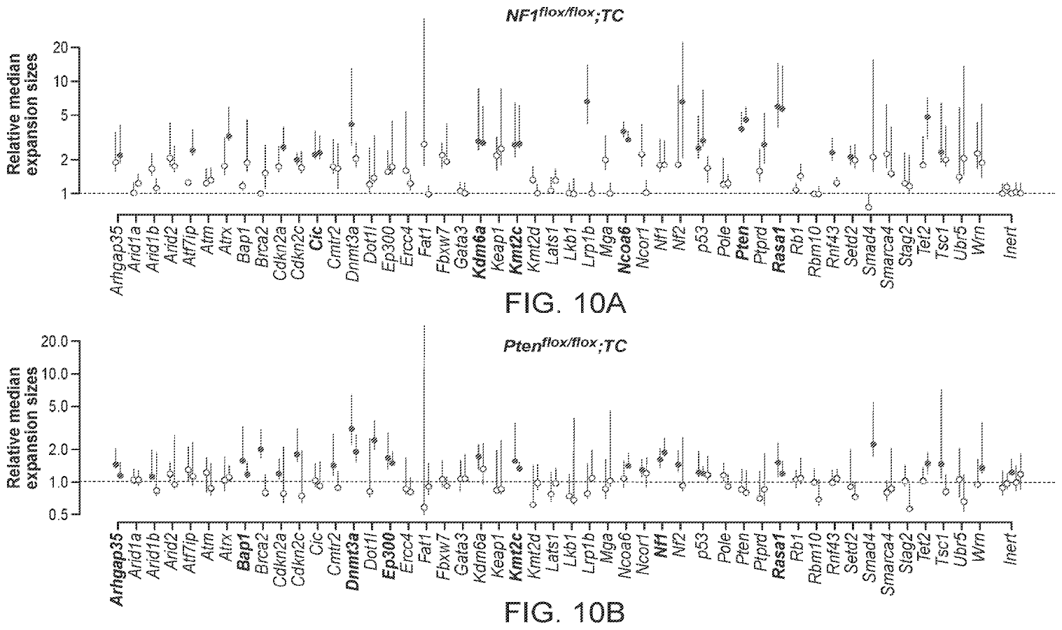

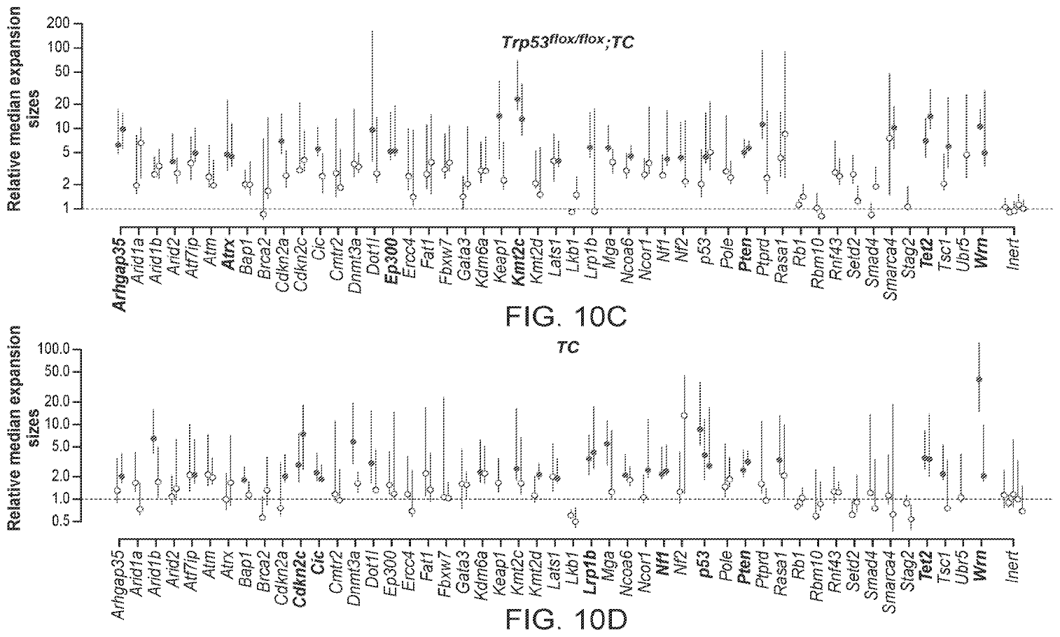

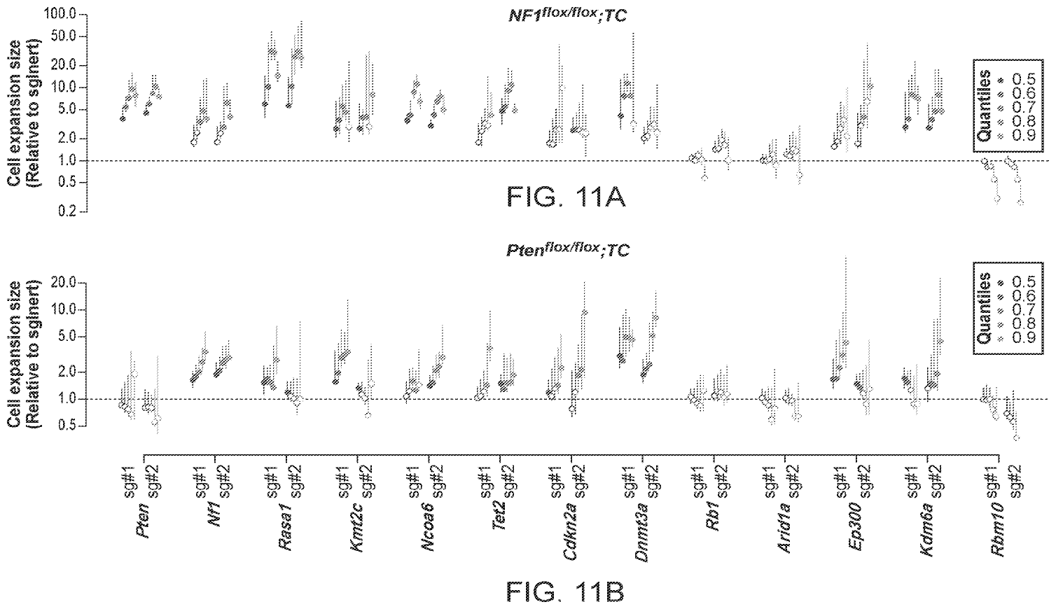

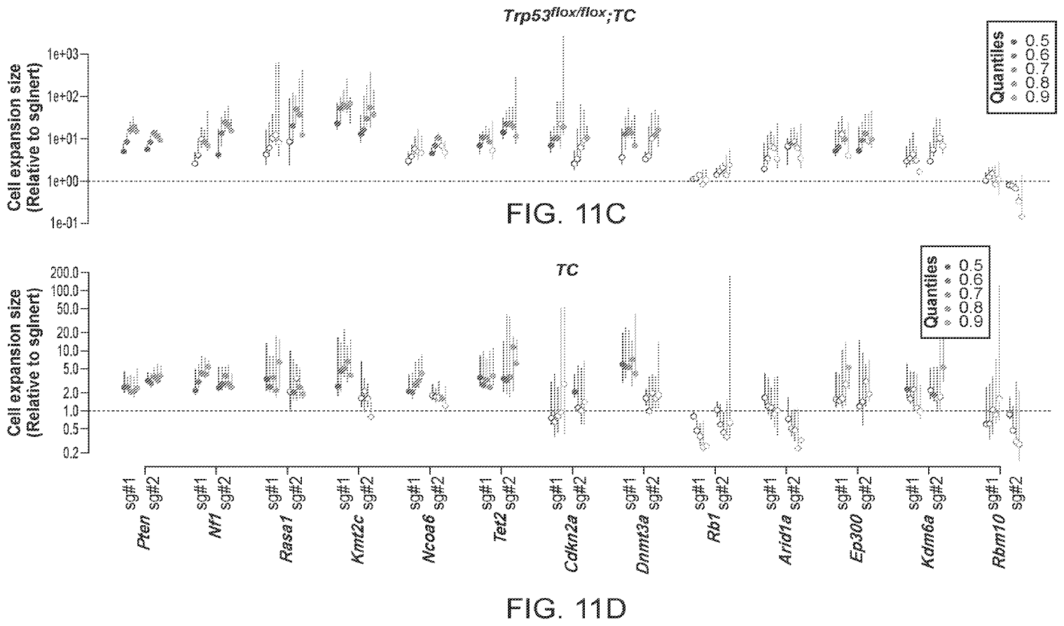

To further quantify the impact of inactivating each tumor suppressor gene on clonal expansion of lung epithelial cells, we performed tumor barcode sequencing (Tuba-seq) on bulk DNA from one lung lobe from each Nf1f/f; TC, Ptenf/f; TC, Trp53f/f; TC, and TC mouse (FIG. 1). Analysis of the number of cells in clonal expansions further nominated tumor suppressor genes that may contribute to tumor initiation and growth (FIGS. 1, 10). Based on these two analyses, we selected 13 genes for further analysis (FIG. 1). The potential importance of these tumor suppressor genes was often supported by both sgRNAs targeting each gene, consistent with on-target effects. Finally, Lenti-sgPten/Cre enrichment in tumors in Nf1f/f; TC mice and Lenti-sgNf1/Cre enrichment in tumors in Ptenf/f; TC mice cross-validate our screen (FIGS. 1,9,10).

### Inactivation of Candidate Tumor Suppressors Efficiently Generates Lung Tumors

To determine the potential of the top candidate tumor suppressor genes to initiate oncogene-negative tumors, we generated a pool of Lenti-sgRNA/Cre vectors targeting each of these tumor suppressor genes and one vector with an inert sgRNA (Lenti-sgTS14/Cre pool; FIG. 2). We targeted each gene with the sgRNA that had the most significant effect on tumor growth and used five times higher titer of each lentiviral vector 174 per mouse than we used in Lenti-sgTS102/Cre pool, thus increasing the potential for the transduction of the initial cell with multiple Lenti-sgRNA/Cre vectors.

We initiated tumors with Lenti-sgTS14/Cre in Nf1f/f; TC, Ptenf/f; TC, Trp53f/f; TC, TC, and KrasLSL-G12D; T (KT) mice. Less than four months after tumor initiation, several Nf1f/f; TC and Ptenf/f; TC mice showed signs of extensive tumor burden. These mice developed many more tumors than mice of the same genotypes one year after transduction with the Lenti-sgTS102/Cre (compare FIG. 2 with FIG. 1, and 18). Thus, this pool of candidate tumor suppressor genes is enriched for those that generate oncogene-negative lung tumors.

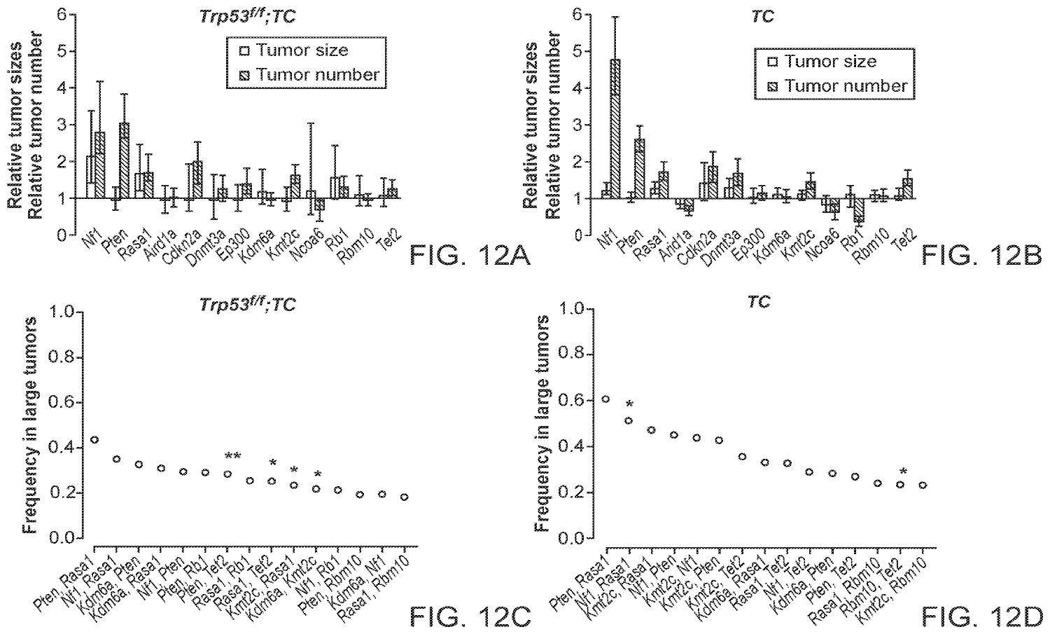

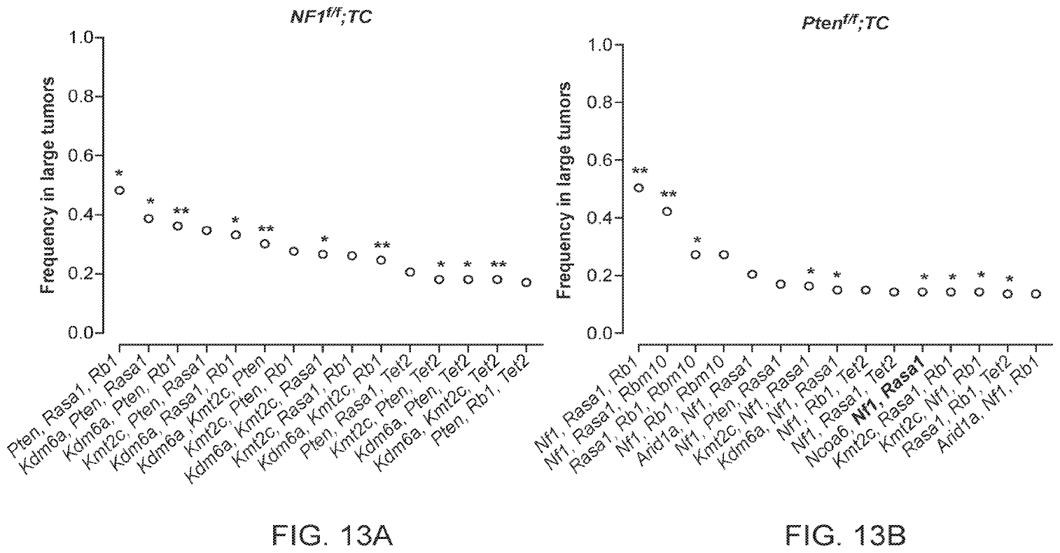

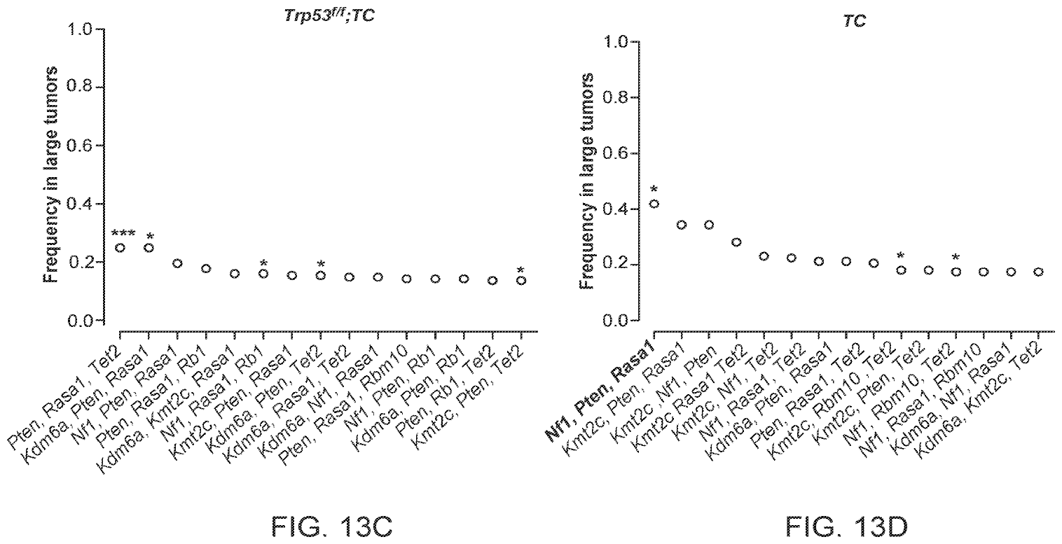

We performed Tuba-seq on DNA from bulk tumor-bearing lungs to determine the number and size of tumors with each barcoded Lenti-sgRNA/Cre vector. Inactivation of Nf1, Rasa1, and Pten most dramatically increased tumor size and/or tumor number across all mouse genotypes (FIGS. 2, 12-13). Inactivation of some of the other tumor suppressor genes less dramatically but significantly increased tumor size and/or tumor number in a genotype-specific manner. This suggests that additional molecular pathways altered by these tumor suppressor genes may also lead to early epithelial expansions.

The largest tumors in Nf1f/f; TC, Ptenf/f; TC, Trp53f/f; TC, and TC mice were frequently generated through the inactivation of multiple tumor suppressor genes. Vectors targeting Nf1, Rasa1, and/or Pten were often present in the largest tumors, and the coincident targeting of Nf1, Rasa1, and Pten was the most frequent combination (FIGS. 2, 12-13). To gain greater insight into the contribution of Nf1, Rasa1, and Pten inactivation to the generation of oncogene-negative tumors, we transduced Nf1f/f; TC, Ptenf/f; TC, Trp53f/f; TC, TC, and KT mice with a pool of Lenti sgRNA/Cre vectors that lacked the vectors targeting Nf1, Rasa1, and Pten (Lenti-sgTS11/Cre) (FIG. 14). Approximately four months after transduction, these mice had many fewer tumors than mice transduced with Lenti-sgTS14/Cre pool (FIGS. 14 and 18). Tuba-seq analysis confirmed a dramatic decrease in tumor burden relative to mice that received the Lenti sgTS14/Cre pool (FIG. 2). Thus, the inactivation of Nf1, Rasa1, and Pten emerged as the most important contributors to the generation of oncogene-negative lung tumors.

Extensive experiments generating single and pairwise inactivation of tumor suppressor genes in individual mice led to the development of very few tumors even after long periods of time (FIGS. 15,16,17). Thus, single and pairwise tumor suppressor gene inactivation is rarely sufficient to generate lung tumors and combinatorial inactivation of three or more tumor suppressor genes increases the efficiency of tumor development and/or accelerates the growth of oncogene-negative lung tumors.

### Combinatorial Inactivation of Nf1, Rasa1, and Pten Drives Lung Adenocarcinoma Development Comparably to Oncogenic Kras Mutation

To dissect the higher-order genetic interactions between Nf1, Rasa1, and Pten, we transduced TC and Trp53f/f; TC mice with a pool of eight lentiviral vectors that would inactivate Nf1, Rasa1, and Pten individually, in pairwise combinations, and all three simultaneously (Lenti sgTSTriple-pool/Cre, FIG. 3). Three months after tumor initiation, TC mice had hundreds of large adenomas and adenocarcinomas (FIG. 3 and FIG. 18). Tuba-seq analysis showed that most of the tumor burden arose as a consequence of concomitant inactivation of all three tumor suppressors, with single and pairwise inactivation of these genes generating very few tumors consistent with our previous observations (FIGS. 3 and 15-16). Additional inactivation of Trp53 in Trp53f/f; TC mice did not increase tumor initiation suggesting that Trp53 is not a major suppressor of oncogene-negative lung adenocarcinoma development at these early stages (FIG. 3 and FIG. 18). Finally, to compare the tumor initiation potential of combinatorial Nf1, Rasa1, and Pten inactivation with that of a known oncogene, we transduced KrasLSL-G12D; T mice (which lack Cas9) with Lenti-sgTSTriple-pool/Cre (FIG. 3). Strikingly, coincident inactivation of Nf1, Rasa1, and Pten in TC mice was nearly as potent as oncogenic KRASG12D in driving lung tumor initiation in vivo (FIG. 3).

In molecular evolution studies, generating combinations of genomic alterations and measuring the fitness of each genotype (growth rate) is used to infer the possible and the most probable paths from a wild-type state to a complex genotype 48. Through the generation of all possible combinatorial alterations of Nf1, Rasa1, and Pten, we quantified the fitness conferred by each mutation and the relative probabilities of different adaptive paths leading to the triple mutant genotype. Our data suggest that inactivation of these three genes can occur in any order, with each additional alteration further increasing the fitness (FIG. 3). The Nf1 Rasa1 Pten mutation sequence emerged as the most probable of all six possible paths.

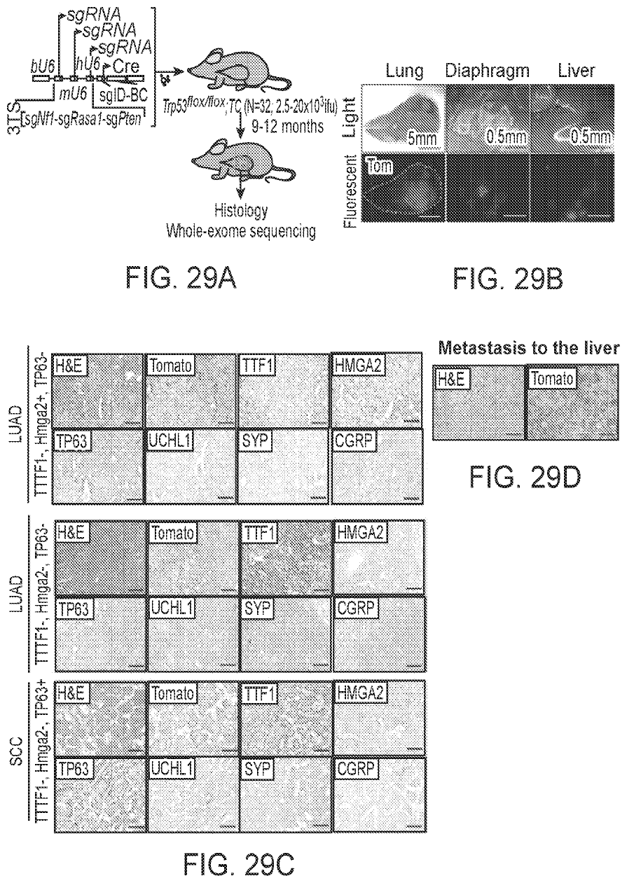

To further analyze tumors driven by inactivation of Nf1, Rasa1, and Pten, we initiated tumors in TC and Trp53f/f; TC mice using only the lentiviral vector that targets all three genes (Lenti-sgNf1-sgRasa1-sgPten/Cre) (FIG. 19). After only three months, these mice developed very large numbers of lung adenomas and adenocarcinomas (FIG. 19). We confirmed the inactivation of Nf1, Rasa1, and Pten in these tumors and whole-exome sequencing uncovered no putative oncogene mutations and only a few putative tumor suppressor mutations, none of which occurred in more than one tumor (FIG. 19). Interestingly, at later timepoints after initiation, tumors in Trp53f/f; TC mice progressed to an invasive NKX2−1 negative HMGA2 positive state and metastasized to other organs such as liver similar to what has been reported in KrasG12D; Trp53 mutant lung adenocarcinoma models (FIG. 29) 49.

Oncogene-Negative Murine Lung Adenocarcinomas have Activated RAS and PI3K Pathways

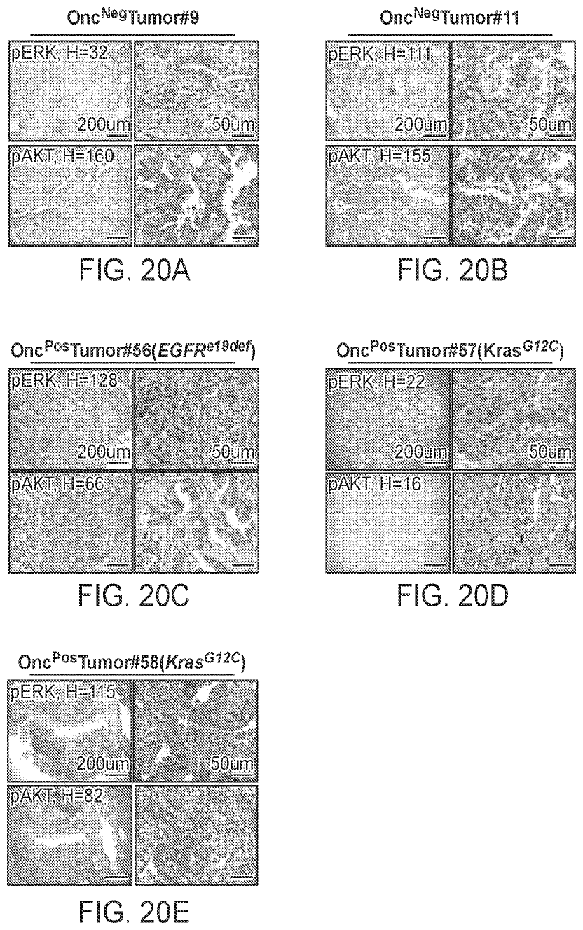

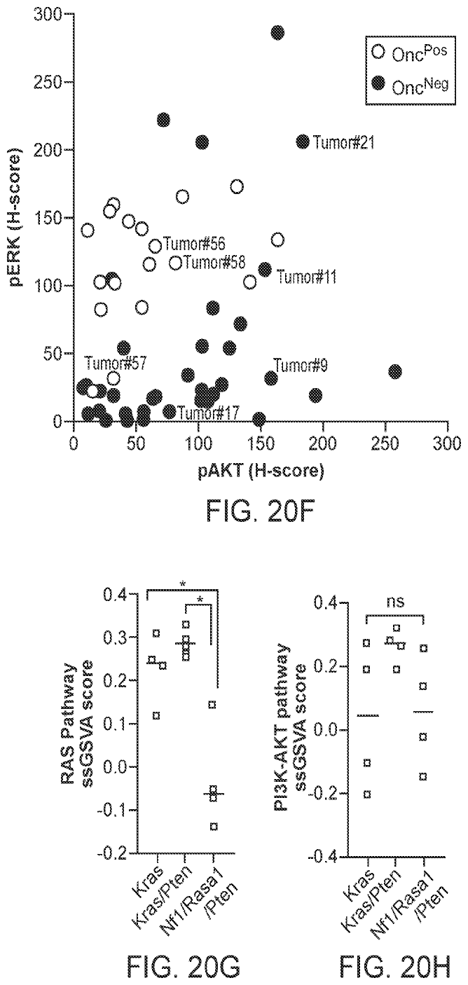

NF1 and RASA1 are negative regulators of RAS, while PTEN is a negative regulator of the PI3K-AKT pathway. Therefore, we investigated the impact of inactivating these tumor suppressor genes on RAS and PI3K pathway activation by immunohistochemistry, as well as by RNA-sequencing (RNA-seq) on FACS-isolated Tomatopositive cancer cells. We generated autochthonous tumors in which Nf1, Rasa1, and Pten were inactivated (TC mice with Lenti sgNf1-sgRasa1-sgPten/Cre; Nf1/Rasa1/Pten tumors), KRASG12D was expressed (KT; H11LSL-Cas9 mice with Lenti-sgInert/Cre; Kras tumors), or KRASG12D was expressed and Pten was inactivated (KT; H11LSL-Cas9 mice with Lenti-sgPten/Cre; Kras/Pten tumors) (FIG. 20). Nf1/Rasa1/Pten tumors had positive staining for pERK (indicative of RAS pathway activation) and pAKT (indicative of PI3K pathway activation) (FIG. 4). Compared with Kras/Pten tumors, the average pERK staining in Nf1/Rasa1/Pten tumors was less intense and pAKT staining was similar (FIG. 4). Single-sample gene set variation analysis (ssGSVA) for previously reported gene sets representing RAS and PI3K-AKT regulated genes 50, 51 on our RNA-seq data confirmed that Nf1/Rasa1/Pten tumors had lower RAS pathway gene signature scores than Kras/Pten tumors (FIG. 20). PI3K-AKT pathway gene signature scores were similar in Nf1/Rasa1/Pten and Kras tumors (FIG. 20). The rare tumors that eventually developed after pairwise inactivation of Nf1, Rasa1, and Pten also had strong activation of RAS and PI3K pathways (FIGS. 15-16 and 20). Based on these analyses, we propose that these tumors represent a subtype of oncogene-negative lung adenocarcinomas with activated RAS and PI3K pathways (Onc-negative RAS/PI3K subtype).

Oncogene-Negative Human Lung Adenocarcinomas Frequently have Activation of RAS and PI3K Pathways

To investigate the activation of RAS and PI3K pathways in human oncogene-negative lung adenocarcinomas, we analyzed oncogene-negative (N=35) and oncogene-positive (N=18) lung adenocarcinomas. Immunohistochemistry for pERK and pAKT showed that ˜45% of oncogene-negative human tumors had moderate to strong activation of both RAS and PI3K pathways and thus represent the Onco-negative RAS/PI3K subtype (FIGS. 4, 20). These tumors were genomically characterized by Stanford's Solid Tumor Actionable Mutation Panel (STAMP)52. Activation of the RAS and PI3K pathways were rarely explained by mutations in NF1, PTEN, or other genes profiled by STAMP, likely due to the noncomprehensive tumor suppressor gene panel characterized by STAMP, as well as epigenetic mechanisms of RAS and PI3K pathway activation. Epigenetic silencing and other non-genomic mechanisms have been well documented to inhibit tumor suppressor genes including PTEN 22, 23, 53, 54. Therefore, we performed immunohistochemistry for PTEN on 20 oncogene-negative lung adenocarcinomas that did not have genomic PTEN mutations. Consistent with previous reports, we observed low PTEN protein levels in 13 out of 20 of these tumors (FIG. 21) 22.

To assess a larger set of oncogene-negative lung adenocarcinomas for alterations that could lead to the activation of RAS and PI3K pathways, we analyzed oncogene-negative tumors in TCGA and GENIE datasets. We queried a set of well-established negative regulators of the RAS and PI3K pathways for alterations in oncogene-negative tumors. Consistent with previous reports, NF1 and RASA1 alterations were enriched in oncogene-negative tumors; however, coincident genomic alterations in NF1, RASA1, and PTEN were rare (FIG. 21) 55, 56. However, over 60% of oncogene-negative lung adenocarcinomas in TCGA had alterations in either the RAS or PI3K pathways, and 22% of these tumors had alterations in components of both pathways, likely representing oncogene-negative RAS/PI3K lung adenocarcinomas (FIG. 4). These frequencies were lower in the GENIE dataset, possibly because only a fraction of the known genes in these pathways were analyzed (FIG. 21). These histological and genomic analyses support a model in which activation of the RAS and PI3K pathways in One negative RAS/PI3K tumors can be generated by diverse genomic and/or epigenetic alterations.

Finally, we assessed whether Onc-negative RAS/PI3K tumors in our mouse model more broadly exhibit transcriptional features that are consistent oncogene-negative human lung adenocarcinoma. We generated a gene expression signature of murine Onc-negative RAS/PI3K tumors comprised of genes that are higher in Nf1/Rasa1/Pten tumors relative to Kras tumors in mice. We then calculated gene signature activity scores for each TCGA lung adenocarcinoma for this Onc-negative RAS/PI3K gene expression signature using single-sample GSEA. Interestingly, the Onc-negative RAS/PI3K signature was highest in oncogene-negative human lung adenocarcinomas relative to lung adenocarcinomas driven by oncogenic KRAS or other known oncogenes (FIG. 4). Collectively, these data indicate that the molecular and biochemical state of mouse Onc-negative RAS/PI3K tumors recapitulates that of a substantial fraction of oncogene-negative human lung adenocarcinomas.

### One-Negative RAS/PI3K Tumors are Vulnerable to Inhibition of RAS and PI3K-AKT Pathways

Understanding the biochemical changes that drive tumor development can nominate potential therapeutic strategies 38. To investigate the therapeutic benefit of targeting key nodes in Onc-negative RAS/PI3K lung cancer, we initiated tumors in TC mice with a smaller pool of barcoded sgRNA viral vectors targeting Nf1, Rasa1, and Pten. We treated these mice with the SHP2 inhibitor RMC-4550 57, AKT1/2 inhibitor capivasertib 58, 59, or a combination of the two (FIGS. 5 and 22). These drugs were chosen based on their ongoing clinical development and ability to reduce activation RAS and PI3K pathways 57, 59.

Direct fluorescence imaging and histology indicated that SHP2 inhibition and combined SHP2 and AKT1/2 inhibition greatly reduced tumor burden (FIGS. 5 and 22). Tuba-seq analysis provided greater insights into the overall and genotype-specific responses of tumors to the therapeutic interventions. Capivasertib monotherapy was ineffective in vivo while RMC-4550 reduced the total tumor burden. The combination of RMC-4550 and capivasertib trended towards being the most efficient therapeutic approach reducing tumor burden by ˜30% compared with RMC-4550 alone (FIGS. 5, 22).

We confirmed the inhibition of RAS and PI3K pathways in oncogene-negative RAS/PI3K tumors in mice treated with RMC-4550 and capivasertib by immunohistochemistry (FIG. 22). Furthermore, global gene expression analysis confirmed the downregulation of RAS and PI3K-AKT gene expression signatures after coincident SHP2 and AKT1/2 inhibition (FIG. 23). Treated tumors tended to have higher expression of an apoptosis gene expression signature and lower expression of a G2/M gene expression signature, suggesting that this combination treatment induces broad cellular changes in oncogene-negative tumors (FIG. 23).

### Inhibition of SHP2 and AKT Synergizes to Reduce the Growth of Onc-Negative RAS/PI3K Lung Adenocarcinoma Cell Lines

To more extensively characterize the responses to SHP2 and AKT inhibition, we generated Nf1/Rasa1/Pten deficient Onc-negative RAS/PI3K cell lines from tumors initiated with Lenti-sgNf1-sgRasa1-sgPten/Cre in Trp53flox/flox; TC mice (FIG. 24). As anticipated, RAS and PI3K signaling was reduced in response to treatment with RMC-4550 and capivasertib, respectively (FIG. 24). RMC-4550 and capivasertib each decreased the overall growth of three oncogene-negative RAS/PI3K cell lines in a dose-dependent manner (FIGS. 5 and 24). Consistent with our in vivo observations, RMC-4550 and capivasertib synergized to inhibit the growth of these cell lines (FIG. 5, and 24). RAS and PI3K signaling can promote cell growth and survival [58, 59], and RMC-4550 and capivasertib inhibited proliferation and induced apoptosis to a greater extent than either RMC-4550 or capivasertib alone (FIG. 5).

Building on these findings, we assessed activation of RAS and PI3K pathways and driver pathway vulnerabilities in two oncogene-negative human lung adenocarcinoma cell lines, NCI H1838 (NF1LOF) and NCI-H1623 (RASA1LOF). H1838 and H1623 had activation of RAS and PI3K pathways (FIG. 24). Consistent with our findings in mouse Onc-negative RAS/PI3K cell lines, RMC-4550 synergizes with capivasertib to inhibit the growth of these human One negative RAS/PI3K lung adenocarcinoma cell lines (FIGS. 5 and 24). These in vivo and cell culture analyses indicate that Onc-negative RAS/PI3K tumors are vulnerable to therapeutic inhibition of these pathways.

### Discussion

It is often overlooked that lung adenocarcinomas without genomic alterations in oncogenes afflict as many patients as those driven by either oncogenic KRAS or EGFR. To identify whether combinatorial inactivation of multiple tumor suppressor genes drives the initiation and growth of lung adenocarcinoma in the absence of oncogene activation, we performed a series of multiplexed in vivo functional genomic screens. By querying an extensive set of tumor suppressor gene alterations, we uncovered combinatorial tumor suppressor inactivation as a key driver of oncogene-negative lung adenocarcinomas. Importantly, combinatorial inactivation of negative regulators of RAS and PI3K pathways are as potent as oncogenic KRASG12D in initiating lung tumors in vivo.

Furthermore, while NF1 inactivation is sometimes suggested to be an “oncogenic driver” in lung adenocarcinoma 4, 29, 60, Nf1 inactivation alone is insufficient to initiate lung tumors (FIG. S8). Even pairwise inactivation of Nf1 and Rasa1, as well as many other tumor suppressor genes, generated very few tumors even after long time periods (FIG. S8). These data suggest that genomic and/or epigenetic alterations in multiple genes within and across pathways may be required to surpass the thresholds necessary for Onc-negative RAS/PI3K lung adenocarcinoma initiation and growth.

Although cancers harbor diverse genomic and epigenomic alterations, these alterations often converge on key pathways and generate similar biochemical changes 15, 61. For example, myeloid leukemia can be driven by gain-of-function mutations in KRAS, NRAS, or the receptor tyrosine kinase FL7T3, or combined inactivation of multiple negative regulators of RAS pathway such as SPRY4 and NF1 62, 63. Pathway activation through genomic and epigenomic inactivation of tumor suppressors can be very diverse, precluding the identification of non-oncogene drivers from gene-centric analysis of human cancer genomic data. Notably, our pathway analysis in oncogene-negative lung adenocarcinomas indicated that mutations in different genes that converge on the RAS and PI3K pathways frequently co-occur (FIGS. 4i and S14i). Furthermore, previous reports and our observations suggest frequent non-genomic mechanisms of downregulation of RAS GAPs and PTEN (FIG. 4f-h, S14a-f) 4, 22-24, 53, 54. Thus, genomic alterations should be viewed as a floor, not a ceiling, in estimating the frequency of pathway alteration.

We assessed the ability of hundreds of complex tumor suppressor genotypes to generate lung tumors. While activation of RAS and PI3K pathway emerged as the most potent driver of oncogene-negative lung adenocarcinomas, our data also suggest that combinatorial inactivation of tumor suppressor genes outside these two pathways can likely initiate tumorigenesis (FIGS. 2 and S6). Given the mutational diversity and complexity of oncogene-negative human lung 388 adenocarcinomas 64, there remain many other mutational combinations to be investigated. We anticipate that additional studies will uncover other oncogene-negative tumor subtypes beyond Onc-negative RAS/PI3K lung adenocarcinomas.

Knowledge of the genes underlying human cancer is a pillar of cancer diagnostics, personalized medicine, and the selection of rational combination therapies. Additionally, our data demonstrate RAS and PI3K pathway activation in the absence of oncogene mutations in a sizable fraction of human lung adenocarcinoma that could predict therapeutic vulnerability to SHP2 and AKT inhibitors. Thus, biochemical assessment of oncogenic pathways in tumors is a strong foundation for rational selection of therapies and clinical trial designs. Beyond SHP2 and AKT, extensive efforts have generated inhibitors for many other components of the RAS and PI3K pathways.

Our findings uncover tumorigenic mechanisms and clinical features of oncogene-negative lung adenocarcinomas. This work identifies biomarkers and new therapeutic targets for Onc-negative RAS/PI3K tumors. The generation of comprehensive molecular and pharmacogenomic maps of oncogene-negative lung adenocarcinomas will transform our understanding of these heretofore poorly characterized lung cancer subtypes

### Methods

### Essentially Identical as Described for Example 1

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of treatment, comprising:
administering a composition comprising an inhibitor of Ras/MAPK pathway activity to an individual who has a cancer previously determined to be oncogene-negative.

2. The method of claim 1, wherein the inhibitor of Ras/MAPK pathway activity comprises an inhibitor of SHP2.

3. The method of claim 2, wherein the inhibitor of SHP2 is RMC-4550 or RMC-4630.

4. The method of any one of claims 1-3, further comprising administering an inhibitor of PI3K-AKT pathway activity to the individual.

5. The method of claim 4, wherein said administering of the inhibitor of PI3K-AKT pathway activity comprises local administration to a tumor of the individual.

6. The method of claim 4 or claim 5, wherein the inhibitor of PI3K-AKT pathway activity comprises an inhibitor of AKT1/2.

7. The method of claim 6, wherein the inhibitor of AKT1/2 is capivasertib.

8. The method of any one of claims 1-7, wherein the oncogene-negative cancer is a lung cancer.

9. The method of claim 8, wherein the lung cancer is lung adenocarcinoma.

10. The method of any one of claim 1-9, wherein said cancer was previously determined to be oncogene-negative by a method comprising genome sequencing.

11. The method of any one of claim 1-9, comprising a step of providing said previous oncogene-negative determination by assaying a biological sample of said cancer.

12. The method of claim 11, wherein said assaying comprises genome sequencing.

13. The method of any one of claims 1-12, wherein, prior to said administering, said cancer of the individual was previously determined to exhibit increased: (i) Ras/MAPK pathway activity or (ii) Ras/MAPK pathway activity and PI3K-AKT pathway activity, compared to a control value.

14. The method of claim 13, wherein said method comprises a step of providing said previous determination of increased pathway activity by assaying a biological sample of said cancer.

15. The method of claim 14, wherein said assaying comprises genome sequencing and/or biomarker analysis.

16. The method of claim 15, wherein said biomarker analysis comprises measuring levels of:
(i) phosphorylated ERK (pERK) or (ii) pERK and phosphorylated AKT (pAKT).

17. The method of claim 16, wherein said measuring comprises immunohistochemistry.

18. A composition formulated for use in a method of treating an individual who has an oncogene-negative cancer, wherein the composition comprises an inhibitor of Ras/MAPK pathway activity.

19. The composition of claim 18, wherein the inhibitor of Ras/MAPK pathway activity comprises an inhibitor of SHP2.

20. The composition of claim 19, wherein the inhibitor of SHP2 is RMC-4550 or RMC-4630.

21. The composition of any one of claims 18-20, further comprising an inhibitor of PI3K-AKT pathway activity.

22. The composition of claim 21, wherein the inhibitor of PI3K-AKT pathway activity comprises an inhibitor of AKT1/2.

23. The composition of claim 22, wherein the inhibitor of AKT1/2 is capivasertib.

24. The composition of any one of claims 18-23, wherein the oncogene-negative cancer is a lung cancer.

25. The composition of claim 24, wherein the lung cancer is lung adenocarcinoma.

26. The composition of any one of claim 18-25, wherein the individual exhibits increased: (i) Ras/MAPK pathway activity or (ii) Ras/MAPK pathway activity and PI3K-AKT pathway activity, compared to a control value.

27. A kit for use in a method of treating an individual who has an oncogene-negative cancer, the kit comprising: (a) an inhibitor of Ras/MAPK pathway activity; and (b) an inhibitor of PI3K-AKT pathway activity, wherein the components of the kit are separated from one another.

28. The kit of claim 27, wherein the inhibitor of Ras/MAPK pathway activity comprises an inhibitor of SHP2.

29. The kit of claim 28, wherein the inhibitor of SHP2 is RMC-4550 or RMC-4630.

30. The kit of any one of claims 27-29, wherein the inhibitor of PI3K-AKT pathway activity comprises an inhibitor of AKT1/2.

31. The kit of claim 30, wherein the inhibitor of AKT1/2 is capivasertib.

32. The kit of any one of claims 27-31, wherein the oncogene-negative cancer is a lung cancer.

33. The kit of claim 32, wherein the lung cancer is lung adenocarcinoma.

34. The kit of any one of claim 17-33, wherein the individual exhibits increased: (i) Ras/MAPK pathway activity or (ii) Ras/MAPK pathway activity and PI3K-AKT pathway activity, compared to a control value.

35. A method for testing candidate therapies, the method comprising:
(a) administering a candidate therapeutic agent to a non-human genetically modified mammal that has an oncogene-negative genomic profile and comprises lung cells with one or more genomic alterations causing increased Ras/MAPK pathway activity and/or increased PI3K-AKT pathway activity; and
(b) determining whether the candidate therapeutic agent prevented or reduced lung cancer in the individual relative to a control.

36. The method of claim 35, wherein said one or more genomic alterations cause increased Ras/MAPK pathway activity and increased PI3K-AKT pathway activity.

37. The method of claim 35 or claim 36, wherein the increased Ras/MAPK pathway activity results from reduced expression of wild type Nf1 and/or wild type Rasa1.

38. The method of any one of claims 35-37, wherein the increased PI3K-AKT pathway activity results from reduced expression of wild type Pten.

39. The method of any one of claims 35-37, wherein the increased PI3K-AKT pathway activity results from a pathway-activating alteration of AKT.

40. The method of any one of claims 35-39, wherein the candidate therapeutic agent is administered locally to a tumor.

41. The method of any one of claims 35-39, wherein the candidate therapeutic agent is administered systemically to the individual.

42. The method of any one of claims 35-41, wherein said control is a predetermined threshold value.

43. The method of any one of claims 35-41, wherein said control is a control tumor in the same individual, wherein the control tumor is an untreated tumor or a tumor treated with a control agent.

44. The method of any one of claims 35-41, wherein said control is a cancer in a different individual, wherein said different individual is an untreated control animal or a control animal treated with a control agent.

45. The method of any one of claims 35-44, wherein the non-human genetically modified mammal is a rodent.

46. The method of any one of claims 35-44, wherein the non-human genetically modified mammal is a non-human primate.

47. A method for testing candidate therapies, the method comprising:
(a) contacting a population of cells with a candidate therapeutic agent, wherein said cells are mammalian cells that have an oncogene-negative genomic profile and comprise one or more genomic alterations causing increased Ras/MAPK pathway activity and/or increased PI3K-AKT pathway activity; and
(b) determining whether the candidate therapeutic agent prevented or reduced proliferation of said population of cells relative to a control.

48. The method of claim 47, wherein said cells are from a non-human genetically modified mammal or are progeny of such cells, wherein said non-human genetically modified mammal has an oncogene-negative genomic profile and comprises one or more genomic alterations causing increased Ras/MAPK pathway activity and/or increased PI3K-AKT pathway activity.

49. The method of claim 47 or claim 48, wherein said cells are rodent cells.

50. The method of claim 47 or claim 48, wherein said cells are non-human primate cells.

51. The method of claim 47, wherein said cells are human cells.

52. The method of any one of claims 47-51, wherein said cells are lung cells.

53. The method of any one of claims 47-52, wherein said one or more genomic alterations cause increased Ras/MAPK pathway activity and increased PI3K-AKT pathway activity.

54. The method of any one of claims 47-53, wherein the increased Ras/MAPK pathway activity results from reduced expression of wild type Nf1 and/or wild type Rasa1.

55. The method of any one of claims 47-54, wherein the increased PI3K-AKT pathway activity results from reduced expression of wild type Pten and/or from a pathway-activating alteration of AKT.

56. The method of any one of claims 47-55, wherein said control is a predetermined threshold value.

57. The method of any one of claims 47-55, wherein said control is a control population of cells that are untreated or treated with a control agent.

58. An oncogene-negative mouse for use as a lung cancer model, comprising:
an oncogene-negative genomic profile and genomic alterations causing increased Ras/MAPK pathway activity and increased PI3K-AKT pathway activity.

59. The oncogene-negative mouse of claim 58, wherein the increased Ras/MAPK pathway activity is caused by reduced expression wild type Nf1 and/or wild type Rasa1.

60. The oncogene-negative mouse of claim 58 or claim 59, wherein the increased PI3K-AKT pathway activity is caused by reduced expression wild type Pten.

61. The oncogene-negative mouse of claim 58 or claim 59, wherein the increased PI3K-AKT pathway activity is caused by a pathway-activating alteration of AKT.

62. The oncogene-negative mouse of any one of claims 58-61, wherein the increased Ras/MAPK pathway activity is caused by reduced expression wild type Nf1 and wild type Rasa1, and the increased PI3K-AKT pathway activity is caused by reduced expression wild type Pten.

63. The oncogene-negative mouse of claim 62, wherein the reduced expression of wild type Nf1, Rasa1, and Pten is caused by a genetic loss-of-function mutations in each of the Nf1, Rasa1, and Pten loci.

64. A cell, or progeny thereof, isolated from the oncogene-negative mouse of claim 58.

65. The cell, or progeny thereof, of claim 64, wherein the cell is a germ cell.

66. The cell, or progeny thereof, of claim 64, wherein the cell is a stem cell.

67. The cell, or progeny thereof, of claim 64, wherein the cell is a lung cell.


SEQUENCE LISTING (29 sequences)

SEQ ID NO: 1 - DNA, 29 bp, Artificial sequence
  synthetic sequence
aatgatacggcgaccaccgagatctacac

SEQ ID NO: 2 - DNA, 33 bp, Artificial sequence
  synthetic sequence
acactctttccctacacgacgctcttccgatct

SEQ ID NO: 3 - DNA, 19 bp, Artificial sequence
  synthetic sequence
gcgcacgtctgccgcgctg

SEQ ID NO: 4 - DNA, 24 bp, Artificial sequence
  synthetic sequence
caagcagaagacggcatacgagat

SEQ ID NO: 5 - DNA, 34 bp, Artificial sequence
  synthetic sequence
gtgactggagttcagacgtgtgctcttccgatct

SEQ ID NO: 6 - DNA, 20 bp, Artificial sequence
  synthetic sequence
caggttcttgcgaacctcat

SEQ ID NO: 7 - DNA, 30 bp, Artificial sequence
  synthetic sequence
  misc_feature (3)..(7): n is a, c, g, or t
  misc_feature (10)..(14): n is a, c, g, or t
  misc_feature (17)..(21): n is a, c, g, or t
  misc_feature (24)..(28): n is a, c, g, or t
gcnnnnntannnnngcnnnnntannnnngc

SEQ ID NO: 8 - DNA, 62 bp, Artificial sequence
  synthetic sequence
  misc_feature (34)..(39): n is a, c, g, or t
acactctttccctacacgacgctcttccgatctnnnnnnttatttttattgtaaggcctgct

SEQ ID NO: 9 - DNA, 58 bp, Artificial sequence
  synthetic sequence
  misc_feature (35)..(41): n is a, c, g, or t
gtgactggagttcagacgtgtgctcttccgatctnnnnnnnttacaagcgcacgcaga

SEQ ID NO: 10 - DNA, 64 bp, Artificial sequence
  synthetic sequence
  misc_feature (34)..(41): n is a, c, g, or t
acactctttccctacacgacgctcttccgatctnnnnnnnncctgtctcttggatattctcgac

SEQ ID NO: 11 - DNA, 65 bp, Artificial sequence
  synthetic sequence
  misc_feature (35)..(43): n is a, c, g, or t
gtgactggagttcagacgtgtgctcttccgatctnnnnnnnnncagttctcatgtactggtccct

SEQ ID NO: 12 - DNA, 61 bp, Artificial sequence
  synthetic sequence
  misc_feature (34)..(41): n is a, c, g, or t
acactctttccctacacgacgctcttccgatctnnnnnnnnccagcggagaagctccaaac

SEQ ID NO: 13 - DNA, 65 bp, Artificial sequence
  synthetic sequence
  misc_feature (35)..(43): n is a, c, g, or t
gtgactggagttcagacgtgtgctcttccgatctnnnnnnnnnatacactgtgccaaatgctccc

SEQ ID NO: 14 - DNA, 63 bp, Artificial sequence
  synthetic sequence
  misc_feature (34)..(39): n is a, c, g, or t
acactctttccctacacgacgctcttccgatctnnnnnntcttcttaatctcagggtctctgg

SEQ ID NO: 15 - DNA, 65 bp, Artificial sequence
  synthetic sequence
  misc_feature (35)..(41): n is a, c, g, or t
gtgactggagttcagacgtgtgctcttccgatctnnnnnnncacgtcaaggatttctttgttggc

SEQ ID NO: 16 - DNA, 64 bp, Artificial sequence
  synthetic sequence
  misc_feature (34)..(41): n is a, c, g, or t
acactctttccctacacgacgctcttccgatctnnnnnnnnttacccagaaagggatatgcgtg

SEQ ID NO: 17 - DNA, 62 bp, Artificial sequence
  synthetic sequence
  misc_feature (35)..(43): n is a, c, g, or t
gtgactggagttcagacgtgtgctcttccgatctnnnnnnnnnggcaaccgtagggcatgag

SEQ ID NO: 18 - DNA, 63 bp, Artificial sequence
  synthetic sequence
  misc_feature (34)..(39): n is a, c, g, or t
acactctttccctacacgacgctcttccgatctnnnnnngtgaagacaccacagcatgtcaag

SEQ ID NO: 19 - DNA, 64 bp, Artificial sequence
  synthetic sequence
  misc_feature (35)..(41): n is a, c, g, or t
gtgactggagttcagacgtgtgctcttccgatctnnnnnnngcttcctgatctactcccaggac

SEQ ID NO: 20 - DNA, 63 bp, Artificial sequence
  synthetic sequence
  misc_feature (34)..(41): n is a, c, g, or t
acactctttccctacacgacgctcttccgatctnnnnnnnngactgggagattcctgatggac

SEQ ID NO: 21 - DNA, 67 bp, Artificial sequence
  synthetic sequence
  misc_feature (35)..(43): n is a, c, g, or t
gtgactggagttcagacgtgtgctcttccgatctnnnnnnnnncgtgttatacataccatgtcccac

SEQ ID NO: 22 - DNA, 63 bp, Artificial sequence
  synthetic sequence
  misc_feature (34)..(39): n is a, c, g, or t
acactctttccctacacgacgctcttccgatctnnnnnngacctcacggtaaaaataggtgac

SEQ ID NO: 23 - DNA, 63 bp, Artificial sequence
  synthetic sequence
  misc_feature (35)..(41): n is a, c, g, or t
gtgactggagttcagacgtgtgctcttccgatctnnnnnnnaactgttcaaactgatgggacc

SEQ ID NO: 24 - DNA, 64 bp, Artificial sequence
  synthetic sequence
  misc_feature (34)..(41): n is a, c, g, or t
acactctttccctacacgacgctcttccgatctnnnnnnnnttctacaggtttttgctggtgtg

SEQ ID NO: 25 - DNA, 64 bp, Artificial sequence
  synthetic sequence
  misc_feature (35)..(43): n is a, c, g, or t
gtgactggagttcagacgtgtgctcttccgatctnnnnnnnnngattagctggatcgtcaaggc

SEQ ID NO: 26 - DNA, 62 bp, Artificial sequence
  synthetic sequence
  misc_feature (34)..(39): n is a, c, g, or t
acactctttccctacacgacgctcttccgatctnnnnnncgaaagcaagtggtgattgatgg

SEQ ID NO: 27 - DNA, 63 bp, Artificial sequence
  synthetic sequence
  misc_feature (35)..(41): n is a, c, g, or t
gtgactggagttcagacgtgtgctcttccgatctnnnnnnnaaatacacagaggaacccttcg

SEQ ID NO: 28 - DNA, 57 bp, Artificial sequence
  synthetic sequence
  misc_feature (30)..(37): n is a, c, g, or t
aatgatacggcgaccaccgagatctacacnnnnnnnnacactctttccctacacgac

SEQ ID NO: 29 - DNA, 50 bp, Artificial sequence
  synthetic sequence
  misc_feature (25)..(30): n is a, c, g, or t
caagcagaagacggcatacgagatnnnnnngtgactggagttcagacgtg